SEQUENCE LISTING (448 sequences)

SEQ ID NO: 1 - DNA, 1131 bp, Homo sapiens
atggagtcctacgatgtgatcgccaaccagcctgtcgtgatcgacaacggatccggtgtgattaaagctggttttgctggtgatcagatccccaaatactgctttccaaactatgtgggccgacccaagcacgttcgtgtcatggcaggagcccttgaaggcgacatcttcattggccccaaagctgaggagcaccgagggctgctttcaatccgctatcccatggagcatggcatcgtcaaggattggaacgacatggaacgcatttggcaatatgtctattctaaggaccagctgcagactttctcagaggagcatcctgtgctcctgactgaggcgcctttaaacccacgaaaaaaccgggaacgagctgccgaagttttcttcgagaccttcaatgtgcccgctcttttcatctccatgcaagctgtactcagcctttacgctacaggcaggaccacaggggtggtgctggattctggggatggagtcacccatgctgtgcccatctatgagggctttgccatgccccactccatcatgcgcatcgacatcgcgggccgggacgtctctcgcttcctgcgcctctacctgcgtaaggagggctacgacttccactcatcctctgagtttgagattgtcaaggccataaaagaaagagcctgttacctatccataaacccccaaaaggatgagacgctagagacagagaaagctcagtactacctgcctgatggcagcaccattgagattggtccttcccgattccgggcccctgagttgctcttcaggccagatttgattggagaggagagtgaaggcatccacgaggtcctggtgttcgccattcagaagtcagacatggacctgcggcgcacgcttttctctaacattgtcctctcaggaggctctaccctgttcaaaggttttggtgacaggctcctgagtgaagtgaagaaactagctccaaaagatgtgaagatcaggatatctgcacctcaggagagactgtattccacgtggattgggggctccatccttgcctccctggacacctttaagaagatgtgggtctccaaaaaggaatatgaggaagacggtgcccgatccatccacagaaaaaccttctaa

SEQ ID NO: 2 - DNA, 1131 bp, Homo sapiens
atggagtcctacgatgtgatcgccaaccagcctgtcgtgatcgacaacggatccggtgtgattaaagctggttttgctggtgatcagatccccaaatactgctttccaaactatgtgggccgacccaagcacgttcgtgtcatggcaggagcccttgaaggcgacatcttcattggccccaaagctgaggagcaccgagggctgctttcaatccgctatcccatggagcatggcatcgtcaaggattggaacgacatggaacgcatttggcaatatgtctattctaaggaccagctgcagactttctcagaggagcatcctgtgctcctgactgaggcgcctttaaacccacgaagaaaccgggaacgagctgccgaagttttcttcgagaccttcaatgtgcccgctcttttcatctccatgcaagctgtactcagcctttacgctacaggcaggaccacaggggtggtgctggattctggggatggagtcacccatgctgtgcccatctatgagggctttgccatgccccactccatcatgcgcatcgacatcgcgggccgggacgtctctcgcttcctgcgcctctacctgcgtaaggagggctacgacttccactcatcctctgagtttgagattgtcaaggccataaaagaaagagcctgttacctatccataaacccccaaaaggatgagacgctagagacagagaaagctcagtactacctgcctgatggcagcaccattgagattggtccttcccgattccgggcccctgagttgctcttcaggccagatttgattggagaggagagtgaaggcatccacgaggtcctggtgttcgccattcagaagtcagacatggacctgcggcgcacgcttttctctaacattgtcctctcaggaggctctaccctgttcaaaggttttggtgacaggctcctgagtgaagtgaagaaactagctccaaaagatgtgaagatcaggatatctgcacctcaggagagactgtattccacgtggattgggggctccatccttgcctccctggacacctttaagaagatgtgggtctccaaaaaggaatatgaggaagacggtgcccgatccatccacagaaaaaccttctaa

SEQ ID NO: 3 - DNA, 8487 bp, Homo sapiens
atgcccaagcgcgcgcactggggggccctctccgtggtgctgatcctgctttggggccatccgcgagtggcgctggcctgcccgcatccttgtgcctgctacgtccccagcgaggtccactgcacgttccgatccctggcttccgtgcccgctggcattgctaaacacgtggaaagaatcaatttggggtttaatagcatacaggccctgtcagaaacctcatttgcaggactgaccaagttggagctacttatgattcacggcaatgagatcccaagcatccccgatggagctttaagagacctcagctctcttcaggttttcaagttcagctacaacaagctgagagtgatcacaggacagaccctccagggtctctctaacttaatgaggctgcacattgaccacaacaagatcgagtttatccaccctcaagctttcaacggcttaacgtctctgaggctactccatttggaaggaaatctcctccaccagctgcaccccagcaccttctccacgttcacatttttggattatttcagactctccaccataaggcacctctacttagcagagaacatggttagaactcttcctgccagcatgcttcggaacatgccgcttctggagaatctttacttgcagggaaatccgtggacctgcgattgtgagatgagatggtttttggaatgggatgcaaaatccagaggaattctgaagtgtaaaaaggacaaagcttatgaaggcggtcagttgtgtgcaatgtgcttcagtccaaagaagttgtacaaacatgagatacacaagctgaaggacatgacttgtctgaagccttcaatagagtcccctctgagacagaacaggagcaggagtattgaggaggagcaagaacaggaagaggatggtggcagccagctcatcctggagaaattccaactgccccagtggagcatctctttgaatatgaccgacgagcacgggaacatggtgaacttggtctgtgacatcaagaaaccaatggatgtgtacaagattcacttgaaccaaacggatcctccagatattgacataaatgcaacagttgccttggactttgagtgtccaatgacccgagaaaactatgaaaagctatggaaattgatagcatactacagtgaagttcccgtgaagctacacagagagctcatgctcagcaaagaccccagagtcagctaccagtacaggcaggatgctgatgaggaagctctttactacacaggtgtgagagcccagattcttgcagaaccagaatgggtcatgcagccatccatagatatccagctgaaccgacgtcagagtacggccaagaaggtgctactttcctactacacccagtattctcaaacaatatccaccaaagatacaaggcaggctcggggcagaagctgggtaatgattgagcctagtggagctgtgcaaagagatcagactgtcctggaagggggtccatgccagttgagctgcaacgtgaaagcttctgagagtccatctatcttctgggtgcttccagatggctccatcctgaaagcgcccatggatgacccagacagcaagttctccattctcagcagtggctggctgaggatcaagtccatggagccatctgactcaggcttgtaccagtgcattgctcaagtgagggatgaaatggaccgcatggtatatagggtacttgtgcagtctccctccactcagccagccgagaaagacacagtgacaattggcaagaacccaggggagtcggtgacattgccttgcaatgctttagcaatacccgaagcccaccttagctggattcttccaaacagaaggataattaatgatttggctaacacatcacatgtatacatgttgccaaatggaactctttccatcccaaaggtccaagtcagtgacagtggttactacagatgtgtggctgtcaaccagcaaggggcagaccattttacggtgggaatcacagtgaccaagaaagggtctggcttgccatccaaaagaggcagacgcccaggtgcaaaggctctttccagagtcagagaagacatcgtggaggatgaagggggctcgggcatgggagatgaagagaacacttcaaggagacttctgcatccaaaggaccaagaggtgttcctcaaaacaaaggatgatgccatcaatggagacaagaaagccaagaaagggagaagaaagctgaaactctggaagcattcggaaaaagaaccagagaccaatgttgcagaaggtcgcagagtgtttgaatctagacgaaggataaacatggcaaacaaacagattaatccggagcgctgggctgatattttagccaaagtccgtgggaaaaatctccctaagggcacagaagtacccccattgattaaaaccacaagtcctccatccttgagtctagaagtcacaccaccttttcctgctatttctcccccctcagcatctcctgtgcagacagtaaccagtgctgaagaatcctcagcagatgtacctctacttggtgaagaagagcacgttttgggtaccatttcctcagccagcatggggctagaacacaaccacaatggagttattcttgttgaacctgaagtaacaagcacacctctggaggaagttgttgatgacctttccgagaagactgaggagataacttccactgaaggagacctgaaggggacagcagcccctacacttatatctgagccttatgaaccatctcctactctgcacacattagacacagtctatgaaaagcccacccatgaagagacggcaacagagggttggtctgcagcagatgttggatcgtcaccagagcccacatccagtgagtatgagcctccattggatgctgtctccttggctgagtctgagcccatgcaatactttgacccagatttggagactaagtcacaaccagatgaggataagatgaaagaagacacctttgcacaccttactccaacccccaccatctgggttaatgactccagtacatcacagttatttgaggattctactataggggaaccaggtgtcccaggccaatcacatctacaaggactgacagacaacatccaccttgtgaaaagtagtctaagcactcaagacaccttactgattaaaaagggtatgaaagagatgtctcagacactacagggaggaaatatgctagagggagaccccacacactccagaagttctgagagtgagggccaagagagcaaatccatcactttgcctgactccacactgggtataatgagcagtatgtctccagttaagaagcctgcggaaaccacagttggtaccctcctagacaaagacaccacaacagcaacaacaacaccaaggcaaaaagttgctccgtcatccaccatgagcactcacccttctcgaaggagacccaacgggagaaggagattacgccccaacaaattccgccaccggcacaagcaaaccccacccacaacttttgccccatcagagactttttctactcaaccaactcaagcacctgacattaagatttcaagtcaagtggagagttctctggttcctacagcttgggtggataacacagttaatacccccaaacagttggaaatggagaagaatgcagaacccacatccaagggaacaccacggagaaaacacgggaagaggccaaacaaacatcgatataccccttctacagtgagctcaagagcgtccggatccaagcccagcccttctccagaaaataaacatagaaacattgttactcccagttcagaaactatacttttgcctagaactgtttctctgaaaactgagggcccttatgattccttagattacatgacaaccaccagaaaaatatattcatcttaccctaaagtccaagagacacttccagtcacatataaacccacatcagatggaaaagaaattaaggatgatgttgccacaaatgttgacaaacataaaagtgacattttagtcactggtgaatcaattactaatgccataccaacttctcgctccttggtctccactatgggagaatttaaggaagaatcctctcctgtaggctttccaggaactccaacctggaatccctcaaggacggcccagcctgggaggctacagacaggcatacctgttaccacttctggggaaaatcttacagaccctccccttcttaaagagcttgaggatgtggatttcacttccgagtttttgtcctctttgacagtctccacaccatttcaccaggaagaagctggttcttccacaactctctcaagcataaaagtggaggtggcttcaagtcaggcagaaaccaccacccttgatcaagatcatcttgaaaccactgtggctattctcctttctgaaactagaccacagaatcacacccctactgctgcccggatgaaggagccagcatcctcgtccccatccacaattctcatgtctttgggacaaaccaccaccactaagccagcacttcccagtccaagaatatctcaagcatctagagattccaaggaaaatgttttcttgaattatgtggggaatccagaaacagaagcaaccccagtgaacaatgaaggaacacagcatatgtcagggccaaatgaattatcaacaccctcttccgaccaggatgcatttaacttgtctacaaagctggaattggaaaagcaagtatttggtagtaggagtctaccacgtggcccagatagccaacgccaggatggaagagttcatgcttctcatcaactaaccagagtccctgccaaacccatcctaccaacagcaacagtgaggctgcctgaaatgtccacacaaagcgcttccagatactttgtaacttcccagtcacctcgtcactggaccaacaaaccggaaataactacatatccttctggggctttgccagagaacaaacagtttacaactccaagattatcaagtacaacaattcctctcccattgcacatgtccaaacccagcattcctagtaagtttactgaccgaagaactgaccaattcaatggctactccaaagtgtttggaaataacaacatccctgaggcaagaaacccagttggaaagcctcccagtccaagaattcctcattattccaatggaagactccctttctttaccaacaagactctttcttttccacagttgggagtcacccggagaccccagatacccacttctcctgccccagtaatgagagagagaaaagttattccaggttcctacaacaggatacattcccatagcaccttccatctggactttggccctccggcacctccgttgttgcacactccgcagaccacgggatcaccctcaactaacttacagaatatccctatggtctcttccacccagagttctatctcctttataacatcttctgtccagtcctcaggaagcttccaccagagcagctcaaagttctttgcaggaggacctcctgcatccaaattctggtctcttggggaaaagccacaaatcctcaccaagtccccacagactgtgtccgtcaccgctgagacagacactgtgttcccctgtgaggcaacaggaaaaccaaagcctttcgttacttggacaaaggtttccacaggagctcttatgactccgaataccaggatacaacggtttgaggttctcaagaacggtaccttagtgatacggaaggttcaagtacaagatcgaggccagtatatgtgcaccgccagcaacctgcacggcctggacaggatggtggtcttgctttcggtcaccgtgcagcaacctcaaatcctagcctcccactaccaggacgtcactgtctacctgggagacaccattgcaatggagtgtctggccaaagggaccccagccccccaaatttcctggatcttccctgacaggagggtgtggcaaactgtgtcccccgtggagggccgcatcaccctgcacgaaaaccggaccctttccatcaaggaggcgtccttctcagacagaggcgtctataagtgcgtggccagcaatgcagccggggcggacagcctggccatccgcctgcacgtggcggcactgccccccgttatccaccaggagaagctggagaacatctcgctgcccccggggctcagcattcacattcactgcactgccaaggctgcgcccctgcccagcgtgcgctgggtgctcggggacggtacccagatccgcccctcgcagttcctccacgggaacttgtttgttttccccaacgggacgctctacatccgcaacctcgcgcccaaggacagcgggcgctatgagtgcgtggccgccaacctggtaggctccgcgcgcaggacggtgcagctgaacgtgcagcgtgcagcagccaacgcgcgcatcacgggcacctccccgcggaggacggacgtcaggtacggaggaaccctcaagctggactgcagcgcctcgggggacccctggccgcgcatcctctggaggctgccgtccaagaggatgatcgacgcgctcttcagttttgatagcagaatcaaggtgtttgccaatgggaccctggtggtgaaatcagtgacggacaaagatgccggagattacctgtgcgtagctcgaaataaggttggtgatgactacgtggtgctcaaagtggatgtggtgatgaaaccggccaagattgaacacaaggaggagaacgaccacaaagtcttctacgggggtgacctgaaagtggactgtgtggccaccgggcttcccaatcccgagatctcctggagcctcccagacgggagtctggtgaactccttcatgcagtcggatgacagcggtggacgcaccaagcgctatgtcgtcttcaacaatgggacactctactttaacgaagtggggatgagggaggaaggagactacacctgctttgctgaaaatcaggtcgggaaggacgagatgagagtcagagtcaaggtggtgacagcgcccgccaccatccggaacaagacttacttggcggttcaggtgccctatggagacgtggtcactgtagcctgtgaggccaaaggagaacccatgcccaaggtgacttggttgtccccaaccaacaaggtgatccccacctcctctgagaagtatcagatataccaagatggcactctccttattcagaaagcccagcgttctgacagcggcaactacacctgcttggtcaggaacagcgcgggagaggataggaagacggtgtggattcacgtcaacgtccagccacccaagatcaacggtaaccccaaccccatcaccactgtgcgggagatagcagccgggggcagtcggaaactgattgactgcaaagctgaaggcatccccaccccgagggtgttatgggcttttcccgagggtgtggttctgccagctccatactatggaaaccggatcactgtccatggcaacggttccctggacatcaggagtttgaggaagagcgactccgtccagctggtatgcatggcacgcaacgagggaggggaggccaggttgatcctgcagctcactgtcctggagcccatggagaaacccatcttccacgacccgatcagcgagaagatcacggccatggcgggccacaccatcagcctcaactgctctgccgcggggaccccgacacccagcctggtgtgggtccttcccaatggcaccgatctgcagagtggacagcagctgcagcgcttctaccacaaggctgacggcatgctacacattagcggtctctcctcggtggacgccggggcctaccgctgcgtggcccgcaatgccgctggccacacggagaggctggtctccctgaaggtgggactgaagccagaagcaaacaagcagtatcataacctggtcagcatcatcaatggtgagaccctgaagctcccctgcacccctcccggggctgggcagggacgtttctcctggacgctccccaatggcatgcatctggagggcccccaaaccctgggacgcgtttctcttctggacaatggcaccctcacggttcgtgaggcctcggtgtttgacaggggtacctatgtatgcaggatggagacggagtacggcccttcggtcaccagcatccccgtgattgtgatcgcctatcctccccggatcaccagcgagcccaccccggtcatctacacccggcccgggaacaccgtgaaactgaactgcatggctatggggattcccaaagctgacatcacgtgggagttaccggataagtcgcatctgaaggcaggggttcaggctcgtctgtatggaaacagatttcttcacccccagggatcactgaccatccagcatgccacacagagagatgccggcttctacaagtgcatggcaaaaaacattctcggcagtgactccaaaacaacttacatccacgtcttctga

SEQ ID NO: 4 - DNA, 8487 bp, Homo sapiens
atgcccaagcgcgcgcactggggggccctctccgtggtgctgatcctgctttggggccatccgcgagtggcgctggcctgcccgcatccttgtgcctgctacgtccccagcgaggtccactgcacgttccgatccctggcttccgtgcccgctggcattgctaaacacgtggaaagaatcaatttggggtttaatagcatacaggccctgtcagaaacctcatttgcaggactgaccaagttggagctacttatgattcacggcaatgagatcccaagcatccccgatggagctttaagagacctcagctctcttcaggttttcaagttcagctacaacaagctgagagtgatcacaggacagaccctccagggtctctctaacttaatgaggctgcacattgaccacaacaagatcgagtttatccaccctcaagctttcaacggcttaacgtctctgaggctactccatttggaaggaaatctcctccaccagctgcaccccagcaccttctccacgttcacatttttggattatttcagactctccaccataaggcacctctacttagcagagaacatggttagaactcttcctgccagcatgcttcggaacatgccgcttctggagaatctttacttgcagggaaatccgtggacctgcgattgtgagatgagatggtttttggaatgggatgcaaaatccagaggaattctgaagtgtaaaaaggacaaagcttatgaaggcggtcagttgtgtgcaatgtgcttcagtccaaagaagttgtacaaacatgagatacacaagctgaaggacatgacttgtctgaagccttcaatagagtcccctctgagacagaacaggagcaggagtattgaggaggagcaagaacaggaagaggatggtggcagccagctcatcctggagaaattccaactgccccagtggagcatctctttgaatatgaccgacgagcacgggaacatggtgaacttggtctgtgacatcaagaaaccaatggatgtgtacaagattcacttgaaccaaacggatcctccagatattgacataaatgcaacagttgccttggactttgagtgtccaatgacccgagaaaactatgaaaagctatggaaattgatagcatactacagtgaagttcccgtgaagctacacagagagctcatgctcagcaaagaccccagagtcagctaccagtacaggcaggatgctgatgaggaagctctttactacacaggtgtgagagcccagattcttgcagaaccagaatgggtcatgcagccatccatagatatccagctgaaccgacgtcagagtacggccaagaaggtgctactttcctactacacccagtattctcaaacaatatccaccaaagatacaaggcaggctcggggcagaagctgggtaatgattgagcctagtggagctgtgcaaagagatcagactgtcctggaagggggtccatgccagttgagctgcaacgtgaaagcttctgagagtccatctatcttctgggtgcttccagatggctccatcctgaaagcgcccatggatgacccagacagcaagttctccattctcagcagtggctggctgaggatcaagtccatggagccatctgactcaggcttgtaccagtgcattgctcaagtgagggatgaaatggaccgcatggtatatagggtacttgtgcagtctccctccactcagccagccgagaaagacacagtgacaattggcaagaacccaggggagtcggtgacattgccttgcaatgctttagcaatacccgaagcccaccttagctggattcttccaaacagaaggataattaatgatttggctaacacatcacatgtatacatgttgccaaatggaactctttccatcccaaaggtccaagtcagtgacagtggttactacagatgtgtggctgtcaaccagcaaggggcagaccattttacggtgggaatcacagtgaccaagaaagggtctggcttgccatccaaaagaggcagacgcccaggtgcaaaggctctttccagagtcagagaagacatcgtggaggatgaagggggctcgggcatgggagatgaagagaacacttcaaggagacttctgcatccaaaggaccaagaggtgttcctcaaaacaaaggatgatgccatcaatggagacaagaaagccaagaaagggagaagaaagctgaaactctggaagcattcggaaaaagaaccagagaccaatgttgcagaaggtcgcagagtgtttgaatctagacgaaggataaacatggcaaacaaacagattaatccggagcgctgggctgatattttagccaaagtccgtgggaaaaatctccctaagggcacagaagtacccccattgattaaaaccacaagtcctccatccttgagtctagaagtcacaccaccttttcctgctatttctcccccctcagcatctcctgtgcagacagtaaccagtgctgaagaatcctcagcagatgtacctctacttggtgaagaagagcacgttttgggtaccatttcctcagccagcatggggctagaacacaaccacaatggagttattcttgttgaacctgaagtaacaagcacacctctggaggaagttgttgatgacctttccgagaagactgaggagataacttccactgaaggagacctgaaggggacagcagcccctacacttatatctgagccttatgaaccatctcctactctgcacacattagacacagtctatgaaaagcccacccatgaagagacggcaacagagggttggtctgcagcagatgttggatcgtcaccagagcccacatccagtgagtatgagcctccattggatgctgtctccttggctgagtctgagcccatgcaatactttgacccagatttggagactaagtcacaaccagatgaggataagatgaaagaagacacctttgcacaccttactccaacccccaccatctgggttaatgactccagtacatcacagttatttgaggattctactataggggaaccaggtgtcccaggccaatcacatctacaaggactgacagacaacatccaccttgtgaaaagtagtctaagcactcaagacaccttactgattaaaaagggtatgaaagagatgtctcagacactacagggaggaaatatgctagagggagaccccacacactccagaagttctgagagtgagggccaagagagcaaatccatcactttgcctgactccacactgggtataatgagcagtatgtctccagttaagaagcctgcggaaaccacagttggtaccctcctagacaaagacaccacaacagcaacaacaacaccaaggcaaaaagttgctccgtcatccaccatgagcactcacccttctcgaaggagacccaacgggagaaggagattacgccccaacaaattccgccaccggcacaagcaaaccccacccacaacttttgccccatcagagactttttctactcaaccaactcaagcacctgacattaagatttcaagtcaagtggagagttctctggttcctacagcttgggtggataacacagttaatacccccaaacagttggaaatggagaagaatgcagaacccacatccaagggaacaccacggagaaaacacgggaagaggccaaacaaacatcgatataccccttctacagtgagctcaagagcgtccggatccaagcccagcccttctccagaaaataaacatagaaacattgttactcccagttcagaaactatacttttgcctagaactgtttctctgaaaactgagggcccttatgattccttagattacatgacaaccaccagaaaaatatattcatcttaccctaaagtccaagagacacttccagtcacatataaacccacatcagatggaaaagaaattaaggatgatgttgccacaaatgttgacaaacataaaagtgacattttagtcactggtgaatcaattactaatgccataccaacttctcgctccttggtctccactatgggagaatttaaggaagaatcctctcctgtaggctttccaggaactccaacctggaatccctcaaggacggcccagcctgggaggctacagacaggcatacctgttaccacttctggggaaaatcttacagaccctccccttcttaaagagcttgaggatgtggatttcacttccgagtttttgtcctctttgacagtctccacaccatttcaccaggaagaagctggttcttccacaactctctcaagcataaaagtggaggtggcttcaagtcaggcagaaaccaccacccttgatcaagatcatcttgaaaccactgtggctattctcctttctgaaactagaccacagaatcacacccctactgctgcccggatgaaggagccagcatcctcgtccccatccacaattctcatgtctttgggacaaaccaccaccactaagccagcacttcccagtccaagaatatctcaagcatctagagattccaaggaaaatgttttcttgaattatgtggggaatccagaaacagaagcaaccccagtgaacaatgaaggaacacagcatatgtcagggccaaatgaattatcaacaccctcttccgaccaggatgcatttaacttgtctacaaagctggaattggaaaagcaagtatttggtagtaggagtctaccacgtggcccagatagccaacgccaggatggaagagttcatgcttctcatcaactaaccagagtccctgccaaacccatcctaccaacagcaacagtgaggctgcctgaaatgtccacacaaagcgcttccagatactttgtaacttcccagtcacctcgtcactggaccaacaaaccggaaataactacatatccttctggggctttgccagagaacaaacagtttacaactccaagattatcaagtacaacaattcctctcccattgcacatgtccaaacccagcattcctagtaagtttactgaccgaagaactgaccaattcaatggctactccaaagtgtttggaaataacaacatccctgaggcaagaaacccagttggaaagcctcccagtccaagaattcctcattattccaatggaagactccctttctttaccaacaagactctttcttttccacagttgggagtcacccggagaccccagatacccacttctcctgccccagtaatgagagagagaaaagttattccaggttcctacaacaggatacattcccatagcaccttccatctggactttggccctccggcacctccgttgttgcacactccgcagaccacgggatcaccctcaactaacttacagaatatccctatggtctcttccacccagagttctatctcctttataacatcttctgtccagtcctcaggaagcttccaccagagcagctcaaagttctttgcaggaggacctcctgcatccaaattctggtctcttggggaaaagccacaaatcctcaccaagtccccacagactgtgtccgtcaccgctgagacagacactgtgttcccctgtgaggcaacaggaaaaccaaagcctttcgttacttggacaaaggtttccacaggagctcttatgactccgaataccaggatacaacggtttgaggttctcaagaacggtaccttagtgatacggaaggttcaagtacaagatcgaggccagtatatgtgcaccgccagcaacctgcacggcctggacaggatggtggtcttgctttcggtcaccgtgcagcaacctcaaatcctagcctcccactaccaggacgtcactgtctacctgggagacaccattgcaatggagtgtctggccaaagggaccccagccccccaaatttcctggatcttccctgacaggagggtgtggcaaactgtgtcccccgtggagggccgcatcaccctgcacgaaaaccggaccctttccatcaaggaggcgtccttctcagacagaggcgtctataagtgcgtggccagcaatgcagccggggcggacagcctggccatccgcctgcacgtggcggcactgccccccgttatccaccaggagaagctggagaacatctcgctgcccccggggctcagcattcacattcactgcactgccaaggctgcgcccctgcccagcgtgcgctgggtgctcggggacggtacccagatccgcccctcgcagttcctccacgggaacttgtttgttttccccaacgggacgctctacatccgcaacctcgcgcccaaggacagcgggcgctatgagtgcgtggccgccaacctggtaggctccgcgcgcaggacggtgcagctgaacgtgcagcgtgcagcagccaacgcgcgcatcacgggcacctccccgcggaggacggacgtcaggtacggaggaaccctcaagctggactgcagcgcctcgggggacccctggccgcgcatcctctggaggctgccgtccaagaggatgatcgacgcgctcttcagttttgatagcagaatcaaggtgtttgccaatgggaccctggtggtgaaatcagtgacggacaaagatgccggagattacctgtgcgtagctcgaaataaggttggtgatgactacgtggtgctcaaagtggatgtggtgatgaaaccggccaagattgaacacaaggaggagaacgaccacaaagtcttctacgggggtgacctgaaagtggactgtgtggccaccgggcttcccaatcccgagatctcctggagcctcccagacgggagtctggtgaactccttcatgcagtcggatgacagcggtggacgcaccaagcgctatgtcgtcttcaacaatgggacactctactttaacgaagtggggatgagggaggaaggagactacacctgctttgctgaaaatcaggtcgggaaggacgagatgagagtcagagtcaaggtggtgacagcgcccgccaccatccggaacaagacttacttggcggttcaggtgccctatggagacgtggtcactgtagcctgtgaggccaaaggagaacccatgcccaaggtgacttggttgtccccaaccaacaaggtgatccccacctcctctgagaagtatcagatataccaagatggcactctccttattcagaaagcccagcgttctgacagcggcaactacacctgcttggtcaggaacagcgcgggagaggataggaagacggtgtggattcacgtcaacgtccagccacccaagatcaacggtaaccccaaccccatcaccactgtgcgggagatagcagccgggggcagtcggaaactgattgactgcaaagctgaaggcatccccaccccgagggtgttatgggcttttcccgagggtgtggttctgccagctccatactatggaaaccggatcactgtccatggcaacggttccctggacatcaggagtttgaggaagagcgactccgtccagctggtatgcatggcacgcaacgagggaggggaggccaggttgatcctgcagctcactgtcctggagcccatggagaaacccatcttccacgacccgatcagcgagaagatcacggccatggcgggccacaccatcagcctcaactgctctgccgcggggaccacgacacccagcctggtgtgggtccttcccaatggcaccgatctgcagagtggacagcagctgcagcgcttctaccacaaggctgacggcatgctacacattagcggtctctcctcggtggacgccggggcctaccgctgcgtggcccgcaatgccgctggccacacggagaggctggtctccctgaaggtgggactgaagccagaagcaaacaagcagtatcataacctggtcagcatcatcaatggtgagaccctgaagctcccctgcacccctcccggggctgggcagggacgtttctcctggacgctccccaatggcatgcatctggagggcccccaaaccctgggacgcgtttctcttctggacaatggcaccctcacggttcgtgaggcctcggtgtttgacaggggtacctatgtatgcaggatggagacggagtacggcccttcggtcaccagcatccccgtgattgtgatcgcctatcctccccggatcaccagcgagcccaccccggtcatctacacccggcccgggaacaccgtgaaactgaactgcatggctatggggattcccaaagctgacatcacgtgggagttaccggataagtcgcatctgaaggcaggggttcaggctcgtctgtatggaaacagatttcttcacccccagggatcactgaccatccagcatgccacacagagagatgccggcttctacaagtgcatggcaaaaaacattctcggcagtgactccaaaacaacttacatccacgtcttctga

SEQ ID NO: 5 - DNA, 345 bp, Homo sapiens
atgtcggccctcagcctcctcattctgggcctgctcacggcagtgccacctgccagctgtcagcaaggcctggggaaccttcagccctggatgcagggccttatcgcggtggccgtgttcctggtcctcgttgcaatcgcctttgcagtcaaccacttctggtgccaggaggagccggagcctgcacacatgatcctgaccgtcggaaacaaggcagatggagtcctggtgggaacagatggaaggtactcttcgatggcggccagtttcaggtccagtgagcatgagaatgcctatgagaatgtgcccgaggaggaaggcaaggtccgcagcaccccgatgtaa

SEQ ID NO: 6 - DNA, 345 bp, Homo sapiens
atgtcggccctcagcctcctcattctgggcctgctcacggcagtgccacctgccagctgtcagcaaggcctggggaaccttcagccctggatgcagggccttatcgcggtggccgtgttcctggtcctcgttgcaatcgcctttgcagtcaaccacttctggtgccaggaggagccggagcctgcacacatgatcctgaccgtcggaaacaaggcagatggagtcctggtgggaatagatggaaggtactcttcgatggcggccagtttcaggtccagtgagcatgagaatgcctatgagaatgtgcccgaggaggaaggcaaggtccgcagcaccccgatgtaa

SEQ ID NO: 7 - DNA, 1137 bp, Homo sapiens
atgaaggacgtaccgggcttcctacagcagagccagaactccgggcccgggcagcccgctgtgtggcaccgtctggaggagctctacacgaagaactttatgaaaactttatcagtgaatttgaacacaggtaaaagcctctcatccgtttttcactttgaaaatgagtgcattgatgctcggcggtgctcaaaggctggtggcttttatttcagggtgaaccctttgtccctcgtggaaatcattcttcatgtagttagacagatgactgatcctaatgtggctcttacttttctggaaaagactcgtgagaaggtgaaaagtagtgatgaggcagtgatcctgtgtaaaacagcaattggagctctaaaattaaacatcggggacctacaggttacaaaggaaacaattgaagatgttgaagaaatgctcaacaaccttcctggtgtgacatcggttcacagtcgtttctatgatctctccagtaaatactatcaaacaatcggaaaccacgcgtcctactacaaagatgctctgcggtttttgggctgtgttgacatcaaggatctaccagtgtctgagcagcaggagagagccttcacgctggggctagcaggacttctcggcgagggagtttttaactttggagaactcctcatgcaccctgtgctggagtccctgaggaatactgaccggcagtggctgattgacaccctctatgccttcaacagtggcaacgtagagcggttccagactctgaagactgcctggggccagcagcctgatttagcagctaatgaagcccagcttctgaggaaaattcagttgttgtgcctcatggagatgactttcacacgacctgccaatcacagacaactcacttttgaagaaattgccaaaagtgctaaaatcacagtgaatgaggtggagcttctggtgatgaaggccctttcggtggggctggtgaaaggcagtatagacgaggtggacaaacgagtccacatgacctggctgcagccccgagtgttggatttgcaacagatcaagggaatgaaggaccgcctggagttctggtgcacggatgtgaagagcatggagatgctggtggagcaccaggcccatgacatcctcacctag

SEQ ID NO: 8 - DNA, 1136 bp, Homo sapiens
atgaaggacgtaccgggcttcctacagcagagccagaactccgggcccgggcagcccgctgtgtggcaccgtctggaggagctctacacgaagaactttatgaaaactttatcagtgaatttgaacacaggtaaaagcctctcatccgtttttcactttgaaaatgagtgcattgatgctcggcggtgctcaaaggctggtggcttttatttcagggtgaaccctttgtccctcgtggaaatcattcttcatgtagttagacagatgactgatcctaatgtggctcttacttttctggaaaagactcgtgagaaggtgaaaagtagtgatgaggcagtgatcctgtgtaaaacagcaattggagctctaaaattaaacatcggggacctacaggttacaaaggaaacaattgaagatgttgaagaaatgctcaacaaccttcctggtgtgacatcggttcacagtcgtttctatgatctctccagtaaatactatcaaacaatcggaaaccacgcgtcctactacaaagatgctctgcggtttttgggctgtgttgacatcaaggatctaccagtgtctgagcagcaggagagagccttcacgctggggctagcaggacttctcggcgagggagtttttaactttggagaactcctcatgcaccctgtgctggagtccctgaggaatactgaccggcagtggctgattgacaccctctatgccttcaacagtggcaacgtagagcggttccagactctgaagactgcctggggccagcagcctgatttagcagctaatgaagcccagcttctgaggaaaattcagttgttgtgcctcatggagatgactttcacacgacctgccaatcacagacaactcacttttgaagaaattgccaaaagtgctaaaatcacagtgaatgaggtggagcttctggtgatgaaggccctttcggtggggctggtgaaaggcagtatagacgaggtggacaaacgagtccacatgacctggagcagccccgagtgttggatttgcaacagatcaagggaatgaaggaccgcctggagttctggtgcacggatgtgaagagcatggagatgctggtggagcaccaggcccatgacatcctcacctag

SEQ ID NO: 9 - DNA, 1443 bp, Homo sapiens
atggcggcgtccgtggtctgtcgggccgctaccgccggggcacaagtgctattgcgcgcccgccgctcgccggccctgctgcggacgccagccttgcggagtacggcaaccttcgctcaggcgctccagttcgtgccggagacgcaggttagcctgctggacaacggcctgcgtgtggcctccgagcagtcctctcagcccacttgcacggtgggagtgtggattgatgttggcagccgttttgagactgagaagaataatggggcaggctactttttggagcatctggctttcaagggaacaaagaatcggcctggcagtgccctggagaaggaggtggagagcatgggggcccatcttaatgcctacagcacccgggagcacacagcttactacatcaaggcgctgtccaaggatctgccgaaagctgtggagctcctgggtgacattgtgcagaactgtagtctggaagactcacagattgagaaggaacgtgatgtgatcctgcgggagatgcaggagaatgatgcatctatgcgagatgtggtctttaactacctgcatgccacagcattccagggcacacctctagcccaggctgtggaggggcccagtgagaatgtcaggaagctgtctcgtgcagacttgaccgagtacctcagcacacattacaaggcccctcgaatggtgctggcagcagctggaggagtggagcaccagcaactgttagacctcgcccagaagcacctcggtggcatcccatggacatatgcagaggacgctgtgcccactcttactccatgccgcttcactggcagtgagatccgccaccgtgatgatgctctaccttttgcccacgtggccattgcagtagagggtcctggctgggccagcccggacaatgtggccttgcaagtggccaatgccatcatcggccactatgactgcacttatggtggtggcgtgcacctgtccagcccactggcttcaggtgctgtggccaacaagctatgccagagtttccagaccttcagcatctgctatgcagagacgggcttgctgggtgcacactttgtctgtgaccgaatgaaaatcgatgacatgatgttcgtcctgcaagggcagtggatgcgcctgtgtaccagtgccacggagagtgaggtggcccggggcaaaaacatcctcagaaatgccctggtatctcatctagatggcactactcctgtgtgtgaggacatcggacgcagcctcctgacctatggccgccgcatccccctggctgaatgggaaagccggattgcggaggtggatgccagtgtggtacgtgagatctgctccaagtacatctatgaccagtgcccagcagtggctggatatggccccattgagcagctcccagactacaaccggatccgtagcggcatgttctggctgcgcttctag

SEQ ID NO: 10 - DNA, 1443 bp, Homo sapiens
atggcggcgtccgtggtctgtcgggccgctaccgccggggcacaagtgctattgcgcgcccgccgctcgccggccctgctgcggacgccagccttgcggagtacggcaaccttcgctcaggcgctccagttcgtgccggagacgcaggttagcctgctggacaacggcctgcgtgtggcctccgagcagtcctctcagcccacttgcacggtgggagtgtggattgatgttggcagccgttttgagactgagaagaataatggggcaggctactttttggagcatctggctttcaagggaacaaagaatcggcctggcagtgccctggagaaggaggtggagagcatgggggcccatcttaatgcctacagcacccgggagcacacagcttactacatcaaggcgctgtccaaggatctgccgaaagctgtggagctcctgggtgacattgtgcagaactgtagtctggaagactcacagattgagaaggaacgtgatgtgatcctgcgggagatgcaggagaatgatgcatctatgcgagatgtggtctttaactacctgcatgccacagcattccagggcacacctctagcccaggctgtggaggggcccagtgagaatgtcaggaagctgtctcgtgcagacttgaccgagtacctcagcacacattacaaggcccctcgaatggtgctggcagcagctggaggagtggagcaccagcaactgttagacctcgcccagaagcacctcggtggcatcccatggacatatgcagaggacgctgtgcccactcttactccatgccacttcactggcagtgagatccgccaccgtgatgatgctctaccttttgcccacgtggccattgcagtagagggtcctggctgggccagcccggacaatgtggccttgcaagtggccaatgccatcatcggccactatgactgcacttatggtggtggcgtgcacctgtccagcccactggcttcaggtgctgtggccaacaagctatgccagagtttccagaccttcagcatctgctatgcagagacgggcttgctgggtgcacactttgtctgtgaccgaatgaaaatcgatgacatgatgttcgtcctgcaagggcagtggatgcgcctgtgtaccagtgccacggagagtgaggtggcccggggcaaaaacatcctcagaaatgccctggtatctcatctagatggcactactcctgtgtgtgaggacatcggacgcagcctcctgacctatggccgccgcatccccctggctgaatgggaaagccggattgcggaggtggatgccagtgtggtacgtgagatctgctccaagtacatctatgaccagtgcccagcagtggctggatatggccccattgagcagctcccagactacaaccggatccgtagcggcatgttctggctgcgcttctag

SEQ ID NO: 11 - DNA, 4500 bp, Homo sapiens
atgatggcaaactgggcggaagcaagacctctcctcattcttattgttttattagggcaatttgtctcaataaaagcccaggaagaagacgaggatgaaggatatggtgaagaaatagcctgcactcagaatggccagatgtacttaaacagggacatttggaaacctgccccttgtcagatctgtgtctgtgacaatggagccattctctgtgacaagatagaatgccaggatgtgctggactgtgccgaccctgtaacgccccctggggaatgctgtcctgtctgttcacaaacacctggaggtggcaatacaaattttggtagaggaagaaagggacaaaagggagaaccaggattagtgcctgttgtaacaggcatacgtggtcgtccaggaccggcaggacctccaggatcacagggaccaagaggagagcgagggccaaaaggaagacctggccctcgtggacctcagggaattgatggagaaccaggtgttcctggtcaacctggtgctccaggacctcctggacatccgtcccacccaggacccgatggcttgagcaggccgttttcagctcaaatggctgggttggatgaaaaatctggacttgggagtcaagtaggactaatgcctggctctgtgggtcctgttggcccaaggggaccacagggtttacaaggacagcaaggtggtgcaggacctacaggacctcctggtgaacctggtgatcctggaccaatgggtccgattggttcacgtggaccagagggccctcctggtaaacctggggaagatggtgaacctggcagaaatggaaatcctggtgaagtgggatttgcaggatctccgggagctcgtggatttcctggggctcctggtcttccaggtctgaagggtcaccgaggacacaaaggtcttgaaggccctaaaggtgaagttggagcacctggttccaagggtgaagctggccccactggtccaatgggtgccatgggtcctctgggtccgaggggaatgccaggagagagagggagacttgggccacagggtgctcctggacaacgaggtgcacatggtatgcctggaaaacctggaccaatgggtcctcttgggataccaggctcttctggttttccaggaaatcctggaatgaagggagaagcaggtcctacaggggcgcgaggccctgaaggtcctcaggggcagagaggtgaaactgggcccccaggtccagttggctctccaggtcttcctggtgcaataggaactgatggtactcctggtgccaaaggcccaacgggctctccgggtacctctggtcctcctggctcagcagggcctcctggatctccaggacctcagggtagcactggtcctcagggaattcgaggccaaccgggtgatccaggagttccaggtttcaaaggagaagctggcccaaaaggggaaccagggccacatggtattcagggtccgataggcccacccggtgaagaaggcaaaagaggtcccagaggtgacccaggaacagttggtcctccagggccagtgggagaaaggggtgctcctggcaatcgtggttttccaggctctgatggtttacctgggccaaagggtgctcaaggagaacggggtcctgtaggttcttcaggacccaaaggaagccagggggatccaggacgtccaggggaacctgggcttccaggtgctcggggtttgacaggaaatcctggtgttcaaggtcctgaaggaaaacttggacctttgggtgcgccaggggaagatggccgtccaggtcctccaggctccataggaatcagagggcagcccgggagcatgggccttccaggccccaaaggtagcagtggtgaccctgggaaacctggagaagcaggaaatgctggagttcctgggcagaggggagctcctggaaaagatggtgaagttggtccttctggtcctgtgggcccgccgggtctagctggtgaaagaggagaacaaggacctccaggccccacaggttttcaggggcttcctggtcctccagggcctcctggagaaggtggaaaaccaggtgatcaaggtgttcctggagatcccggagcagttggcccgttaggacctagaggagaacgaggaaatcctggggaaagaggagaacctgggataactggactccctggtgagaagggaatggctggaggacatggtcctgatggcccaaaaggcagtccaggtccatctgggacccctggagatacaggcccaccaggtcttcaaggtatgccgggagaaagaggaattgcaggaactcctggccccaagggtgacagaggtggcataggagaaaaaggtgctgaaggcacagctggaaatgatggtgcaagaggtcttccaggtcctttgggccctccaggtccggcaggtcctactggagaaaagggtgaacctggtcctcgaggtttagttggccctcctggctcccggggcaatcctggttctcgaggtgaaaatgggccaactggagctgttggttttgccggaccccagggtcctgacggacagcctggagtaaaaggtgaacctggagagccaggacagaagggagatgctggttctcctggaccacaaggtttagcaggatcccctggccctcatggtcctaatggtgttcctggactaaaaggtggtcgaggaacccaaggtccgcctggtgctacaggatttcctggttctgcgggcagagttggacctccaggccctgctggagctccaggacctgcgggacccctaggggaacccgggaaggagggacctccaggtcttcgtggggaccctggctctcatgggcgtgtgggagatcgaggaccagctggcccccctggtggcccaggagacaaaggggacccaggagaagatgggcaacctggtccagatggcccccctggtccagctggaacgaccgggcagagaggaattgttggcatgcctgggcaacgtggagagagaggcatgcccggcctaccaggcccagcgggaacaccaggaaaagtaggaccaactggtgcaacaggagataaaggtccacctggacctgtggggcccccaggctccaatggtcctgtaggggaacctggaccagaaggtccagctggcaatgatggtaccccaggacgggatggtgctgttggagaacgtggtgatcgtggagaccctgggcctgcaggtctgccaggctctcagggtgcccctggaactcctggccctgtgggtgctccaggagatgcaggacaaagaggagatccgggttctcggggtcctataggaccacctggtcgagctgggaaacgtggattacctggaccccaaggacctcgtggtgacaaaggtgatcatggagaccgaggcgacagaggtcagaagggccacagaggctttactggtcttcagggtcttcctggccctcctggtccaaatggtgaacaaggaagtgctggaatccctggaccatttggcccaagaggtcctccaggcccagttggtccttcaggtaaagaaggaaaccctgggccacttgggccaattggacctccaggtgtacgaggcagtgtaggagaagcaggacctgagggccctcctggtgagcctggcccacctggccctccgggtccccctggccaccttacagctgctcttggggatatcatggggcactatgatgaaagcatgccagatccacttcctgagtttactgaagatcaggcggctcctgatgacaaaaacaaaacggacccaggggttcatgctaccctgaagtcactcagtagtcagattgaaaccatgcgcagccccgatggctcgaaaaagcacccagcccgcacgtgtgatgacctaaagctttgccattccgcaaagcagagtggtgaatactggattgatcctaaccaaggatctgttgaagatgcaatcaaagtttactgcaacatggaaacaggagaaacatgtatttcagcaaacccatccagtgtaccacgtaaaacctggtgggccagtaaatctcctgacaataaacctgtttggtatggtcttgatatgaacagagggtctcagttcgcttatggagaccaccaatcacctaatacagccattactcagatgacttttttgcgccttttatcaaaagaagcctcccagaacatcacttacatctgtaaaaacagtgtaggatacatggacgatcaagctaagaacctcaaaaaagctgtggttctcaaaggggcaaatgacttagatatcaaagcagagggaaatattagattccggtatatcgttcttcaagacacttgctctaagcggaatggaaatgtgggcaagactgtctttgaatatagaacacagaatgtggcacgcttgcccatcatagatcttgctcctgtggatgttggcggcacagaccaggaattcggcgttgaaattgggccagtttgttttgtgtaa

SEQ ID NO: 12 - DNA, 4500 bp, Homo sapiens
atgatggcaaactgggcggaagcaagacctctcctcattcttattgttttattagggcaatttgtctcaataaaagcccaggaagaagacgaggatgaaggatatggtgaagaaatagcctgcactcagaatggccagatgtacttaaacagggacatttggaaacctgccccttgtcagatctgtgtctgtgacaatggagccattctctgtgacaagatagaatgccaggatgtgctggactgtgccgaccctgtaacgccccctggggaatgctgtcctgtctgttcacaaacacctggaggtggcaatacaaattttggtagaggaagaaagggacaaaagggagaaccaggattagtgcctgttgtaacaggcatacgtggtcgtccaggaccggcaggacctccaggatcacagggaccaagaggagagcgagggccaaaaggaagacctggccctcgtggacctcagggaattgatggagaaccaggtgttcctggtcaacctggtgctccaggacctcctggacatccgtcccacccaggacccgatggcttgagcaggccgttttcagctcaaatggctgggttggatgaaaaatctggacttgggagtcaagtaggactaatgcctggctctgtgggtcctgttggcccaaggggaccacagggtttacaaggacagcaaggtggtgcaggacctacaggacctcctggtgaacctggtgatcctggaccaatgggtccgattggttcacgtggaccagagggccctcctggtaaacctggggaagatggtgaacctggcagaaatggaaatcctggtgaagtgggatttgcaggatctccgggagctcgtggatttcctggggctcctggtcttccaggtctgaagggtcaccgaggacacaaaggtcttgaaggccctaaaggtgaagttggagcacctggttccaagggtgaagctggccccactggtccaatgggtgccatgggtcctctgggtccgaggggaatgccaggagagagagggagacttgggccacagggtgctcctggacaacgaggtgcacatggtatgcctggaaaacctggaccaatgggtcctcttgggataccaggctcttctggttttccaggaaatcctggaatgaagggagaagcaggtcctacaggggcgcgaggccctgaaggtcctcaggggcagagaggtgaaactgggcccccaggtccagttggctctccaggtcttcctggtgcaataggaactgatggtactcctggtgccaaaggcccaacgggctctccgggtacctctggtcctcctggctcagcagggcctcctggatctccaggacctcagggtagcactggtcctcagggaattcgaggccaaccgggtgatccaggagttccaggtttcaaaggagaagctggcccaaaaggggaaccagggccacatggtattcagggtccgataggcccacccggtgaagaaggcaaaagaggtcccagaggtgacccaggaacagttggtcctccagggccagtgggagaaaggggtgctcctggcaatcgtggttttccaggctctgatggtttacctgggccaaagggtgctcaaggagaacggggtcctgtaggttcttcaggacccaaaggaagccagggggatccaggacgtccaggggaacctgggcttccaggtgctcggggtttgacaggaaatcctggtgttcaaggtcctgaaggaaaacttggacctttgggtgcgccaggggaagatggccgtccaggtcctccaggctccataggaatcagagggcagcccgggagcatgggccttccaggccccaaaggtagcagtggtgaccctgggaaacctggagaagcaggaaatgctggagttcctgggcagaggggagctcctggaaaagatggtgaagttggtccttctggtcctgtgggcccgccgggtctagctggtgaaagaggagaacaaggacctccaggccccacaggttttcaggggcttcctggtcctccagggcctcctggagaaggtggaaaaccaggtgatcaaggtgttcctggagatcccggagcagttggcccgttaggacctagaggagaacgaggaaatcctggggaaagaggagaacctgggataactggactccctggtgagaagggaatggctggaggacatggtcctgatggcccaaaaggcagtccaggtccatctgggacccctggagatacaggcccaccaggtcttcaaggtatgccgggagaaagaggaattgcaggaactcctggccccaagggtgacagaggtggcataggagaaaaaggtgctgaaggcacagctggaaatgatggtgcaagaggtcttccaggtcctttgggccctccaggtccggcaggtcctactggagaaaagggtgaacctggtcctcgaggtttagttggccctcctggctcccggggcaatcttggttctcgaggtgaaaatgggccaactggagctgttggttttgccggaccccagggtcctgacggacagcctggagtaaaaggtgaacctggagagccaggacagaagggagatgctggttctcctggaccacaaggtttagcaggatcccctggccctcatggtcctaatggtgttcctggactaaaaggtggtcgaggaacccaaggtccgcctggtgctacaggatttcctggttctgcgggcagagttggacctccaggccctgctggagctccaggacctgcgggacccctaggggaacccgggaaggagggacctccaggtcttcgtggggaccctggctctcatgggcgtgtgggagatcgaggaccagctggcccccctggtggcccaggagacaaaggggacccaggagaagatgggcaacctggtccagatggcccccctggtccagctggaacgaccgggcagagaggaattgttggcatgcctgggcaacgtggagagagaggcatgcccggcctaccaggcccagcgggaacaccaggaaaagtaggaccaactggtgcaacaggagataaaggtccacctggacctgtggggcccccaggctccaatggtcctgtaggggaacctggaccagaaggtccagctggcaatgatggtaccccaggacgggatggtgctgttggagaacgtggtgatcgtggagaccctgggcctgcaggtctgccaggctctcagggtgcccctggaactcctggccctgtgggtgctccaggagatgcaggacaaagaggagatccgggttctcggggtcctataggaccacctggtcgagctgggaaacgtggattacctggaccccaaggacctcgtggtgacaaaggtgatcatggagaccgaggcgacagaggtcagaagggccacagaggctttactggtcttcagggtcttcctggccctcctggtccaaatggtgaacaaggaagtgctggaatccctggaccatttggcccaagaggtcctccaggcccagttggtccttcaggtaaagaaggaaaccctgggccacttgggccaattggacctccaggtgtacgaggcagtgtaggagaagcaggacctgagggccctcctggtgagcctggcccacctggccctccgggtccccctggccaccttacagctgctcttggggatatcatggggcactatgatgaaagcatgccagatccacttcctgagtttactgaagatcaggcggctcctgatgacaaaaacaaaacggacccaggggttcatgctaccctgaagtcactcagtagtcagattgaaaccatgcgcagccccgatggctcgaaaaagcacccagcccgcacgtgtgatgacctaaagctttgccattccgcaaagcagagtggtgaatactggattgatcctaaccaaggatctgttgaagatgcaatcaaagtttactgcaacatggaaacaggagaaacatgtatttcagcaaacccatccagtgtaccacgtaaaacctggtgggccagtaaatctcctgacaataaacctgtttggtatggtcttgatatgaacagagggtctcagttcgcttatggagaccaccaatcacctaatacagccattactcagatgacttttttgcgccttttatcaaaagaagcctcccagaacatcacttacatctgtaaaaacagtgtaggatacatggacgatcaagctaagaacctcaaaaaagctgtggttctcaaaggggcaaatgacttagatatcaaagcagagggaaatattagattccggtatatcgttcttcaagacacttgctctaagcggaatggaaatgtgggcaagactgtctttgaatatagaacacagaatgtggcacgcttgcccatcatagatcttgctcctgtggatgttggcggcacagaccaggaattcggcgttgaaattgggccagtttgttttgtgtaa

SEQ ID NO: 13 - DNA, 1830 bp, Homo sapiens
atgtcagggtgggagtcatattacaaaaccgagggcgatgaagaagcagaggaagaacaagaagagaaccttgaagcaagtggagactataaatattcaggaagagatagtttgatttttttggttgatgcctccaaggctatgtttgaatctcagagtgaagatgagttgacaccttttgacatgagcatccagtgtatccaaagtgtgtacatcagtaagatcataagcagtgatcgagatctcttggctgtggtgttctatggtaccgagaaagacaaaaattcagtgaattttaaaaatatttacgtcttacaggagctggataatccaggtgcaaaacgaattctagagcttgaccagtttaaggggcagcagggacaaaaacgtttccaagacatgatgggccacggatctgactactcactcagtgaagtgctgtgggtctgtgccaacctctttagtgatgtccaattcaagatgagtcataagaggatcatgctgttcaccaatgaagacaacccccatggcaatgacagtgccaaagccagccgggccaggaccaaagccggtgatctccgagatacaggcatcttccttgacttgatgcacctgaagaaacctgggggctttgacatatccttgttctacagagatatcatcagcatagcagaggatgaggacctcagggttcactttgaggaatccagcaagctagaagacctgttgcggaaggttcgcgccaaggagaccaggaagcgagcactcagcaggttaaagctgaagctcaacaaagatatagtgatctctgtgggcatttataatctggtccagaaggctctcaagcctcctccaataaagctctatcgggaaacaaatgaaccagtgaaaaccaagacccggacctttaatacaagtacaggcggtttgcttctgcctagcgataccaagaggtctcagatctatgggagtcgtcagattatactggagaaagaggaaacagaagagctaaaacggtttgatgatccaggtttgatgctcatgggtttcaagccgttggtactgctgaagaaacaccattacctgaggccctccctgttcgtgtacccagaggagtcgctggtgattgggagctcaaccctgttcagtgctctgctcatcaagtgtctggagaaggaggttgcagcattgtgcagatacacaccccgcaggaacatccctccttattttgtggctttggtgccacaggaagaagagttggatgaccagaaaattcaggtgactcctccaggcttccagctggtctttttaccctttgctgatgataaaaggaagatgccctttactgaaaaaatcatggcaactccagagcaggtgggcaagatgaaggctatcgttgagaagcttcgcttcacatacagaagtgacagctttgagaaccccgtgctgcagcagcacttcaggaacctggaggccttggccttggatttgatggagccggaacaagcagtggacctgacattgcccaaggttgaagcaatgaataaaagactgggctccttggtggatgagtttaaggagcttgtttacccaccagattacaatcctgaagggaaagttaccaagagaaaacacgataatgaaggttctggaagcaaaaggcccaaggtggagtattcagaagaggagctgaagacccacatcagcaagggtacgctgggcaagttcactgtgcccatgctgaaagaggcctgccgggcttacgggctgaagagtgggctgaagaagcaggagctgctggaagccctcaccaagcacttccaggactga

SEQ ID NO: 14 - DNA, 1830 bp, Homo sapiens
atgtcagggtgggagtcatattacaaaaccgagggcgatgaagaagcagaggaagaacaagaagagaaccttgaagcaagtggagactataaatattcaggaagagatagtttgatttttttggttgatgcctccaaggctatgtttgaatctcagagtgaagatgagttgacaccttttgacatgagcatccagtgtatccaaagtgtgtacatcagtaagatcataagcagtgatcgagatctcttggctgtggtgttctatggtaccgagaaagacaaaaattcagtgaattttaaaaatatttacgtcttacaggagctggataatccaggtgcaaaacgaattctagagcttgaccagtttaaggggcagcagggacaaaaacgtttccaagacatgatgggccacggatctgactactcactcagtgaagtgctgtgggtctgtgccaacctctttagtgatgtccaattcaagatgagtcataagaggatcatgctgttcaccaatgaagacaacccccatggcaatgacagtgccaaagccagccgggccaggaccaaagccggtgatctccgagatacaggcatcttccttgacttgatgcacctgaagaaacctgggggctttgacatatccttgttctacagagatatcatcagcatagcagaggatgaggacctcagggttcactttgaggaatccagcaagctagaagacctgttgcggaaggttcgcgccaaggagaccaggaagcgagcactcagcaggttaaagctgaagctcaacaaagatatagtgatctctgtgggcatttataatctggtccagaaggctctcaagcctcctccaataaagctctatcgggaaacaaatgaaccaatgaaaaccaagacccggacctttaatacaagtacaggcggtttgcttctgcctagcgataccaagaggtctcagatctatgggagtcgtcagattatactggagaaagaggaaacagaagagctaaaacggtttgatgatccaggtttgatgctcatgggtttcaagccgttggtactgctgaagaaacaccattacctgaggccctccctgttcgtgtacccagaggagtcgctggtgattgggagctcaaccctgttcagtgctctgctcatcaagtgtctggagaaggaggttgcagcattgtgcagatacacaccccgcaggaacatccctccttattttgtggctttggtgccacaggaagaagagttggatgaccagaaaattcaggtgactcctccaggcttccagctggtctttttaccctttgctgatgataaaaggaagatgccctttactgaaaaaatcatggcaactccagagcaggtgggcaagatgaaggctatcgttgagaagcttcgcttcacatacagaagtgacagctttgagaaccccgtgctgcagcagcacttcaggaacctggaggccttggccttggatttgatggagccggaacaagcagtggacctgacattgcccaaggttgaagcaatgaataaaagactgggctccttggtggatgagtttaaggagcttgtttacccaccagattacaatcctgaagggaaagttaccaagagaaaacacgataatgaaggttctggaagcaaaaggcccaaggtggagtattcagaagaggagctgaagacccacatcagcaagggtacgctgggcaagttcactgtgcccatgctgaaagaggcctgccgggcttacgggctgaagagtgggctgaagaagcaggagctgctggaagccctcaccaagcacttccaggactga

SEQ ID NO: 15 - DNA, 2142 bp, Homo sapiens
atgctgttggccaccctcctcctcctcctccttggaggcgctctggcccatccagaccggattatttttccaaatcatgcttgtgaggaccccccagcagtgctcttagaagtgcagggcaccttacagaggcccctggtccgggacagccgcacctcccctgccaactgcacctggctcatcctgggcagcaaggaacagactgtcaccatcaggttccagaagctacacctggcctgtggctcagagcgcttaaccctacgctcccctctccagccactgatctccctgtgtgaggcacctcccagccctctgcagctgcccgggggcaacgtcaccatcacttacagctatgctggggccagagcacccatgggccagggcttcctgctctcctacagccaagattggctgatgtgcctgcaggaagagtttcagtgcctgaaccaccgctgtgtatctgctgtccagcgctgtgatggggttgatgcctgtggcgatggctctgatgaagcaggttgcagctcagaccccttccctggcctgaccccaagacccgtcccctccctgccttgcaatgtcaccttggaggacttctatggggtcttctcctctcctggatatacacacctagcctcagtctcccacccccagtcctgccattggctgctggacccccatgatggccggcggctggccgtgcgcttcacagccctggacttgggctttggagatgcagtgcatgtgtatgacggccctgggccccctgagagctcccgactactgcgtagtctcacccacttcagcaatggcaaggctgtcactgtggagacactgtctggccaggctgttgtgtcctaccacacagttgcttggagcaatggtcgtggcttcaatgccacctaccatgtgcggggctattgcttgccttgggacagaccctgtggcttaggctctggcctgggagctggcgaaggcctaggtgagcgctgctacagtgaggcacagcgctgtgacggctcatgggactgtgctgacggcacagatgaggaggactgcccaggctgcccacctggacacttcccctgtggggctgctggcacctctggtgccacagcctgctacctgcctgctgaccgctgcaactaccagactttctgtgctgatggagcagatgagagacgctgtcggcattgccagcctggcaatttccgatgccgggacgagaagtgcgtgtatgagacgtgggtgtgcgatgggcagccagactgtgcggacggcagtgatgagtgggactgctcctatgttctgccccgcaaggtcattacagctgcagtcattggcagcctagtgtgcggcctgctcctggtcatcgccctgggctgcacctgcaagctctatgccattcgcacccaggagtacagcatctttgcccccctctcccggatggaggctgagattgtgcagcagcaggcacccccttcctacgggcagctcattgcccagggtgccatcccacctgtagaagactttcctacagagaatcctaatgataactcagtgctgggcaacctgcgttctctgctacagatcttacgccaggatatgactccaggaggtggcccaggtgcccgccgtcgtcagcggggccgcttgatgcgacgcctggtacgccgtctccgccgctggggcttgctccctcgaaccaacaccccggctcgggcctctgaggccagatcccaggtcacaccttctgctgctccccttgaggccctagatggtggcacaggtccagcccgtgagggcggggcagtgggtgggcaagatggggagcaggcacccccactgcccatcaaggctcccctcccatctgctagcacgtctccagcccccactactgtccctgaagccccagggccactgccctcactgcccctagagccatcactattgtctggagtggtgcaggccctgcgaggccgcctgttgcccagcctggggcccccaggaccaacccggagcccccctggaccccacacagcagtcctggccctggaagatgaggacgatgtgctactggtgccactggctgagccgggggtgtgggtagctgaggcagaggatgagccactgcttacctga

SEQ ID NO: 16 - DNA, 2142 bp, Homo sapiens
atgctgttggccaccctcctcctcctcctccttggaggcgctctggcccatccagaccggattatttttccaaatcatgcttgtgaggaccccccagcagtgctcttagaagtgcagggcaccttacagaggcccctggtccgggacagccgcacctcccctgccaactgcacctggctcatcctgggcagcaaggaacagactgtcaccatcaggttccagaagctacacctggcctgtggctcagagcgcttaaccctacgctcccctctccagccactgatctccctgtgtgaggcacctcccagccctctgcagctgcccgggggcaacgtcaccatcacttacagctatgctggggccagagcacccatgggccagggcttcctgctctcctacagccaagattggctgatgtgcctgcaggaagagtttcagtgcctgaaccaccgctgtgtatctgctgtccagcgctgtgatggggttgatgcctgtggcgatggctctgatgaagcaggttgcagctcagaccccttccctggcctgaccccaagacccgtcccctccctgccttgcaatgtcaccttggaggacttctatggggtcttctcctctcctggatatacacacctagcctcagtctcccacccccagtcctgccattggctgctggacccccatgatggccggcggctggccgtgcgcttcacagccctggacttgggctttggagatgcagtgcatgtgtatgacggccctgggccccctgagagctcccgactactgcgtagtctcacccacttcagcaatggcaaggctgtcactgtggagacactgtctggccaggctgttgtgtcctaccacacagttgcttggagcaatggtcgtggcttcaatgccacctaccatgtgcggggctattgcttgccttgggacagaccctgtggcttaggctctggcctgggagctggcgaaggcctaggtgagcgctgctacagtgaggcacagcgctgtgacggctcatgggactgtgctgacggcacagatgaggaggactgcccaggctgcccacctggacacttcccctgtggggctgctggcacctctggtgccacagcctgctacctgcctgctgaccgctgcaactaccagactttctgtgctgatggagcagatgagagacgctgtcggcattgccagcctggcaatttccgatgccgggacgagaagtgcgtgtatgagacgtgggtgtgcgatgggcagccagactgtgcggacggcagtgatgagtgggactgctcctatgttctgccccgcaaggtcattacagctgcagtcattggcagcctagtgtgcggcctgctcctggtcatcgccctgggctgcacctgcaagctctatgccattcgcacccaggagtacagcatctttgcccccctctcccagatggaggctgagattgtgcagcagcaggcacccccttcctacgggcagctcattgcccagggtgccatcccacctgtagaagactttcctacagagaatcctaatgataactcagtgctgggcaacctgcgttctctgctacagatcttacgccaggatatgactccaggaggtggcccaggtgcccgccgtcgtcagcggggccgcttgatgcgacgcctggtacgccgtctccgccgctggggcttgctccctcgaaccaacaccccggctcgggcctctgaggccagatcccaggtcacaccttctgctgctccccttgaggccctagatggtggcacaggtccagcccgtgagggcggggcagtgggtgggcaagatggggagcaggcacccccactgcccatcaaggctcccctcccatctgctagcacgtctccagcccccactactgtccctgaagccccagggccactgccctcactgcccctagagccatcactattgtctggagtggtgcaggccctgcgaggccgcctgttgcccagcctggggcccccaggaccaacccggagcccccctggaccccacacagcagtcctggccctggaagatgaggacgatgtgctactggtgccactggctgagccgggggtgtgggtagctgaggcagaggatgagccactgcttacctga

SEQ ID NO: 17 - DNA, 1851 bp, Homo sapiens
atgcccttcacactccacctgaggtcccgccttccctctgccataaggagtttgattctacaaaagaaaccaaacatcagaaatacatccagcatggctggagagctccgaccagccagcctggtggtcctgcccaggtcccttgctccagcttttgaaagattctgccaggtcaacactggtcctctacccctgctgggccagagtgagccagaaaagtggatgctgccccctcaaggtgctatctcagagaccaggatgggccatccccagttctggaaatacgagttcggtgcctgcaccggtagcctggcttcgctggagcagtactcggagcagctgaaggacatggtggccttcttcctgggctgcagcttctccctggaggaggccttggagaaagcggggctccccagaagagacccagcaggtcacagcccgacaacagtgccttgtgttacccatgctggcttctgctgccctctggtggtcacgatgaggcccattcccaaggacaagctggaagggctggtgcgggcctgctgctccctcggaggtgagcaggggcaacctgttcacatgggcgacccagaactgttgggaatcaaagagctttccaaacctgcctacggggatgccatggtgtgtcccccaggggaggttccagtgttctggccttctccgctgaccagtctcggagctgtcagcagctgtgagaccccactggcttttgccagcatcccaggctgcacagttatgactgacctgaaggatgcaaaggctccacctggttgtctcaccccagagagaattccagaggtccatcacatttcccaagatcctctgcactacagcatcgcgtcagtctctgcttctcagaagatcagagaactagagtctatgatcggcatagacccagggaaccgggggattgggcacctgctctgtaaagatgagctgctgaaggcctctctctcgctgtcccatgcccgctcagtgctcatcaccactgggttccccacacatttcaatcatgagcctccagaagagacagatggcccaccaggagctgttgctctggttgccttcctgcaggccttggagaaggaggtcgccataatcgttgaccagagagcctggaacttgcaccagaagattgttgaagatgctgttgagcaaggtgttctgaagacgcagatcccgatattaacttaccaaggtggatcagtggaagctgctcaggcattcctgtgcaaaaatggggacccgcagacacctagatttgaccacctggtggccatagagcgtgccggaagagctgctgatggcaattactacaatgcaaggaagatgaacatcaagcacttggttgaccccattgacgatctttttcttgctgcgaagaagattcctggaatctcatcaactggagtcggtgatggaggcaacgagcttgggatgggtaaagtcaaggaggctgtgaggaggcacatacggcacggggatgtcatcgcctgcgacgtggaggctgactttgccgtcattgctggtgtttctaactggggaggctatgccctggcctgcgcactctacatcctgtactcatgtgctgtccacagtcagtacctgaggaaagcagtcggaccctccagggcacctggagatcaggcctggactcaggccctcccgtcggtcattaaggaagaaaaaatgctgggcatcttggtgcagcacaaagtccggagtggcgtctcgggcatcgtgggcatggaggtggatgggctgcccttccacaacacccacgccgagatgatccagaagctggtggacgtcaccacggcacaggtgtaa

SEQ ID NO: 18 - DNA, 1851 bp, Homo sapiens
atgcccttcacactccacctgaggtcccgccttccctctgccataaggagtttgattctacaaaagaaaccaaacatcagaaatacatccagcatggctggagagctccgaccagccagcctggtggtcctgcccaggtcccttgctccagcttttgaaagattctgccaggtcaacactggtcctctacccctgctgggccagagtgagccagaaaagtggatgctgccccctcaaggtgctatctcagagaccaggatgggccatccccagttctggaaatacgagttcggtgcctgcaccggtagcctggcttcgctggagcagtactcggagcagctgaaggacatggtggccttcttcctgggctgcagcttctccctggaggaggccttggagaaagcggggctccccagaagagacccagcaggtcacagcccgacaacagtgccttgtgttacccatgctggcttctgctgccctctggtggtcacgatgaggcccattcccaaggacaagctggaagggctggtgcgggcctgctgctccctcggaggtgagcaggggcaacctgttcacatgggcgacccagaactgttgggaatcaaagagctttccaaacctgcctacggggatgccatggtgtgtcccccaggggaggttccagtgttctggccttctccgctgaccagtctcggagctgtcagcagctgtgagaccccactggcttttgccagcatcccaggctgcacagttatgactgacctgaaggatgcaaaggctccacctggttgtctcaccccagagagaattccagaggtccatcacatttcccaagatcctctgcactacagcatcgcgtcagtctctgcttctcagaagatcagagaactagagtctatgatcggcatagacccagggaaccgggggattgggcacctgctctgtaaagatgagctgctgaaggcctctctctcgctgtcccatgcccgctcagtgctcatcaccactgggttccccacacatttcaatcatgagcctccagaagagacagatggcccaccaggagctgttgctctggttgccttcctgcaggccttggagaaggaggtcgccataatcggtgaccagagagcctggaacttgcaccagaagattgttgaagatgctgttgagcaaggtgttctgaagacgcagatcccgatattaacttaccaaggtggatcagtggaagctgctcaggcattcctgtgcaaaaatggggacccgcagacacctagatttgaccacctggtggccatagagcgtgccggaagagctgctgatggcaattactacaatgcaaggaagatgaacatcaagcacttggttgaccccattgacgatctttttcttgctgcgaagaagattcctggaatctcatcaactggagtcggtgatggaggcaacgagcttgggatgggtaaagtcaaggaggctgtgaggaggcacatacggcacggggatgtcatcgcctgcgacgtggaggctgactttgccgtcattgctggtgtttctaactggggaggctatgccctggcctgcgcactctacatcctgtactcatgtgctgtccacagtcagtacctgaggaaagcagtcggaccctccagggcacctggagatcaggcctggactcaggccctcccgtcggtcattaaggaagaaaaaatgctgggcatcttggtgcagcacaaagtccggagtggcgtctcgggcatcgtgggcatggaggtggatgggctgcccttccacaacacccacgccgagatgatccagaagctggtggacgtcaccacggcacaggtgtaa

SEQ ID NO: 19 - DNA, 1821 bp, Homo sapiens
atgacagtcgtagggaaccctcgaagttggagctgccagtggttgccaatcctgatactgttgctgggcacaggccatgggccaggggtggaaggcgtgacacactacaaggccggcgaccctgttattctgtatgtcaacaaagtgggaccctaccataaccctcaggaaacttaccactactatcagcttccagtctgctgccctgagaagatacgtcacaaaagccttagcctgggtgaagtgctggatggggaccgaatggctgagtctttgtatgagatccgctttcgggaaaacgtggagaagagaattctgtgccacatgcagctcagttctgcacaggtggagcagctgcgccaggccattgaagaactgtactactttgaatttgtggtagatgacttgccaatccggggctttgtgggctacatggaggagagtggtttcctgccacacagccacaagataggactctggacccatttggacttccacctagaattccatggagaccgaattatatttgccaatgtttcagtgcgggacgtcaagccccacagcttggatgggttacgacctgacgagttcctaggccttacccacacttatagcgtgcgctggtctgagacttcagtggagcgtcggagtgacaggcgccgtggtgacgatggtggtttctttcctcgaacactggaaatccattggttgtccatcatcaactccatggtgcttgtgtttttactggtgggttttgtggctgtcattctaatgcgtgtgcttcggaatgacctggctcggtacaacttagatgaggagaccacctctgcaggttctggtgatgactttgaccagggtgacaatggctggaaaattatccatacagatgtcttccgcttccccccataccgtggtctgctctgtgctgtgcttggcgtgggtgcccagttcctggcccttggcactggcattattgtcatggcactgctgggcatgttcaatgtgcaccgtcatggggccattaactcagcagccatcttgttgtatgccctgacctgctgcatctctggctacgtgtccagccacttctaccggcagattggaggcgagcgttgggtgtggaacatcattctcaccaccagtctcttctctgtgcctttcttcctgacgtggagtgtggtgaactcagtgcattgggccaatggttcgacacaggctctgccagccacaaccatcctgctgcttctgacggtttggctgctggtgggctttcccctcactgtcattggaggcatctttgggaagaacaacgccagcccctttgatgcaccctgtcgcaccaagaacatcgcccgggagattccaccccagccctggtacaagtctactgtcatccacatgactgttggaggcttcctgcctttcagtgccatctctgtggagctgtactacatctttgccacagtatggggtcgggagcagtacactttgtacggcatcctcttctttgtcttcgccatcctgctgagtgtgggggcttgcatctccattgcactcacctacttccagttgtctggggaggattaccgctggtggtggcgatctgtgctgagtgttggctccaccggcctcttcatcttcctctactcagttttctattatgcccggcgctccaacatgtctggggcagtacagacagtagagttcttcggctactccttactcactggttatgtcttcttcctcatgctgggcaccatctcctttttttcttccctaaagttcatccggtatatctatgttaacctcaagatggactga

SEQ ID NO: 20 - DNA, 1821 bp, Homo sapiens
atgacagtcgtagggaaccctcgaagttggagctgccagtggttgccaatcctgatactgttgctgggcacaggccatgggccaggggtggaaggcgtgacacactacaaggccggcgaccctgttattctgtatgtcaacaaagtgggaccctaccataaccctcaggaaacttaccactactatcagcttccagtctgctgccctgagaagatacgtcacaaaagccttagcctgggtgaagtgctggatggggaccgaatggctgagtctttgtatgagatccgctttcgggaaaacgtggagaagagaattctgtgccacatgcagctcagttctgcacaggtggagcagctgcgccaggccattgaagaactgtactactttgaatttgtggtagatgacttgccaatccggggctttgtgggctacatggaggagagtggtttcctgccacacagccacaagataggactctggacccatttggacttccacctagaattccatggagaccgaattatatttgccaatgtttcagtgcgggacgtcaagccccacagcttggatgggttacgacctgacgagttcctaggccttacccacacttatagcgtgcgctggtctgagacttcagtggagcgtcggagtgacaggcgccgtggtgacgatggtggtttctttcctcgaacactggaaatccattggttgtccatcatcaactccatggtgcttgtgtttttactggtgggttttgtggctgtcattctaatgcgtgtgcttcggaatgacctggctcggtacaacttagatgaggagaccacctctgcaggttctggtgatgactttgaccagggtgacaatggctggaaaattatccatacagatgtcttccgcttccccccataccgtggtctgctctgtgctgtgcttggcgtgggtgcccagttcctggcccttggcactggcattattgtcatggcactgctgggcatgttcaatgtgcaccgtcatggggccattaactcagcagccatcttgttgtatgccctgacctgctgcatctctggctacgtgtccagccacttctaccggcagattggaggcgagcgttgggtgtggaacatcattctcaccaccagtctcttctctgtgcctttcttcctgacgtggagtgtggtgaactcagtgcattgggccaatggttcgacacaggctctgccagccacaaccatcctgctgcttctgacggtttggctgctggtgggctttcccctcactgtcattggaggcatctttgggaagaacaacgccagcccctttgatgcaccctgtcgcaccaagaacatcgcccgggagattccaccccagccctggtacaagtctactgtcatccacatgactgttggaggcttcctgcctttcagtgccatctctgtggagctgtactacatctttgccacagtatggggtcgggagcagtacactttgtacggcatcctcttctttgtcttcgccatcctgctgagtgtgggggcttgcatctccattgcactcacctacttccagttgtctggggaggattaccgctggtggtggcgatctgtgctgagtgttggctccaccggcctcttcatcttcctctactcagttttttattatgcccggcgctccaacatgtctggggcagtacagacagtagagttcttcggctactccttactcactggttatgtcttcttcctcatgctgggcaccatctcctttttttcttccctaaagttcatccggtatatctatgttaacctcaagatggactga

SEQ ID NO: 21 - DNA, 2190 bp, Homo sapiens
atgttttccgccctgaagaagctggtggggtcggaccaggccccgggccgggacaagaacatccccgccgggctgcagtccatgaaccaggcgttgcagaggcgcttcgccaagggggtgcagtacaacatgaagatagtgatccggggagacaggaacacgggcaagacagcgctgtggcaccgcctgcagggccggccgttcgtggaggagtacatccccacacaggagatccaggtcaccagcatccactggagctacaagaccacggatgacatcgtgaaggttgaagtctgggatgtagtagacaaaggaaaatgcaaaaagcgaggcgacggcttaaagatggagaacgacccccaggaggcggagtctgaaatggccctggatgctgagttcctggacgtgtacaagaactgcaacggggtggtcatgatgttcgacattaccaagcagtggaccttcaattacattctccgggagcttccaaaagtgcccacccacgtgccagtgtgcgtgctgggaaactaccgggacatgggcgagcaccgagtcatcctgccggacgacgtgcgtgacttcatcgacaacctggacagacctccaggttcctcctacttccgctatgctgagtcttccatgaagaacagcttcggcctaaagtaccttcataagttcttcaatatcccatttttgcagcttcagagggagacgctgttgcggcagctggagacgaaccagctggacatggacgccacgctggaggagctgtcggtgcagcaggagacggaggaccagaactacggcatcttcctggagatgatggaggctcgcagccgtggccatgcgtccccactggcggccaacgggcagagcccatccccgggctcccagtcaccagtggtgcctgcaggcgctgtgtccacggggagctccagccccggcacaccccagcccgccccacagctgcccctcaatgccgccccaccatcctctgtgccccctgtaccaccctcagaggccctgcccccacctgcgtgcccctcagcccccgccccacggcgcagcatcatctctaggctgtttgggacgtcacctgccaccgaggcagcccctccacctccagagccagtcccggccgcagagggcccagcaacggtccagagtgtggaggactttgttcctgacgaccgcctggaccgcagcttcctggaagacacaacccccgccagggacgagaagaaggtgggggccaaggctgcccagcaggacagcgacagtgatggggaggccctgggcggcaacccgatggtggcagggttccaggacgatgtggacctcgaagaccagccacgtgggagtcccccgctgcctgcaggccccgtccccagtcaagacatcactctttcgagtgaggaggaagcagaagtggcagctcccacaaaaggccctgccccagctccccagcagtgctcagagccagagaccaagtggtcctccataccagcttcgaagccacggagggggacagctcccacgaggaccgcagcacccccctggccaggcggtgtctctgttcgcacaggtccggagaagcgcagcagcaccaggccccctgctgagatggagccggggaagggtgagcaggcctcctcgtcggagagtgaccccgagggacccattgctgcacaaatgctgtccttcgtcatggatgaccccgactttgagagcgagggatcagacacacagcgcagggcggatgactttcccgtgcgagatgacccctccgatgtgactgacgaggatgagggccctgccgagccgcccccaccccccaagctccctctccccgccttcagactgaagaatgactcggacctcttcgggctggggctggaggaggccggacccaaggagagcagtgaggaaggtaaggagggcaaaaccccctctaaggagaagaagaagaagaagaaaaaaggcaaagaggaagaagaaaaagctgccaagaagaagagcaaacacaagaagagcaaggacaaggaggagggcaaggaggagcggcgacggcggcagcagcggcccccgcgcagcagggagaggacggctgccgatgagctggaggctttcctggggggcggggccccgggcggccgccaccctgggggtggcgactacgaggagctctag

SEQ ID NO: 22 - DNA, 2190 bp, Homo sapiens
atgttttccgccctgaagaagctggtggggtcggaccaggccccgggccgggacaagaacatccccgccgggctgcagtccatgaaccaggcgttgcagaggcgcttcgccaagggggtgcagtacaacatgaagatagtgatccggggagacaggaacacgggcaagacagcgctgtggcaccgcctgcagggccggccgttcgtggaggagtacatccccacacaggagatccaggtcaccagcatccactggagctacaagaccacggatgacatcgtgaaggttgaagtctgggatgtagtagacaaaggaaaatgcaaaaagcgaggcgacggcttaaagatggagaacgacccccaggaggcggagtctgaaatggccctggatgctgagttcctggacgtgtacaagaactgcaacggggtggtcatgatgttcgacattaccaagcagtggaccttcaattacattctccgggagcttccaaaagtgcccacccacgtgccagtgtgcgtgctgggaaactaccgggacatgggcgagcaccgagtcatcctgccggacgacgtgcgtgacttcatcgacaacctggacagacctccaggttcctcctacttccgctatgctgagtcttccatgaagaacagcttcggcctaaagtaccttcataagttcttcaatatcccatttttgcagcttcagagggagacgctgttgcggcagctggagacgaaccagctggacatggacgccacgctggaggagctgtcggtgcagcaggagacggaggaccagaactacggcatcttcctggagatgatggaggctcgcagccgtggccatgcgtccccactggcggccaacgggcagagcccatccccgggctcccagtcaccagtggtgcctgcaggcgctgtgtccacggggagctccagccccggcacaccccagcccgccccacagctgcccctcaatgccgccccaccatcctctgtgccccctgtaccaccctcagaggccctgcccccacctgcgtgcccctcagcccccgccccacggcgcagcatcatctctaggctgtttgggacgtcacctgccaccgaggcagcccctccacctccagagccagtcccggccgcagagggcccagcaacggtccagagtgtggaggactttgttcctgacgaccgcctggaccgcagcttcctggaagacacaacccccgccagggacgagaagaaggtgggggccaaggctgcccagcaggacagcgacagtgatggggaggccctgggcggcaacccgatggtggcagggttccaggacgatgtggacctcgaagaccagccacgtgggagtcccccgctgcctgcaggccccgtccccagtcaagacatcactctttcgagtgaggaggaagcagaagtggcagctcccacaaaaggccctgccccagctccccagcagtgctcagagccagagaccaagtggtcctccataccagcttcgaagccacggagggggacagctcccacgaggaccgcagcacccccctggccaggcggtgtctctgttcgcacaggtccggagaagcgcagcagcaccaggccccctgctgagatggagccggggaagggtgagcaggcctcctcgtcggagagtgaccccgagggacccattgctgcacaaatgctgtccttcgtcatggatgaccccgactttgagagcgagggatcagacacacagcgcagggcggatgactttcccgtgcgagatgacccctccgatgtgactgacgaggatgagggccctgccgagccgcccccaccccccaagctgcctctccccgccttcagactgaagaatgactcggacctcttcgggctggggctggaggaggccggacccaaggagagcagtgaggaaggtaaggagggcaaaaccccctctaaggagaagaagaagaagaagaaaaaaggcaaagaggaagaagaaaaagctgccaagaagaagagcaaacacaagaagagcaaggacaaggaggagggcaaggaggagcggcgacggcggcagcagcggcccccgcgcagcagggagaggacggctgccgatgagctggaggctttcctggggggcggggccccgggcggccgccaccctgggggtggcgactacgaggagctctag

SEQ ID NO: 23 - DNA, 1095 bp, Homo sapiens
ggccagatgcaggcggagcgaggagctcggggaggccgtgggcggcggccaggccgcggccggcctggcggagatcgccacagcgagcggcccggagccgcagcggcggtagccagaggcggcggcggaggcggcggcggggacggaggcggacgccggggccgtggccgtggccggggcttccgcggcgctcgcggaggccgaggaggaggaggcgccccgcgaggcagccgccgggagccgggaggctggggcgcaggggccagcgcgccggttgaagatgacagcgatgcagagacctatggagaagagaatgatgaacagggaaattattctaaaagaaagattgtctctaactgggatcgatatcaagatattgaaaaagaggtcaataatgaaagtggagagtcacagaggggaacagatttcagtgtcctccttagctctgcaggggactcattctcacagttccggtttgctgaggagaaagaatgggatagtgaagcttcttgtccaaaacagaattcagcattttatgtggatagtgagttattggttcgagcccttcaagagctgcctctctgcctccgactcaacgttgctgccgaactggtccagggtacagttcctttagaggttcctcaggtgaaaccaaagagaactgatgatggcaagggattagggatgcagttaaaggggcccttggggcctggaggaagggggcccatctttgagctgaaatctgtggctgctggctgccctgtgttgctgggcaaagacaacccaagcccgggtccttcaagggattctcagaaacccacttccccactgcagtcagcaggagaccatttggaagaagaactagatctgttgcttaatttagatgcacctataaaagagggagataacatcttaccagatcagacgtctcaggacctgaaatccaaggaagatggggaggtggtccaagaggaagaagtttgtgcaaaaccatctgtgactgaagaaaaaaacatggaacctgagcaaccaagtacctccaaaaatgttaccgaggaagagctggaagactggttggacagcatgatttcctaa

SEQ ID NO: 24 - DNA, 1095 bp, Homo sapiens; identical to SEQ ID NO: 23

SEQ ID NO: 25 - DNA, 1239 bp, Homo sapiens
atggctccagtggagcacgttgtggcggatgctggggctttcctgcggcatgcggctctgcaggacatcgggaagaacatttacaccatccgggaggtggtcactgagattcgggacaaggccacacgcaggcggctcgctgtcctgccctacgagctgcggttcaaggagcccttaccggaatacgtgcggctggtgactgagttttcaaagaaaacaggagactaccccagcctctctgccacggacatccaagtgcttgcactcacataccagttggaagcagagtttgttggggtgtctcacctaaaacaagaaccacagaaggttaaggtgagctcatcgattcagcacccagaaacacctctgcacatttctggtttccatctgccctacaagcctaaacccccacaagaaacagaaaaaggacactcagcttgtgagcctgagaacctggaatttagttccttcatgttctggagaaaccctttgcccaacatcgatcatgaactgcaggagctgctgattgacagaggtgaggacgttccaagtgaggaggaggaggaggaagaaaacgggtttgaagacagaaaagatgacagcgatgacgacgggggtggctggataacccccagtaacatcaagcagatccagcaggagctggagcagtgtgacgtccccgaggacgtgcgggttggctgcctgaccacagacttcgccatgcagaatgttctgctgcagatggggctgcacgtgctggcggtgaacggcatgctgattcgtgaggcccggagctacatcttgcgctgccatggctgtttcaagacaacgtctgacatgagccgagtgttctgctcacactgtgggaacaagaccctgaagaaagtgtccgtgaccgtcagcgacgacggcaccctgcacatgcacttctcccgcaaccccaaggtgctgaacccccgcggcctccggtactcgcttcccactcccaaagggggcaaatacgccatcaacccccatctcaccgaggatcagcgcttccctcagctgcgactctcccaaaaggccaggcagaaaaccaacgtgttcgcccctgactacatcgccggggtgtcaccctttgtcgagaatgacatctccagccgctcagctaccctgcaggtccgggacagcaccttgggagctgggcggagacgcttaaatcccaacgcttccagaaagaagtttgtgaagaaaaggtga

SEQ ID NO: 26 - DNA, 1239 bp, Homo sapiens
atggctccagtggagcacgttgtggcggatgctggggctttcctgcggcatgcggctctgcaggacatcgggaagaacatttacaccatccgggaggtggtcactgagattcgggacaaggccacacgcaggcggctcgctgtcctgccctacgagctgcggttcaaggagcccttaccggaatacgtgcggctggtgactgagttttcaaagaaaacaggagactaccccagcctctctgccacggacatccaagtgcttgcactcacataccagttggaagcagagtttgttggggtgtctcacctaaaacaagaaccacagaaggttaaggtgagctcatcgattcagcacccagaaacacctctgcacatttctggtttccatctgccctacaagcctaaacccccacaagaaacagaaaaaggacactcagcttgtgagcctgagaacctggaatttagttccttcatgttctggagaaaccctttgcccaacatcgatcatgaactgcaggagctgctgattgacagaggtgaggacgttccaagtgaggaggaggaggaggaagaaaacgggtttgaagacagaaaagatgacagcgatgacgacgggggtggctggataacccccagtaacatcaagcagatccagcaggagctggagcagtgtgacgtccccgaggacgtgcgggttggctgcctgaccacagacttcgccatgcagaatgttctgctgcagatggggctgcacgtgctggcggtgaacggcatgctgattcgtgaggcccggagctacatcttgcgctgccatggctgtttcaagacaacgtctgacatgagccgagtgttctgctcacactgtgggaacaagaccctgaagaaagtgtccgtgaccgtcagcgacgacggcaccctgcacatgcacttctcccgcaaccccaaggtgctgaacccccgcggcctccggtactcgcttcccactcccaaagggggcaaatacgccatcaacccccatctcaccgaggatcagcgcttccctcagctgcgactctcccgaaaggccaggcagaaaaccaacgtgttcgcccctgactacatcgccggggtgtcaccctttgtcgagaatgacatctccagccgctcagctaccctgcaggtccgggacagcaccttgggagctgggcggagacgcttaaatcccaacgcttccagaaagaagtttgtgaagaaaaggtga

SEQ ID NO: 27 - DNA, 1515 bp, Homo sapiens
atggcaggccttaagagaagagtcccactgcacagcctcagatacttcatctccatggtgggtctcttctccaaaccaggactgcttccctggtatgccagaaatccaccaggatggtcacagctctttctgggcacagtatgtaagggagatttcacccgtgtgatagccacgaaatgtcagaaaggacaaaaaagtcagaagaaaccaagccatcttggaccactagatggttcctggcaggaaaggctggctgatgttgtgacaccactctggaggttgagctatgaagaacagctcaaggtgaaatttgcagctcagaagaaaattttacaaagactagagtcttacatccaaatgctcaatggagtcagtgtgacaacggctgtacccaaatctgagaggctctcttgtcttctccatcctattataccctctcctgtcatcaatggttaccgaaataagtccaccttctctgtgaaccgaggtccagatggcaatccaaagactgtggggttctacctgggaacttggagagatgggaacgttgtctgtgtgcagtctaatcatctgaaaaacatccctgagaaacacagtcaagtggcgcagtactatgaagtattccttcgacagtctccattggagccctgccttgtatttcatgaaggtggatactggcgtgagctcacagtccgcaccaatagccaagggcacacaatggctatcatcactttccatccccagaaattaagtcaggaggagctccatgttcagaaggagattgtaaaggaatttttcatcagaggtcctggagcagcctgtggcttgacctcactttacttccaggaaagtaccatgacccgttgcagccatcagcagtctccctatcagcttctgtttggggaaccctacatctttgaagaacttctgagcttgaagatccgcatctctccagatgcctttttccagattaacacagctggtgcagagatgctgtatcggactgtgggggagctgactggagtgaactctgacaccatccttcttgacatctgctgtggaactggtgtgattggcctctctctggctcagcatacatctcgggtccttgggattgaattgttggagcaggcagtggaggatgcaagatggactgcagccttcaatggcatcaccaactctgaatttcatactggtcaagcagagaagattttgccagggctgctaaagtcaaaggaagatggacagtcaattgttgctgtggtgaacccagcccgtgccggactgcattacaaggtgattcaagccattcgaaacttcagggccatccacacgctagtttttgtttcctgcaagctccatggtgaatccactaggaatgtcattgagctgtgctgtcctccagaccctgctaagaagctcttaggcgagccctttgtcctgcagcaagctgtacctgtggatttgttccctcacaccccacattgtgagctggtgctcctctttactcgataa

SEQ ID NO: 28 - DNA, 1515 bp, Homo sapiens
atggcaggccttaagagaagagtcccactgcacagcctcagatacttcatctccatggtgggtctcttctccaaaccaggactgcttccctggtatgccagaaatccaccaggatggtcacagctctttctgggcacagtatgtaagggagatttcacccgtgtgatagccacgaaatgtcagaaaggacaaaaaagtcagaagaaaccaagccatcttggaccactagatggttcctggcaggaaaggctggctgatgttgtgacaccactctggaggttgagctatgaagaacagctcaaggtgaaatttgcagctcagaagaaaattttacaaagactagagtcttacatccaaatgctcaatggagtcagtgtgacaacggctgtacccaaatctgagaggctctcttgtcttctccatcctattataccctctcctgtcatcaatggttaccgaaataagtccaccttctctgtgaaccgaggtccagatggcaatccaaagactgtggggttctacctgggaacttggagagatgggaacgttgtctgtgtgcagtctaatcatctgaaaaacatccctgagaaacacagtcaagtggcgcagtactatgaagtattccttcgacagtctccattggagccctgccttgtatttcatgaaggtggatactggcgtgagctcacagtccgcaccaatagccaagggcacacaatggctatcatcactttccatccccagaaattaagtcaggaggagctccatgttcagaaggagattgtaaaggaatttttcatcagaggtcctggagcagcctgtggcttgacctcactttacttccaggaaagtaccatgacccgttgcagccatcagcagtctccctatcagcttctgtttggggaaccctacatctttgaagaacttctgagcttgaagatccgcatctctccagatgcctttttccagattaacacagctggtgcagagatgctgtatcggactgtgagggagctgactggagtgaactctgacaccatccttcttgacatctgctgtggaactggtgtgattggcctctctctggctcagcatacatctcgggtccttgggattgaattgttggagcaggcagtggaggatgcaagatggactgcagccttcaatggcatcaccaactctgaatttcatactggtcaagcagagaagattttgccagggctgctaaagtcaaaggaagatggacagtcaattgttgctgtggtgaacccagcccgtgccggactgcattacaaggtgattcaagccattcgaaacttcagggccatccacacgctagtttttgtttcctgcaagctccatggtgaatccactaggaatgtcattgagctgtgctgtcctccagaccctgctaagaagctcttaggcgagccctttgtcctgcagcaagctgtacctgtggatttgttccctcacaccccacattgtgagctggtgctcctctttactcgataa

SEQ ID NO: 29 - DNA, 1929 bp, Homo sapiens
atggctgctgctgtacagcctgctgaggtgactgttgaagttggtgaggacctccacatgcaccaggttcgtgaccgggagatgcctgaagctttggagtttaacctttctgccaatccagaggcaagcacaatattccagaggaactctcaaacagatgctttggagtttaacccttctgccaatccagaggcaagcacaatattccagaggaactctcaaacagatgttgtagaaataagaagaagcaactgtacaaaccatgtatctactgtgcgtttcagtcaacaatacagcttgtgttcgacaatattccttgatgacagcacagccatccagcattatcttacaatgacaataatatctgtgaccttggagatacctcatcatatcacacaaagagatgcagatagatctttgagcatacctgatgaacagttacactcatttgcggtttccaccgtgcacattatgaagaaaagaaatggaggtgggagtttaaataactattcctcctccattccaccgactcccagcaccagccaggaggaccctcagttcagtgttcctcccactgccaacacacccacgcccgtttgcaagcggtccatgcgctggtccaacctgtttacatctgagaaagggagtgacccagacaaagagaggaaagccccggagaatcatgctgacaccatcgggagcggcagagccatccccattaaacagggcatgctcttaaagcgaagtgggaaatggctgaagacatggaaaaagaaatacgtcaccctgtgttccaatggcgtgctcacctattattcaagcttaggtgattatatgaagaatattcataaaaaagagattgaccttcagacatctaccatcaaagtcccaggaaagtggccatccctagccacatcggcctgcgcacccatctccagctctaaaagcaatggcctatccaaggacatggacatcgggctgggtgactccatatgcttcagccccagtatctccagcaccaccagccccaagctcaacccacccccctcccctcatgccaataaaaagaaacacctaaagaagaaaagcaccaacaactttatgattgtgtctgccactggccaaacgtggcactttgaagccacgacgtatgaggagcgggatgcatgggtccaagccatccagagccagatcctggccagcctgcagtcatgcgagagcagtaaaagcaagtcccagctgaccagccagagcgaggccatggccctgcagtcgatccaaaacatgcgtgggaacgcccactgtgtggactgtgagacccagaatcctaagtgggccagtttgaacttgggagtcctcatgtgtattgaatgctcaggtatccaccgcagtcttggcacccgcctttcccgtgtgcgatctctggagctggatgactggccagttgagctcaggaaggttatgtcatctattggcaatgacctagccaacagcatctgggaagggagcagccaggggcagacaaaaccctcagaaaagtccacgagggaagagaaagaacggtggatccgttccaaatatgaggagaagctctttctggccccactaccctgcactgagctgtccctgggccagcagctgctgcgggccaccgctgatgaggacctgcagacagccatcctgctgctggcacatggctcccgtgaggaggtgaacgagacctgtggggagggagacggctgcacggcgctccatctggcctgccgcaaggggaatgtggtcctggcgcagctcctgatctggtacggggtggacgtcatggcccgagatgcccacgggaacacagcgctgacctacgcccggcaggcctccagccaggagtgcatcaacgtgcttctgcagtacggctgccccgacgagtgcgtgtag

SEQ ID NO: 30 - DNA, 1929 bp, Homo sapiens
atggctgctgctgtacagcctgctgaggtgactgttgaagttggtgaggacctccacatgcaccaggttcgtgaccgggagatgcctgaagctttggagtttaacctttctgccaatccagaggcaagcacaatattccagaggaactctcaaacagatgctttggagtttaacccttctgccaatccagaggcaagcacaatattccagaggaactcacaaacagatgttgtagaaataagaagaagcaactgtacaaaccatgtatctactgtgcgtttcagtcaacaatacagcttgtgttcgacaatattccttgatgacagcacagccatccagcattatcttacaatgacaataatatctgtgaccttggagatacctcatcatatcacacaaagagatgcagatagatctttgagcatacctgatgaacagttacactcatttgcggtttccaccgtgcacattatgaagaaaagaaatggaggtgggagtttaaataactattcctcctccattccaccgactcccagcaccagccaggaggaccctcagttcagtgttcctcccactgccaacacacccacgcccgtttgcaagcggtccatgcgctggtccaacctgtttacatctgagaaagggagtgacccagacaaagagaggaaagccccggagaatcatgctgacaccatcgggagcggcagagccatccccattaaacagggcatgctcttaaagcgaagtgggaaatggctgaagacatggaaaaagaaatacgtcaccctgtgttccaatggcgtgctcacctattattcaagcttaggtgattatatgaagaatattcataaaaaagagattgaccttcagacatctaccatcaaagtcccaggaaagtggccatccctagccacatcggcctgcgcacccatctccagctctaaaagcaatggcctatccaaggacatggacatcgggctgggtgactccatatgcttcagccccagtatctccagcaccaccagccccaagctcaacccacccccctcccctcatgccaataaaaagaaacacctaaagaagaaaagcaccaacaactttatgattgtgtctgccactggccaaacgtggcactttgaagccacgacgtatgaggagcgggatgcatgggtccaagccatccagagccagatcctggccagcctgcagtcatgcgagagcagtaaaagcaagtcccagctgaccagccagagcgaggccatggccctgcagtcgatccaaaacatgcgtgggaacgcccactgtgtggactgtgagacccagaatcctaagtgggccagtttgaacttgggagtcctcatgtgtattgaatgctcaggtatccaccgcagtcttggcacccgcctttcccgtgtgcgatctctggagctggatgactggccagttgagctcaggaaggttatgtcatctattggcaatgacctagccaacagcatctgggaagggagcagccaggggcagacaaaaccctcagaaaagtccacgagggaagagaaagaacggtggatccgttccaaatatgaggagaagctctttctggccccactaccctgcactgagctgtccctgggccagcagctgctgcgggccaccgctgatgaggacctgcagacagccatcctgctgctggcacatggctcccgtgaggaggtgaacgagacctgtggggagggagacggctgcacggcgctccatctggcctgccgcaaggggaatgtggtcctggcgcagctcctgatctggtacggggtggacgtcatggcccgagatgcccacgggaacacagcgctgacctacgcccggcaggcctccagccaggagtgcatcaacgtgcttctgcagtacggctgccccgacgagtgcgtgtag

SEQ ID NO: 31 - DNA, 20 bp, Artificial Sequence
  Description of Artificial Sequence Synthetic primer
acgtctctgcattccctgtc

SEQ ID NO: 32 - DNA, 20 bp, Artificial Sequence
  Description of Artificial Sequence Synthetic primer
gctgacatccatgctgaaaa

SEQ ID NO: 33 - DNA, 21 bp, Artificial Sequence
  Description of Artificial Sequence Synthetic primer
agggagggctcactgagtaac

SEQ ID NO: 34 - DNA, 20 bp, Artificial Sequence
  Description of Artificial Sequence Synthetic primer
atgcacaaggaggaacatga

SEQ ID NO: 35 - DNA, 20 bp, Artificial Sequence
  Description of Artificial Sequence Synthetic primer
acctccctctgcttcgtgta

SEQ ID NO: 36 - DNA, 20 bp, Artificial Sequence
  Description of Artificial Sequence Synthetic primer
gcaggctatggaccaactgt

SEQ ID NO: 37 - DNA, 20 bp, Artificial Sequence
  Description of Artificial Sequence Synthetic primer
atgaggatgcccagaatcag

SEQ ID NO: 38 - DNA, 20 bp, Artificial Sequence
  Description of Artificial Sequence Synthetic primer
tgtatctaggggcaggacca

SEQ ID NO: 39 - DNA, 20 bp, Artificial Sequence
  Description of Artificial Sequence Synthetic primer
ctggtgtgtgtgtgtgtgga

SEQ ID NO: 40 - DNA, 20 bp, Artificial Sequence
  Description of Artificial Sequence Synthetic primer
gccgatcaaagacaaagcat

SEQ ID NO: 41 - DNA, 20 bp, Artificial Sequence
  Description of Artificial Sequence Synthetic primer
tggatgcctcctgaaagagt

SEQ ID NO: 42 - DNA, 20 bp, Artificial Sequence
  Description of Artificial Sequence Synthetic primer
ggcaaactacagcccaagac

SEQ ID NO: 43 - DNA, 20 bp, Artificial Sequence
  Description of Artificial Sequence Synthetic primer
agttagcggcccagctagaa

SEQ ID NO: 44 - DNA, 20 bp, Artificial Sequence
  Description of Artificial Sequence Synthetic primer
tcatcagatcgttgctcagg

SEQ ID NO: 45 - DNA, 20 bp, Artificial Sequence
  Description of Artificial Sequence Synthetic primer
tcctctgcactacagcatcg

SEQ ID NO: 46 - DNA, 20 bp, Artificial Sequence
  Description of Artificial Sequence Synthetic primer
ttccccacacatttcaatca

SEQ ID NO: 47 - DNA, 20 bp, Artificial Sequence
  Description of Artificial Sequence Synthetic primer
agccatcagcagtctcccta

SEQ ID NO: 48 - DNA, 20 bp, Artificial Sequence
  Description of Artificial Sequence Synthetic primer
aagggcacacaatggctatc

SEQ ID NO: 49 - DNA, 20 bp, Artificial Sequence
  Description of Artificial Sequence Synthetic primer
tcagtgccatctctgtggag

SEQ ID NO: 50 - DNA, 20 bp, Artificial Sequence
  Description of Artificial Sequence Synthetic primer
ggcatctttgggaagaacaa

SEQ ID NO: 51 - DNA, 20 bp, Artificial Sequence
  Description of Artificial Sequence Synthetic primer
tggtcctccataccagcttc

SEQ ID NO: 52 - DNA, 20 bp, Artificial Sequence
  Description of Artificial Sequence Synthetic primer
agtgctcagagccagagacc

SEQ ID NO: 53 - DNA, 20 bp, Artificial Sequence
  Description of Artificial Sequence Synthetic primer
tgacaggaaggctctggtct

SEQ ID NO: 54 - DNA, 20 bp, Artificial Sequence
  Description of Artificial Sequence Synthetic primer
tggttaggggtgacaggaag

SEQ ID NO: 55 - DNA, 20 bp, Artificial Sequence
  Description of Artificial Sequence Synthetic primer
aattgccatcagcagctctt

SEQ ID NO: 56 - DNA, 20 bp, Artificial Sequence
  Description of Artificial Sequence Synthetic primer
ctccatcaccgactccagtt

SEQ ID NO: 57 - DNA, 20 bp, Artificial Sequence
  Description of Artificial Sequence Synthetic primer
cgaatggcttgaatcacctt

SEQ ID NO: 58 - DNA, 20 bp, Artificial Sequence
  Description of Artificial Sequence Synthetic primer
gtccatcttgcatcctccac

SEQ ID NO: 59 - DNA, 20 bp, Artificial Sequence
  Description of Artificial Sequence Synthetic primer
caaaagggaaggcaacaatg

SEQ ID NO: 60 - DNA, 20 bp, Artificial Sequence
  Description of Artificial Sequence Synthetic primer
ggcatgaagaaagggagaga

SEQ ID NO: 61 - DNA, 20 bp, Artificial Sequence
  Description of Artificial Sequence Synthetic primer
ctccttagagggggttttgc

SEQ ID NO: 62 - DNA, 20 bp, Artificial Sequence
  Description of Artificial Sequence Synthetic primer
tttgccctccttaccttcct

SEQ ID NO: 63 - DNA, 19 bp, Artificial Sequence
  Description of Artificial Sequence Synthetic primer
acttacgaggcccgttgac

SEQ ID NO: 64 - DNA, 20 bp, Artificial Sequence
  Description of Artificial Sequence Synthetic primer
tttgtaggaggcaggtcagc

SEQ ID NO: 65 - DNA, 20 bp, Artificial Sequence
  Description of Artificial Sequence Synthetic primer
gcttggtcaccttggtgttt

SEQ ID NO: 66 - DNA, 20 bp, Artificial Sequence
  Description of Artificial Sequence Synthetic primer
gtgcctgtagcccagttgtt

SEQ ID NO: 67 - DNA, 20 bp, Artificial Sequence
  Description of Artificial Sequence Synthetic primer
ctcccaaagtgctgggatta

SEQ ID NO: 68 - DNA, 19 bp, Artificial Sequence
  Description of Artificial Sequence Synthetic primer
gtaccatgacccgttgcag

SEQ ID NO: 69 - DNA, 20 bp, Artificial Sequence
  Description of Artificial Sequence Synthetic primer
cttctttgtcttcgccatcc

SEQ ID NO: 70 - DNA, 20 bp, Artificial Sequence
  Description of Artificial Sequence Synthetic primer
tcacaatgacccagcaacat

SEQ ID NO: 71 - DNA, 20 bp, Artificial Sequence
  Description of Artificial Sequence Synthetic primer
ggaggaggcttctggaagag

SEQ ID NO: 72 - DNA, 20 bp, Artificial Sequence
  Description of Artificial Sequence Synthetic primer
gagagcgagggatcagacac

SEQ ID NO: 73 - DNA, 20 bp, Artificial Sequence
  Description of Artificial Sequence Synthetic primer
ccaagacaaatcatggcaaa

SEQ ID NO: 74 - DNA, 20 bp, Artificial Sequence
  Description of Artificial Sequence Synthetic primer
ggaggctgtcagtttctcca

SEQ ID NO: 75 - DNA, 20 bp, Artificial Sequence
  Description of Artificial Sequence Synthetic primer
cctgaacctcacctccaaaa

SEQ ID NO: 76 - DNA, 20 bp, Artificial Sequence
  Description of Artificial Sequence Synthetic primer
atttccacctgggaaaatcc

SEQ ID NO: 77 - DNA, 20 bp, Artificial Sequence
  Description of Artificial Sequence Synthetic primer
ctgagctggaaggagaatgc

SEQ ID NO: 78 - DNA, 20 bp, Artificial Sequence
  Description of Artificial Sequence Synthetic primer
acttggctgcagttgtaggg

SEQ ID NO: 79 - DNA, 20 bp, Artificial Sequence
  Description of Artificial Sequence Synthetic primer
ctgccttgcttcacaaagtg

SEQ ID NO: 80 - DNA, 20 bp, Artificial Sequence
  Description of Artificial Sequence Synthetic primer
ttaacagcagggctcgagtt

SEQ ID NO: 81 - DNA, 20 bp, Artificial Sequence
  Description of Artificial Sequence Synthetic primer
cccagcatgaggaagaagac

SEQ ID NO: 82 - DNA, 20 bp, Artificial Sequence
  Description of Artificial Sequence Synthetic primer
gcatcctggactcaggagag

SEQ ID NO: 83 - DNA, 20 bp, Artificial Sequence
  Description of Artificial Sequence Synthetic primer
ccacgttctacccaaactcc

SEQ ID NO: 84 - DNA, 20 bp, Artificial Sequence
  Description of Artificial Sequence Synthetic primer
gtggtggagaaggcagacat

SEQ ID NO: 85 - DNA, 20 bp, Artificial Sequence
  Description of Artificial Sequence Synthetic primer
ggagaagcgaatggacaaag

SEQ ID NO: 86 - DNA, 20 bp, Artificial Sequence
  Description of Artificial Sequence Synthetic primer
ccgtggagacagaaaggaag

SEQ ID NO: 87 - DNA, 20 bp, Artificial Sequence
  Description of Artificial Sequence Synthetic primer
gcagcctatttcacccactc

SEQ ID NO: 88 - DNA, 20 bp, Artificial Sequence
  Description of Artificial Sequence Synthetic primer
agatgcggtagtcgattggt

SEQ ID NO: 89 - DNA, 20 bp, Artificial Sequence
  Description of Artificial Sequence Synthetic primer
catggctcctccttcatcag

SEQ ID NO: 90 - DNA, 20 bp, Artificial Sequence
  Description of Artificial Sequence Synthetic primer
agctcctcggcagatcaata

SEQ ID NO: 91 - DNA, 20 bp, Artificial Sequence
  Description of Artificial Sequence Synthetic primer
ccgtctaaccgctacgagac

SEQ ID NO: 92 - DNA, 20 bp, Artificial Sequence
  Description of Artificial Sequence Synthetic primer
gggcagaatgatctggaaaa

SEQ ID NO: 93 - DNA, 20 bp, Artificial Sequence
  Description of Artificial Sequence Synthetic primer
gtggatcttgaacgcacctt

SEQ ID NO: 94 - DNA, 20 bp, Artificial Sequence
  Description of Artificial Sequence Synthetic primer
gagggagcatgtggagaaac

SEQ ID NO: 95 - DNA, 20 bp, Artificial Sequence
  Description of Artificial Sequence Synthetic primer
ggctcaggtggaaaacaatc

SEQ ID NO: 96 - DNA, 20 bp, Artificial Sequence
  Description of Artificial Sequence Synthetic primer
ccagctctctgggaactcac

SEQ ID NO: 97 - DNA, 20 bp, Artificial Sequence
  Description of Artificial Sequence Synthetic primer
tttcggtggctctttcaagt

SEQ ID NO: 98 - DNA, 20 bp, Artificial Sequence
  Description of Artificial Sequence Synthetic primer
gggggaacatatggtgactg

SEQ ID NO: 99 - DNA, 20 bp, Artificial Sequence
  Description of Artificial Sequence Synthetic primer
actgacccatgcacacaaag

SEQ ID NO: 100 - DNA, 20 bp, Artificial Sequence
  Description of Artificial Sequence Synthetic primer
ttctcgttggggcaagtaac

SEQ ID NO: 101 - DNA, 20 bp, Artificial Sequence
  Description of Artificial Sequence Synthetic primer
aagctgggaccaaggaactt

SEQ ID NO: 102 - DNA, 20 bp, Artificial Sequence
  Description of Artificial Sequence Synthetic primer
agctctgcgtactccacgat

SEQ ID NO: 103 - DNA, 20 bp, Artificial Sequence
  Description of Artificial Sequence Synthetic primer
tactgcaggattgtgccttg

SEQ ID NO: 104 - DNA, 20 bp, Artificial Sequence
  Description of Artificial Sequence Synthetic primer
aaatctgctccagctccaaa

SEQ ID NO: 105 - DNA, 20 bp, Artificial Sequence
  Description of Artificial Sequence Synthetic primer
aacattcctcccagcatgac

SEQ ID NO: 106 - DNA, 20 bp, Artificial Sequence
  Description of Artificial Sequence Synthetic primer
tgtgcagtagggttgtcctg

SEQ ID NO: 107 - DNA, 20 bp, Artificial Sequence
  Description of Artificial Sequence Synthetic primer
aacaccatctcctgcagctc

SEQ ID NO: 108 - DNA, 20 bp, Artificial Sequence
  Description of Artificial Sequence Synthetic primer
aaaggtgggagcctttccta

SEQ ID NO: 109 - DNA, 18 bp, Artificial Sequence
  Description of Artificial Sequence Synthetic primer
gtgcaggagccagagagg

SEQ ID NO: 110 - DNA, 20 bp, Artificial Sequence
  Description of Artificial Sequence Synthetic primer
ggtggaggaatgtgggtcta

SEQ ID NO: 111 - DNA, 20 bp, Artificial Sequence
  Description of Artificial Sequence Synthetic primer
ccatcacaaccaccacaaag

SEQ ID NO: 112 - DNA, 20 bp, Artificial Sequence
  Description of Artificial Sequence Synthetic primer
ggtgaggcgtttgttgtttt

SEQ ID NO: 113 - DNA, 20 bp, Artificial Sequence
  Description of Artificial Sequence Synthetic primer
gctcctcggcagatcaatag

SEQ ID NO: 114 - DNA, 20 bp, Artificial Sequence
  Description of Artificial Sequence Synthetic primer
tgttgcatgtgtcttgagca

SEQ ID NO: 115 - DNA, 20 bp, Artificial Sequence
  Description of Artificial Sequence Synthetic primer
gcaggaagtcatggagaagc

SEQ ID NO: 116 - DNA, 20 bp, Artificial Sequence
  Description of Artificial Sequence Synthetic primer
ggagcatgtggagaaactgg

SEQ ID NO: 117 - DNA, 20 bp, Artificial Sequence
  Description of Artificial Sequence Synthetic primer
ggtttcctccctcctcagac

SEQ ID NO: 118 - DNA, 20 bp, Artificial Sequence
  Description of Artificial Sequence Synthetic primer
agtctcctcccccatcagac

SEQ ID NO: 119 - DNA, 20 bp, Artificial Sequence
  Description of Artificial Sequence Synthetic primer
agcagtggggagatgctcta

SEQ ID NO: 120 - DNA, 20 bp, Artificial Sequence
  Description of Artificial Sequence Synthetic primer
ggtgaccctgcagatttgac

SEQ ID NO: 121 - DNA, 20 bp, Artificial Sequence
  Description of Artificial Sequence Synthetic primer
ggaagccttcgcttagatcc

SEQ ID NO: 122 - DNA, 20 bp, Artificial Sequence
  Description of Artificial Sequence Synthetic primer
gcgaattcgatctctcctca

SEQ ID NO: 123 - DNA, 20 bp, Artificial Sequence
  Description of Artificial Sequence Synthetic primer
tcccaaagtgctgggattac

SEQ ID NO: 124 - DNA, 20 bp, Artificial Sequence
  Description of Artificial Sequence Synthetic primer
cctgaggtcaggagttcgag

SEQ ID NO: 125 - DNA, 20 bp, Artificial Sequence
  Description of Artificial Sequence Synthetic primer
aaaaattgcagccaacatcc

SEQ ID NO: 126 - DNA, 20 bp, Artificial Sequence
  Description of Artificial Sequence Synthetic primer
ggctctactcaccaccatgc

SEQ ID NO: 127 - DNA, 19 bp, Artificial Sequence
  Description of Artificial Sequence Synthetic primer
gagagagaggcggggatct

SEQ ID NO: 128 - DNA, 20 bp, Artificial Sequence
  Description of Artificial Sequence Synthetic primer
gagcaccaggacaacgtctt

SEQ ID NO: 129 - DNA, 20 bp, Artificial Sequence
  Description of Artificial Sequence Synthetic primer
tcgggagaaaaaggaggaat

SEQ ID NO: 130 - DNA, 20 bp, Artificial Sequence
  Description of Artificial Sequence Synthetic primer
catctacgtgtcgcctctca

SEQ ID NO: 131 - DNA, 20 bp, Artificial Sequence
  Description of Artificial Sequence Synthetic primer
cctgacctccctcagcaata

SEQ ID NO: 132 - DNA, 20 bp, Artificial Sequence
  Description of Artificial Sequence Synthetic primer
ttcgcaacatgatcaactcc

SEQ ID NO: 133 - DNA, 20 bp, Artificial Sequence
  Description of Artificial Sequence Synthetic primer
atgaaacctacagcggcatc

SEQ ID NO: 134 - DNA, 20 bp, Artificial Sequence
  Description of Artificial Sequence Synthetic primer
agcgcactcatcaggaaact

SEQ ID NO: 135 - DNA, 20 bp, Artificial Sequence
  Description of Artificial Sequence Synthetic primer
aagcagggctacatgagtgg

SEQ ID NO: 136 - DNA, 20 bp, Artificial Sequence
  Description of Artificial Sequence Synthetic primer
acgaccctgaagaacccatt

SEQ ID NO: 137 - DNA, 20 bp, Artificial Sequence
  Description of Artificial Sequence Synthetic primer
agcgaacaaggacgtaatgg

SEQ ID NO: 138 - DNA, 20 bp, Artificial Sequence
  Description of Artificial Sequence Synthetic primer
aagtgaacaaggctgggatg

SEQ ID NO: 139 - DNA, 20 bp, Artificial Sequence
  Description of Artificial Sequence Synthetic primer
ggggtgtacacattcccaac

SEQ ID NO: 140 - DNA, 20 bp, Artificial Sequence
  Description of Artificial Sequence Synthetic primer
cctgccgtagagaaatggaa

SEQ ID NO: 141 - DNA, 19 bp, Artificial Sequence
  Description of Artificial Sequence Synthetic primer
cctcacgctcattcctcag

SEQ ID NO: 142 - DNA, 20 bp, Artificial Sequence
  Description of Artificial Sequence Synthetic primer
tgctttcaagtgggagcttt

SEQ ID NO: 143 - DNA, 20 bp, Artificial Sequence
  Description of Artificial Sequence Synthetic primer
atttgggggtacctcctttg

SEQ ID NO: 144 - DNA, 20 bp, Artificial Sequence
  Description of Artificial Sequence Synthetic primer
tcctaggaggtggtccagtg

SEQ ID NO: 145 - DNA, 20 bp, Artificial Sequence
  Description of Artificial Sequence Synthetic primer
ctagaggcgcctgagatttg

SEQ ID NO: 146 - DNA, 20 bp, Artificial Sequence
  Description of Artificial Sequence Synthetic primer
tttcaaaacccaaacccttg

SEQ ID NO: 147 - DNA, 20 bp, Artificial Sequence
  Description of Artificial Sequence Synthetic primer
tcaaactcacgaaggtgcag

SEQ ID NO: 148 - DNA, 20 bp, Artificial Sequence
  Description of Artificial Sequence Synthetic primer
actgcaagatcagagcagca

SEQ ID NO: 149 - DNA, 20 bp, Artificial Sequence
  Description of Artificial Sequence Synthetic primer
cagcaaccaggaccctctac

SEQ ID NO: 150 - DNA, 20 bp, Artificial Sequence
  Description of Artificial Sequence Synthetic primer
tacgtcgtgcaggtcttcag

SEQ ID NO: 151 - DNA, 20 bp, Artificial Sequence
  Description of Artificial Sequence Synthetic primer
tccccacagttctctgctct

SEQ ID NO: 152 - DNA, 20 bp, Artificial Sequence
  Description of Artificial Sequence Synthetic primer
caacatgtggtgagcattcc

SEQ ID NO: 153 - DNA, 20 bp, Artificial Sequence
  Description of Artificial Sequence Synthetic primer
ggtcatgtccttggcagtct

SEQ ID NO: 154 - DNA, 20 bp, Artificial Sequence
  Description of Artificial Sequence Synthetic primer
gcaccctcaaaagactctgc

SEQ ID NO: 155 - DNA, 20 bp, Artificial Sequence
  Description of Artificial Sequence Synthetic primer
tccctcctgatcccttttct

SEQ ID NO: 156 - DNA, 20 bp, Artificial Sequence
  Description of Artificial Sequence Synthetic primer
catgagtggaggagcaggat

SEQ ID NO: 157 - DNA, 20 bp, Artificial Sequence
  Description of Artificial Sequence Synthetic primer
cacgttccctcatttcttgg

SEQ ID NO: 158 - DNA, 20 bp, Artificial Sequence
  Description of Artificial Sequence Synthetic primer
caggctggtcttgaactcct

SEQ ID NO: 159 - DNA, 20 bp, Artificial Sequence
  Description of Artificial Sequence Synthetic primer
ttctacaagggctggtgtcc

SEQ ID NO: 160 - DNA, 20 bp, Artificial Sequence
  Description of Artificial Sequence Synthetic primer
ggctcttgtgttgccttagc

SEQ ID NO: 161 - DNA, 20 bp, Artificial Sequence
  Description of Artificial Sequence Synthetic primer
gaggctgaggctgaggtcta

SEQ ID NO: 162 - DNA, 20 bp, Artificial Sequence
  Description of Artificial Sequence Synthetic primer
gctgtccaagcaaggaatgt

SEQ ID NO: 163 - DNA, 20 bp, Artificial Sequence
  Description of Artificial Sequence Synthetic primer
cattctcagtgcctggcata

SEQ ID NO: 164 - DNA, 20 bp, Artificial Sequence
  Description of Artificial Sequence Synthetic primer
tgctgatgtccctgttctca

SEQ ID NO: 165 - DNA, 20 bp, Artificial Sequence
  Description of Artificial Sequence Synthetic primer
tggagctgcctctctgtagg

SEQ ID NO: 166 - DNA, 19 bp, Artificial Sequence
  Description of Artificial Sequence Synthetic primer
gcccgagtaaggacgacat

SEQ ID NO: 167 - DNA, 20 bp, Artificial Sequence
  Description of Artificial Sequence Synthetic primer
cacttgtttggttgggttga

SEQ ID NO: 168 - DNA, 20 bp, Artificial Sequence
  Description of Artificial Sequence Synthetic primer
cagctcctgggtctgtaagc

SEQ ID NO: 169 - DNA, 20 bp, Artificial Sequence
  Description of Artificial Sequence Synthetic primer
ttttcctggattgcgaattt

SEQ ID NO: 170 - DNA, 8 bp, Artificial Sequence
  Description of Artificial Sequence Synthetic primer
aggacacg

SEQ ID NO: 171 - DNA, 20 bp, Artificial Sequence
  Description of Artificial Sequence Synthetic primer
gcgcctctcgctgaagtagt

SEQ ID NO: 172 - DNA, 20 bp, Artificial Sequence
  Description of Artificial Sequence Synthetic primer
ttgtggacgtgattcccttt

SEQ ID NO: 173 - DNA, 20 bp, Artificial Sequence
  Description of Artificial Sequence Synthetic primer
caagtatgagcgcaccttca

SEQ ID NO: 174 - DNA, 20 bp, Artificial Sequence
  Description of Artificial Sequence Synthetic primer
gtgctcagccagaacacaaa

SEQ ID NO: 175 - DNA, 20 bp, Artificial Sequence
  Description of Artificial Sequence Synthetic primer
ctggaggacatggtcctgat

SEQ ID NO: 176 - DNA, 20 bp, Artificial Sequence
  Description of Artificial Sequence Synthetic primer
ttgcccactatgaacagcag

SEQ ID NO: 177 - DNA, 20 bp, Artificial Sequence
  Description of Artificial Sequence Synthetic primer
accctgaacacccacaaagt

SEQ ID NO: 178 - DNA, 20 bp, Artificial Sequence
  Description of Artificial Sequence Synthetic primer
cgaaaatgctgaaatgtcca

SEQ ID NO: 179 - DNA, 20 bp, Artificial Sequence
  Description of Artificial Sequence Synthetic primer
gggaagcagatggagttcaa

SEQ ID NO: 180 - DNA, 21 bp, Artificial Sequence
  Description of Artificial Sequence Synthetic primer
tcggttttcatttttcacagc

SEQ ID NO: 181 - DNA, 7 bp, Artificial Sequence
  Description of Artificial Sequence Synthetic primer
tgtccct

SEQ ID NO: 182 - DNA, 18 bp, Artificial Sequence
  Description of Artificial Sequence Synthetic primer
cgagtcgcccttctctcc

SEQ ID NO: 183 - DNA, 20 bp, Artificial Sequence
  Description of Artificial Sequence Synthetic primer
agggtgtttgctgtggtctc

SEQ ID NO: 184 - DNA, 21 bp, Artificial Sequence
  Description of Artificial Sequence Synthetic primer
atgtggcatggcttacttcct

SEQ ID NO: 185 - DNA, 20 bp, Artificial Sequence
  Description of Artificial Sequence Synthetic primer
acaatgcgagggttcttgat

SEQ ID NO: 186 - DNA, 20 bp, Artificial Sequence
  Description of Artificial Sequence Synthetic primer
ggggatcctgctaaaccttg

SEQ ID NO: 187 - DNA, 20 bp, Artificial Sequence
  Description of Artificial Sequence Synthetic primer
tgaagcctgtgcagactcag

SEQ ID NO: 188 - DNA, 20 bp, Artificial Sequence
  Description of Artificial Sequence Synthetic primer
gggttccttttgtcctttcc

SEQ ID NO: 189 - DNA, 20 bp, Artificial Sequence
  Description of Artificial Sequence Synthetic primer
aggaatatccgacgcatcaa

SEQ ID NO: 190 - DNA, 19 bp, Artificial Sequence
  Description of Artificial Sequence Synthetic primer
ctgaaaaacgtccccgact

SEQ ID NO: 191 - DNA, 10 bp, Artificial Sequence
  Description of Artificial Sequence Synthetic primer
atgtaccgga

SEQ ID NO: 192 - DNA, 19 bp, Artificial Sequence
  Description of Artificial Sequence Synthetic primer
gtcttgtgtgccccacgtc

SEQ ID NO: 193 - DNA, 20 bp, Artificial Sequence
  Description of Artificial Sequence Synthetic primer
ctgagcctctccttgtcctg

SEQ ID NO: 194 - DNA, 20 bp, Artificial Sequence
  Description of Artificial Sequence Synthetic primer
cgtggaaagaacttggaacc

SEQ ID NO: 195 - DNA, 20 bp, Artificial Sequence
  Description of Artificial Sequence Synthetic primer
agggaattaaggcaccgtct

SEQ ID NO: 196 - DNA, 22 bp, Artificial Sequence
  Description of Artificial Sequence Synthetic primer
tcctacacagtgaccaatgtcc

SEQ ID NO: 197 - DNA, 20 bp, Artificial Sequence
  Description of Artificial Sequence Synthetic primer
aaaaagcgggatgacttttg

SEQ ID NO: 198 - DNA, 20 bp, Artificial Sequence
  Description of Artificial Sequence Synthetic primer
tgcagaagcctggagaagtt

SEQ ID NO: 199 - DNA, 20 bp, Artificial Sequence
  Description of Artificial Sequence Synthetic primer
aggtttcaaaagccacagga

SEQ ID NO: 200 - DNA, 21 bp, Artificial Sequence
  Description of Artificial Sequence Synthetic primer
gctgcataagaacctcctggt

SEQ ID NO: 201 - DNA, 20 bp, Artificial Sequence
  Description of Artificial Sequence Synthetic primer
gattgcttcctacagcagcc

SEQ ID NO: 202 - DNA, 20 bp, Artificial Sequence
  Description of Artificial Sequence Synthetic primer
agtccatcatgtccctccac

SEQ ID NO: 203 - DNA, 20 bp, Artificial Sequence
  Description of Artificial Sequence Synthetic primer
tggcatctaagttggctgtg

SEQ ID NO: 204 - DNA, 20 bp, Artificial Sequence
  Description of Artificial Sequence Synthetic primer
cagtggcatgtgcctgtagt

SEQ ID NO: 205 - DNA, 20 bp, Artificial Sequence
  Description of Artificial Sequence Synthetic primer
gtctcaaaggagcctccaaa

SEQ ID NO: 206 - DNA, 20 bp, Artificial Sequence
  Description of Artificial Sequence Synthetic primer
acaggaaaagcccagctgta

SEQ ID NO: 207 - DNA, 20 bp, Artificial Sequence
  Description of Artificial Sequence Synthetic primer
gccggttatgaaaaggaagg

SEQ ID NO: 208 - DNA, 20 bp, Artificial Sequence
  Description of Artificial Sequence Synthetic primer
tgcccactatcaatgagcac

SEQ ID NO: 209 - DNA, 18 bp, Artificial Sequence
  Description of Artificial Sequence Synthetic primer
aaaaacgtccccgactcc

SEQ ID NO: 210 - DNA, 18 bp, Artificial Sequence
  Description of Artificial Sequence Synthetic primer
acgaggcacacaggaagg

SEQ ID NO: 211 - DNA, 20 bp, Artificial Sequence
  Description of Artificial Sequence Synthetic primer
gccatcccggactatacaga

SEQ ID NO: 212 - DNA, 20 bp, Artificial Sequence
  Description of Artificial Sequence Synthetic primer
cgcagatcaaggccaactat

SEQ ID NO: 213 - DNA, 20 bp, Artificial Sequence
  Description of Artificial Sequence Synthetic primer
ggggagaacaccaaaacaaa

SEQ ID NO: 214 - DNA, 20 bp, Artificial Sequence
  Description of Artificial Sequence Synthetic primer
gtgagaaggcctcaggacag

SEQ ID NO: 215 - DNA, 20 bp, Artificial Sequence
  Description of Artificial Sequence Synthetic primer
ttcctagggctggaaggttt

SEQ ID NO: 216 - DNA, 20 bp, Artificial Sequence
  Description of Artificial Sequence Synthetic primer
ccgagatacaggcatcttcc

SEQ ID NO: 217 - DNA, 20 bp, Artificial Sequence
  Description of Artificial Sequence Synthetic primer
tcgtgggtaacttgctggat

SEQ ID NO: 218 - DNA, 20 bp, Artificial Sequence
  Description of Artificial Sequence Synthetic primer
aggccattctcttccttggt

SEQ ID NO: 219 - DNA, 20 bp, Artificial Sequence
  Description of Artificial Sequence Synthetic primer
ataatgttgcagcccagcag

SEQ ID NO: 220 - DNA, 20 bp, Artificial Sequence
  Description of Artificial Sequence Synthetic primer
gctgagagcctgttccatgt

SEQ ID NO: 221 - DNA, 20 bp, Artificial Sequence
  Description of Artificial Sequence Synthetic primer
gtgaggagagaaccgactgg

SEQ ID NO: 222 - DNA, 20 bp, Artificial Sequence
  Description of Artificial Sequence Synthetic primer
tagctgccaaagaaacagca

SEQ ID NO: 223 - DNA, 20 bp, Artificial Sequence
  Description of Artificial Sequence Synthetic primer
aaacccatgagcatcaaacc

SEQ ID NO: 224 - DNA, 20 bp, Artificial Sequence
  Description of Artificial Sequence Synthetic primer
caggtgcactggaaggaact

SEQ ID NO: 225 - DNA, 20 bp, Artificial Sequence
  Description of Artificial Sequence Synthetic primer
tgtgagatcactggctttgg

SEQ ID NO: 226 - DNA, 20 bp, Artificial Sequence
  Description of Artificial Sequence Synthetic primer
aggccttcacagtgtctggt

SEQ ID NO: 227 - DNA, 20 bp, Artificial Sequence
  Description of Artificial Sequence Synthetic primer
tcaaatcgtgctcatggaaa

SEQ ID NO: 228 - DNA, 20 bp, Artificial Sequence
  Description of Artificial Sequence Synthetic primer
agagggggtgcattagtcct

SEQ ID NO: 229 - DNA, 20 bp, Artificial Sequence
  Description of Artificial Sequence Synthetic primer
tttctggtcctaagcaagca

SEQ ID NO: 230 - DNA, 20 bp, Artificial Sequence
  Description of Artificial Sequence Synthetic primer
agggagacccctagattcca

SEQ ID NO: 231 - DNA, 20 bp, Artificial Sequence
  Description of Artificial Sequence Synthetic primer
tccagtgctcatgccaagta

SEQ ID NO: 232 - DNA, 20 bp, Artificial Sequence
  Description of Artificial Sequence Synthetic primer
gattgggaaacactgctgaa

SEQ ID NO: 233 - DNA, 20 bp, Artificial Sequence
  Description of Artificial Sequence Synthetic primer
ccgtatcattgtgtgggaca

SEQ ID NO: 234 - DNA, 20 bp, Artificial Sequence
  Description of Artificial Sequence Synthetic primer
tccctttgacccacagtctc

SEQ ID NO: 235 - DNA, 20 bp, Artificial Sequence
  Description of Artificial Sequence Synthetic primer
tgctagaaccccaagactcc

SEQ ID NO: 236 - DNA, 20 bp, Artificial Sequence
  Description of Artificial Sequence Synthetic primer
ctgtgcctcctaaccagctc

SEQ ID NO: 237 - DNA, 20 bp, Artificial Sequence
  Description of Artificial Sequence Synthetic primer
gtggagagagatgcttggag

SEQ ID NO: 238 - DNA, 20 bp, Artificial Sequence
  Description of Artificial Sequence Synthetic primer
agacaccaaattggctggtg

SEQ ID NO: 239 - DNA, 20 bp, Artificial Sequence
  Description of Artificial Sequence Synthetic primer
aggacccagaatgggaagac

SEQ ID NO: 240 - DNA, 20 bp, Artificial Sequence
  Description of Artificial Sequence Synthetic primer
cacttgggaagaagggatga

SEQ ID NO: 241 - DNA, 20 bp, Artificial Sequence
  Description of Artificial Sequence Synthetic primer
ctgcaggactccatctgctt

SEQ ID NO: 242 - DNA, 20 bp, Artificial Sequence
  Description of Artificial Sequence Synthetic primer
ttgagtccaggaggtcaagg

SEQ ID NO: 243 - DNA, 20 bp, Artificial Sequence
  Description of Artificial Sequence Synthetic primer
taccatctgccttccaggtc

SEQ ID NO: 244 - DNA, 20 bp, Artificial Sequence
  Description of Artificial Sequence Synthetic primer
accatgcaggagaaggtgtc

SEQ ID NO: 245 - DNA, 20 bp, Artificial Sequence
  Description of Artificial Sequence Synthetic primer
tgggctataatcccatgcac

SEQ ID NO: 246 - DNA, 20 bp, Artificial Sequence
  Description of Artificial Sequence Synthetic primer
agggttcacaaaaggtgtcg

SEQ ID NO: 247 - DNA, 20 bp, Artificial Sequence
  Description of Artificial Sequence Synthetic primer
ctggggacacattccatctt

SEQ ID NO: 248 - DNA, 20 bp, Artificial Sequence
  Description of Artificial Sequence Synthetic primer
ggtacttcaccaccgtgctc

SEQ ID NO: 249 - DNA, 20 bp, Artificial Sequence
  Description of Artificial Sequence Synthetic primer
tagctctgcaggggactcat

SEQ ID NO: 250 - DNA, 20 bp, Artificial Sequence
  Description of Artificial Sequence Synthetic primer
agtttggtctccctggtcct

SEQ ID NO: 251 - DNA, 20 bp, Artificial Sequence
  Description of Artificial Sequence Synthetic primer
tagctgcaagtttccggttt

SEQ ID NO: 252 - DNA, 20 bp, Artificial Sequence
  Description of Artificial Sequence Synthetic primer
ggttgctcaggttccatgtt

SEQ ID NO: 253 - DNA, 20 bp, Artificial Sequence
  Description of Artificial Sequence Synthetic primer
ttttcacccttaggcccttt

SEQ ID NO: 254 - DNA, 20 bp, Artificial Sequence
  Description of Artificial Sequence Synthetic primer
tctgggtgcagcatgataag

SEQ ID NO: 255 - DNA, 20 bp, Artificial Sequence
  Description of Artificial Sequence Synthetic primer
ctgaggcctgtctggttagc

SEQ ID NO: 256 - DNA, 20 bp, Artificial Sequence
  Description of Artificial Sequence Synthetic primer
gacttcctgtcttggcggta

SEQ ID NO: 257 - DNA, 20 bp, Artificial Sequence
  Description of Artificial Sequence Synthetic primer
taaaaagcagccctccaaca

SEQ ID NO: 258 - DNA, 20 bp, Artificial Sequence
  Description of Artificial Sequence Synthetic primer
ggcaggattcaaagaccaag

SEQ ID NO: 259 - DNA, 20 bp, Artificial Sequence
  Description of Artificial Sequence Synthetic primer
ggggtctggtttctcatcct

SEQ ID NO: 260 - DNA, 20 bp, Artificial Sequence
  Description of Artificial Sequence Synthetic primer
ttcataaccggcacctcttc

SEQ ID NO: 261 - DNA, 20 bp, Artificial Sequence
  Description of Artificial Sequence Synthetic primer
cttggaggacaggcatcatt

SEQ ID NO: 262 - DNA, 20 bp, Artificial Sequence
  Description of Artificial Sequence Synthetic primer
ctgccttctagccctgtgag

SEQ ID NO: 263 - DNA, 20 bp, Artificial Sequence
  Description of Artificial Sequence Synthetic primer
aaccgtggaacttgtccttg

SEQ ID NO: 264 - DNA, 20 bp, Artificial Sequence
  Description of Artificial Sequence Synthetic primer
tggcttcgtttagcctccta

SEQ ID NO: 265 - DNA, 20 bp, Artificial Sequence
  Description of Artificial Sequence Synthetic primer
ggtcttctaggccacagtcg

SEQ ID NO: 266 - DNA, 20 bp, Artificial Sequence
  Description of Artificial Sequence Synthetic primer
atcagtgtgtgggggttttt

SEQ ID NO: 267 - DNA, 20 bp, Artificial Sequence
  Description of Artificial Sequence Synthetic primer
agtgagctcgtgaccctgag

SEQ ID NO: 268 - DNA, 20 bp, Artificial Sequence
  Description of Artificial Sequence Synthetic primer
ctctctggctcagggttcag

SEQ ID NO: 269 - DNA, 20 bp, Artificial Sequence
  Description of Artificial Sequence Synthetic primer
gggacccaggaaattgtacc

SEQ ID NO: 270 - DNA, 20 bp, Artificial Sequence
  Description of Artificial Sequence Synthetic primer
attcgccacttggaaaaatg

SEQ ID NO: 271 - DNA, 20 bp, Artificial Sequence
  Description of Artificial Sequence Synthetic primer
accagaaaaaggcctccagt

SEQ ID NO: 272 - DNA, 20 bp, Artificial Sequence
  Description of Artificial Sequence Synthetic primer
ggccaaccacgtgactctat

SEQ ID NO: 273 - DNA, 20 bp, Artificial Sequence
  Description of Artificial Sequence Synthetic primer
gtagcagcaatccccaaaaa

SEQ ID NO: 274 - DNA, 20 bp, Artificial Sequence
  Description of Artificial Sequence Synthetic primer
cacacatccaggtcttgtgc

SEQ ID NO: 275 - DNA, 20 bp, Artificial Sequence
  Description of Artificial Sequence Synthetic primer
actcttggcctcaagcaatc

SEQ ID NO: 276 - DNA, 20 bp, Artificial Sequence
  Description of Artificial Sequence Synthetic primer
agcagagacgtgagcacaga

SEQ ID NO: 277 - DNA, 20 bp, Artificial Sequence
  Description of Artificial Sequence Synthetic primer
agcatttgtttcctgctgct

SEQ ID NO: 278 - DNA, 20 bp, Artificial Sequence
  Description of Artificial Sequence Synthetic primer
cccgagtagctgggattaca

SEQ ID NO: 279 - DNA, 20 bp, Artificial Sequence
  Description of Artificial Sequence Synthetic primer
ccaggaaagaattgccagag

SEQ ID NO: 280 - DNA, 20 bp, Artificial Sequence
  Description of Artificial Sequence Synthetic primer
gatctgttgtggctggcttt

SEQ ID NO: 281 - DNA, 20 bp, Artificial Sequence
  Description of Artificial Sequence Synthetic primer
agtcgcggagtagtcctcat

SEQ ID NO: 282 - DNA, 20 bp, Artificial Sequence
  Description of Artificial Sequence Synthetic primer
aaggtacccacctcgatcct

SEQ ID NO: 283 - DNA, 20 bp, Artificial Sequence
  Description of Artificial Sequence Synthetic primer
ccatggggaacatactgacc

SEQ ID NO: 284 - DNA, 20 bp, Artificial Sequence
  Description of Artificial Sequence Synthetic primer
tgcctgaagctttggagttt

SEQ ID NO: 285 - DNA, 20 bp, Artificial Sequence
  Description of Artificial Sequence Synthetic primer
cctgctgaggtgactgttga

SEQ ID NO: 286 - DNA, 20 bp, Artificial Sequence
  Description of Artificial Sequence Synthetic primer
acagaggaagcacggagaaa

SEQ ID NO: 287 - DNA, 20 bp, Artificial Sequence
  Description of Artificial Sequence Synthetic primer
cctgcatacttccccaaaaa

SEQ ID NO: 288 - DNA, 20 bp, Artificial Sequence
  Description of Artificial Sequence Synthetic primer
tttgccacactgccaataaa

SEQ ID NO: 289 - DNA, 20 bp, Artificial Sequence
  Description of Artificial Sequence Synthetic primer
tggcaagttttcattccaca

SEQ ID NO: 290 - DNA, 21 bp, Artificial Sequence
  Description of Artificial Sequence Synthetic primer
gcagtagacacagggccagaa

SEQ ID NO: 291 - DNA, 20 bp, Artificial Sequence
  Description of Artificial Sequence Synthetic primer
gctgtgctgtcatcaaggaa

SEQ ID NO: 292 - DNA, 20 bp, Artificial Sequence
  Description of Artificial Sequence Synthetic primer
ccatttcccacttcgcttta

SEQ ID NO: 293 - DNA, 20 bp, Artificial Sequence
  Description of Artificial Sequence Synthetic primer
cgatggtgtcagcatgattc

SEQ ID NO: 294 - DNA, 20 bp, Artificial Sequence
  Description of Artificial Sequence Synthetic primer
ttttcagtccaaagggcatc

SEQ ID NO: 295 - DNA, 20 bp, Artificial Sequence
  Description of Artificial Sequence Synthetic primer
aaaactggctctgccttgaa

SEQ ID NO: 296 - DNA, 20 bp, Artificial Sequence
  Description of Artificial Sequence Synthetic primer
tggctcttgtcgaataagca

SEQ ID NO: 297 - DNA, 20 bp, Artificial Sequence
  Description of Artificial Sequence Synthetic primer
ccacttcgagctcctttctg

SEQ ID NO: 298 - DNA, 20 bp, Artificial Sequence
  Description of Artificial Sequence Synthetic primer
tggcatgggaccatttattt

SEQ ID NO: 299 - DNA, 20 bp, Artificial Sequence
  Description of Artificial Sequence Synthetic primer
ccaccgagtccagtcagttt

SEQ ID NO: 300 - DNA, 20 bp, Artificial Sequence
  Description of Artificial Sequence Synthetic primer
gctggagtgtagtggcatga

SEQ ID NO: 301 - DNA, 20 bp, Artificial Sequence
  Description of Artificial Sequence Synthetic primer
ggactgtgccttgccataat

SEQ ID NO: 302 - DNA, 20 bp, Artificial Sequence
  Description of Artificial Sequence Synthetic primer
cccgagtagctgggactaca

SEQ ID NO: 303 - DNA, 20 bp, Artificial Sequence
  Description of Artificial Sequence Synthetic primer
gtggcaacctccattctgat

SEQ ID NO: 304 - DNA, 20 bp, Artificial Sequence
  Description of Artificial Sequence Synthetic primer
tgagaaaccccaaacacaca

SEQ ID NO: 305 - DNA, 20 bp, Artificial Sequence
  Description of Artificial Sequence Synthetic primer
tggcaacctccattctgatt

SEQ ID NO: 306 - DNA, 21 bp, Artificial Sequence
  Description of Artificial Sequence Synthetic primer
gtgggcaacctccattctgat

SEQ ID NO: 307 - DNA, 20 bp, Artificial Sequence
  Description of Artificial Sequence Synthetic primer
gcacttacctggttccttca

SEQ ID NO: 308 - DNA, 20 bp, Artificial Sequence
  Description of Artificial Sequence Synthetic primer
tctatgtgggcctggaagat

SEQ ID NO: 309 - DNA, 20 bp, Artificial Sequence
  Description of Artificial Sequence Synthetic primer
ttctatgtgggcctggaaga

SEQ ID NO: 310 - DNA, 20 bp, Artificial Sequence
  Description of Artificial Sequence Synthetic primer
ttgtcgtggatttgcacagt

SEQ ID NO: 311 - DNA, 20 bp, Artificial Sequence
  Description of Artificial Sequence Synthetic primer
gaagcatttcctgggatcag

SEQ ID NO: 312 - DNA, 19 bp, Artificial Sequence
  Description of Artificial Sequence Synthetic primer
gaaagccttcctgctcctg

SEQ ID NO: 313 - DNA, 20 bp, Artificial Sequence
  Description of Artificial Sequence Synthetic primer
gaggaggaggaggaggaaga

SEQ ID NO: 314 - DNA, 20 bp, Artificial Sequence
  Description of Artificial Sequence Synthetic primer
ccaccagaggaaagtggtgt

SEQ ID NO: 315 - DNA, 20 bp, Artificial Sequence
  Description of Artificial Sequence Synthetic primer
gacagtgctgtgaagccaga

SEQ ID NO: 316 - DNA, 20 bp, Artificial Sequence
  Description of Artificial Sequence Synthetic primer
aacacagccagtgtgaatgc

SEQ ID NO: 317 - DNA, 20 bp, Artificial Sequence
  Description of Artificial Sequence Synthetic primer
ctcccgcaaaaatccttaca

SEQ ID NO: 318 - DNA, 20 bp, Artificial Sequence
  Description of Artificial Sequence Synthetic primer
gctgctttcaatccgctatc

SEQ ID NO: 319 - DNA, 20 bp, Artificial Sequence
  Description of Artificial Sequence Synthetic primer
taattgcaggagctgtcacg

SEQ ID NO: 320 - DNA, 17 bp, Artificial Sequence
  Description of Artificial Sequence Synthetic primer
gagcggaatgcagcggc

SEQ ID NO: 321 - DNA, 20 bp, Artificial Sequence
  Description of Artificial Sequence Synthetic primer
caagccaatgttctgcttca

SEQ ID NO: 322 - DNA, 20 bp, Artificial Sequence
  Description of Artificial Sequence Synthetic primer
gtgctcctggtttcattggt

SEQ ID NO: 323 - DNA, 20 bp, Artificial Sequence
  Description of Artificial Sequence Synthetic primer
atactcgggggcaagtttct

SEQ ID NO: 324 - DNA, 20 bp, Artificial Sequence
  Description of Artificial Sequence Synthetic primer
cttctttctggaagcgttgg

SEQ ID NO: 325 - DNA, 20 bp, Artificial Sequence
  Description of Artificial Sequence Synthetic primer
ccctgctgagtctgaaaagc

SEQ ID NO: 326 - DNA, 20 bp, Artificial Sequence
  Description of Artificial Sequence Synthetic primer
tgcagaggtttgggtcttct

SEQ ID NO: 327 - DNA, 20 bp, Artificial Sequence
  Description of Artificial Sequence Synthetic primer
agacaagggatggcagaaga

SEQ ID NO: 328 - DNA, 20 bp, Artificial Sequence
  Description of Artificial Sequence Synthetic primer
cgaatgtggcatttcaacag

SEQ ID NO: 329 - DNA, 20 bp, Artificial Sequence
  Description of Artificial Sequence Synthetic primer
atccttttgggggtttatgg

SEQ ID NO: 330 - DNA, 20 bp, Artificial Sequence
  Description of Artificial Sequence Synthetic primer
ctcccttcagctctccagtg

SEQ ID NO: 331 - DNA, 20 bp, Artificial Sequence
  Description of Artificial Sequence Synthetic primer
caggcaggtgagaacagaca

SEQ ID NO: 332 - DNA, 20 bp, Artificial Sequence
  Description of Artificial Sequence Synthetic primer
gtgcctggcccatttaagta

SEQ ID NO: 333 - DNA, 20 bp, Artificial Sequence
  Description of Artificial Sequence Synthetic primer
catccttccaggaccacact

SEQ ID NO: 334 - DNA, 18 bp, Artificial Sequence
  Description of Artificial Sequence Synthetic primer
taaggagcgacgggctgt

SEQ ID NO: 335 - DNA, 20 bp, Artificial Sequence
  Description of Artificial Sequence Synthetic primer
ctgcagtgtcattgcgtctt

SEQ ID NO: 336 - DNA, 20 bp, Artificial Sequence
  Description of Artificial Sequence Synthetic primer
tgtgaagccagacaggtgag

SEQ ID NO: 337 - DNA, 20 bp, Artificial Sequence
  Description of Artificial Sequence Synthetic primer
aaaactgcatggcatcatca

SEQ ID NO: 338 - DNA, 20 bp, Artificial Sequence
  Description of Artificial Sequence Synthetic primer
gcagggtcttctcccctaag

SEQ ID NO: 339 - DNA, 20 bp, Artificial Sequence
  Description of Artificial Sequence Synthetic primer
ggcacagctgaggacagaat

SEQ ID NO: 340 - DNA, 20 bp, Artificial Sequence
  Description of Artificial Sequence Synthetic primer
cttgggcagggatagttgac

SEQ ID NO: 341 - DNA, 20 bp, Artificial Sequence
  Description of Artificial Sequence Synthetic primer
tctttggtgtggggctactt

SEQ ID NO: 342 - DNA, 20 bp, Artificial Sequence
  Description of Artificial Sequence Synthetic primer
gaccataatggatgcccttt

SEQ ID NO: 343 - DNA, 20 bp, Artificial Sequence
  Description of Artificial Sequence Synthetic primer
acctgcatctcacctgaacc

SEQ ID NO: 344 - DNA, 22 bp, Artificial Sequence
  Description of Artificial Sequence Synthetic primer
ccatcactagttctggagctga

SEQ ID NO: 345 - DNA, 20 bp, Artificial Sequence
  Description of Artificial Sequence Synthetic primer
ccttcccaaacaaacaccac

SEQ ID NO: 346 - DNA, 20 bp, Artificial Sequence
  Description of Artificial Sequence Synthetic primer
gccacatgtccttctttgct

SEQ ID NO: 347 - DNA, 20 bp, Artificial Sequence
  Description of Artificial Sequence Synthetic primer
gctgagtacagcttgcatgg

SEQ ID NO: 348 - DNA, 20 bp, Artificial Sequence
  Description of Artificial Sequence Synthetic primer
ctagcagagggggctcaagt

SEQ ID NO: 349 - DNA, 20 bp, Artificial Sequence
  Description of Artificial Sequence Synthetic primer
ctgggctcaacatgttcctt

SEQ ID NO: 350 - DNA, 20 bp, Artificial Sequence
  Description of Artificial Sequence Synthetic primer
gctgggacgaacttttcctc

SEQ ID NO: 351 - DNA, 20 bp, Artificial Sequence
  Description of Artificial Sequence Synthetic primer
tggtcaaatttcagccatca

SEQ ID NO: 352 - DNA, 19 bp, Artificial Sequence
  Description of Artificial Sequence Synthetic primer
gttgacgctgcagggagag

SEQ ID NO: 353 - DNA, 20 bp, Artificial Sequence
  Description of Artificial Sequence Synthetic primer
caacctcaagatcccaggaa

SEQ ID NO: 354 - DNA, 20 bp, Artificial Sequence
  Description of Artificial Sequence Synthetic primer
caggacatgactgcacaggt

SEQ ID NO: 355 - DNA, 20 bp, Artificial Sequence
  Description of Artificial Sequence Synthetic primer
ctgctgtgtttggggacttc

SEQ ID NO: 356 - DNA, 20 bp, Artificial Sequence
  Description of Artificial Sequence Synthetic primer
agggcacaggtacattgagg

SEQ ID NO: 357 - DNA, 20 bp, Artificial Sequence
  Description of Artificial Sequence Synthetic primer
gtttgcctcgcaggagttag

SEQ ID NO: 358 - DNA, 20 bp, Artificial Sequence
  Description of Artificial Sequence Synthetic primer
ttaagagcagccaggaggaa

SEQ ID NO: 359 - DNA, 20 bp, Artificial Sequence
  Description of Artificial Sequence Synthetic primer
tgctgtggacaagagtacgg

SEQ ID NO: 360 - DNA, 20 bp, Artificial Sequence
  Description of Artificial Sequence Synthetic primer
cttccacgtaggcgtctctc

SEQ ID NO: 361 - DNA, 20 bp, Artificial Sequence
  Description of Artificial Sequence Synthetic primer
gcgatcctcaaatgcaatct

SEQ ID NO: 362 - DNA, 20 bp, Artificial Sequence
  Description of Artificial Sequence Synthetic primer
tccagctcagagacgtagca

SEQ ID NO: 363 - DNA, 20 bp, Artificial Sequence
  Description of Artificial Sequence Synthetic primer
gggatgtgctcatcattgaa

SEQ ID NO: 364 - DNA, 20 bp, Artificial Sequence
  Description of Artificial Sequence Synthetic primer
ctgaggtctctcccgaacag

SEQ ID NO: 365 - DNA, 20 bp, Artificial Sequence
  Description of Artificial Sequence Synthetic primer
tattggccctgtctttggac

SEQ ID NO: 366 - DNA, 20 bp, Artificial Sequence
  Description of Artificial Sequence Synthetic primer
gtgaactcactgtcgccgta

SEQ ID NO: 367 - DNA, 20 bp, Artificial Sequence
  Description of Artificial Sequence Synthetic primer
tgcactctggtacagcttgg

SEQ ID NO: 368 - DNA, 18 bp, Artificial Sequence
  Description of Artificial Sequence Synthetic primer
gaggaaagggaggccacg

SEQ ID NO: 369 - DNA, 18 bp, Artificial Sequence
  Description of Artificial Sequence Synthetic primer
gaatgcagcggcccgagg

SEQ ID NO: 370 - DNA, 20 bp, Artificial Sequence
  Description of Artificial Sequence Synthetic primer
tcacacgcacatacacatgc

SEQ ID NO: 371 - DNA, 21 bp, Artificial Sequence
  Description of Artificial Sequence Synthetic primer
ccaataactgttgggcttcaa

SEQ ID NO: 372 - DNA, 18 bp, Artificial Sequence
  Description of Artificial Sequence Synthetic primer
ctgcagggagagggtggt

SEQ ID NO: 373 - DNA, 20 bp, Artificial Sequence
  Description of Artificial Sequence Synthetic primer
tctgagcagaggaggaggtt

SEQ ID NO: 374 - DNA, 20 bp, Artificial Sequence
  Description of Artificial Sequence Synthetic primer
ggctaggaggaatcccagtg

SEQ ID NO: 375 - DNA, 20 bp, Artificial Sequence
  Description of Artificial Sequence Synthetic primer
ttggcaactaccttccttgg

SEQ ID NO: 376 - DNA, 18 bp, Artificial Sequence
  Description of Artificial Sequence Synthetic primer
gttctgagttggctgctc

SEQ ID NO: 377 - DNA, 21 bp, Artificial Sequence
  Description of Artificial Sequence Synthetic primer
caaacggacccagaacttcag

SEQ ID NO: 378 - DNA, 20 bp, Artificial Sequence
  Description of Artificial Sequence Synthetic primer
atcactggggaaaggcttct

SEQ ID NO: 379 - DNA, 20 bp, Artificial Sequence
  Description of Artificial Sequence Synthetic primer
catggacttgagcatctgga

SEQ ID NO: 380 - DNA, 19 bp, Artificial Sequence
  Description of Artificial Sequence Synthetic primer
tacccggaaagcagaggtc

SEQ ID NO: 381 - DNA, 19 bp, Artificial Sequence
  Description of Artificial Sequence Synthetic primer
tcagcttcagcaggtccac

SEQ ID NO: 382 - DNA, 20 bp, Artificial Sequence
  Description of Artificial Sequence Synthetic primer
gcagaaacccagtctggaag

SEQ ID NO: 383 - DNA, 20 bp, Artificial Sequence
  Description of Artificial Sequence Synthetic primer
ggacaggcaaggtagtggac

SEQ ID NO: 384 - DNA, 20 bp, Artificial Sequence
  Description of Artificial Sequence Synthetic primer
ccttcccctgtgcatatgtt

SEQ ID NO: 385 - DNA, 20 bp, Artificial Sequence
  Description of Artificial Sequence Synthetic primer
accgctgcaactaccagact

SEQ ID NO: 386 - DNA, 20 bp, Artificial Sequence
  Description of Artificial Sequence Synthetic primer
gggacgagaagtgcgtgtat

SEQ ID NO: 387 - DNA, 20 bp, Artificial Sequence
  Description of Artificial Sequence Synthetic primer
gcagtcattggcagcctagt

SEQ ID NO: 388 - DNA, 20 bp, Artificial Sequence
  Description of Artificial Sequence Synthetic primer
gagaaacccatcttccacga

SEQ ID NO: 389 - DNA, 20 bp, Artificial Sequence
  Description of Artificial Sequence Synthetic primer
ccatcagcctcaactgctct

SEQ ID NO: 390 - DNA, 20 bp, Artificial Sequence
  Description of Artificial Sequence Synthetic primer
ctgacggcatgctacacatt

SEQ ID NO: 391 - DNA, 20 bp, Artificial Sequence
  Description of Artificial Sequence Synthetic primer
ccagaggacaccaatcacct

SEQ ID NO: 392 - DNA, 20 bp, Artificial Sequence
  Description of Artificial Sequence Synthetic primer
ttgcagtcaaccacttctgg

SEQ ID NO: 393 - DNA, 20 bp, Artificial Sequence
  Description of Artificial Sequence Synthetic primer
caggcggacaccaatagact

SEQ ID NO: 394 - DNA, 19 bp, Artificial Sequence
  Description of Artificial Sequence Synthetic primer
ccctcagcctcctcattct

SEQ ID NO: 395 - DNA, 20 bp, Artificial Sequence
  Description of Artificial Sequence Synthetic primer
cgtagagcggttccagactc

SEQ ID NO: 396 - DNA, 20 bp, Artificial Sequence
  Description of Artificial Sequence Synthetic primer
gttgttgtgcctcatggaga

SEQ ID NO: 397 - DNA, 20 bp, Artificial Sequence
  Description of Artificial Sequence Synthetic primer
acgaggtggacaaacgagtc

SEQ ID NO: 398 - DNA, 20 bp, Artificial Sequence
  Description of Artificial Sequence Synthetic primer
aaggtgtgacctgggatctg

SEQ ID NO: 399 - DNA, 20 bp, Artificial Sequence
  Description of Artificial Sequence Synthetic primer
tgcccagcactgagttatca

SEQ ID NO: 400 - DNA, 20 bp, Artificial Sequence
  Description of Artificial Sequence Synthetic primer
ggcttcagggacagtagtgg

SEQ ID NO: 401 - DNA, 20 bp, Artificial Sequence
  Description of Artificial Sequence Synthetic primer
caccattgatgatgctgacc

SEQ ID NO: 402 - DNA, 20 bp, Artificial Sequence
  Description of Artificial Sequence Synthetic primer
tgccattgtccagaagagaa

SEQ ID NO: 403 - DNA, 20 bp, Artificial Sequence
  Description of Artificial Sequence Synthetic primer
ccatgcagttcagtttcacg

SEQ ID NO: 404 - DNA, 20 bp, Artificial Sequence
  Description of Artificial Sequence Synthetic primer
gaacaaccccctctttgtca

SEQ ID NO: 405 - DNA, 20 bp, Artificial Sequence
  Description of Artificial Sequence Synthetic primer
actggacatccatcccatgt

SEQ ID NO: 406 - DNA, 20 bp, Artificial Sequence
  Description of Artificial Sequence Synthetic primer
tcagctgactgtcctctcca

SEQ ID NO: 407 - DNA, 20 bp, Artificial Sequence
  Description of Artificial Sequence Synthetic primer
ggagggagtcagcccactat

SEQ ID NO: 408 - DNA, 20 bp, Artificial Sequence
  Description of Artificial Sequence Synthetic primer
ctggcacgttctttctctcc

SEQ ID NO: 409 - DNA, 20 bp, Artificial Sequence
  Description of Artificial Sequence Synthetic primer
ttcaatgtcgaccgtctgag

SEQ ID NO: 410 - DNA, 20 bp, Artificial Sequence
  Description of Artificial Sequence Synthetic primer
gcaggagagacccacaaaga

SEQ ID NO: 411 - DNA, 20 bp, Artificial Sequence
  Description of Artificial Sequence Synthetic primer
aggtccagatcctgaaagca

SEQ ID NO: 412 - DNA, 20 bp, Artificial Sequence
  Description of Artificial Sequence Synthetic primer
attcgcacccaggagtacag

SEQ ID NO: 413 - DNA, 20 bp, Artificial Sequence
  Description of Artificial Sequence Synthetic primer
acaggagaccacgaaagtgc

SEQ ID NO: 414 - DNA, 20 bp, Artificial Sequence
  Description of Artificial Sequence Synthetic primer
ccaaagacagaatccccaga

SEQ ID NO: 415 - DNA, 20 bp, Artificial Sequence
  Description of Artificial Sequence Synthetic primer
gtgagaaccaggacccagaa

SEQ ID NO: 416 - DNA, 20 bp, Artificial Sequence
  Description of Artificial Sequence Synthetic primer
ccactatggagtggggagaa

SEQ ID NO: 417 - DNA, 20 bp, Artificial Sequence
  Description of Artificial Sequence Synthetic primer
ggacagagagaggctttgga

SEQ ID NO: 418 - DNA, 20 bp, Artificial Sequence
  Description of Artificial Sequence Synthetic primer
aatgcgatcctgtggatttc

SEQ ID NO: 419 - DNA, 20 bp, Artificial Sequence
  Description of Artificial Sequence Synthetic primer
gatgacaatggcactggttg

SEQ ID NO: 420 - DNA, 20 bp, Artificial Sequence
  Description of Artificial Sequence Synthetic primer
agaattaacccggcaagtcc

SEQ ID NO: 421 - DNA, 20 bp, Artificial Sequence
  Description of Artificial Sequence Synthetic primer
atgaggaggcctcagagtca

SEQ ID NO: 422 - DNA, 20 bp, Artificial Sequence
  Description of Artificial Sequence Synthetic primer
gggttgtggggagtgactta

SEQ ID NO: 423 - DNA, 20 bp, Artificial Sequence
  Description of Artificial Sequence Synthetic primer
ccaaaggctgaatgaaggag

SEQ ID NO: 424 - DNA, 20 bp, Artificial Sequence
  Description of Artificial Sequence Synthetic primer
gaggacctgggggagtagag

SEQ ID NO: 425 - DNA, 20 bp, Artificial Sequence
  Description of Artificial Sequence Synthetic primer
cataccccagtgaacccatc

SEQ ID NO: 426 - DNA, 20 bp, Artificial Sequence
  Description of Artificial Sequence Synthetic primer
gcttttgcccacatgagtct

SEQ ID NO: 427 - DNA, 20 bp, Artificial Sequence
  Description of Artificial Sequence Synthetic primer
cctatgagtcccctccccta

SEQ ID NO: 428 - DNA, 20 bp, Artificial Sequence
  Description of Artificial Sequence Synthetic primer
tgcatgtctgacggagaaag

SEQ ID NO: 429 - DNA, 20 bp, Artificial Sequence
  Description of Artificial Sequence Synthetic primer
ccttacaagcctgtccacca

SEQ ID NO: 430 - DNA, 20 bp, Artificial Sequence
  Description of Artificial Sequence Synthetic primer
tgtctcgtgcagacttgacc

SEQ ID NO: 431 - DNA, 20 bp, Artificial Sequence
  Description of Artificial Sequence Synthetic primer
ggtcaccaacgacagatcct

SEQ ID NO: 432 - DNA, 20 bp, Artificial Sequence
  Description of Artificial Sequence Synthetic primer
gtgtccacaccaacatccag

SEQ ID NO: 433 - DNA, 20 bp, Artificial Sequence
  Description of Artificial Sequence Synthetic primer
cagggcatttctgaggatgt

SEQ ID NO: 434 - DNA, 20 bp, Artificial Sequence
  Description of Artificial Sequence Synthetic primer
ttataccctcctccccatcc

SEQ ID NO: 435 - DNA, 20 bp, Artificial Sequence
  Description of Artificial Sequence Synthetic primer
cagggctcagaacctacagc

SEQ ID NO: 436 - DNA, 20 bp, Artificial Sequence
  Description of Artificial Sequence Synthetic primer
cttttgctctcctgggactg

SEQ ID NO: 437 - DNA, 20 bp, Artificial Sequence
  Description of Artificial Sequence Synthetic primer
actgttagacctcgcccaga

SEQ ID NO: 438 - DNA, 20 bp, Artificial Sequence
  Description of Artificial Sequence Synthetic primer
cagggtctgtctgaggagga

SEQ ID NO: 439 - DNA, 20 bp, Artificial Sequence
  Description of Artificial Sequence Synthetic primer
ttacaggctgggattcaagg

SEQ ID NO: 440 - DNA, 21 bp, Artificial Sequence
  Description of Artificial Sequence Synthetic primer
tccacagacactggaacatga

SEQ ID NO: 441 - DNA, 20 bp, Artificial Sequence
  Description of Artificial Sequence Synthetic primer
caggtgtgggtagagggtgt

SEQ ID NO: 442 - DNA, 20 bp, Artificial Sequence
  Description of Artificial Sequence Synthetic primer
cctcttgtcttcaacgcaca

SEQ ID NO: 443 - DNA, 20 bp, Artificial Sequence
  Description of Artificial Sequence Synthetic primer
tgggcaaaaggtagagcatc

SEQ ID NO: 444 - DNA, 19 bp, Artificial Sequence
  Description of Artificial Sequence Synthetic oligonucleotide
gcctccctcgcgccatcag

SEQ ID NO: 445 - DNA, 23 bp, Artificial Sequence
  Description of Artificial Sequence Synthetic oligonucleotide
nannactgatggcgcgagggagg

SEQ ID NO: 446 - DNA, 25 bp, Artificial Sequence
  Description of Artificial Sequence Synthetic oligonucleotide
gccttgccagcccgctcagnnnnnn

SEQ ID NO: 447 - DNA, 18 bp, Artificial Sequence
  Description of Artificial Sequence Synthetic oligonucleotide
ctgagcgggctggcaagg

SEQ ID NO: 448 - DNA, 26 bp, Artificial Sequence
  Description of Artificial Sequence Synthetic oligonucleotide
tttttttttttttttttttttttttv

(19) United States
(12) Patent Application Publication
(10) Pub. No.: US 2012/0214163 A1
(43) Pub. Date: Aug. 23, 2012

(54) DISEASE-ASSOCIATED GENETIC VARIATIONS AND METHODS FOR OBTAINING AND USING SAME

(75) Inventors: David J. Sugarbaker, Milton, MA (US); Raphael Bueno, Brookline, MA (US)

(73) Assignee: Brigham & Women's Hospital, Boston, MA (US)

(21) Appl. No.: 13/446,464

(22) Filed: Apr. 13, 2012

Related U.S. Application Data
(63) Continuation of application No. 12/601,726, filed on Apr. 16, 2010.
(60) Provisional application No. 60/931,529, filed on May 24, 2007.

(51) Int. Cl. C12Q 1/68; C12N 1/21; C07H 21/04; C12N 15/63; C40B 30/04; C12N 5/10; C12N 1/19

(52) U.S. Cl. 435/6.11; 435/6.1; 435/6.12; 435/325; 435/348; 435/358; 435/366; 435/419; 435/252.3; 435/252.33; 435/254.2; 435/254.21; 435/320.1; 506/9; 536/23.4

(57) ABSTRACT

The invention provides a comprehensive, rapid, unbiased, and accurate method for identifying and/or discovering disease-associated genetic variations, e.g., disease-associated variations. The present invention further provides novel disease-associated genetic variations for use as genetic markers of disease, e.g., cancer. The invention further provides methods for assessing an individual's risk for developing a disease, e.g., cancer, by detecting the presence the novel disease-associated genetic variations of the invention.

### CROSS-REFERENCE TO RELATED APPLICATIONS/PATENTS & INCORPORATION BY REFERENCE

This application is a continuation of U.S. Utility application Ser. No. 12/601,726, filed Apr. 16, 2010, pending, which is the U.S. national phase, pursuant to 35 U.S.C. §371, of PCT international application Ser. No. PCT/US2008/064807, filed May 24, 2008, designating the United States and published in English on Dec. 4, 2008 as publication WO 2008/148072 A2, which claims priority to U.S. Provisional Application Ser. No. 60/931,529, filed May 24, 2007. The entire contents of the aforementioned patent applications are hereby incorporated herein, by this reference.

Any and all references cited in the text of this patent application, including any U.S. or foreign patents or published patent applications, International patent applications, as well as, any non-patent literature references, including any manufacturer's instructions, are hereby expressly incorporated herein by reference.

### STATEMENT OF RIGHTS TO INVENTIONS MADE UNDER FEDERALLY SPONSORED RESEARCH

The work leading to the present invention was funded in part by contract/grant numbers P20CA9057801-A1, U01 CA65170-07, U24CA114725-01, RO1 CA 120528, and R21/R33CA 100315 from the National Cancer Institute. Accordingly, the United States Government has certain rights to this invention.

### BACKGROUND OF THE INVENTION

Genetic factors contribute to virtually every disease, conferring susceptibility or resistance, or by influencing interactions with environmental factors. As genome mapping and sequencing projects continue to advance, more attention is being directed to the problem of sequence variability. In the area of human health, it is believed that a detailed understanding of the correlation between genotype and disease susceptibility, responsiveness to therapy, likelihood of treatment-related side-effects, and other complex traits, will lead to improved therapies, to improved application of existing therapies, to better preventative measures, and to better diagnostic procedures. The ability to scan the human genome to identify the locations of genetic variations which underlie the pathology of human disease and its response to treatment would be an enormously powerful tool in medicine and human biology.

A variety of types of genetic variations exist, including insertions and deletions (indels), differences in the number of repeated sequences, single nucleotide polymorphisms (SNPs), and chromosomal malformations and rearrangements, each of which, either alone or in some combination, may contribute to the genetic basis of disease, e.g., cancer. While there are different kinds of genetic variation, SNPs are the most frequent type in the human genome, occurring at approximately 1 in 10³ bases. A SNP is a genomic position at which at least two or more alternative nucleotide alleles occur at a relatively high frequency (greater than 1%) in a population. In addition, SNPs are well-suited for studying sequence variations because they are relatively stable (i.e., exhibit low mutation rates) and may be responsible for inheritable traits. Notwithstanding the importance of SNPs, it is increasingly becoming apparent that an understanding of all types of possible genetic variations and/or their complex interactions will be necessary to fully progress our current understanding of the genetic influence on diseases, such as cancer.

A multitude of techniques have been developed to detect and study genetic variation, including, for example, Sanger-based sequencing, ligation-based assays, restriction fragment length analysis, allele-specific polymerase chain reaction, assays based on differential electrophoretic mobilities, primer extension assays, mismatch repair enzyme analysis, and hybridization. However, most of these techniques are not directed to large-scale identification and analysis of genetic variation and tend to be focused on single classes of mutations, e.g., point mutations. Accordingly, such methods are ultimately inadequate for understanding diseases having greater degrees of genetic complexity and in particular, those diseases characterized as involving interaction at a multitude of different genetic loci, e.g., cancer.

Cancer continues to have a major impact on world health. Each year, millions of people living in the United States alone are diagnosed with some form of cancer. Although some cancers, such as certain breast cancers and eye-related cancers, are known to be caused by a single gene mutation, the majority of cancers are thought to be more genetically complex and may involve an interaction of a variety of genetic factors. It is generally accepted that because cancer is a genetic disorder, an individual's particular genetic makeup can influence whether and when that individual will develop cancer. Studies of cancer predisposition have focused on high penetrance genes, i.e., those genes in which mutations have a major impact on cancer formation and/or progression. Such genes, although relatively easy to identify, account for only a small portion of cancer risk. The majority of cancer risk is associated with more difficult to find genes, i.e., the low penetrance genes. Notwithstanding such studies, the exact genetic bases underlying cancer are largely unknown.

A particularly poorly understood cancer is malignant pleural mesothelioma (MPM). Malignant pleural mesothelioma is an asbestos-related, rapidly fatal cancer. Its genetic basis is unknown but appears to involve multiple types of chromosomal abnormalities. Central mechanisms underlying MPM are unclear, although MPM tumors evoke a strong inflammatory response thought to contribute to tumorigenesis. In addition, tumor cell survival promoted by TNF-α responsive antiapoptotic proteins such as Inhibitor of Apoptosis-1 (IAP-1) facilitates the resistance of MPM to most cytotoxic chemotherapeutic drugs. Expression profiling with microarrays has supported the general role of inflammation in MPM etiology and has provided some molecular markers for diagnosis and prognosis.

MPM therapies are severely limited and largely ineffective, with the exception of patients how are in the early stages of the disease. MPM is frequently misdiagnosed as lung adenocarcinoma, a tobacco-related malignancy. While most MPM tumors exhibit multiple chromosomal abnormalities, including loss-of-heterozygosity (LOH), the precise genetic basis of this cancer remains unknown, MPM tumors have not been reported to harbor mutations in known oncogenes and tumor suppressor genes commonly involved in other malignancies such as p53, Rb, K-ras and H-ras. Gene expression profiling has also demonstrated that MPM is dissimilar to most other cancers at the molecular level.

Although these studies represent major steps toward characterizing cancer genomes, e.g., the genetic basis for cancers like MPM, additional approaches are needed to unravel the complex connection between genetic variations and complex diseases, such as cancer.

Accordingly, it would be highly desirable to have an approach to the discovery of new genetic variations associated with diseases having complex genetic bases, especially cancer, which are particularly unbiased, rapid and suited for large-scale identification of such variations. It would also be desirable that such methods not be limited to the identification of only a single type of mutation or variation (e.g., SNPs) and be rapid and powerful enough to identify disease-associated mutations in an individual and in a timeframe that is suitable for the treatment and/or monitoring and/or diagnosis of the disease in that individual.

### SUMMARY OF THE INVENTION

The present invention provides a comprehensive, rapid, unbiased, and accurate method for identifying and/or discovering disease-associated genetic variations, e.g., disease-associated variations, single nucleotide polymorphisms (SNPs), allelic variants, indel variants, or loss of heterozygosity variants. The method of the invention can be advantageously performed on the basis of the expressed portion of the genome of a diseased cell or tissue, i.e., the transcriptome. The present invention further provides novel disease-associated genetic variations for use as genetic markers of disease, e.g., cancer. The invention further provides methods for assessing an individual's risk for developing a disease, e.g., cancer, by detecting the presence the novel disease-associated genetic variations of the invention. Still further, the invention provides methods for monitoring the diagnosis and/or prognosis of a disease before, during or after treatment. The present invention further provides isolated nucleic acid molecules containing the novel disease-associated genetic variations of the invention, and methods for obtaining and expressing same. Further still, the invention provides purified proteins encoded by the genes containing the disease-associated genetic variations of the invention, and to methods for using the purified proteins for generating antibodies, or in methods for identifying ligands or antibodies that specifically bind to the proteins which, in turn, may be useful in the treatment or diagnosis of a disease, e.g., cancer, in particular, malignant pleural mesothelioma.

The present invention provides an isolated nucleic acid molecule comprising a nucleotide sequence that includes SEQ ID NOS: 1, 3, 5, 7, 9, 11, 13, 15, 17, 19, 21, 23, 25, 27, 29 or a homolog thereof, wherein each of the nucleotide sequences further comprise at least one genetic variation which predisposes a person to a disease, e.g., malignant pleural mesothelioma.

The invention also provides an isolated nucleic acid molecule comprising a nucleotide sequence that includes SEQ ID NOS: 2, 4, 6, 8, 10, 12, 14, 16, 18, 20, 22, 24, 26, 28, 30 or homolog thereof, wherein each nucleotide sequence comprises a genetic variation which predisposes a person to malignant pleural mesothelioma.

The present invention also provides methods for producing a protein encoded by the nucleotide sequences of SEQ ID NOS: 1, 3, 5, 7, 9, 11, 13, 15, 17, 19, 21, 23, 25, 27, 29 or homolog thereof, or by those sequences of SEQ ID NOS: 2, 4, 6, 8, 10, 12, 14, 16, 18, 20, 22, 24, 26, 28 30 or homolog thereof, comprising culturing a host cell containing an expression vector that includes one of the above nucleotide sequences under conditions sufficient to achieve expression of the nucleotide sequence, followed by recovering the protein from the host cell.

The instant invention further provides a combination that includes a plurality of nucleic acid molecules that include a nucleotide sequence of SEQ ID NOS: 1, 3, 5, 7, 9, 11, 13, 15, 17, 19, 21, 23, 25, 27, 29 or homolog thereof, wherein the nucleotide sequences each further comprises at least one genetic variation which predisposes a person to malignant pleural mesothelioma. The invention also provides a combination that includes a plurality of nucleic acid molecules that include a nucleotide sequence of SEQ ID NOS: 2, 4, 6, 8, 10, 12, 14, 16, 18, 20, 22, 24, 26, 28 30, or homolog thereof, wherein each nucleotide sequence comprises a genetic variation which predisposes a person to malignant pleural mesothelioma.

The invention also provides a microarray containing a plurality of nucleic acid molecules each comprising a nucleotide sequence selected from the group consisting of SEQ ID NOS: 1, 3, 5, 7, 9, 11, 13, 15, 17, 19, 21, 23, 25, 27, 29 and a homolog thereof, or SEQ ID NOS: 2, 4, 6, 8, 10, 12, 14, 16, 18, 20, 22, 24, 26, 28 30 and a homolog thereof, wherein the nucleotide sequences each further comprises at least one genetic variation which predisposes a person to malignant pleural mesothelioma.

The invention still further provides methods for identifying a person having an increased risk for developing malignant pleural mesothelioma, comprising the steps of: (a) hybridizing a substrate or microarray containing any of SEQ ID NOS: 1, 3, 5, 7, 9, 11, 13, 15, 17, 19, 21, 23, 25, 27, 29 or homolog thereof, or SEQ ID NOS: 2, 4, 6, 8, 10, 12, 14, 16, 18, 20, 22, 24, 26, 28, 30 or homolog thereof, wherein the nucleotide sequences each contain at least one genetic variation predisposing a person to malignant pleural mesothelioma, with a nucleic acid sample from the person, thereby forming one or more hydridization complexes; (b) detecting the hydridization complexes, wherein the presence of hybridization complexes indicates the presence of nucleic acid molecules in the sample that are complementary to a nucleotide sequence of the combination, thereby indicating an increased risk for developing malignant pleural mesothelioma in the person.

The present invention also provides a method for identifying a person having an increased risk of developing malignant pleural mesothelioma on the basis of genetic predisposition, comprising the steps of: (a) obtaining a nucleic acid sample from the person; and (b) detecting a genetic variation in a marker gene selected from the group consisting of ACTR1A, MXRA5, PDZK1Ip1, PSMD13, UQCRC1, COL5A2, XRCC6, LRP10, C14orf159, TM9SF1, C9orf86, AVEN, PSMD8BP1/NOB1, Cxorf34, and FLJ00312/CTGLF6, wherein the presence of a genetic variation in at least one marker gene is indicative of an increased risk of developing malignant pleural mesothelioma.

The detecting step, in certain aspects, can involve the sequencing of the nucleic acid sample. In other aspect, the detecting step can involve the hybridization of the nucleic acid sample against the marker genes and detecting hybridization. In still further aspects, the step of detecting can include (a) hybridizing the nucleic acid sample with a microarray of the invention; and (b) detecting hybridization between a nucleic acid sample and the microarray, wherein hybridization indicates the presence of the genetic variation in the nucleic acid sample, thereby detecting an increased risk of developing malignant pleural mesothelioma.

In certain aspects, the genetic variation in the nucleotide sequences of the invention is a single nucleotide polymorphism. In other aspect, the genetic variations of the nucleotide sequences of the invention can be a single nucleotide polymorphism, a somatic mutation, an inversion, a deletion, an insertion, or an LOH mutation.

In other aspects, where the genetic variation is an LOH mutation, the LOH mutation can be due to a deletion, epigenetic silencing, X inactivation, or RNA editing.

In certain other aspects, the present invention provides homologs of the sequences of the invention (e.g., homologs of SEQ ID NOS: 1, 3, 5, 7, 9, 11, 13, 15, 17, 19, 21, 23, 25, 27, 29 or homologs of SEQ ID NOS: 2, 4, 6, 8, 10, 12, 14, 16, 18, 20, 22, 24, 26, 28, 30), the homologous sequence has at least about 85% sequence identity, or about 90% sequence identity, or even about 95% or even 99% sequence identify with its reference sequence.

In certain other aspects, the present invention provides expression vectors that may be contracted to include or encompass the sequences of the invention (e.g., sequences of the invention (e.g., SEQ ID NOS: 1, 3, 5, 7, 9, 11, 13, 15, 17, 19, 21, 23, 25, 27, 29 or homologs thereof, or SEQ ID NOS: 2, 4, 6, 8, 10, 12, 14, 16, 18, 20, 22, 24, 26, 28, 30 or homologs thereof), wherein the nucleotide sequences are operably linked to a promoter sufficient to carry expression of the inventive sequences. The invention also provides host cells which contain the expression vectors of the invention and which can be used to express the nucleotide sequences of the invention in order to obtain the encoded proteins thereof.

In aspects relating to combinations of the invention, the plurality of nucleic acid molecules (e.g., SEQ ID NOS: 1, 3, 5, 7, 9, 11, 13, 15, 17, 19, 21, 23, 25, 27, 29 or homologs thereof, or SEQ ID NOS: 2, 4, 6, 8, 10, 12, 14, 16, 18, 20, 22, 24, 26, 28, 30, or homologs thereof) can be immobilized on a substrate in a manner suitable for hybridization analysis.

In certain other aspects, the nucleic acid samples can be obtained from any bodily tissue, bodily fluid, or cell of a person. In some aspects, the sample is obtained from a bodily tissue, bodily fluid, or cell exhibiting a disease, such as, cancer.

The present invention also provides a method for identifying a person having an increased risk of developing malignant pleural mesothelioma on the basis of genetic predisposition, comprising the steps of: (a) obtaining a sample from the person; (b) contacting the sample with antibodies specific for the proteins encoded by marker genes SEQ ID NOS: 2, 4, 6, 8, 10, 12, 14, 16, 18, 20, 22, 24, 26, 28 and 30, thereby forming antibody complexes; and (c) detecting antibody complexes, wherein the presence of an antibody complex is indicative of an increased risk of developing malignant pleural mesothelioma.

Still further, the present invention provides a method for monitoring the response of malignant pleural mesothelioma to a therapy, comprising the steps of: (a) administering a therapy to the subject in need thereof; (b) obtaining a nucleic acid sample from the subject; and (c) measuring the level of expression of marker genes SEQ ID NOS: 2, 4, 6, 8, 10, 12, 14, 16, 18, 20, 22, 24, 26, 28, 30 or a homolog thereof. The step of measuring can involve, in certain aspects, (a) hybridizing the nucleic acid sample with a microarray comprising a plurality of nucleic acid molecules each comprising a nucleotide sequence selected from the group consisting of SEQ ID NOS: 2, 4, 6, 8, 10, 12, 14, 16, 18, 20, 22, 24, 26, 28, 30 or homolog thereof, wherein each nucleotide sequence comprises a genetic variation which predisposes a person to malignant pleural mesothelioma; and (b) quantitating the hybridization between the nucleic acid sample and the microarray.

The present invention also provides kits for identifying a human subject having an increased risk of developing malignant pleural mesothelioma, comprising the combinations or microarrays of SEQ ID NOS: 1, 3, 5, 7, 9, 11, 13, 15, 17, 19, 21, 23, 25, 27, 29 or homologs thereof, or of SEQ ID NOS: 2, 4, 6, 8, 10, 12, 14, 16, 18, 20, 22, 24, 26, 28, 30, or homologs thereof, and a set of instructions. In certain aspects, the kits can include antibodies specific for the proteins encoded by marker genes SEQ ID NOS: 1, 3, 5, 7, 9, 11, 13, 15, 17, 19, 21, 23, 25, 27, 29 or homologs thereof, or of SEQ ID NOS: 2, 4, 6, 8, 10, 12, 14, 16, 18, 20, 22, 24, 26, 28, 30, or homologs thereof, and a set of instructions.

The present invention also provides a method for detecting disease-associated genetic variations in a sample, comprising the steps of: (a) obtaining the nucleic acids from the sample; (b) pyrosequencing the nucleic acids to generate a sequence data set; (c) analyzing the sequence data set using parameters capable of identifying candidate genetic variations; (d) validating the candidate genetic variations to identify the disease-associated variations, wherein the parameters comprise one or more of the following criteria: a genetic variation must (1) be present in at least 4 reads, (2) be present in at least 30% of the total reads covering genetic variation, and (3) be present in a read that is at least 90% identical to a reference sequence.

The sequence data set can comprises about 4-5× gene coverage. In certain aspects, the pyrosequencing is carried out by a GS20 pyrosequencer. The selection parameters can further comprise the criteria that the genetic variation must have a GS20 quality score of at least 20 and that the genetic variation must be observed in both orientations of sequence reads. In other aspects, the validating step can be achieved by re-sequencing the genetic variation in the sample by Sanger sequencing or any other method of sequence analysis.

In various aspects above, the genetic variations can be single nucleotide polymorphisms, loss of heterozygosity (LOH) mutations, inversions, deletions and insertions. The loss of heterozygosity mutations can be due to a deletion, epigenetic silencing, or X inactivation.

The nucleic acid samples in various aspects can be obtained from cancerous tissue or cells. The cancer can be any type of cancer, e.g., malignant pleural mesothelioma, leukemia, brain cancer, prostate cancer, liver cancer, ovarian cancer, stomach cancer, colorectal cancer, throat cancer, breast cancer, skin cancer, melanoma, lung cancer, lung adenocarcinoma, sarcoma, cervical cancer, testicular cancer, bladder cancer, endocrine cancer, endometrial cancer, esophageal cancer, glioma, lymphoma, neuroblastoma, osteosarcoma, pancreatic cancer, pituitary cancer, or renal cancer.

Other aspects of the invention are described in or are obvious from the following disclosure, and are within the ambit of the invention.

### BRIEF DESCRIPTION OF THE FIGURES

The following Detailed Description, given by way of example, but not intended to limit the invention to specific embodiments described, may be understood in conjunction with the accompanying drawings, in which:

FIG. 1 is a schematic representing the method of the invention.

FIG. 2 shows a table of sequences sequentially mapped to multiple human and chimpanzee databases at NCBI. Only 2% of all transcriptome-derived sequence could not be reliably aligned to any database.

FIG. 3 shows a table depicting global gene and variant analysis. The upper portion addresses the depth of transcriptome sequencing. Approximately 10,000 genes were observed with ˜1× coverage. Approximately 4,000 genes were observed at ˜4-5× coverage, the depth needed to reliably detect a SNP as empirically determined. The lower portion enumerates coding region variants in each specimen.

FIG. 4A shows a graph depicting the number of “Known RefSeq Genes” detected by at least 1 read (solid lines) and 20 reads (dashed lines) as a function of increasing depth of transcriptome sequencing (i.e., Number of Reads) for the six patient specimens. The horizontal asymptote represents the ˜17,000 Known RefSeq Genes detected by at least one read in any of the 4 MPM samples which encompassed a total of 7 million reads.

FIG. 4B shows a bar graph depicting the classification of tumor specimens using read counts to calculate gene expression ratios for six known diagnostic genes and their geometric mean (Gordon et al., Cancer Res (2002), 62:4963-4967. The ratios correctly identified each tumor type (i.e., >1=MPM; <1=ADCA).

FIG. 4C shows a graph depicting the results of analysis of percentage of reads containing known coding region variants in the six tissue samples. Known variants were selected based on >16 reads of coverage in the region of interest (FIG. 5). The distribution of reads around 50% showing heterozygous expression of the variant is consistent with a binomial distribution.

FIG. 5 shows a table of known coding region SNPs in six patients (Patients 1-6) as represented in FIG. 4C.

FIG. 6 shows a table listing PCR primers used for SNP variant validation.

FIG. 7 shows a table depicting the number of genes with candidate mutations in MPM tumors. Known and novel sSNPs and nsSNPs are enumerated in each MPM in the upper portion of the table. The lower portion of the table enumerates those SNPs that were unique to each MPM patient. Unique, patient-specific novel nsSNPs were chosen for further analysis as candidate somatic mutations.

FIG. 8 shows a table listing SNPs in MPM tumors of Patients 1-4 passing all selection rules and not previously recorded in NCBI RefSeq_RNA or dbSNP.

FIG. 9 shows a table listing candidate mutations in the four MPM tumors from Patients 1-4.

FIG. 10 shows a table listing cancer-associated genetic variations identified in MPM Patients 1-4. Of the 69 nsSNPs unique to one of the four MPM patients, 15 were found to represent mutations of the stated type. Approximately half of these variants represented non-conservative amino acid changes (i.e., BLOSUM score<0). *Replaced by Accession #XR_{—}015233.

FIG. 11 provides (A) the nucleotide sequence of GenBank Accession No. NM_{—}005736.2 encoding ACTR1A (SEQ ID NO: 1) and (B) the nucleotide sequence of the ACTR1A a413g mutation (SEQ ID NO: 2).

FIG. 12 provides (A) the nucleotide sequence of GenBank Accession No. NM_{—}015419.1 encoding MXRA5 (SEQ ID NO: 3) and (B) the nucleotide sequence of the MXRA5 c7862a mutation (SEQ ID NO: 4).

FIG. 13 provides (A) the nucleotide sequence of GenBank Accession No. NM_{—}005704.3 encoding PDZK1IP1 (SEQ ID NO: 5) and (B) the nucleotide sequence of the PDZK1IP1 c403t mutation (SEQ ID NO: 6).

FIG. 14 provides (A) the nucleotide sequence of GenBank Accession No. NM_{—}175932.1 encoding PSMD13 (SEQ ID NO: 7) and (B) the nucleotide sequence of the PSMD13 c1254a mutation (SEQ ID NO: 8).

FIG. 15 provides (A) the nucleotide sequence of GenBank Accession No. NM_{—}003365.2 encoding UQCRC1 (SEQ ID NO: 9) and (B) the nucleotide sequence of the UQCRC1g851a mutation (SEQ ID NO: 10).

FIG. 16 provides (A) the nucleotide sequence of GenBank Accession No. NM_{—}000393.3 encoding COL5A2 (SEQ ID NO: 11) and (B) the nucleotide sequence of the COL5A2 c2773t mutation (SEQ ID NO: 12).

FIG. 17 provides (A) the nucleotide sequence of GenBank Accession No. NM_{—}001469.3 encoding XRCC6 (SEQ ID NO: 13) and (B) the nucleotide sequence of the XRCC6g956a mutation (SEQ ID NO: 14).

FIG. 18 provides (A) the nucleotide sequence of GenBank Accession No. NM_{—}014045.3 encoding LRP10 (SEQ ID NO: 15), (B) the nucleotide sequence of the LRP10g1998a mutation (SEQ ID NO: 16).

FIG. 19 provides (A) the nucleotide sequence of GenBank Accession No. NM_{—}024952.4 encoding C14orf159 (SEQ ID NO: 17) and (B) the nucleotide sequence of the C14orf159 μl 727g mutation (SEQ ID NO: 18).

FIG. 20 provides (A) the nucleotide sequence of GenBank Accession No. NM_{—}006405.5 encoding TM9SF1 (SEQ ID NO: 19) and (B) the nucleotide sequence of the TM9SF1 c2014t mutation (SEQ ID NO: 20).

FIG. 21 provides (A) the nucleotide sequence of GenBank Accession No. NM_{—}024718.2 encoding C9orf86 (SEQ ID NO: 21) and (B) the nucleotide sequence of the C9orf86 c2110g mutation (SEQ ID NO: 22).

FIG. 22 provides (A) the nucleotide sequence of GenBank Accession No. NM_{—}020371.2 encoding AVEN (SEQ ID NO: 23) and (B) the nucleotide sequence of the AVEN a784c mutation (SEQ ID NO: 24).

FIG. 23 provides (A) the nucleotide sequence of GenBank Accession No. NM_{—}014062.1 encoding PSMD8BP1/NOB1 (SEQ ID NO: 25) and (B) the nucleotide sequence of the PSMD8BP1/NOB1 a1074g mutation (SEQ ID NO: 26).

FIG. 24 provides (A) the nucleotide sequence of GenBank Accession No. NM_{—}024917.4 encoding Cxorf34 (SEQ ID NO: 27) and (B) the nucleotide sequence of the Cxorf34g1780a mutation (SEQ ID NO: 28).

FIG. 25 provides (A) the nucleotide sequence of GenBank Accession No. XM_{—}374801.3 encoding FLI00312/CTGLF6 (SEQ ID NO: 29) and (B) the nucleotide sequence of the FLI00312/CTGLF6 t1721a mutation (SEQ ID NO: 30).

### DETAILED DESCRIPTION

The present invention relates is part on the surprising discovery that transcriptome sequencing of patient tumors can result in discovery of previously uncharacterized human cancer mutations. By using an integrated approach that includes specimen enrichment for tumor cells, pyrosequencing, and rule-driven informatics, rare mutations were discovered among thousands of expressed genes. In addition to the advantages of speed and cost, this approach enriches for mutations in expressed genes and identifies multiple classes of mutations. In addition, transcriptome sequencing provides information about mRNA expression levels.

Accordingly, the present invention provides a comprehensive, rapid, unbiased, and accurate method for identifying and/or discovering disease-associated genetic variations, e.g., single nucleotide polymorphisms (SNPs), allelic variants, indel variants, or loss of heterozygosity variants, which can then be used as genetic markers to identify those persons who have a risk of being genetically predisposed to disease, e.g., malignant pleural mesothelioma. The invention also provides isolated nucleic acid molecules containing the novel disease-associated genetic variations identified by the method above, which can be used in methods for screening patients at risk for a disease, e.g., malignant pleural mesothelioma, or for monitoring the diagnosis and/or prognosis of a disease before, during or after a treatment. The invention also provides purified proteins encoded by the genes containing the disease-associated genetic variations of the invention, and to methods for using the purified proteins for generating antibodies, or in methods for identifying ligands or antibodies that specifically bind to the proteins which, in turn, may be useful in the treatment or diagnosis of a disease, e.g., cancer, in particular, malignant pleural mesothelioma. The invention is not limited to the above general description, and other aspects will be apparent from the herein description.

It is understood that this invention is not limited to the particular materials and methods described herein. It is also to be understood that the terminology used herein is for the purpose of describing particular aspects and is not intended to limit the scope of the present invention which will be limited only by the appended claims. As used herein, the singular forms “a”, “an”, and “the” include plural reference unless the context clearly dictates otherwise. For example, a reference to “a host cell” includes a plurality of such host cells known to those skilled in the art.

Unless defined otherwise, all technical and scientific terms used herein have the same meanings as commonly understood by one of ordinary skill in the art to which this invention belongs. All publications mentioned herein are cited for the purpose of describing and disclosing the cell lines, protocols, reagents and vectors which are reported in the publications and which might be used in connection with the invention. Nothing herein is to be construed as an admission that such cited references are prior art.

As used herein, “SNV” refers to a single nucleotide variation. An SNV is a single base substitution that differs from the human mRNA reference sequence obtained from the NCBI RefSeq mRNA database. The term, “SNP,” refers to a single nucleotide polymorphism. SNP can refer to those SNVs either present in the NCBI dbSNP database or observed in the patient's nontumor DNA or cDNA.

As used herein, the term “genetic variations,” or equivalently, “mutations,” refers to any mutation that is discoverable by the method of the present invention and which includes, but are not limited to, small nucleotide variations (SNVs), small nucleotide polymorphisms (SNPs), insertion/deletion (“indels”) mutations, and loss-of-heterozygosity (“LOH”) mutations. The genetic variations of the invention can occur at any physical location in the genome of a cell, including in both intragenic (within gene sequences) or intergenic (between gene sequences) sites, or at sites within or outside of polypeptide-encoding regions. The genetic variations discoverable by the present invention can be associated with a disease or a condition. In particular embodiments, the genetic variations are associated with a tumor or carcinoma, such as, malignant pleural mesothelioma (“MPM”).

The term, “disease-associated,” as in disease-associated genetic variation, refers to those genetic variations that occur in a diseased tissue or cell.

As used herein, the term “epigenetic silencing” refers to a type of cancer mutation involving inactivation of gene expression through DNA methylation. Epigenetic inactivation or silencing of genes is widely accepted as one of the major hallmarks of cancer. There are two key elements associated with this type of transcriptional repression: the appearance of DNA methylation at normally methylation-free gene promoter sequences (CpG islands) and the presence of specific histone modifications such as methylation at lysine 9 (K9) of histone H3 that marks inactivated loci (Feinberg et al., “Epigentic mechanisms in human disease,” Cancer Research (2002) 62: 6784-6787, incorporated herein by reference).

As used herein, the term “LOH mutation,” refers to where an individual inherits a heterozygous locus comprising a first allele, which has a mutation (e.g., point mutation) and is flawed, and a second corresponding allele, which is not flawed, wherein the individual gains in the second allele a mutation which results in the second allele becoming flawed. It will be appreciated the LOH mutations can occur in a carcinoma, such as, for example, mesothelioma tumors.

One example of a LOH mutation is in the disease retinoblastoma, in which one parent's contribution of the tumor suppressor Rb1 is flawed. Although most cells will have a functional second copy, chance loss of heterozygosity events in individual cells almost invariably lead to the development of this retinal cancer in an individual.

LOH mutations can arise via several pathways, including, but not limited to, chromosomal deletions, epigenetic silencing, RNA editing errors, formation of chimeric transcripts due to translocations, gene conversion, mitotic recombination and chromosome loss. The latter event is sometimes followed by duplication of the remaining chromosome. LOH mutations can be identified in a disease, e.g., a carcinoma, by noting the presence of heterozygosity at a genetic locus in an individual's germline DNA or a non-diseased tissue, and the absence of heterozygosity at that locus in the individual's diseased cells, e.g., cancer cells.

As used herein, a “cancer driver mutation” refers to a mutation which causes cancer to proliferate. A “cancer passenger mutation” refers to a mutation which does not control cell proliferation and is just ‘along for the ride.’ The meaning of both terms are meant to be consistent with their commonly known meanings.

As used herein, a “reference sequence” refers to a known amino acid or polynucleotide sequence, which can be obtained from a public sequence database, such as, RefSeq database, GenBank database, or the EMBL nucleotide sequence database. A reference sequence can be identified and obtained based on an accession number.

As used herein, “patient-specific” refers to a characteristic or feature which is unique to a given particular patient relative to other patients. In the context of a patient-specific genetic variation, the genetic variation is unique to the particular patient in which the variation was identified. For example, the invention relates in part to MPM tumors having patient-specific genetic variations, i.e. variations that are unique to that patient.

As used herein, the term “tumor-specific” is meant to refer to a feature or characteristic that appears or manifests only in tumor cells or tissue, but not appear or manifest in healthy cells or tissues. A tumor-specific genetic variation refers to a variation that occurs in a tumor, but not in healthy tissue. For example, the invention relates in part to MPM tumors having tumor-specific genetic variations, i.e. variations that are expressed or present in the MPM tumor, but not expressed or present in healthy tissue.

As used herein, the term “carcinoma,” or equivalently, “cancer,” refers to any oncological disorder, including, for example, malignant pleural mesothelioma (MPM), leukemia, brain cancer, prostate cancer, liver cancer, ovarian cancer, stomach cancer, colorectal cancer, throat cancer, breast cancer, skin cancer, melanoma, lung cancer, lung adenocarcinoma, sarcoma, cervical cancer, testicular cancer, bladder cancer, endocrine cancer, endometrial cancer, esophageal cancer, glioma, lymphoma, neuroblastoma, osteosarcoma, pancreatic cancer, pituitary cancer, renal cancer, and the like.

The term “polynucleotide” is used broadly herein to mean a sequence of deoxyribonucleotides or ribonucleotides that are linked together by a phosphodiester bond. For convenience, the term “oligonucleotide” is used herein to refer to a polynucleotide that is used as a primer or a probe. Generally, an oligonucleotide useful as a probe or primer that selectively hybridizes to a selected nucleotide sequence is at least about 15 nucleotides in length, usually at least about 18 nucleotides, and particularly about 21 nucleotides or more in length.

A polynucleotide can be RNA or can be DNA, which can be a gene or a portion thereof, a cDNA, a synthetic polydeoxyribonucleic acid sequence, or the like, and can be single stranded or double stranded, as well as a DNA/RNA hybrid. In various embodiments, a polynucleotide, including an oligonucleotide (e.g., a probe or a primer) can contain nucleoside or nucleotide analogs, or a backbone bond other than a phosphodiester bond. In general, the nucleotides comprising a polynucleotide are naturally occurring deoxyribonucleotides, such as adenine, cytosine, guanine or thymine linked to 2′-deoxyribose, or ribonucleotides such as adenine, cytosine, guanine or uracil linked to ribose. However, a polynucleotide or oligonucleotide also can contain nucleotide analogs, including non-naturally occurring synthetic nucleotides or modified naturally occurring nucleotides. Such nucleotide analogs are well known in the art and commercially available, as are polynucleotides containing such nucleotide analogs (Lin et al., Nucl. Acids Res. 22:5220-5234 (1994); Jellinek et al., Biochemistry 34:11363-11372 (1995); Pagratis et al., Nature Biotechnol. 15:68-73 (1997), each of which is incorporated herein by reference).

The covalent bond linking the nucleotides of a polynucleotide generally is a phosphodiester bond. However, the covalent bond also can be any of numerous other bonds, including a thiodiester bond, a phosphorothioate bond, a peptide-like bond or any other bond known to those in the art as useful for linking nucleotides to produce synthetic polynucleotides (see, for example, Tam et al., Nucl. Acids Res. 22:977-986 (1994); Ecker and Crooke, BioTechnology 13:351360 (1995), each of which is incorporated herein by reference). The incorporation of non-naturally occurring nucleotide analogs or bonds linking the nucleotides or analogs can be particularly useful where the polynucleotide is to be exposed to an environment that can contain a nucleolytic activity, including, for example, a tissue culture medium or upon administration to a living subject, since the modified polynucleotides can be less susceptible to degradation.

A polynucleotide or oligonucleotide comprising naturally occurring nucleotides and phosphodiester bonds can be chemically synthesized or can be produced using recombinant DNA methods, using an appropriate polynucleotide as a template. In comparison, a polynucleotide or oligonucleotide comprising nucleotide analogs or covalent bonds other than phosphodiester bonds generally are chemically synthesized, although an enzyme such as T7 polymerase can incorporate certain types of nucleotide analogs into a polynucleotide and, therefore, can be used to produce such a polynucleotide recombinantly from an appropriate template (Jellinek et al., supra, 1995). Thus, the term polynucleotide as used herein includes naturally occurring nucleic acid molecules, which can be isolated from a cell, as well as synthetic molecules, which can be prepared, for example, by methods of chemical synthesis or by enzymatic methods such as by the polymerase chain reaction (PCR).

In various embodiments, it can be useful to detectably label a polynucleotide or oligonucleotide. Detectable labeling of a polynucleotide or oligonucleotide is well known in the art. Particular non-limiting examples of detectable labels include chemiluminescent labels, radiolabels, enzymes, haptens, or even unique oligonucleotide sequences.

A “composition” comprises at least two sequences selected from the sequences disclosed herein.

The term “derivative” refers to a polynucleotide or a protein that has been subjected to a chemical modification. Derivatization of a polynucleotide can involve substitution of a nontraditional base such as queosine or of an analog such as hypoxanthine. These substitutions are well known in the art. Derivatization of a protein involves, for example, the replacement of a hydrogen by an acetyl, acyl, alkyl, amino, formyl, or morpholino group, or other similar modifications. Derivative molecules retain the biological activities of the naturally occurring molecules but may confer advantages such as longer lifespan or enhanced activity.

A “hybridization complex” is formed between a polynucleotide and a nucleic acid of a sample when the purines of one molecule hydrogen bond with the pyrimidines of the complementary molecule, e.g., 5′-A-G-T-C-3′ base pairs with 3′-T-C-A-G-5′. The degree of complementarity and the use of nucleotide analogs affect the efficiency and stringency of hybridization reactions.

The term “ligand” refers to any agent, molecule, or compound which will bind specifically to a complementary site on a polynucleotide, or to an epitope of a protein or polypeptide.

The term “protein” refers to a polypeptide or any portion thereof. A “portion” of a protein retains at least one biological or antigenic characteristic of a native protein.

The term “purified” refers to any molecule or compound that is separated from its natural environment and is from about 60% free to about 90% free from other components with which it is naturally associated.

The term “sample” is used in its broadest sense as containing nucleic acids, proteins, antibodies, and the like. A sample may comprise a bodily fluid, e.g., the soluble fraction of a cell preparation, or an aliquot of media in which cells were grown; a cell; a chromosome, an organelle, or membrane isolated or extracted from a cell; genomic DNA, RNA, or cDNA in solution or bound to a substrate; a tissue; a tissue print; skin, or hair; and the like.

The term “specific binding” refers to a special and precise interaction between two molecules which is dependent upon their structure, particularly their molecular side groups. For example, the intercalation of a regulatory protein into the major groove of a DNA molecule, the hydrogen bonding along the backbone between two single stranded nucleic acids, or the binding between an epitope of a protein and an agonist, antagonist, or antibody.

The term “similarity” as applied to sequences, refers to the quantification (usually percentage) of nucleotide or residue matches between at least two sequences aligned using a standardized algorithm such as Smith-Waterman alignment (Smith and Waterman (1981) J Mol Biol 147:195-197) or BLAST2 (Altschul et al. (1997) Nucleic Acids Res 25:3389-3402). BLAST2 may be used in a standardized and reproducible way to insert gaps in one of the sequences in order to optimize alignment and to achieve a more meaningful comparison between them.

The term “substrate” refers to any rigid or semi-rigid support to which cDNAs or proteins are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

As used herein, the term “obtain” or “obtaining” or the like is meant to encompass any means by which to come into possession of any of the materials used in the method of the invention, for example, the obtaining of tumor samples or sub-samples from a subject. Obtaining can also refer to the process by which one obtains any other product or material used in the method of the present invention.

### Detection of Disease-Associated Genetic Variations

The method of the present invention provides for a comprehensive, rapid, unbiased, and accurate approach to identifying and/or discovering disease-associated genetic variations, e.g., disease-associated SNPs, reflecting the genetic basis of a disease of interest, such as, for example, cancer, e.g., mesothelioma. The method involves the comprehensive, unbiased and rapid nucleotide sequencing of the expressed portion of the genome of a diseased sample, i.e., the transcriptome, and the analysis of the resulting sequences to identify genetic variations not previously recognized or known in any public sequence databases. The genetic variations can be unique to a particular individual's tumor specimen, e.g., a mesothelioma tumor from a patient.

The disease-associated genetic variations include those that not previously recognized or known in any public sequence databases. The genetic variations can be unique to a particular individual's tumor specimen, e.g., a mesothelioma tumor from a patient. Alternatively, the genetic variations can be present in more than one tumor of the same or different type (e.g. lung tumors isolated from two different patients, or a lung tumor and breast tumor isolated from different patients). The inventive method can identify not only those genetic variations common to particular kinds of diseases, such as, those mutations common to liver cancers, as well as those genetic variations that uniquely occur in an individual's cancer, such as, mutations in a mesothelioma tumor isolated from an individual patient which may not occur in tumors of different individuals.

In certain embodiments, the inventive method is carried out using specimens of diseased tissues or cells obtained from individuals, such as, a tumor specimen obtained from an individual during a medical procedure, such as, a biopsy or surgery. Surgical and/or biopsy methods, or the like, for retrieving or obtaining cells and/or tissues from individuals are well-known to those of ordinary skill in the art, and include, for example, needle-based biopsies, cone biopsies, and surgical resection. The cells and/or tissues removed and processed by the present invention can be from any available source, including directly from a subject (e.g., during surgery to remove a tumor), or from a stored or a commercial source of samples (e.g., tumor samples obtained from prior procedures and stored in biological freezer), and when removed from a patient, specimens can be processed by any suitable known means in the art, such as, for example, rapid freezing. Excess portions of any sample can be banked for future use.

Specimens and/or their individual RNAs can also be pooled together and sequenced and analyzed in accordance with the present invention in order to study the allelic distribution frequencies and patterns of the genetic variations within the population. The specimens can also be kept separate and/or analyzed separately by the methods of the invention to identify the mutations from individual tumors or specimens.

As controls, the invention contemplates removing or obtaining other tissue samples from healthy tissue or from tissue which at least does not manifest the same disease under study from individuals to provide a reference transcriptome for comparisons.

The specimens analyzed by the present invention can also be from any diseased or non-diseased (e.g., healthy) individual. In preferred embodiments, the specimens are obtained from a diseased individual, for example, a patient with a tumor, such as, a mesothelioma tumor.

The specimens can be obtained from any target tissue or bodily fluid of the individual. In a preferred aspect, the specimens are obtained from tumor tissues, such as, for example, MPM tumors. The individual can include any mammal, which may include a human, or pigs, goats, rabbits, dogs, cats, or livestock animals.

Tumor specimens used in the present invention can be optimized for desirable characteristics, such as, for example, high tumor cellularity and low necrosis, using known methods, such as by microaliquoting as described by Richards et al., Biotech Histochem., In Press (2007). Such methods generally involve histologic procedures which examine cross-sectional samples of an isolated tumor tissue in order to evaluate its histologic parameters as an aid to selecting sub-samples of a tumor sample that can be used in the present invention. Example 1 describes one embodiment used to isolate sub-samples of a removed tumor sample, the sub-samples having higher tumor cellularity and lower necrosis than the original tumor.

In preferred embodiments, the present invention relates to identifying genetic variations on the basis of the expressed portion of the genome of a target cell or tissue, i.e., the transcriptome. As used herein, the term “transcriptome” refers to the population or collection of nucleic acid transcripts for both coding and non-coding nucleic acid sequences, expressed in a particular cell or tissue at a particular time or under a particular set of conditions, e.g., in response to an environmental stimulus or a tissue in a diseased state. The transcriptome can be of diseased or non-diseased cells or tissues. Since the present invention preferably is focused on the analysis of genetic variation at the transcriptome level, the method of the invention next contemplates obtaining the RNA of the cell or tissue of interest.

Any suitable conventional method known to a skilled artisan for obtaining, extracting, and/or purifying RNA is contemplated by the present invention. For example, RNA can be isolated and purified from a tissue samples by conventional techniques, such as, by acid phenol chloroform extraction, ethanol precipitation, column chromatography (with silica beads, e.g. Qiagen® RNeasy columns), or any known method in the art, for example, TRIzol extraction procedure, as described in Gramza et. al. “Efficient Method for Preparing Normal and Tumor Tissue for RNA Extraction” BioTechniques, volume 18, page 218 (1995) (incorporated herein by reference in its entirety). RNA can also be isolated and/or purified by any suitable method described in a molecular biology handbook, such as, for example, Sambrook et al., Molecular Cloning: A Laboratory Manual, Cold Spring Harbor Laboratory Press, Third Edition, 2001 (incorporated herein by reference in its entirety). It will be appreciated that RNA isolation and/or purification protocols may be tailored to a given tissue, or cell type. Particular methods for RNA isolation and/or purification from particular cell and/or tissue types are well known in the art.

The RNA can be high-quality RNA, i.e., where a substantial portion of the RNA comprises full-length transcripts, a substantially low portion of RNA fragments, and a substantially low amount of contaminants, such as DNA. The quality of isolated RNA can be evaluated by any suitable conventional method, such as, for example, by using an Agilent 2100 Bioanalyzer (Agilent, Palo Alto, Calif.) with an RNA 6000 Pico LabChip. The quantity and/or concentration of the RNA preparation can be quantified by any conventional means, such as, for example, spectrophotometric means or with commercial kits, such as, for example, Quant-iT RiboGreen RNA Assay kit (Invitrogen, Carlsbad, Calif.). Optionally, contaminating DNA can be removed by various procedures, including, for example, treatment with a DNase (e.g. from Promega, Madison, Wis.). Where the Agilant 2100 Bioanalyzer is used to determine RNA quality, an RNA Integrity Number (RIN) which is preferably greater than about 5, more preferably which is greater that about 6, still more preferably which is greater than 7, or 7.5, 7.8, or even 8 or 9.

Preferably, information regarding the pathology of the source tissue (e.g. type of cancer and its pathology), demographic of the individual from whom the tissue is removed (e.g. age, gender, type of removal procedure, health condition, whether or not medicated, family history, etc.) is recovered. For example, Table 1 of Example 1 exemplifies the recording of demographic and tumor information for six patients.

One advantage of the method of the invention is that no a priori knowledge of mutations is necessary since the method is based on de novo nucleic acid sequencing. In addition, no cloning is required for sequencing.

After isolation of the RNA, reverse transcription can be performed to generate cDNA from the mRNA population. cDNA synthesis can be performed by any method known to those of skill in the art. The various dNTPs, buffer medium, and enzyme with reverse transcriptase activity may be purchased commercially from various sources, e.g. Superscript™ (e.g. Superscript™ III Platinum® Two-Step qRT-PCR Kits, Invitrogen, Carlsbad, Calif.). First strand synthesis may be directed by an oligo(dT) primer that hybridizes to all polyadenylated RNA species. The oligo(dT) primer is usually 10-30 bases long, more preferably 12-18 bases long, and may comprise a mixture of primers of different lengths. Other suitable polythymine primers include the non-replicable dT primer described in U.S. Pat. No. 6,027,923, and oligo (dT)ₙ V (V=A, C, or G) primers. Methods for making cDNA libraries are well known. See Gubler and Hoffman (1983) Gene 25:263-269 or Sambrook, et al. (supra), each of which are incorporated herein by reference.

Poly-A+ RNA for cDNA synthesis can be prepared by any suitable conventional means, such as, for example chromotography or bead separation technology using oligo(dT) primers as a binding agent for mRNA.

Construction methods for cDNA libraries in a variety of different vectors, including, for example, bacteriophage, plasmids, and viruses capable of infecting eukaryotic cells, are well known in the art. Any known library production method resulting in largely full-length clones of expressed genes may be used.

Once obtained, the cDNA library is then sequenced using a high-throughput nucleotide sequencing method. Preferably, the present invention utilizes a pyrophosphate-based nucleic acid sequencing method.

Pyrophosphate-based nucleic acid sequencing method (also known as pyrosequencing) was first described by Hyman (U.S. Pat. No. 4,971,903, which is incorporated herein by reference). This technique is based on the observation that pyrophosphate (PPi) can be detected by a number of assays. In a polymerase reaction, a sequencing primer is annealed to the template. If a nucleotide complements the next base in the template (i.e., next correct base 3′ of the primer sequence), it is incorporated into the growing primer chain, and PPi is released. When only one of the four nucleotides is introduced into the reaction at a time, PPi is generated only when the correct nucleotide is introduced. Thus, the production of PPi reveals the identity of the next correct base. In this way, a sequence from a template is obtained or confirmed. Additional nucleotides of the sequence are obtained by cycling of the polymerase reaction, in the presence of a single nucleotide at a time.

Thus, pyrosequencing is a real-time, sequencing by synthesis method based on the detection of released pyrophosphate during DNA synthesis. Pyrosequencing does not require the separation or sizing of the reaction products by such methods as electrophoresis. It is capable of being performed in a massively parallel fashion. Pyrosequencing has been used successfully for a number of applications, including in clinical microbiology and is further described in U.S. Publication Nos. 2006/0051807, 2006/0228721, 2006/0040297, 2005/0130173, Margulies et al., Nature, 2005, 437:376-380, and Bainbridge et al., BMC Genomics, 2006, 7:246; Ng et al., Nucleic Acids Research, 2006, 34, published online; Pinard et al., BMC Genomics, 7:216; Leamon et al., Gene Therapy and Regulation, 2007, 3:15-31; Tombetti et al., BMC Bioinformatics, 2007, 8:S22, each of which are incorporated herein by reference in their entireties.

Recently, pyrosequencing has also been used to achieve ultra-high throughput sequencing (up to 10 Mega base-pairs (Mbp) per hour) (Tombetti et al., BMC Bioinformatics, 2007, 8:S22), using template-carrying microbeads deposited in microfabricated picoliter-sized reaction wells (Margulies et al., Nature, 2005, 437:376-380). In preferred aspects, 454 Life Sciences Corporation's GS20 pyrosequencer (herein can be referred to as the “GS20 pyrosequencer”) was used for sequencing in the present invention (see http://www.454.html, and references utilizing the GS20 pyrosequencer, Bainbridge et al., BMC Genomics, 2006, 7:246; Ng et al., Nucleic Acids Research, 2006, 34, published online; Pinard et al., BMC Genomics, 7:216; Leamon et al., Gene Therapy and Regulation, 2007, 3:15-31; Tombetti et al., BMC Bioinformatics, 2007, 8:S22, each of which are incorporated herein by reference).

While the throughput is very high (e.g. up to 10 Mbp per hour), the lengths of the sequenced fragments are substantially shorter (94 bp) than the sequence read lengths generated by traditional Sanger sequencing methods (200-300 bp). Software systems for DNA sequence variant discovery that are based on Sanger chemistry, including base-calling algorithms, are generally inadequate for novel DNA sequencing technologies like pyrosequencing, which feature short read lengths, different error profiles than Sanger-generated sequence reads, and large amounts of data (10 Mbp of sequence per hour).

Accordingly, a further aspect of the present invention relates to a new and useful bioinformatics approach to identifying genetic variations in the transcriptome sequences of interest. This new and useful bioinformatics approach can be referred to as the “mutation filter” of the invention, which is essentially a set of criteria or “parameter” which must be met by the sequence data (transcriptome pyrosequencing data) in order for a particular putative mutation or genetic variation to constitute a genuine mutation or genetic variation of the invention.

Assessment of putative sequence variants identified by analysis of unfiltered high-throughput pyrosequencing data revealed an unacceptably high number of false positive SNPs. To minimize this problem, an empiric rule set, i.e., the mutation filter, was developed by the present inventors, for use with high-throughput pyrosequencing technology as a tool for mutation discovery in disease-associated tissues, such as, tumors, e.g., mesothelioma tumors. In a first preferred aspect, these rules require in whole or in part that the genetic variation meet certain parameters relating to read coverage (how frequently the genetic variation is sequenced), percentage of sequences covering the variation which show the presence of the variation, instrument-related quality scores (e.g. GS20 quality scores), whether variation is observed bidirectionally, and whether the variation is within a read that corresponds to a known sequence, and the degree of the similarity to the known sequence.

In a first embodiment, the genetic mutation must, in whole or in part, be: (1) present in at least 3 reads, more preferably in at least 4 reads (this effectively requires 4-5× gene coverage), or even up to 5, 6 or 7 reads; (2) present in at least 30% of the total number of reads covering the genetic variation; (3) of GS20 quality score ≧20 for the relevant nucleotide; (4) observed in reads obtained from both orientations; and (5) within a read that is >90% identical along its entire length to the target RefSeq mRNA sequence.

In another embodiment, the mutation filter includes the criteria that the mutation must be present in at least 2 reads, more preferably at least 3 reads, or even 4 reads, more preferably still at least 5 reads or even 6 reads, still more preferably at least 7 or 8 reads or more.

In yet another embodiment, the mutation filter includes the criteria that the mutation must be present in at least 20% of the total number of reads, more preferably in at least 25%, still more preferably in at least 30%, more preferably in at least 35% or even 40% or more of the reads.

In another embodiment, the mutation filter includes the criteria that the mutation must have a GS20 quality score of at least 15%, or more preferably at least 17%, or more preferably still at least 20% or 25% or even 30% or more.

In still another embodiment, the mutation filter includes the criteria that the mutation must be in a read that is at least 75% identical along its entire length to the target RefSeq mRNA sequence or other reference sequence, or more preferably at least about 80% or even 85% identical, or more preferably still at least about 90% or even 95% or even 99% identical.

As used herein, the term “read” refers to a single unit of contiguous sequence generated by the pyrosequencing method of the invention. Typically, the average read length is about 94 bp.

The present inventors have found that using the empiric rule set or the mutation filter of the invention surprisingly resulted in the identification of true-positive genetic variations, and thus, enabling greater specificity in the discovery of genuine variations or mutations. Example 3, for example, indicates that 94 SNPs identified by the present inventive method and mutation filter were 100% confirmed as genuine mutations upon subsequent analysis by conventional Sanger sequencing. Further, Example 3 indicates that the mutation filter exhibited 96% sensitivity in the identification of 2,465 well-annotated SNPs among 1,415 genes with ≧4× coverage of 454 sequencing reads in the normal lung control sample. Thus, these rules surprisingly provide sufficient specificity and sensitivity for discovery of true mutations, i.e., genuine mutations, among thousands of potential candidate variants and should assist in the discovery and validation of genetic variants and tumor mutations.

As used herein, a “genuine” mutation or variation refers to any polymorphism or mutation or genetic variation which is not the result of a sequencing error.

In a further aspect of the present invention, sequence analysis can be performed on the sequences generated from the pyrosequencing reactions such the sequences can be compared and annotated with respect to reference sequence databases, such as, for example, NCBI's RefSeq transcript database (a set of 40,545 transcripts). Such comparisons can be carried out using well-known alignment software and algorithms, such as MegaBLAST version 2.2.13, using parameters that will be well-known or determinable by the skilled artisan without undue experimentation. Once the sequences from the pyrosequencing reactions have been aligned and identified, the mismatches and genetic variations can be mapped with respect to the reference sequences. Mutations or variations detected can be “novel,” i.e., not previously recorded in a reference transcript database. Once mapped to a reference sequence database, the data can be further analyzed using the mutation filter of the invention described above, which is based on user-specified criteria relating to, but not limited to, transcript abundance, positional coverage, variant frequencies, and variant putative functional properties, etc. For purposes of the present invention, any mutation or variation that survives the mutation filter can be referred to as a “candidate mutation” or “candidate genetic variation.”

In yet another aspect, any candidate mutation or variation can be validated by any suitable method. For example, the candidate mutations can be validated by obtaining a nucleic acid sample from the same individual from whom the transcriptome was prepared and re-sequencing the region of the nucleic acid encompassing the candidate mutation. Validation can also include determining whether the candidate mutation is present in healthy tissue of the same individual. If the mutation is present only in the tumor tissue and not in the healthy tissue, then the mutation can be a somatic mutation. If the mutation is confirmed to be present in the diseased tissue as well as the healthy tissue, then the mutation is an inherited mutation or polymorphism, i.e., a germ-line mutation. Detection of the candidate mutation can be achieved by any suitable means, such as by using PCR primers designed to amplify a PCR fragment encompassing the variation. Methods of preparing PCR primers are well-known in the art, and exemplified in one aspect in the Examples.

The Examples presented herewith illustrate an embodiment of the present invention relating to mutation discovery in mesothelioma lung tumors obtained from individual human subjects. In the Examples, mesothelioma tumors were obtained from each of four individual patients, from which the RNA was obtained and sequenced by a pyrosequencing technology. The approximately 250 megabases of sequence per patient was mapped to reference transcript databases, the putative variations was characterized, and then the variations were filtered using an embodiment of the mutation filter of the present invention to provide candidate mutations. The candidate mutations are then validated by re-sequencing the genetic regions encompassing the variations from the diseased and healthy tissues of the individual. The re-sequencing performed by standard Sanger sequencing of PCR fragments covering the mutations amplified from the healthy and diseased tissue. The novel variations (variations not previously recorded in transcript reference databases) were explored further.

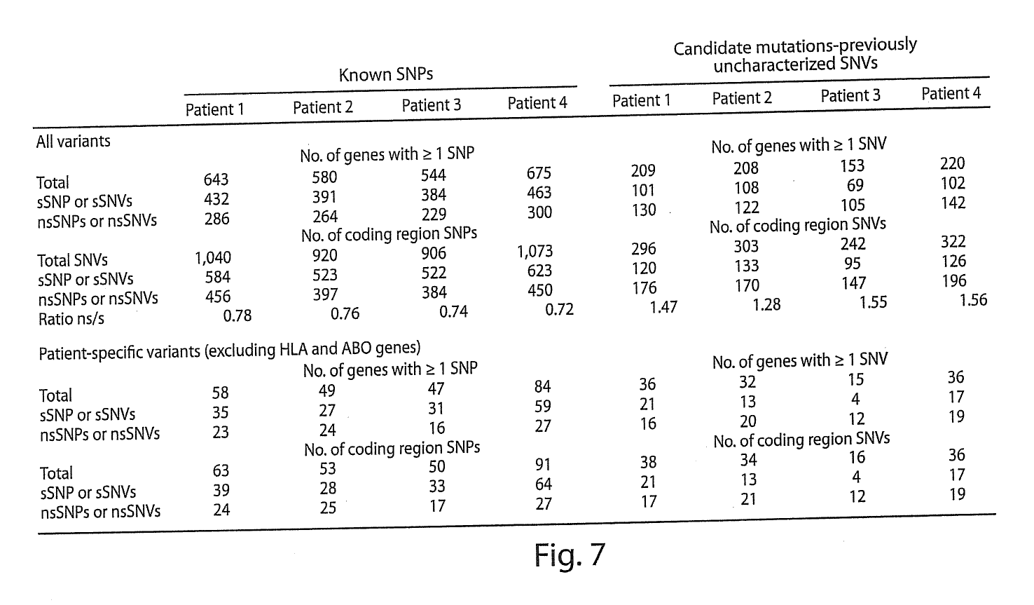

Example 4 indicates that each MPM tumor isolated from one of the 4 MPM patients was identified with between 153-220 genes containing at least 1 novel. SNP (see FIG. 7). And, for all 4 MPM tumors, the study revealed a total of 619 non-redundant and novel coding region SNPs, which are likely to be functionally-relevant, tumor-related mutations. In addition, the 4 MPM tumors, together, were found to contain 67 genes (12-20 per MPM sample) having a total of 69 patient-specific novel SNPs, i.e., SNPs that were novel (not previously recorded in a reference database) and unique for a given tumor (relative to other tumors) from which it was sequenced (FIG. 7). Further analysis was carried out to determine their mutational profiles. It was found that of the 69 SNPs, 54 (78%) were also present in normal genomic DNA, indicating that they were gene-line polymorphisms and not somatic mutations, although they may still have a role in cancer development. The remaining 15 (22%) were found to be tumor-specific variants representing multiple types of mutations including: somatic mutations, LOH mutations (7 mutations) due to chromosomal deletions, epigenetic silencing, and RNA editing. The 7 LOH mutations were further determined to be homozygous in the tumor mRNA and heterozygous in the normal genomic DNA. These results emphasize the diversity of mutations that exist in MPM tumors and emphasize the power of the method of the present invention for uncovering the mutations, i.e., shows that the method of the invention is unbiased with respect to the types of mutations which are detectable.

The LOH mutations occur in genes not previously implicated in tumorigenesis. The genes harboring the detected 7 LOH mutations include (a) ACTR1A (abundant subunit of dynactin and potential role in disruption of p53 pathway), (b) MXRA5 (overexpressed in colon cancer), (c) UQCRC1 (overexpressed in colon cancer), (d) PDZK1IP1 (overexpressed in human carcinomas of diverse origin and associated with tumor suppressor phenotype in culture colon cells), (e) PSMD13 (proteasome subunit responsible for intracellular protein degradation and target of new class of anti-cancer drugs), (f) COL5A2 (alpha chain for one of the low abundance fibrillar collagens and is up-regulated in colon cancer), and (g) XRCC6 (forms heterodimer with XRCC5 and mediates repair of DNA damage).

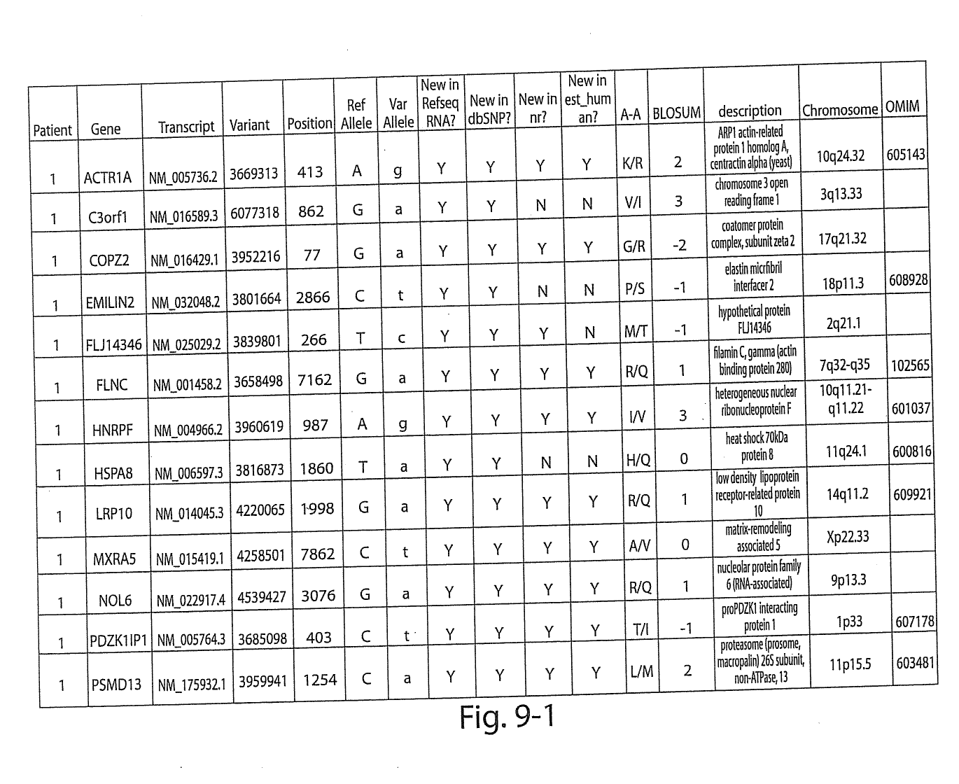

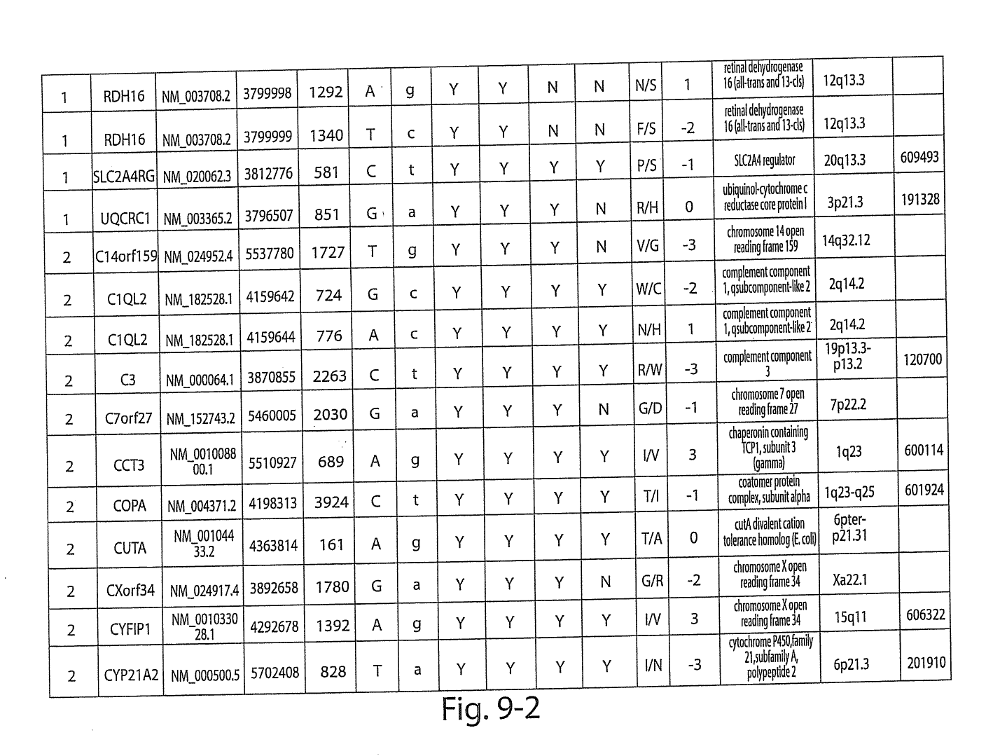

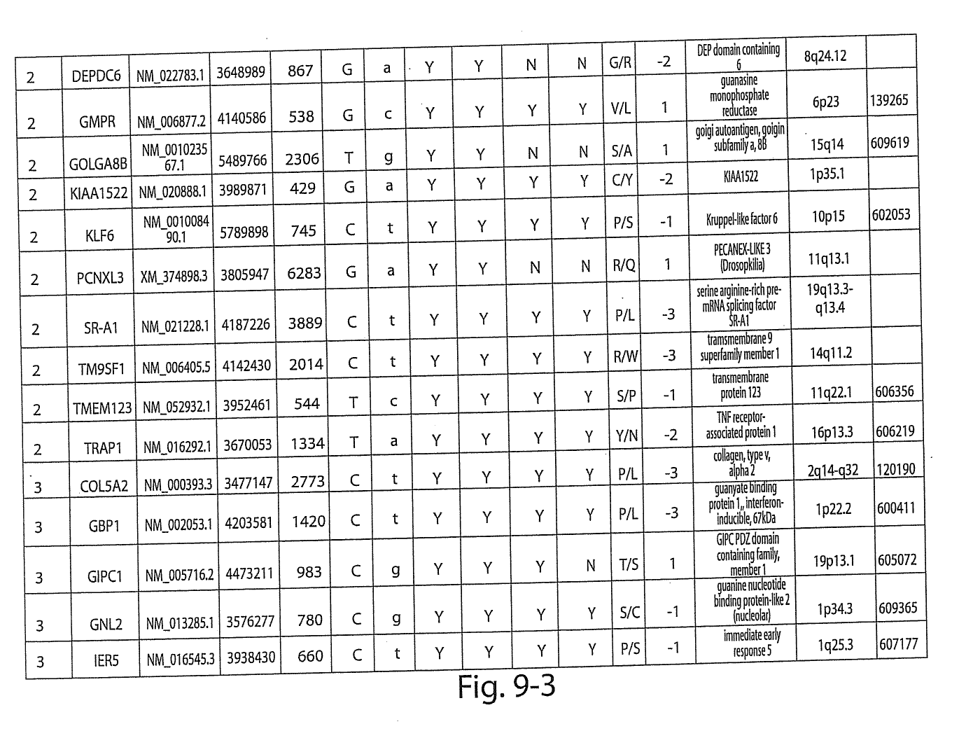

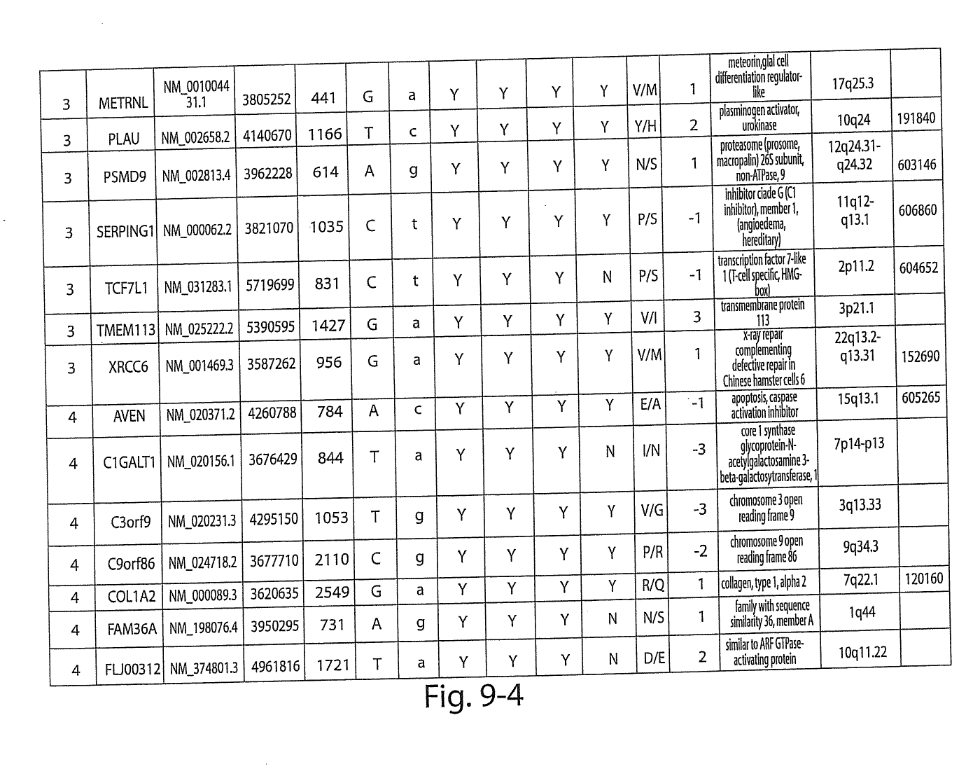

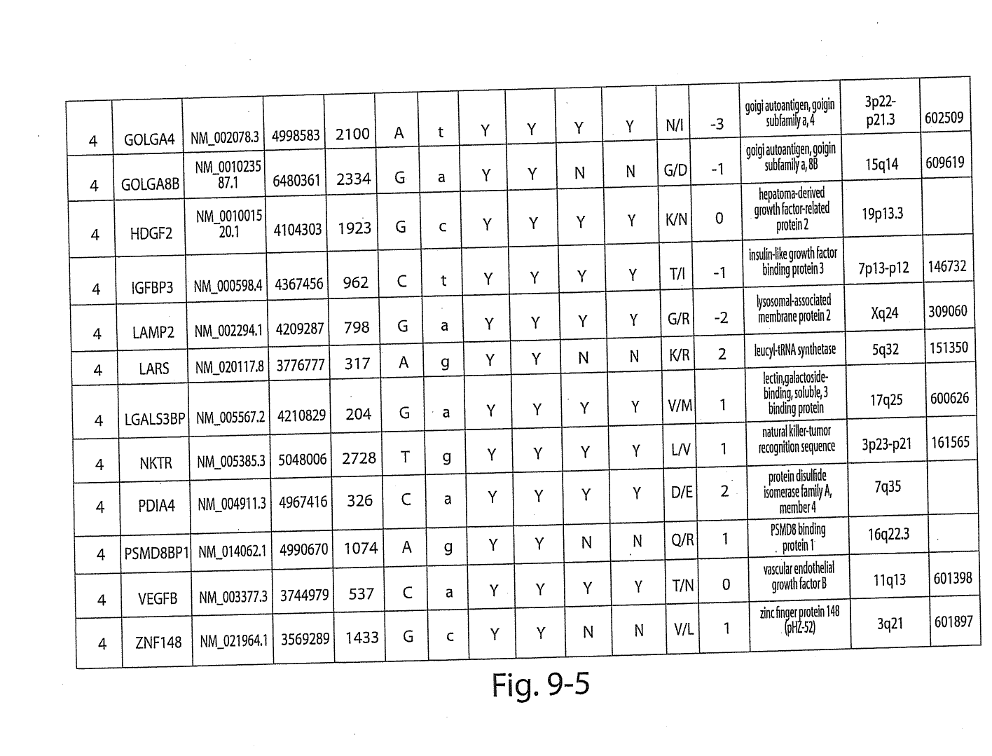

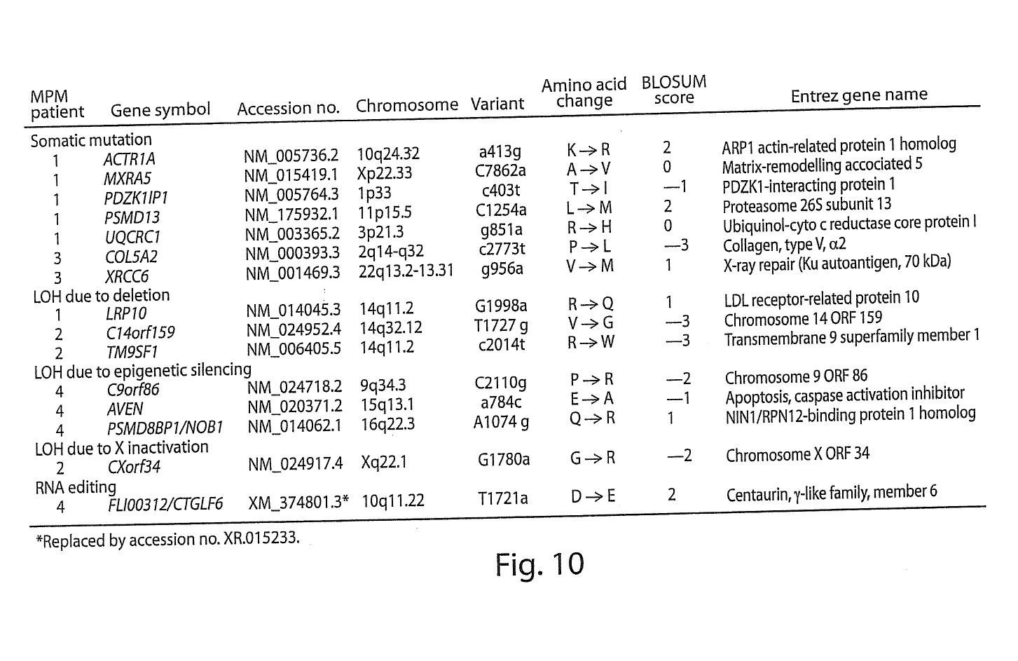

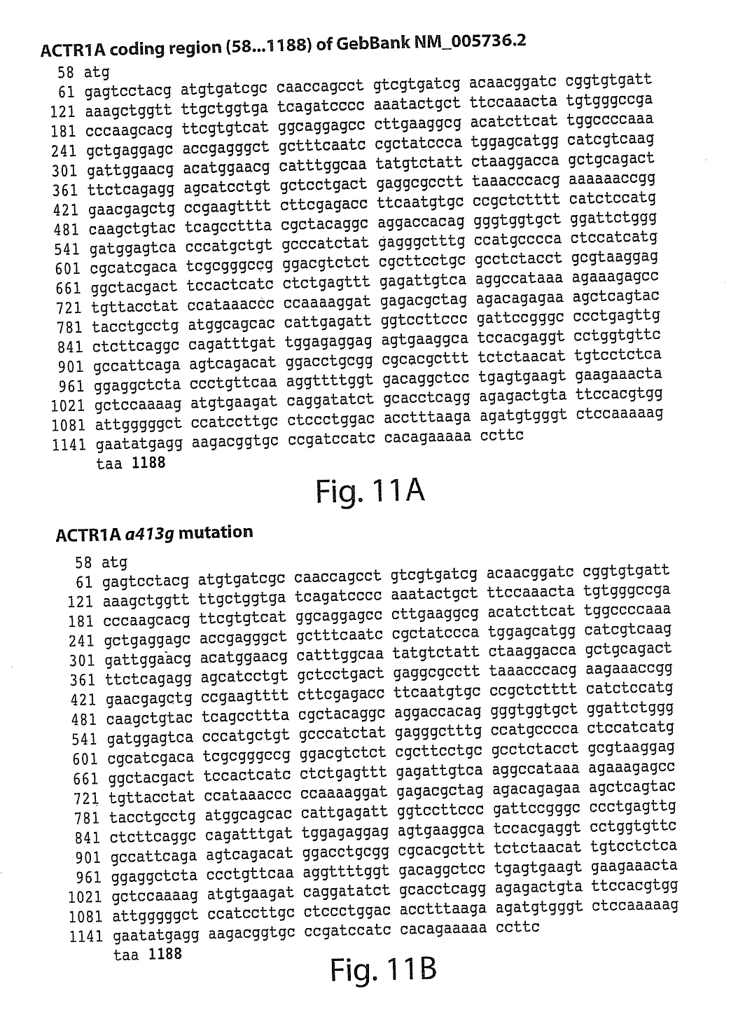

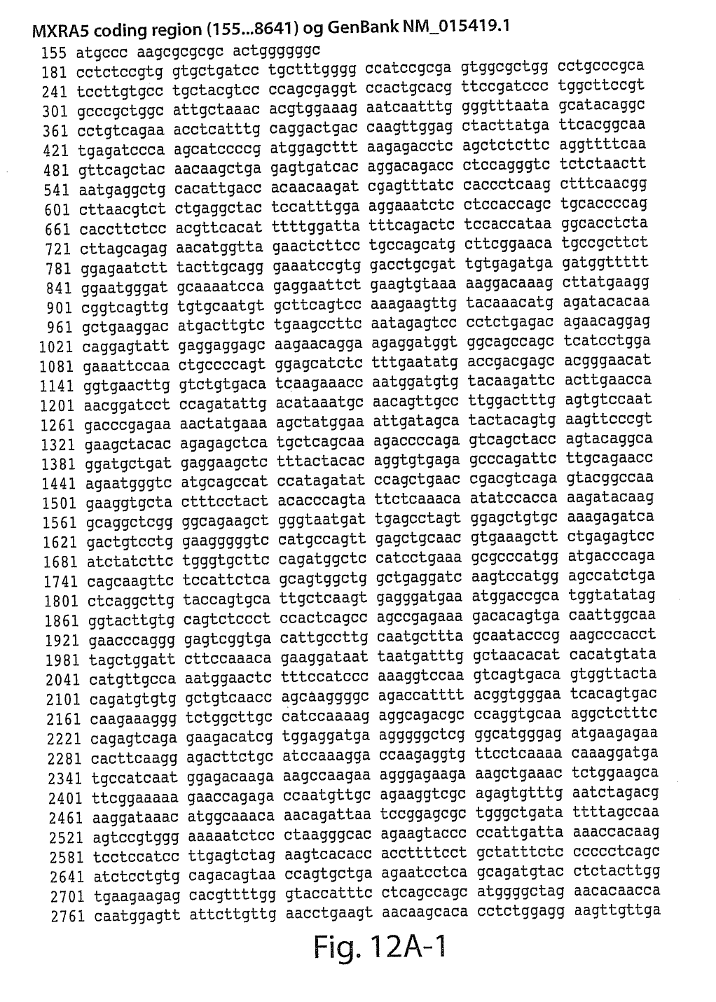

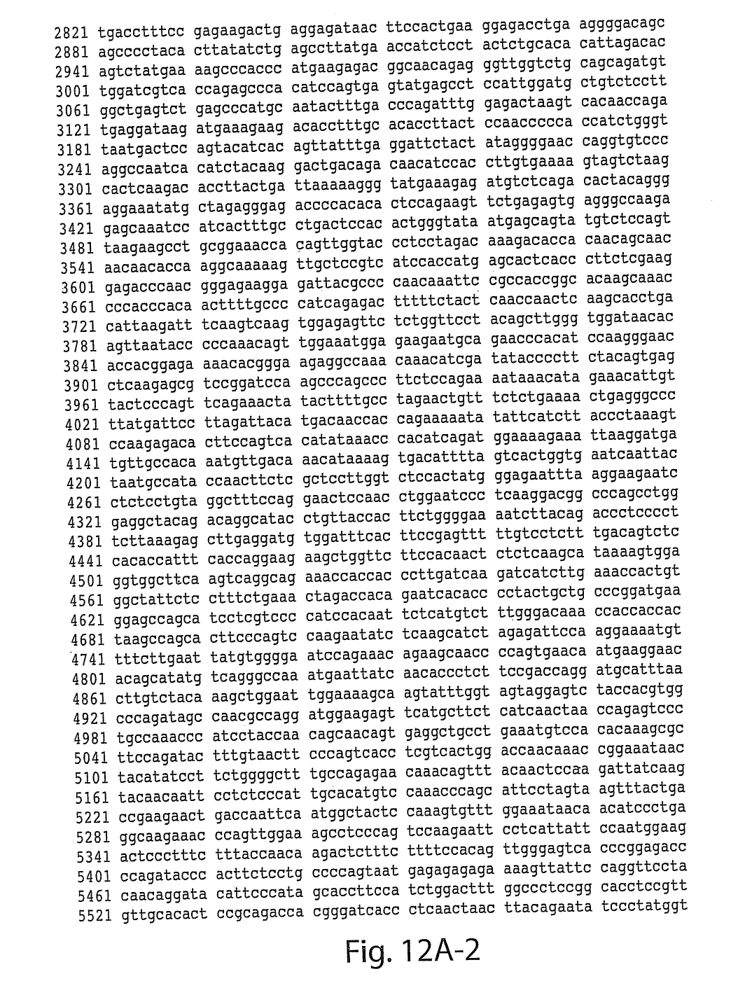

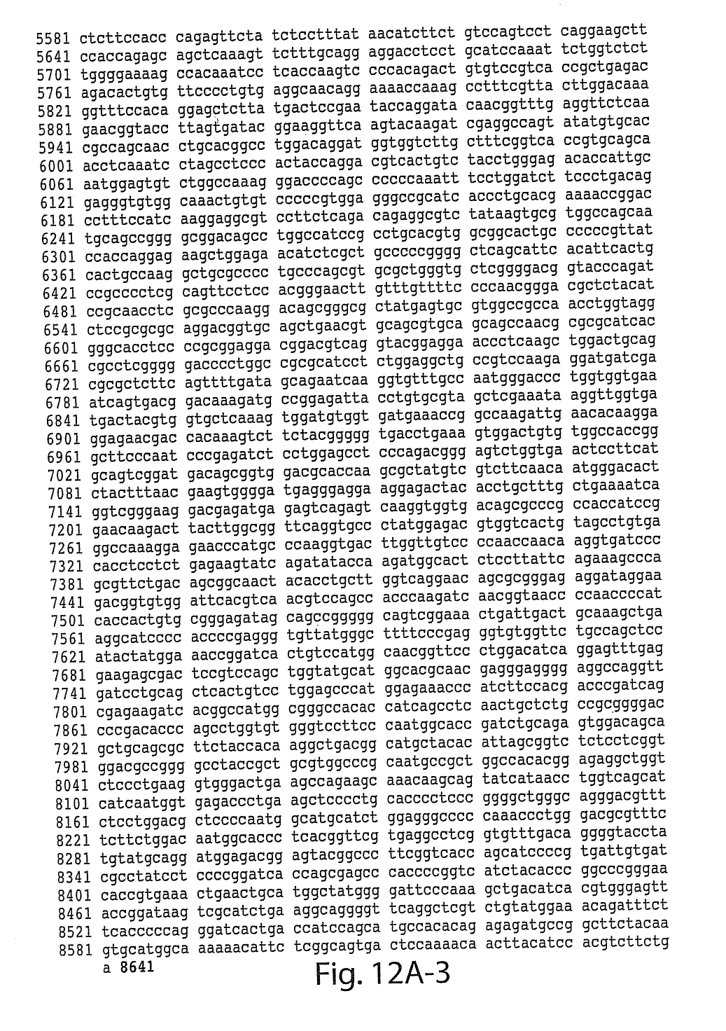

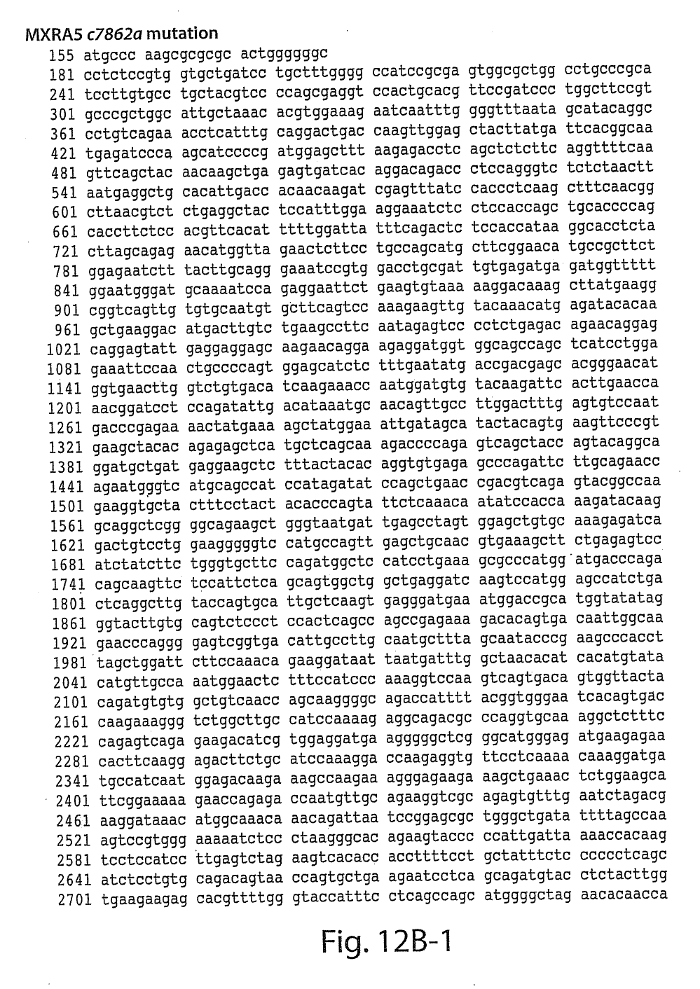

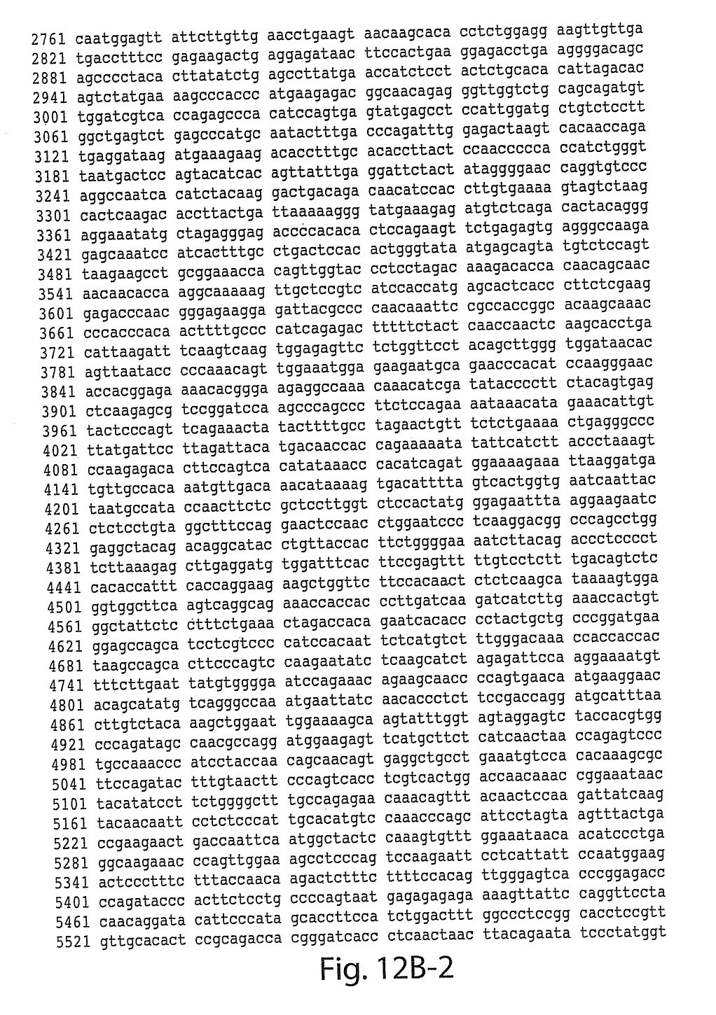

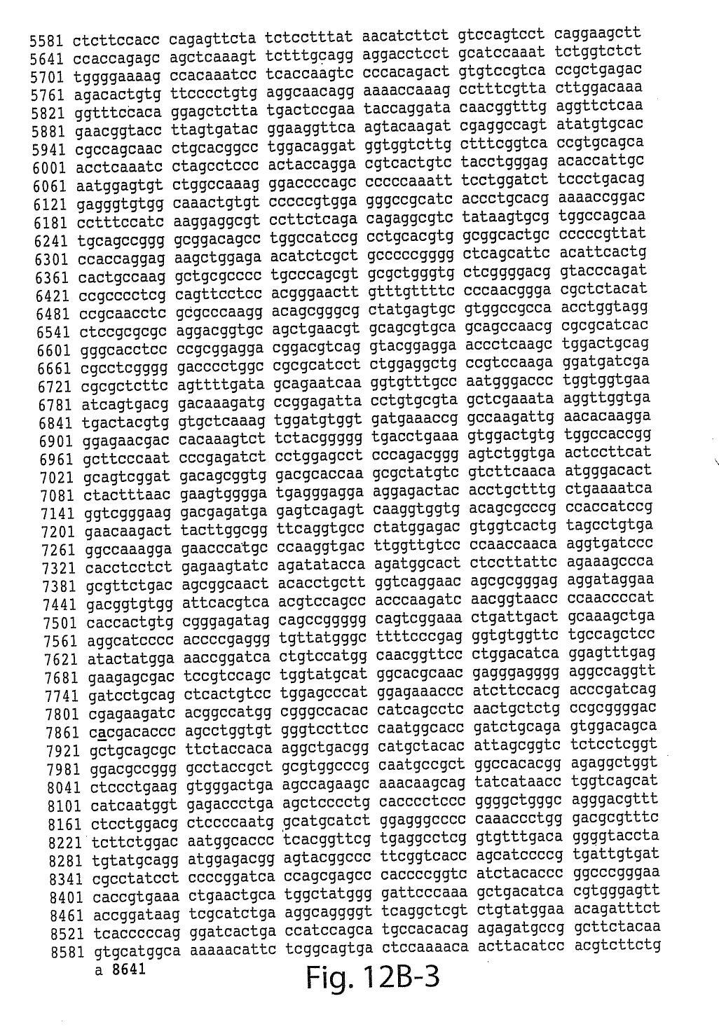

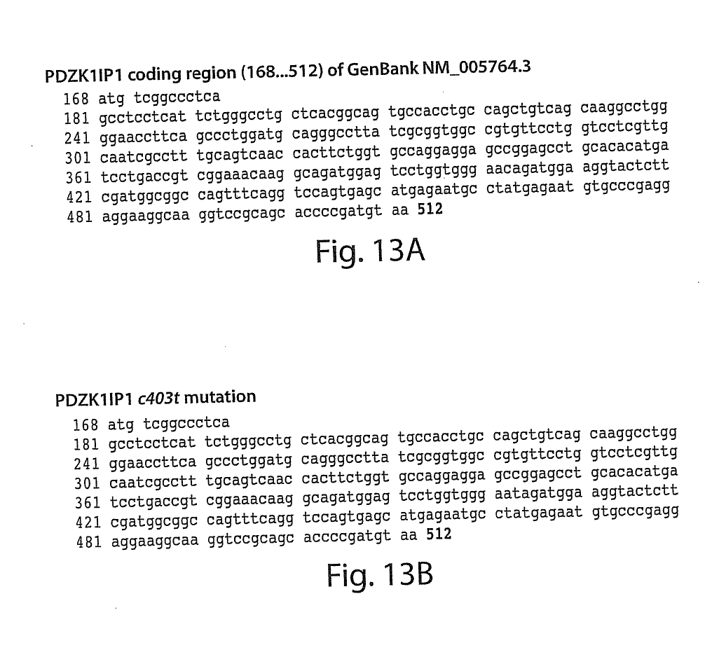

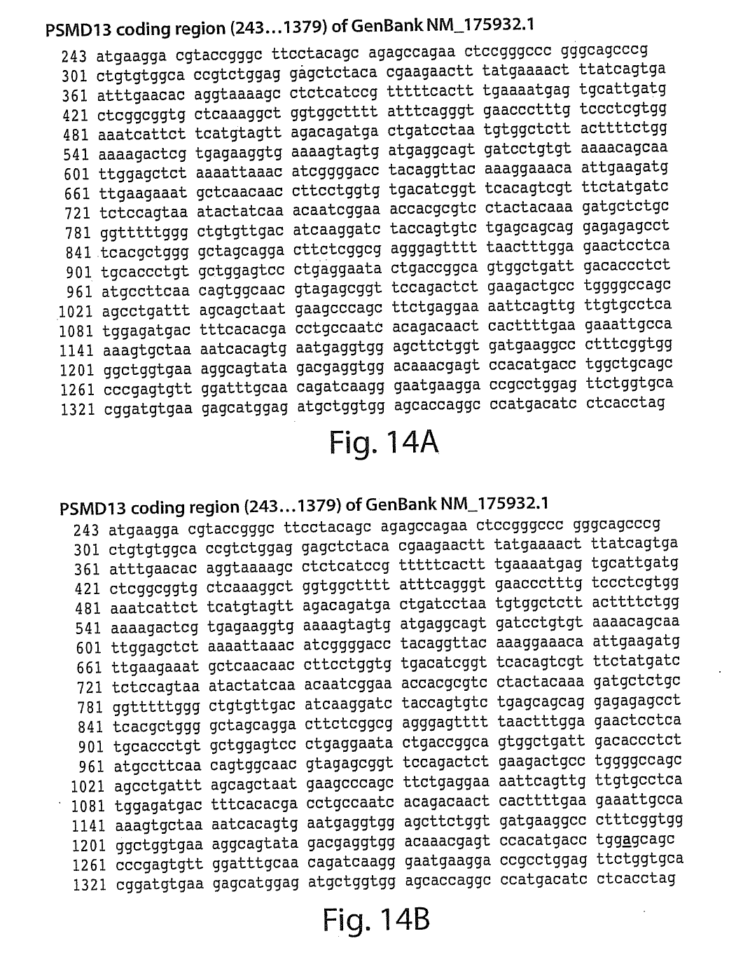

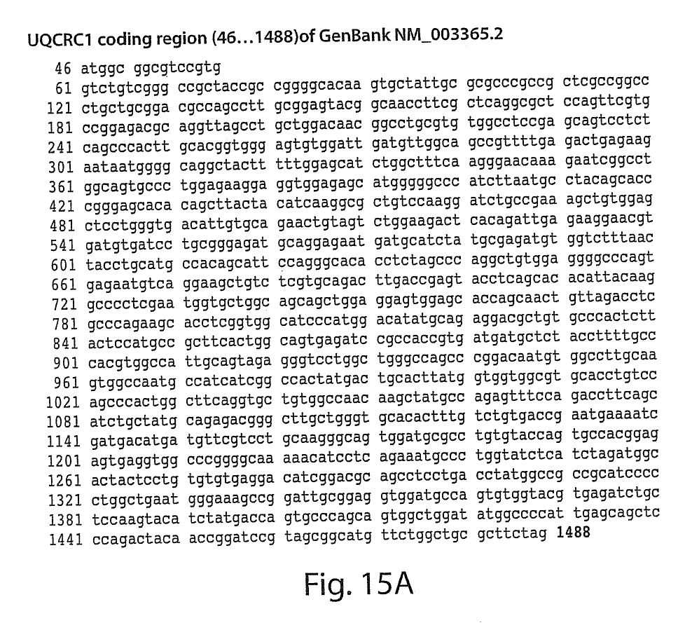

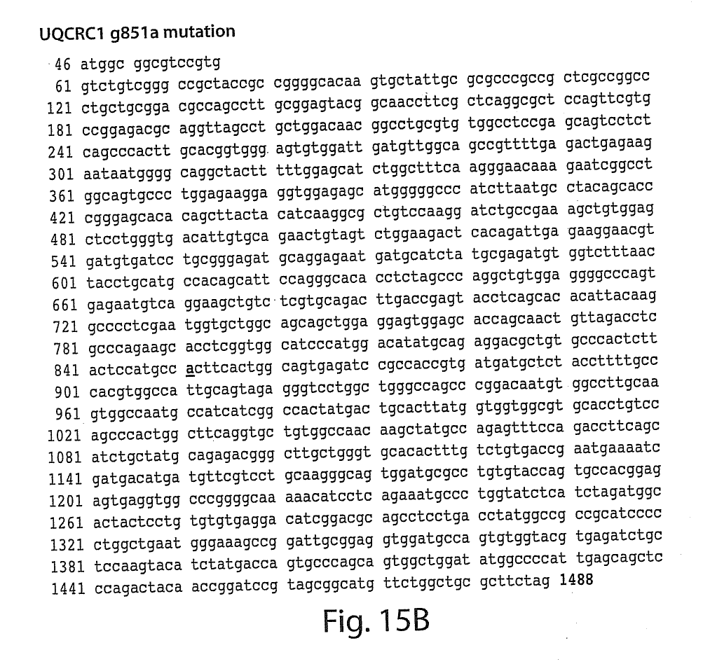

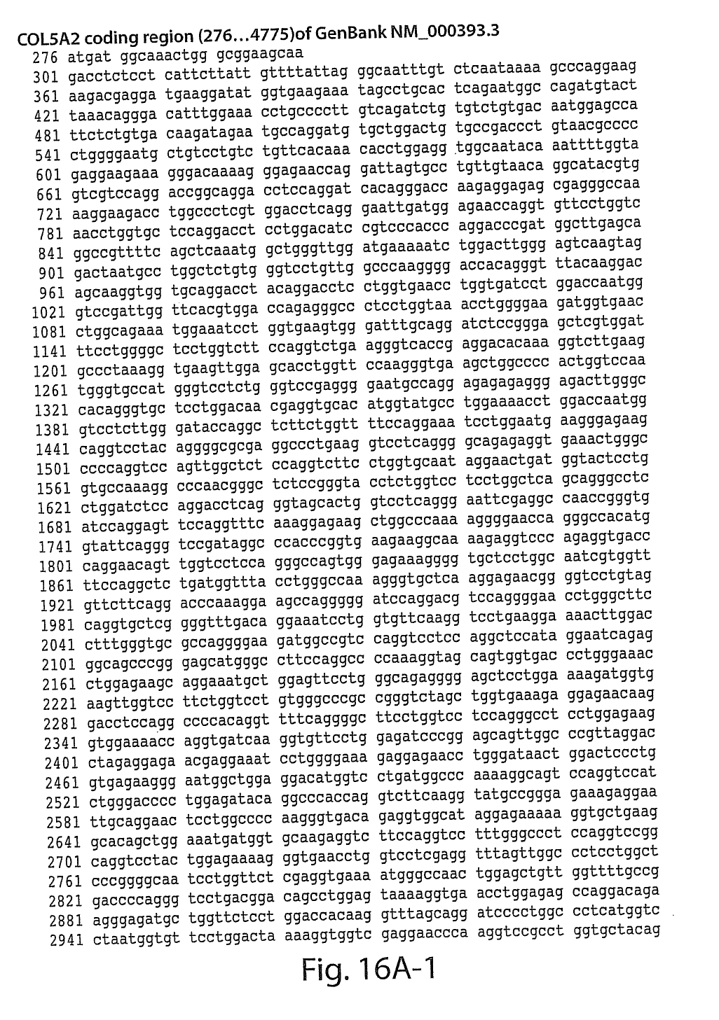

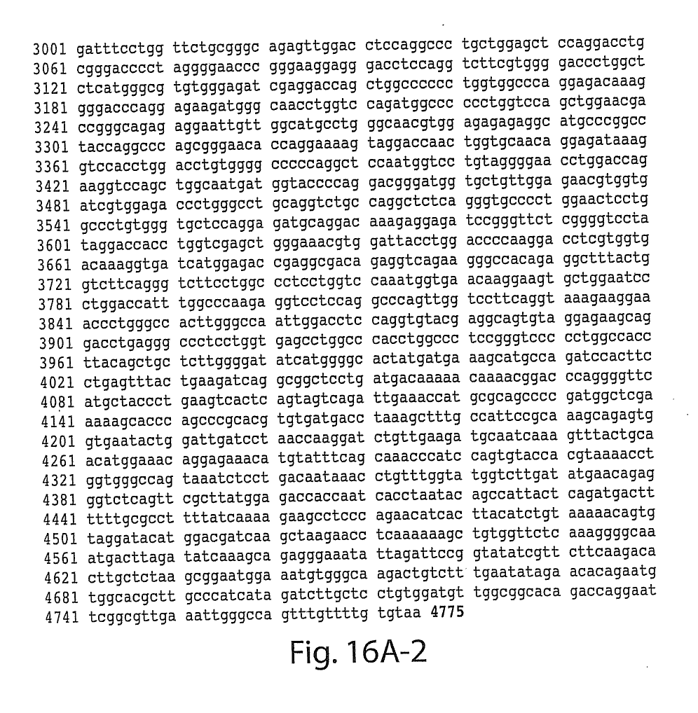

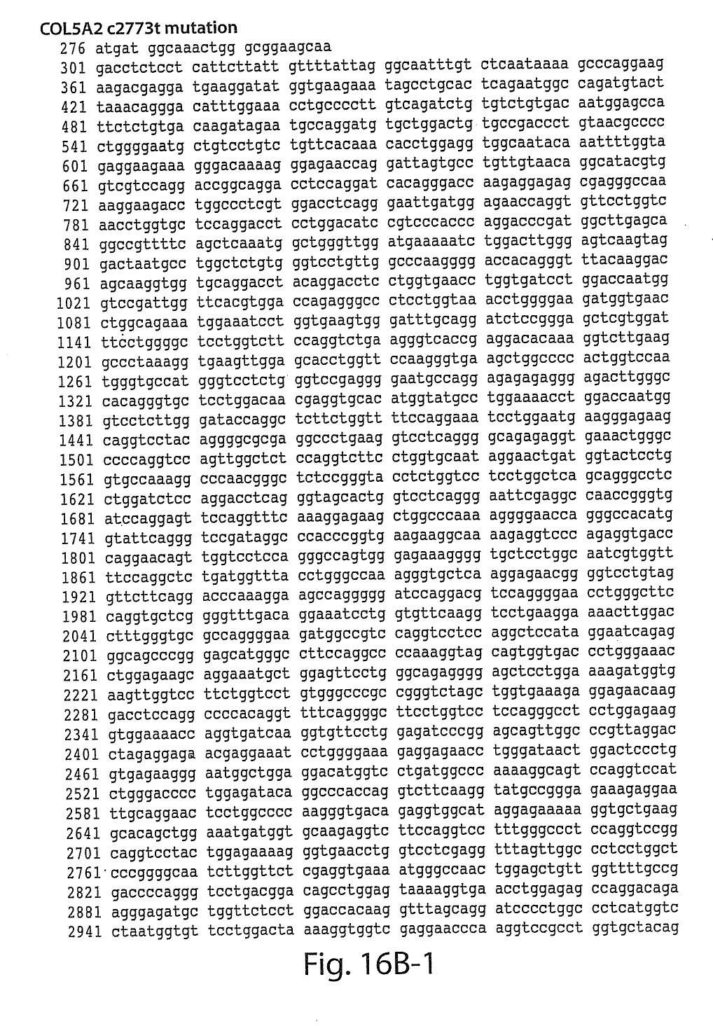

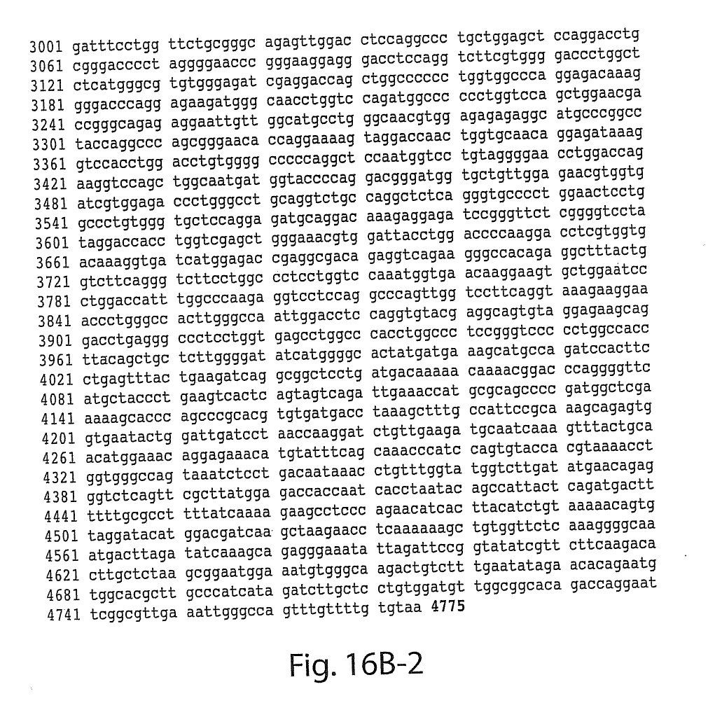

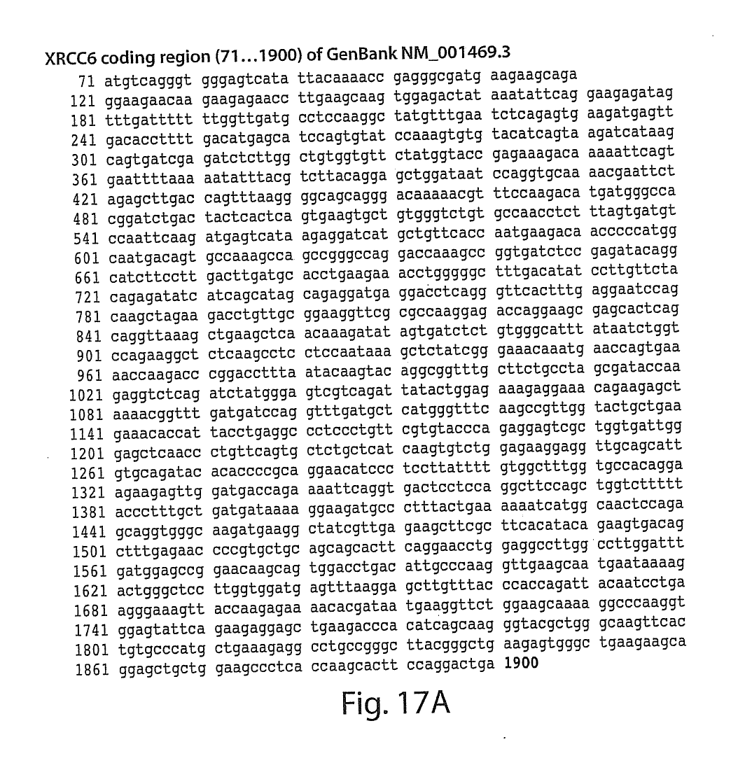

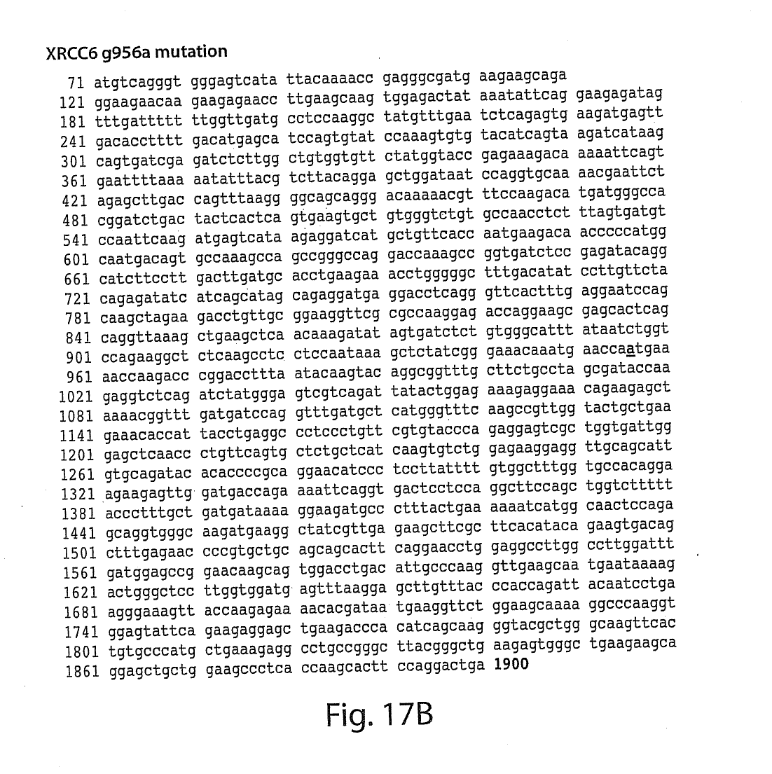

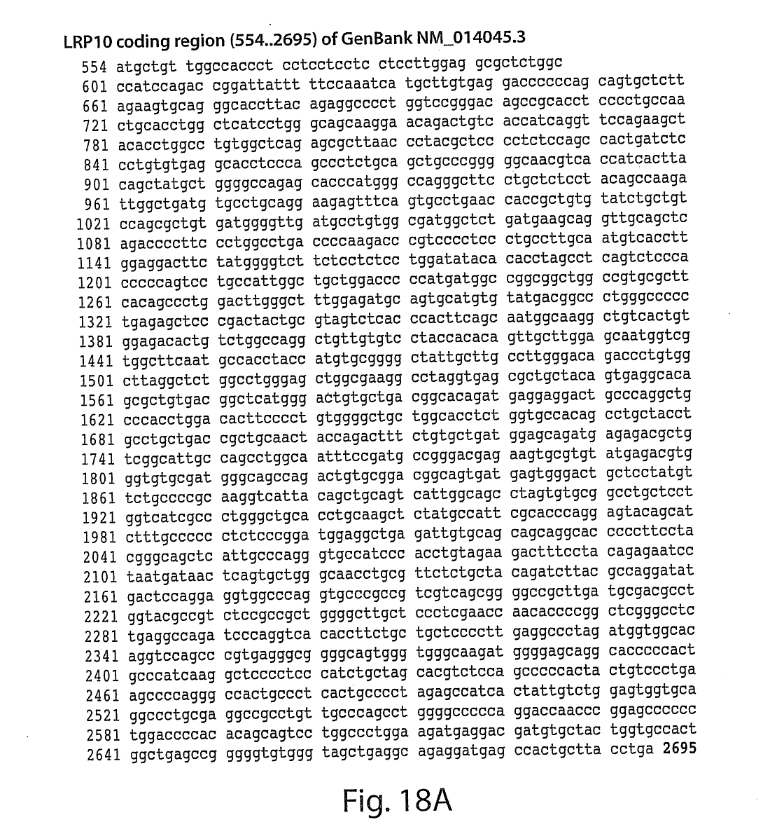

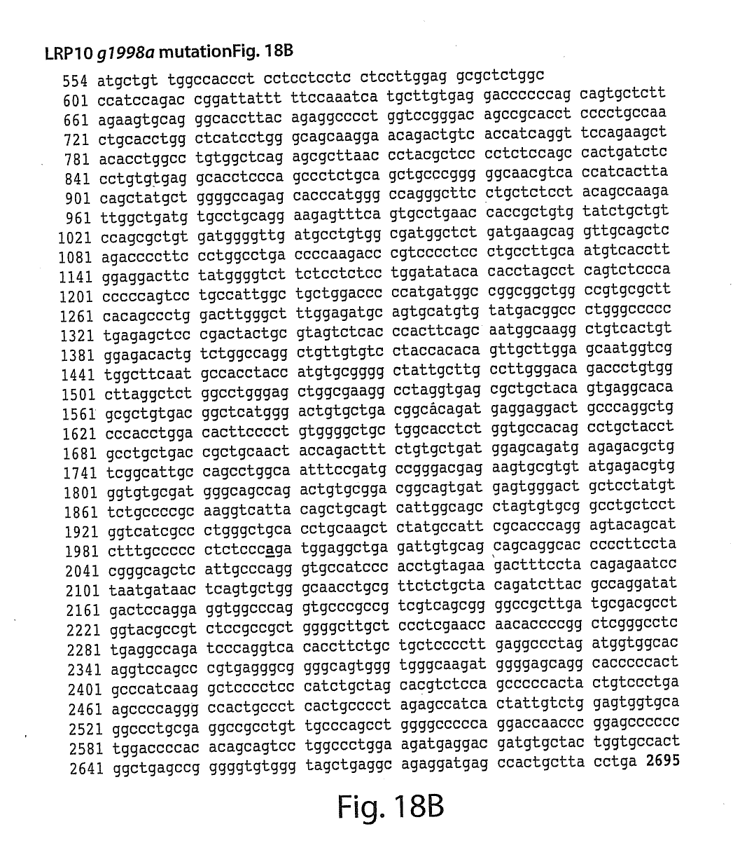

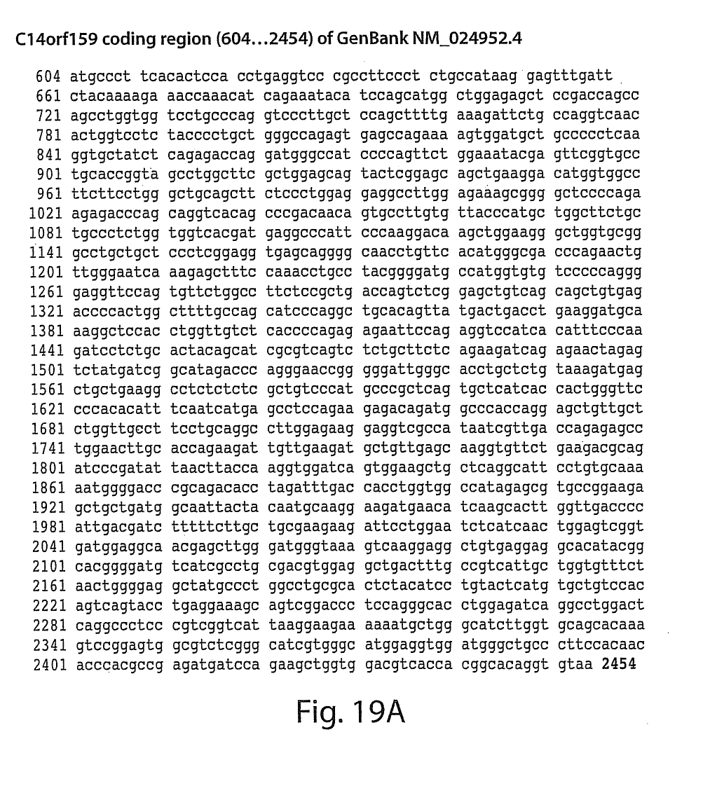

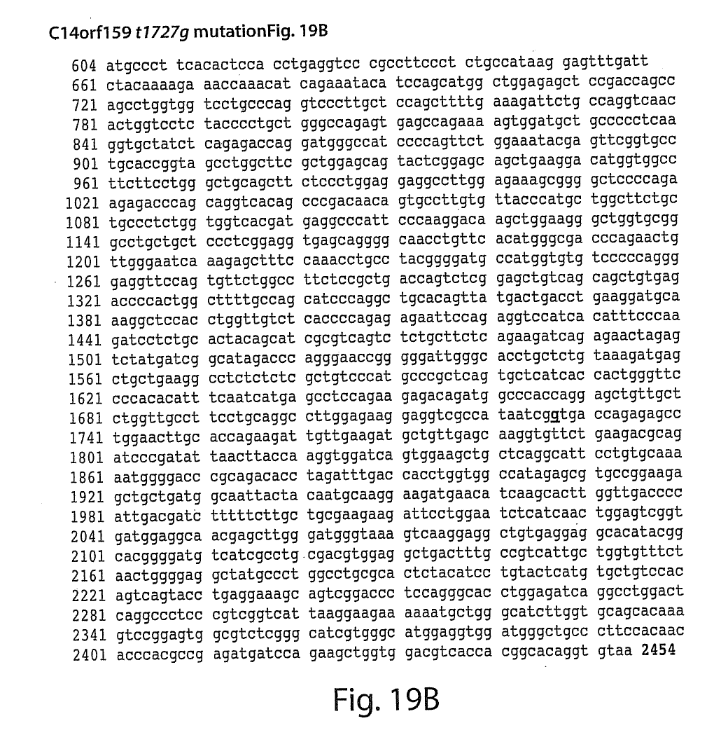

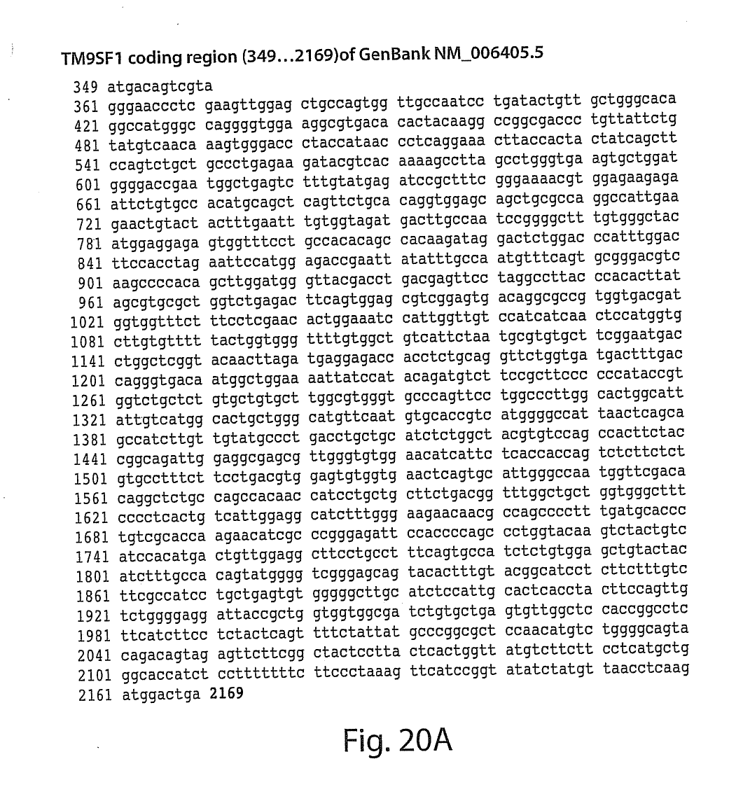

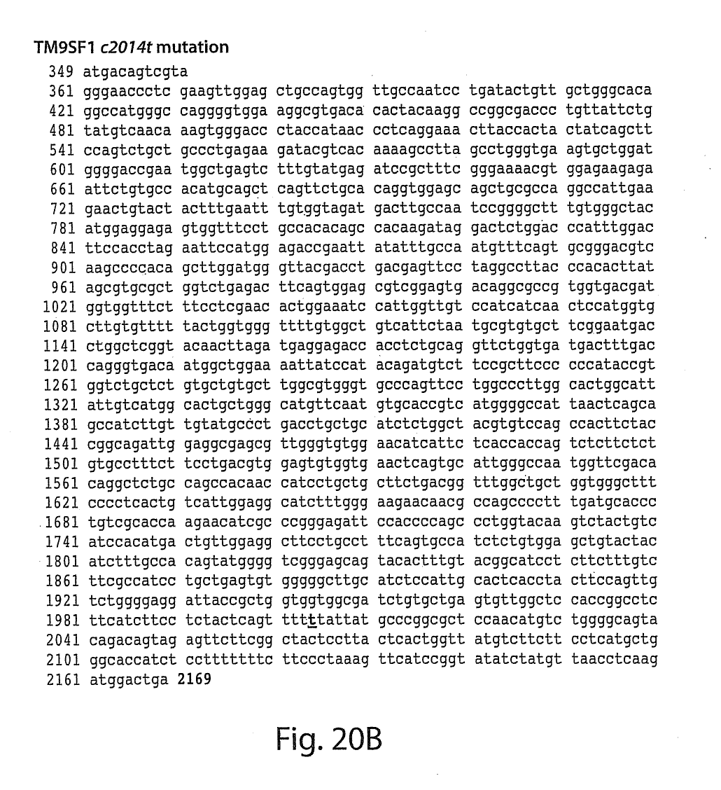

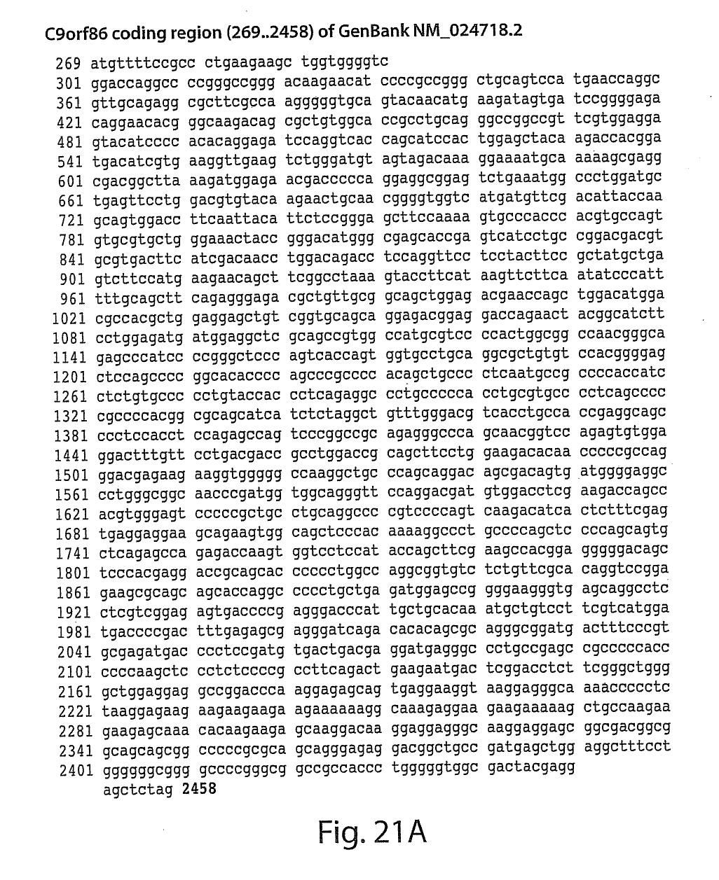

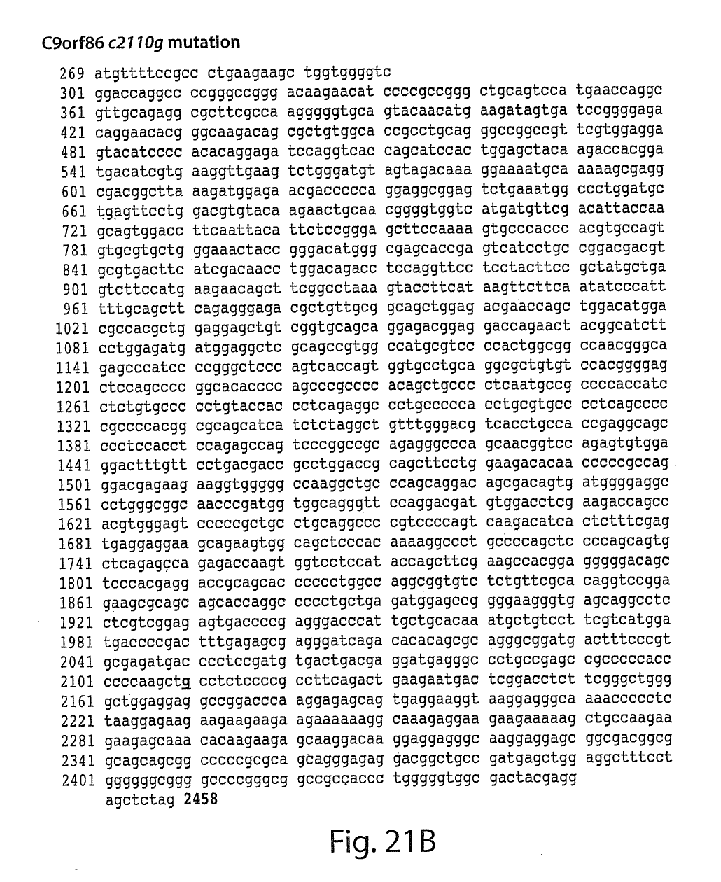

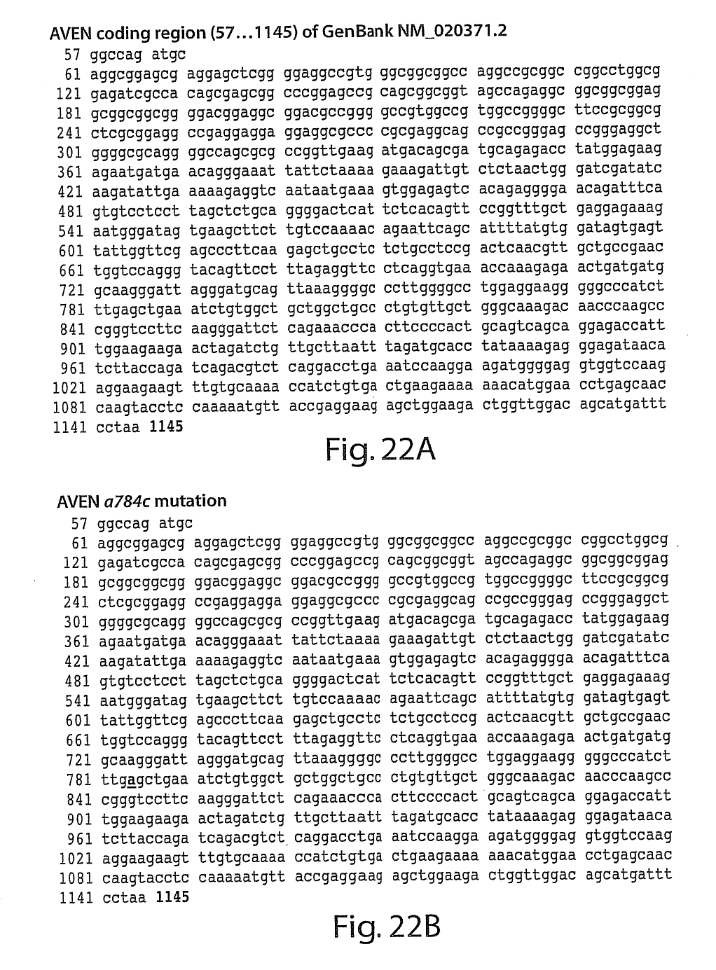

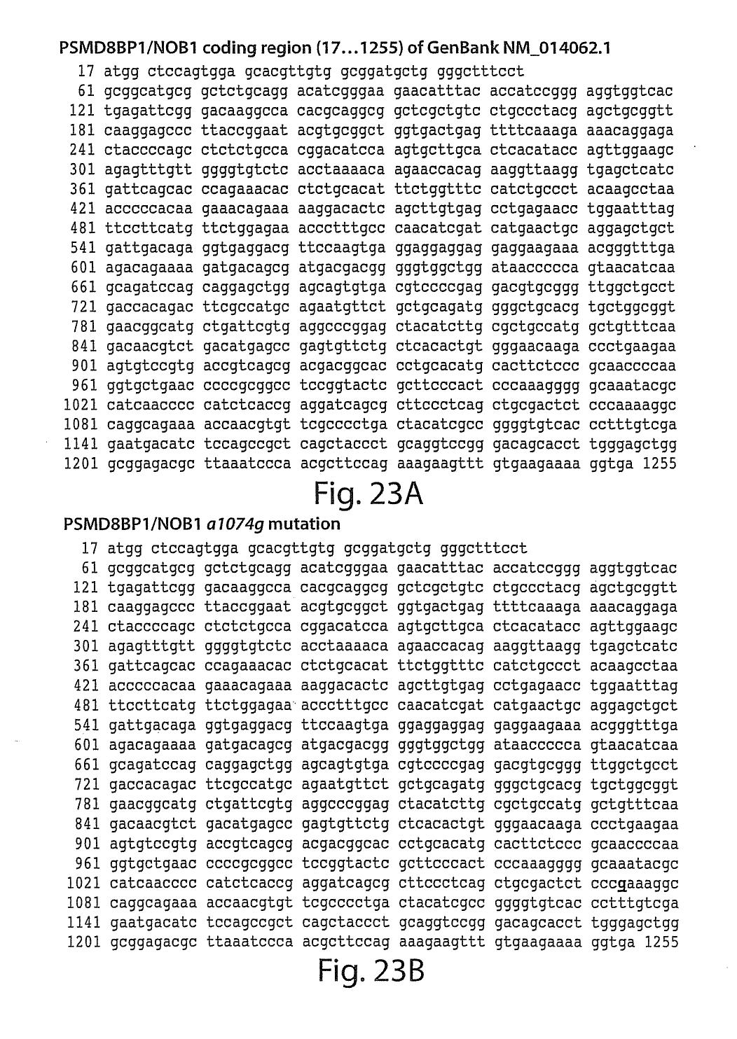

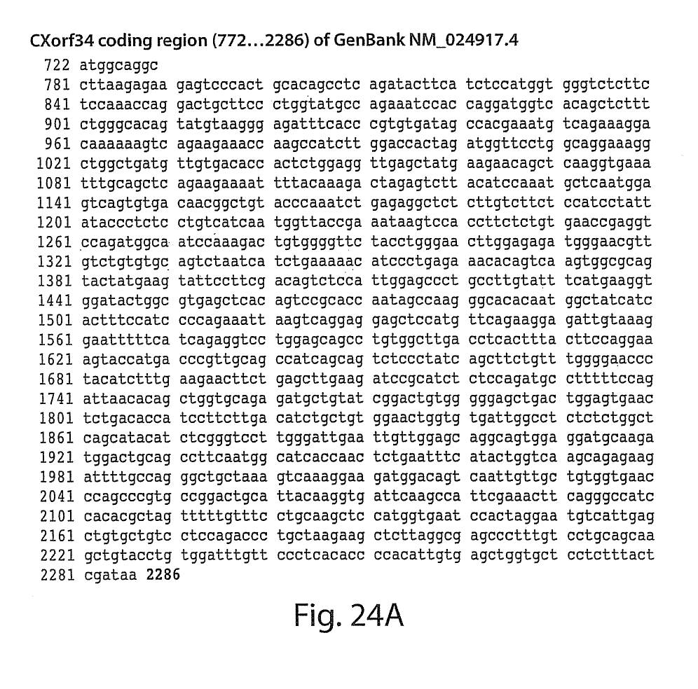

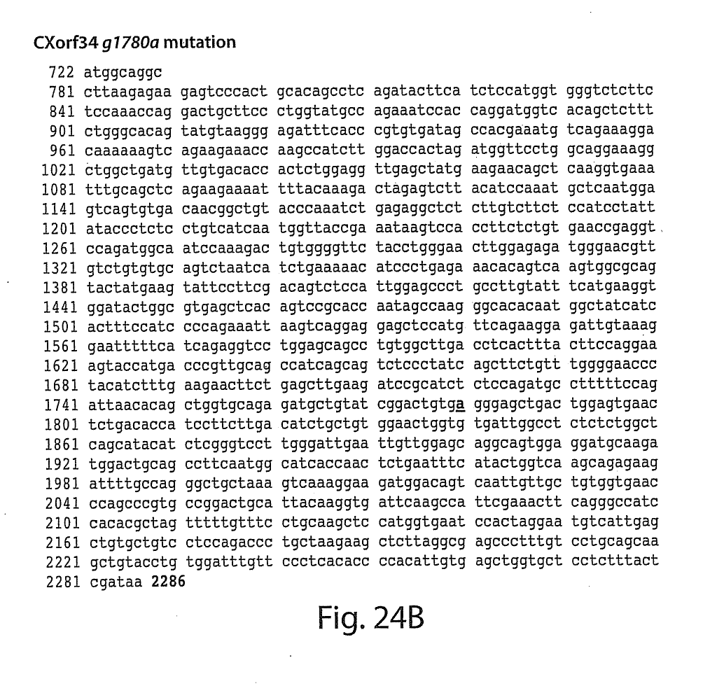

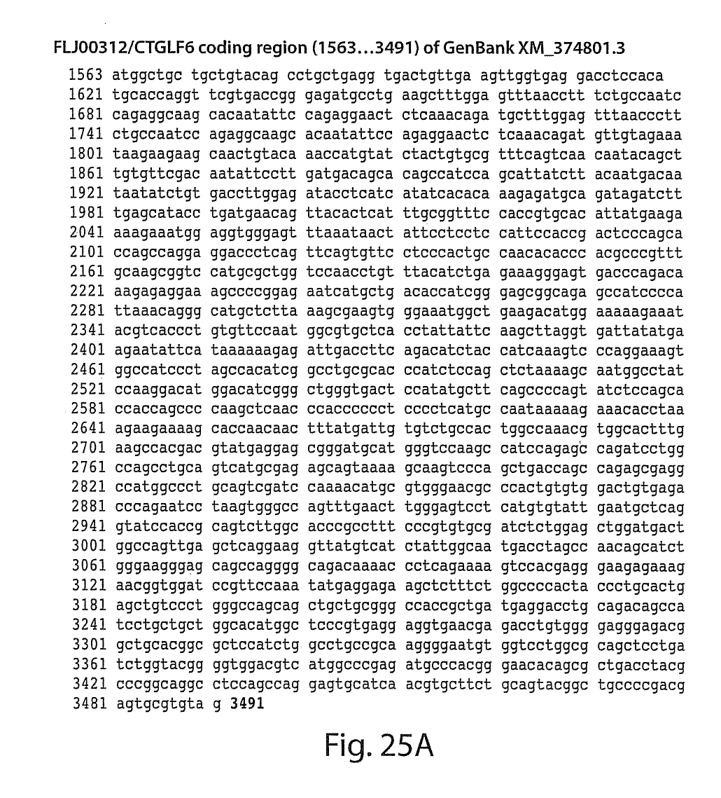

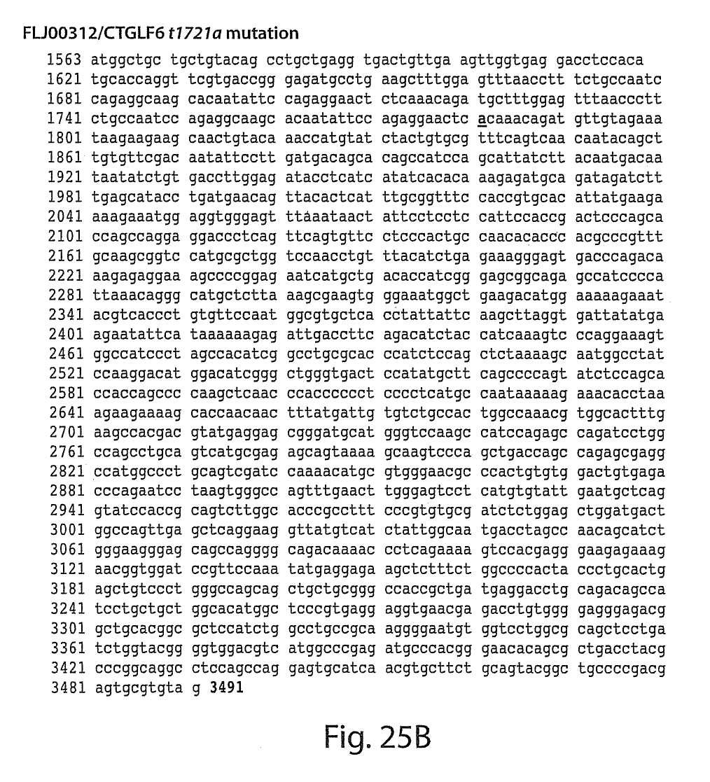

FIG. 9 lists 69 candidate mutations identified in the Examples. The mutations can be identified by reference to the column labeled “Gene,” which indicates the gene and/or name of the corresponding protein encoded by the gene in which the mutation maps. In addition, the FIG. 9 indicates the corresponding RefSeq accession number sequence for each gene. For example, gene/protein ACTR1A, the first entry on the chart, corresponds with RefSeq number NM_{—}005736.2. The mutation can be identified in relation to the nucleotide sequence of the RefSeq entry. For example, the column labeled “Position” indicates the nucleotide position in the RefSeq at which a mutation exists. The column labeled “Ref Allele” indicates the nucleotide at that position, e.g. at position 413 of NM_{—}005736.2. The column “Var Allele” indicates the identity of the mutation. Thus, for NM_{—}005736.2, the mutation discovered by the invention is a SNP which substituted a G for an A in the original sequence. Thus, by reference to the publicly available RefSeq, the identity of the particular genetic variation discovered by the invention as shown in FIG. 9 are easily ascertainable. FIGS. 11-25 provide the nucleotide sequences of the reference sequences and their mutated counterparts of the MPM markers of the invention shown in FIG. 10.

FIG. 10 indicates those genetic variations of FIG. 9 that have been shown to be novel, patient-specific and tumor-specific. That is, these mutations occur only in 1 of the 4 tumors originally evaluated and only are present in the tumor tissue, not in the healthy tissue of the individual. Like with FIG. 9, each mutation can be ascertained based on the given RefSeq accession number, the position of the nucleotide change, the change itself, and the consequential change in the encoded amino acids. For example, in the first entry the syntax “a413g” means that the mutation occurs at position 413 of the RefSeq NM_{—}005736.2 and changes the A in the reference sequence to a G in the transcriptome sequence.

### Oligonucleotides

The present invention further relates to the use of oligonucleotides for use in, inter alia, validating and detecting the genetic variations of the invention. In a first embodiment, as noted above, oligonucleotide primers can be used to amplify nucleic acid fragments which cover the mutation of interest such that the mutation can be verified and/or detected by sequencing the resulting PCR fragments (“amplicons”) and comparing the sequence to a reference sequence to determine whether or not the mutation is present. In addition, oligonucleotides can be prepared to contain the mutation itself and can be used in hybridization methods to detect the mutation in a target nucleic acid sample, e.g. a sample of DNA from a tissue suspected of being at risk of the disease for which the marker is associated with.

Oligonucleotides which are used in the detection methods of the present invention as primers and/or probes may be prepared based on the nucleotide sequences described in FIG. 9 or 10, for example, when SNPs are to be detected in tissue samples suspected of having MPM. The sequences per se may be synthesized, or primers and/or probes may be designed and synthesized so that they contain a part of these sequences. However, it should be noted here that the nucleotide sequences of such primers or probes must contain a genetic variation, e.g., a SNP, listed on FIG. 9 or 10. The present invention also includes complementary strands to such sequences.

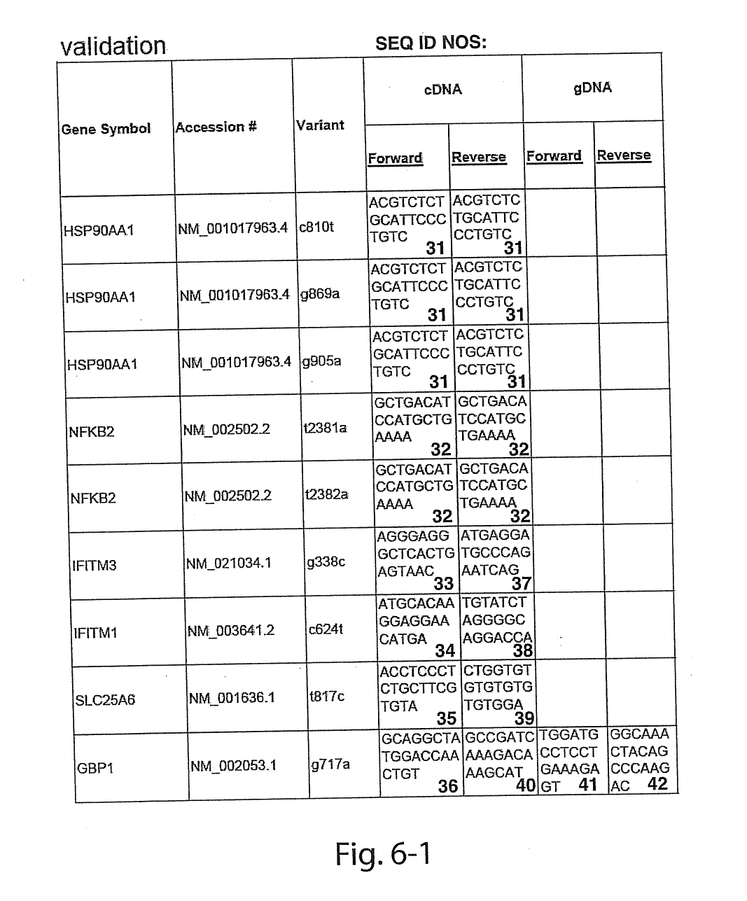

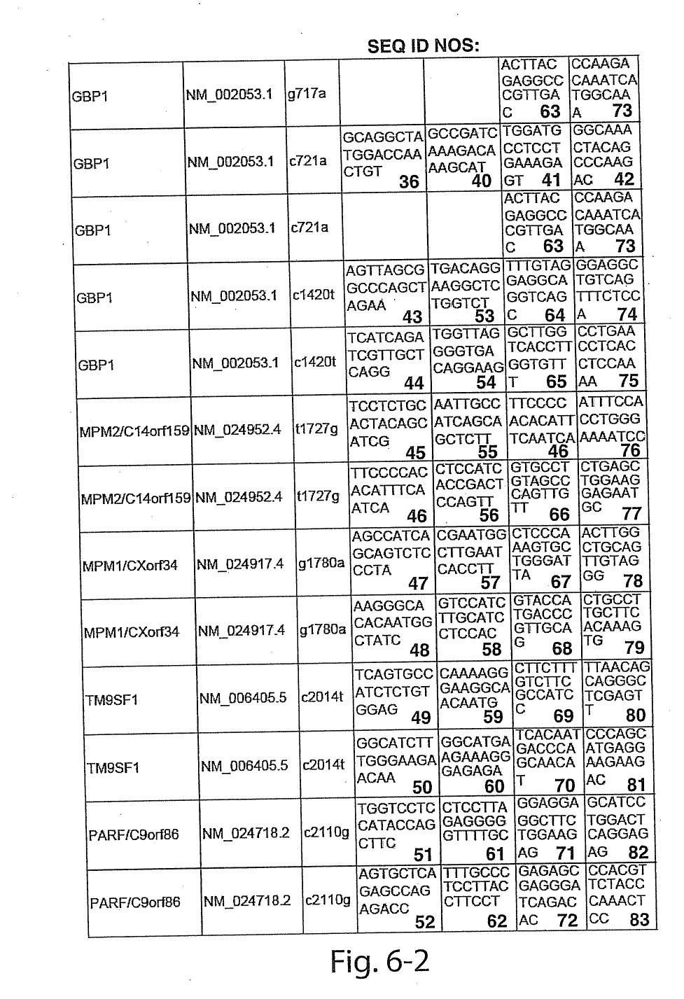

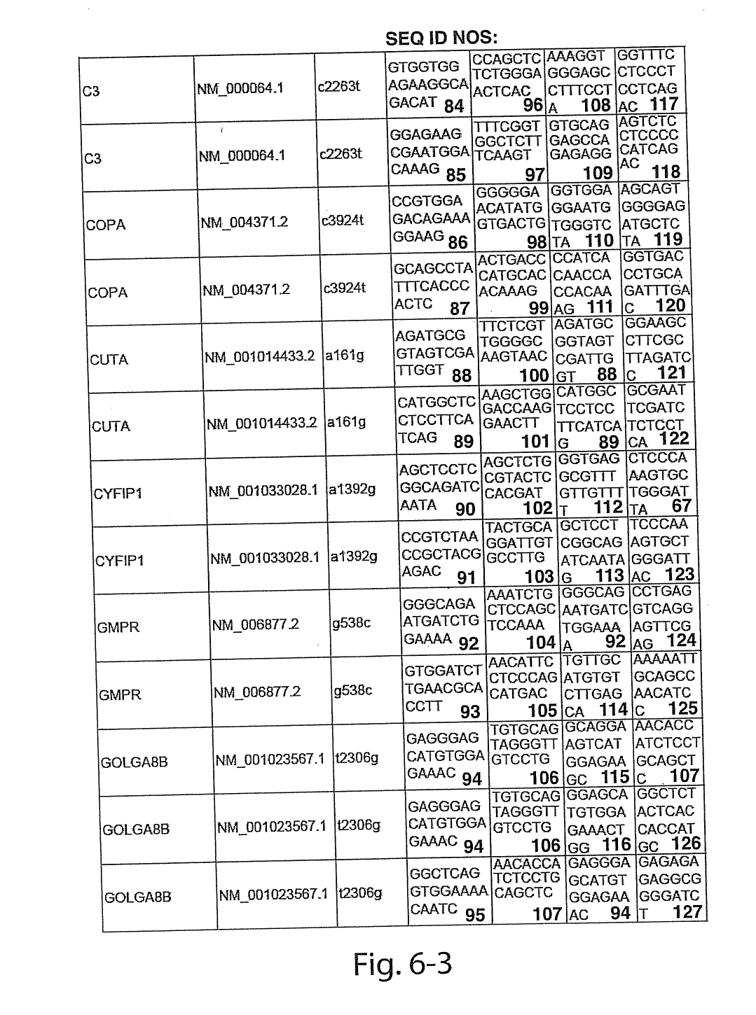

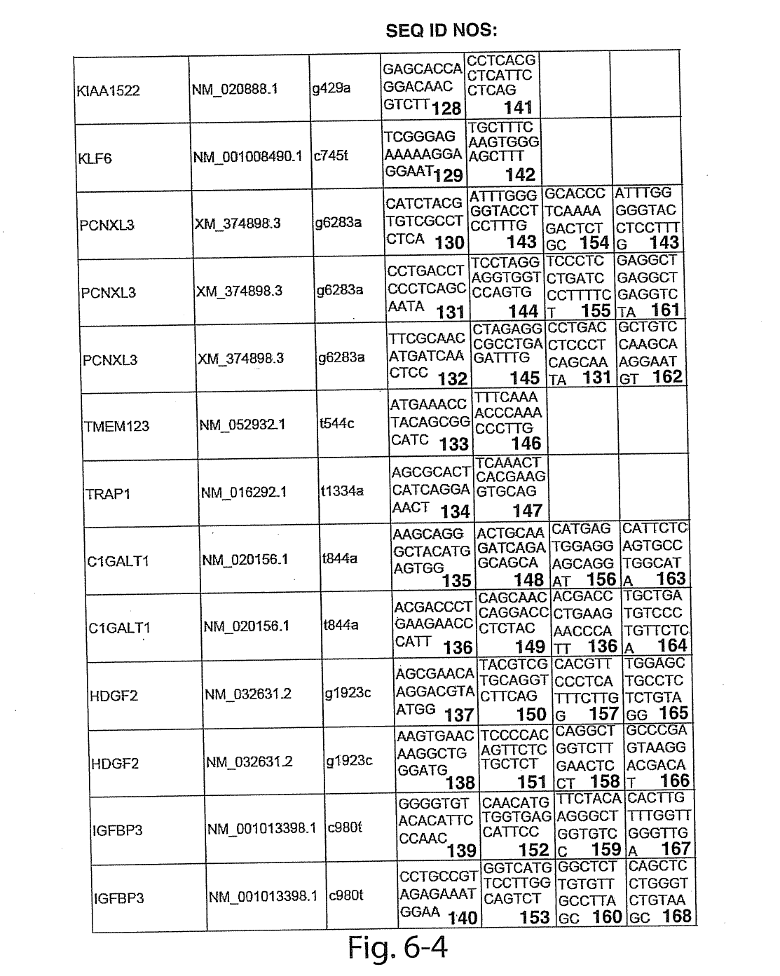

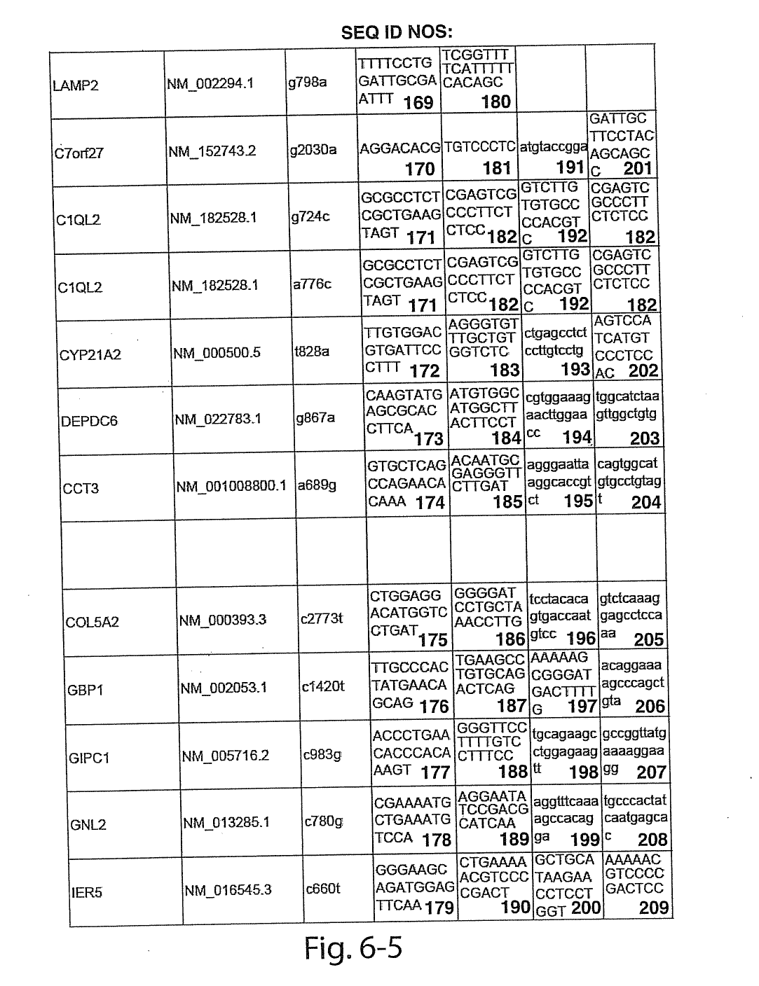

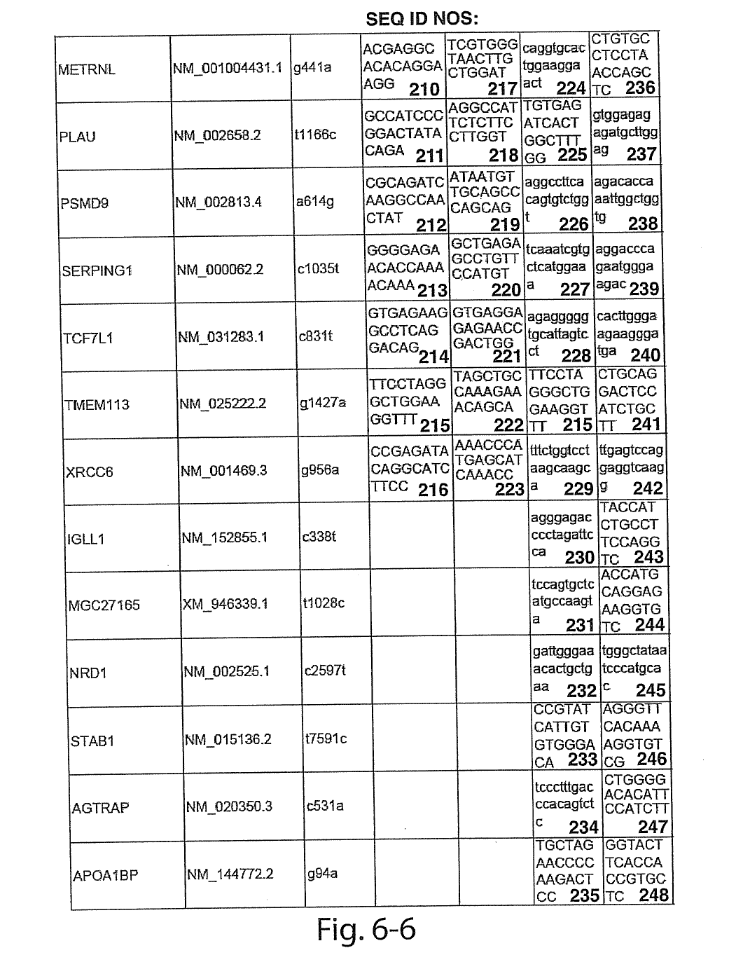

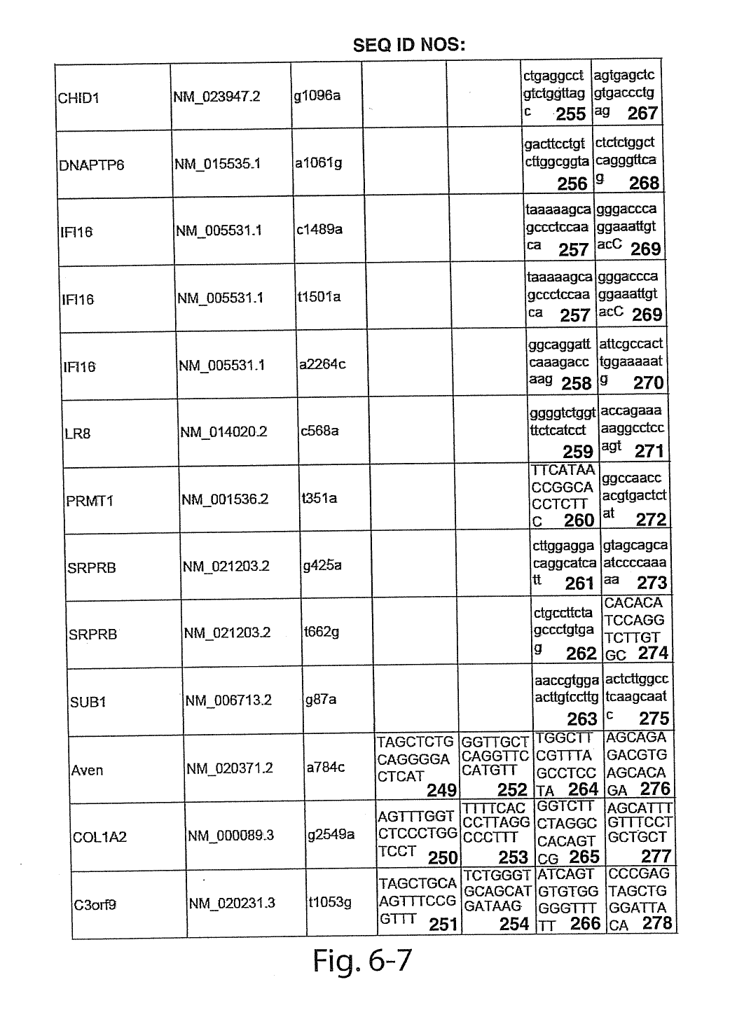

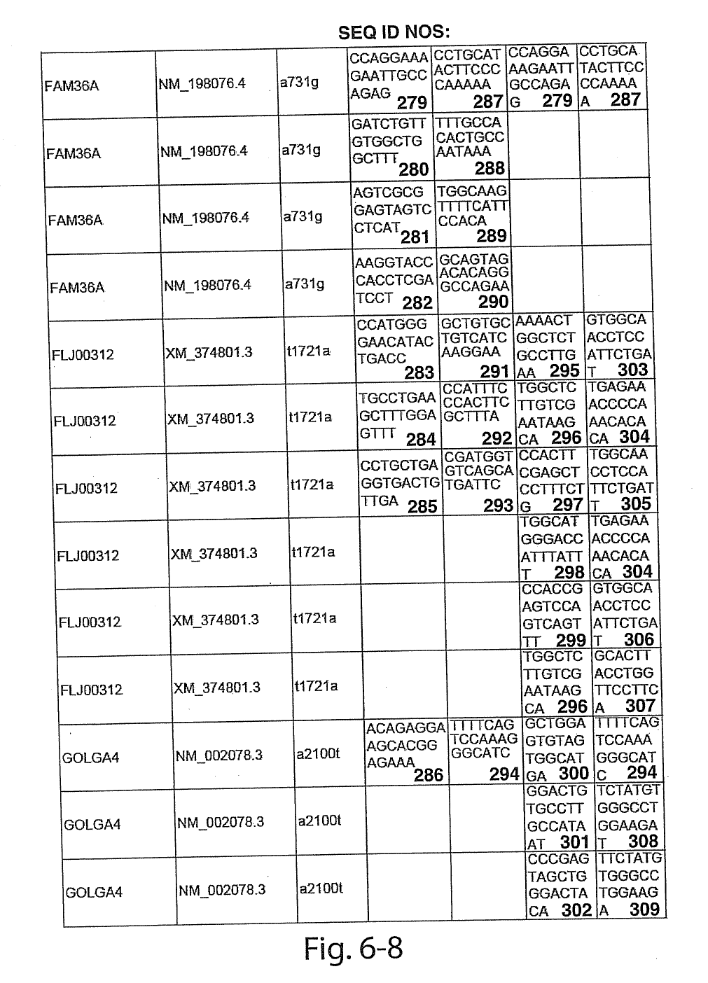

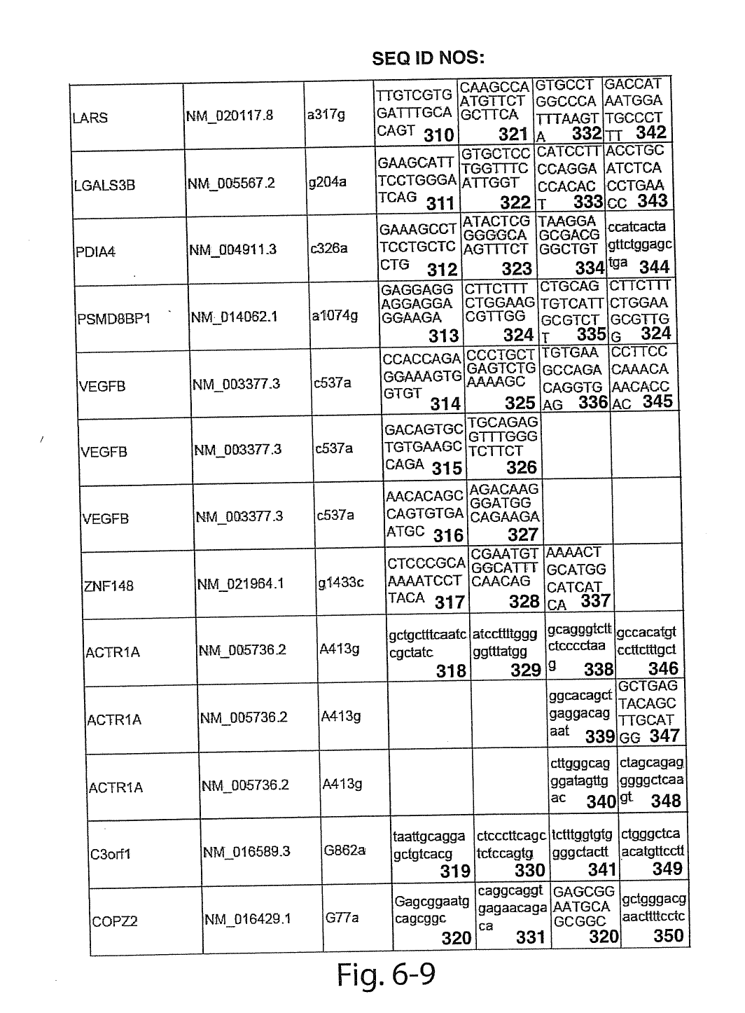

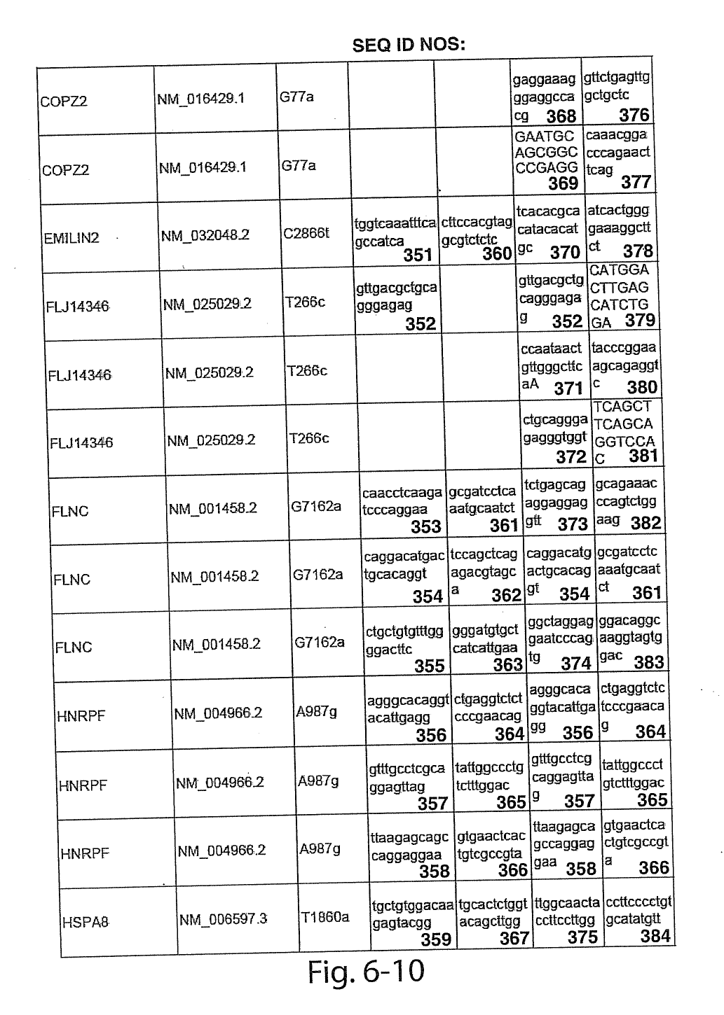

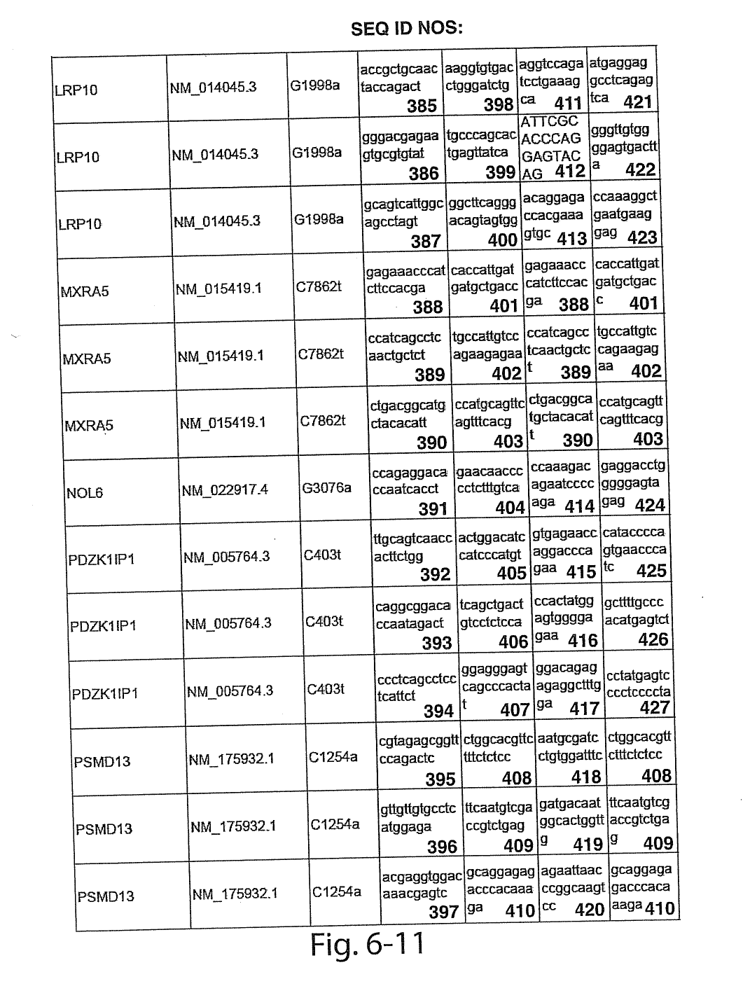

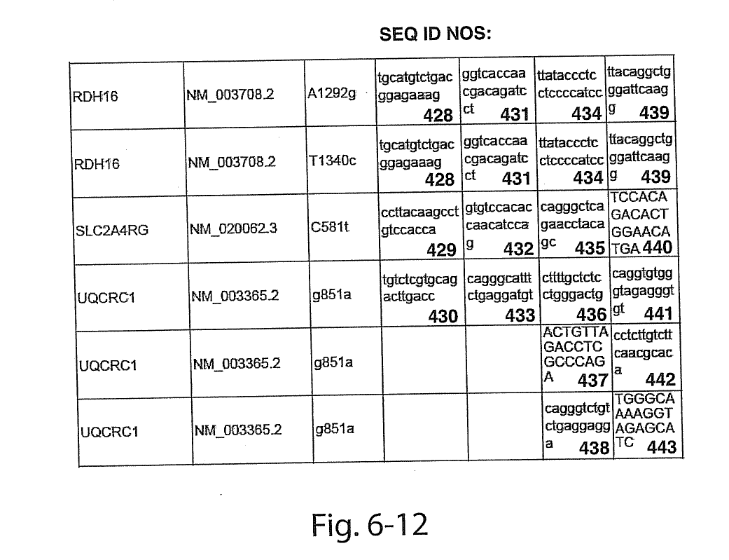

Oligonucleotide synthesis and design is well known in the art. Guidance can be found in molecular biology handbook. Taking SNPs as an example, however, for the purpose of illustration, a primer or probe is designed so that an SNP site is located at the 3′ or 5′ end of the nucleotide sequence of the primer or probe; or a primer or probe is designed so that an SNP site is located at the 3′ or 5′ end of the sequence complementary to its nucleotide sequence; or a primer or probe is designed so that an SNP site is located within four nucleotides, preferably two nucleotides, from the 3′ or 5′ end of its nucleotide sequence or the sequence complementary thereto. Alternatively, a primer or probe is designed so that an SNP site is located at the center of the full-length nucleotide sequence of the oligonucleotide. The “center” refers to a central region where the number of nucleotides counted from there toward the 5′ end and the number of nucleotides counted from there toward the 3′ end are almost equal. If the number of nucleotides of the oligonucleotide is an odd number, the “center” is the central five nucleotides, preferably the central three nucleotides, more preferably the single nucleotide at the very center. For example, if the oligonucleotide consists of 41 nucleotides, the “center” is from position 19 to position 23 nucleotides, preferably from position 20 to position 22 nucleotides, more preferably the nucleotide at position 21. If the number of nucleotides of the oligonucleotide is an even number, the “center” refers to the central four nucleotides, preferably the central two nucleotides. For example, if the oligonucleotide consists of 40 nucleotides, the “center” is from position 19 to position 22 nucleotides, preferably the nucleotide at position 20. In the nucleotide sequences shown in the “Sequence” column in FIG. 6, if the polymorphism is deletion polymorphism, the actual length of such sequences is 40, an even number. Therefore, if an oligonucleotide consisting of 40 nucleotides is designed based on such sequences, the “center” is from position 19 to position 22 nucleotides, preferably the nucleotide at position 20.

When a polymorphic site is composed of a plurality of nucleotides, a probe or primer is designed and prepared so that the entire or a partial nucleotide sequence of the polymorphic site or a sequence complementary thereto is contained in the nucleotide sequence of the probe or primer. When the thus prepared oligonucleotide is used as a probe, it is possible to determine alleles using the presence or absence of hybridization or difference of hybridization. Those nucleotides in a probe or primer DNA which form a complementary strand with the polymorphic site or its peripheral site are called “corresponding nucleotides.” A probe or primer may be designed so that corresponding nucleotides are located on any nucleotide(s) on the sequence constituting a polymorphism. The “peripheral site” means a region one to three nucleotides outside (5′ side) of the 5′ utmost end of the sequence constituting a polymorphism, or a region one to three nucleotides outside (3′ side) of the 3′ utmost end of the sequence constituting a polymorphism. In particular, the corresponding nucleotides in a probe or primer can be designed so that the 5′ or 3′ end nucleotide when forming a complementary strand is located on the 5′ terminal side, 3′ terminal side, or the center of the sequence constituting a polymorphism. In the present invention, it is preferred that the above-mentioned 5′ or 3′ end nucleotide is located on the center of the sequence constituting a polymorphism. It is also possible to design corresponding nucleotides so that they are located on a peripheral region of the sequence constituting a polymorphism.

The length of the nucleotide sequence can be designed with any suitable length, but preferably so that at least 13 nucleotides, preferably 13 to 60 nucleotides, more preferably 15 to 40 nucleotides, and most preferably 18-30 nucleotides are contained. This oligonucleotide sequence may be used as a probe for detecting a target gene, and it may be used as either a forward (sense) primer or a reverse (antisense) primer.

The oligonucleotide used in the invention may be an oligonucleotide composed of two regions connected in tandem, one region being hybridizable to the genomic DNA and the other region being not hybridizable thereto. The order of connection is not particularly limited; either region may be located upstream or downstream. The hybridizable region of this oligonucleotide can be designed based on the information on SNP-containing sequences described in FIG. 9 or 10. The oligonucleotide can be prepared so that the nucleotide located at the 5′ or 3′ utmost end of the region hybridizable to the genomic DNA corresponds to an SNP of interest. The region of the above oligonucleotide not hybridizable to the genomic DNA is designed at random so that it does not hybridize to the SNP-containing sequence described in FIG. 9 or 10. This oligonucleotide may be used as a probe mainly for detecting SNPs.

Further, the primers used in the present invention can be designed so that a nucleotide sequence given in FIG. 9 or 10 contains an SNP when amplified by PCR. The length of the primer is designed so that at least 15 nucleotides, preferably 15 to 30 nucleotides, more preferably 18 to 24 nucleotides are contained in the primer. The primer sequence is appropriately selected from the template DNA so that the amplified fragment has a length of 1000 bp or less, preferably within 500 bp (e.g. 120 to 500 bp), more preferably within 200 bp (120 to 200 bp).

FIG. 6 provides the sequences of primers that can be used to amplify fragments spanning the SNPs of FIG. 9.

The oligonucleotide primers or probes of the invention may be synthesized chemically according to known techniques. Usually, such primers or probes are synthesized with a commercial chemical synthesizer.

It is also possible to label probes with fluorescent substances (e.g. FAM, VIC, Redmond Dye, etc.) in advance to thereby automate detection procedures. The labels may be incorporated before, during or after hybridization by any suitable means of attaching labels to nucleic acids known in the art. Suitable means may include addition of a label directly to the original transcript-specific element of the sample (e.g., mRNA, polyA mRNA, cDNA, etc.) or to an amplification product during or after amplification of the transcript-specific element of the sample, e.g. using labelled primers or labelled nucleotides.

Labels suitable for use in the methods described herein include, but are not limited to, biotin for staining with labelled streptavidin conjugate, magnetic beads (e.g., Dynabeads), fluorescent dyes (e.g., fluorescein, Texas red, rhodamine, green fluorescent protein, and the like), radiolabels (e.g., ³H, ¹²⁵I, ³⁵S, ¹⁴C, or ³²P), r) enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads.

Depending on the choice of label, the skilled person will be able to choose suitable means for detection of the label well known in the art. For a detailed review of methods of labelling nucleic acids and detecting labelled hybridized nucleic acids see LABORATORY TECHNIQUES IN BIOCHEMISTRY AND MOLECULAR BIOLOGY, Vol. 24: Hybridization With Nucleic Acid Probes, P. Tijssen, ed. Elsevier, N.Y., (1993), which is incorporated herein by reference.

The probe prepared as described above can be hybridized to template DNAs to thereby detect those DNAs having the genetic polymorphism or variation of interest. The template DNA may be prepared according to conventional methods, e.g. cesium chloride gradient centrifugation, the SDS lysis method, or phenol/chloroform extraction.

### (1) Detection by PCR

Amplification may be performed by polymerase chain reaction (PCR). Specific examples of useful DNA polymerase include LA Taq DNA polymerase (Takara), Ex Taq polymerase (Takara), Gold Taq polymerase (Perkin Elmer), AmpliTaq (Perkin Elmer), Pfu. DNA polymerase (Stratagene) and the like.

Subsequently, the amplified product can be subjected to agarose gel electrophoresis, followed by staining with ethidium bromide, SYBR Green solution or the like to thereby detect the amplified product as a band or two to three bands (DNA fragments). Thus, a part of a gene encoding a receptor, containing a genetic polymorphism can be detected as a DNA fragment. Instead of agarose gel electrophoresis, polyacrylamide gel electrophoresis or capillary electrophoresis may be performed. It is also possible to perform PCR using primers labeled in advance with a substance such as fluorescent dye and to detect the amplified product. A detection method which does not require electrophoresis may also be employed; in such a method, the amplified product is bound to a solid support such as a microplate, and a DNA fragment of interest is detected by means of fluorescence, enzyme reaction, or the like.

### (2) Detection by TaqMan PCR

TaqMan PCR is a method using PCR reaction with fluorescently labeled allele-specific oligos and Taq DNA polymerase. The allele-specific oligo used in TaqMan PCR (called “TaqMan probe”) may be designed based on the SNP information described above, e.g. in FIG. 9 or 10. The 5′ end of TaqMan probe is labeled with fluorescence reporter dye R (e.g. FAM or VIC), and at the same time, the 3′ end thereof is labeled with quencher Q (quenching substance). Thus, under these conditions, fluorescence is not detectable since the quencher absorbs fluorescence energy. Since the 3′ end of TaqMan probe is phosphorylated, no extension reaction occurs from TaqMan probe during PCR reaction. However, when PCR reaction is performed using this TaqMan probe together with Taq DNA polymerase and primers designed so that an SNP-containing region is amplified, the reaction described below occurs.

First, a TaqMan probe hybridizes to a specific sequence in the template DNA, and at the same time, an extension reaction occurs from a PCR primer. At this time, Taq DNA polymerase having 5′ nuclease activity cleaves the hybridized TaqMan probe as the extension reaction of PCR primer proceeds. When the TaqMan probe has been cleaved, the fluorescent dye becomes free from the influence of the quencher. Then, fluorescence can be detected.

For example, two alleles are supposed: one allele has A at the SNP site (allele 1) and the other allele has G at the SNP site (allele 2). A TaqMan probe specific to allele I is labeled with FAM and another TaqMan probe specific to allele 2 is labeled with VIC. These two allele specific oligos are added to PCR reagents, and then TaqMan PCR is performed with a template DNA whose SNP is to be detected. Subsequently, fluorescence intensities of FAM and VIC are determined with a fluorescence detector. When the SNP site of the allele is complementary to the site within TaqMan probe corresponding to the SNP, the probe hybridizes to the allele; and Taq polymerase cleaves the fluorescent dye of the probe, which becomes free form the influence of the quencher. As a result, fluorescence intensity is detected.

### (3) Other PCR Methods

Any suitable PCR method for detecting SNPs in target DNA are contemplated by the present invention, including new methods, such as the invader PCR method (e.g. Allawi et al., J Clin Microbio, 2006, 44: 3443-3447, incorporated herein by reference) or the SniPer PCR method (Huentelman et al., BMC Genomics, 2005, 6:149, incorporated herein by reference), both of which can be used to detect SNPs.

### (4) Detection by DNA Sequencing Method

In the present invention, polymorphisms may be detected by using single nucleotide extension reactions. Briefly, four types of dideoxynucleotides labeled with different fluorescent compounds are added to a reaction system containing a gene of interest. Then, single nucleotide extension reactions are performed. In this case, the nucleotide to be extended is the polymorphic site. Also, two reactions of DNA synthesis termination and the fluorescent labeling of the 3′ end of DNA molecules are operated. Four types of reaction solutions are subjected to electrophoresis on the same lane of a sequencing gel or on capillary. Difference in the fluorescent dyes used for labeling is detected with a fluorescence detector to thereby sequence the DNA band. Alternatively, the one-nucleotide extended oligonucleotide is examined with a fluorescence detection system or a mass spectrometry system or the like to thereby determine which nucleotide was extended using the difference in the fluorescent dyes. Instead of fluorescently labeled dideoxynucleotides, primers may be fluorescently labeled and used with unlabeled dideoxynucleotides.

### (5) Detection in DNA Microarray

DNA microarrays are solid supports onto which nucleotide probes are immobilized, and they include DNA chips, gene chips, microchips, beads arrays, and the like.

As a specific example of a DNA microarray (e.g. DNA chip) assay, GeneChip assay (Affymetrix; U.S. Pat. Nos. 6,045,996; 5,925,525; and 5,858,659, incorporated herein by reference) may be given. GeneChip technology uses small sized, high density microarrays of oligonucleotide probes affixed to chips. Probe arrays are manufactured, for example, by the light irradiation chemical synthesis method (Affymetrix) which is a combination of solid chemical synthesis method and photolithography production technology used in the semiconductor industry. High density arrays to which oligonucleotide probes are affixed on designed place can be constructed by using photolithography masks in order to make the boundary of the chemical reaction site of chips definite and by performing a specific chemical synthesis step. Multiple-probe arrays are synthesized simultaneously on a large glass baseboard. Subsequently, this baseboard is dried, and individual probe arrays are packed in injection-molded plastic cartridges. This cartridge protects the array from the outer environment and also serves as a hybridization chamber.

First, a polynucleotide to be analyzed is isolated, amplified by PCR, and labeled with a fluorescent reporter group. Then, the labeled DNA is incubated with an array using a fluid station. This array is inserted into a scanner to detect a hybridization pattern. Hybridization data are collected as luminescence from fluorescent reporter group bound to the probe array (i.e. taken into the target sequence). Generally, probes which completely matched with the target sequence generate stronger signal than those probes which have portions not matching with the target sequence. Since the sequences and locations of individual probes on the array are known, it is possible to determine the sequence of the target polynucleotide reacted with the probe array on the basis of complementation.

In the present invention, it is also possible to use DNA microchips with electrically captured probes (Nanogen; see, for example, U.S. Pat. Nos. 6,017,696; 6,068,818; and 6,051,380, incorporated herein by reference). By using microelectronics, the technology of Nanogen is capable of transferring charged molecules to and from specific test sites on semiconductor microchips and concentrating them. DNA capturing type probes specific to certain SNPs or variations are arranged on specific sites on microchips electrically or assigned addresses. Since DNA is strongly negatively charged, it is capable of moving electronically to a positively charged area.

Further, in the present invention, it is also possible to use an array technology utilizing fluid separation phenomenon on a plane surface (chip) because of difference in surface tensions (ProtoGene; see, for example. U.S. Pat. Nos. 6,001,311; 5,985,551; and 5,474,796, incorporated herein by reference). The technology of ProtoGene is based on a fact that fluids are separated from each other on a plane surface because of difference in surface tensions given by chemical coating. Since oligonucleotide probes may be separated based on the above-mentioned principle, it is possible to synthesize probes directly on a chip by the ink-jet printing of a reagent containing probes. An array having reaction sites defined by surface tension is mounted on X/Y movable stage located under one set (4) of piezoelectric nozzles. Each piezoelectric nozzle contains four standard DNA nucleotides, respectively. This movable stage moves along each row of the array to supply an appropriate reagent (e.g. amidite) to each reaction site. The entire surface of the array is soaked in a reagent common to the test sites in the array and then in a washing solution. Subsequently, the array is rotated to remove these solutions.

DNA probes specific to the SNPs, e.g. those in FIG. 9 or 10, or variations to be detected are affixed to a chip using the technology of Protogene. Then, the chip is contacted with PCR-amplified gene of interest. After hybridization, unbound DNA is removed, followed by detection of hybridization using an appropriate method.

Further, it is possible to detect polymorphisms using “bead arrays” (Illumina Inc.; see, for example, PCT International Publication Nos. WO 99/67641 and WO 00/39587). Illumina Inc. utilizes Bead Array technology which uses a combination of optical fiber bundles and beads that undergo self-association with the array. Each optical fiber bundle has several millions of fibers depending on the diameter of the bundle. Beads are coated with oligonucleotides specific for the detection of certain SNPs or variations. Various types of beads are mixed in specific amounts to allow the formation of an array-specific pool. For assay, a bead array is contacted with a sample prepared from a subject. Then, hybridization is detected by any appropriate method.

The arrays provided herein for use in the assays described herein are constructed using suitable techniques known in the art. See, for example, U.S. Pat. Nos. 5,486,452; 5,830,645; 5,807,552; 5,800,992 and 5,445,934, each of which are incorporated herein by reference. In each array, individual nucleic acid elements may appear only once or may be replicated. The arrays may optionally also include control nucleic acid elements.

Any suitable substrate can be used as the solid phase to which the nucleic acid elements are immobilized or bound. For example, the substrate can be glass, plastics, metal, a metal-coated substrate or a filter of any material. The substrate surface may be of any suitable configuration. For example the surface may be planar or may have ridges or grooves to separate the nucleic acid elements immobilized on the substrate. In an alternative embodiment, the nucleic acids are attached to beads, which are separately identifiable. The nucleic acid elements are attached to the substrate in any suitable manner that makes them available for hybridization, including covalent or non-covalent binding.

The microarrays of the invention can comprise one or more oligonucleotide primers or probes that correspond with the genetic variations of the invention, including, for example, the genetic variations of FIG. 9 or 10. For example, the microarrays can comprise one or more oligonucleotides corresponding with the genetic variations of FIG. 10 relating to the human genes ACTR1A, MXRA5, UQCRC1, PDZK1Ip1, PSMD13, COL5A2, XRCC6, LRP10, C14orf159, TM9SF, C9orf86, AVEN, PSMD8BP1, Cxorf34, and FLJ00312. The microarrays can be used in the detection of the genetic variations of the invention in nucleotide samples from a subject of interest. A subject of interest can be any individual or patient who, for example, may be at risk of developing a particular disorder, e.g., a cancer, such as MPM, or is being tested for the diagnosis or prognosis of a disorder, such as, cancer, or is being tested in response to a therapy given to ameliorate or treat the disorder, e.g., cancer.

In a particular embodiment, the detection of a single genetic variation by hybridizing a nucleic acid sample of interest (e.g. a subject's tissue suspected of being cancerous) against a microarray comprising the genetic variations of FIG. 10, i.e. the genetic variations in ACTR1A, MXRA5, UQCRC1, PDZK1Ip1, PSMD13, COL5A2, XRCC6, LRP10, C14orf159, TM9SF, C9orf86, AVEN, PSMD8BP1, Cxorf34, and FLJ00312 of FIG. 10, is indicative of an increased risk of MPM. In another embodiment, the detection of at least two, preferably at least three or four, more preferably at least five or six or seven, still more preferably at least eight or nine or ten, and more preferably even up to eleven or twelve or more genetic variations of FIG. 10 is indicative of an increased risk of MPM.

In embodiments involving use of the genetic variations of the invention, it should be noted that those genetic variations identified by the method of the present invention as occurring only in tumor or diseased tissues, e.g., tumor-specific mutations (e.g., FIG. 10), can be used to assess risk, diagnose, or evaluate an individual's diseased tissues. However, because many diseases, including cancer, sometimes involve the interaction of many different mutations, those genetic variations of the invention that are not necessarily tumor-specific, but may be present in both tumor and non-tumor or diseased and non-diseased tissues, also are useful in detecting risk, or diagnosing and evaluating an individual's disease, e.g., cancer. Accordingly, although 54 of the genetic variations of FIG. 9 (i.e. excluding the variations of FIG. 10) were found in both tumor and healthy tissues, they are likely to be useful cancer markers because cancer involves an interaction of many mutations, including those in both FIG. 10 (those mutations found in MPM tumors but not in healthy tissue) and those in FIG. 9 (those mutations found in both MPM tumors and in healthy tissue). Accordingly, the present invention contemplates microarrays for detecting mutations identified by the present invention which are tumor-specific and those that are not.

The methods above can utilize any known hydridization technologies. The polynucleotides of the invention may be labeled using a variety of reporter molecules by either PCR, recombinant, or enzymatic techniques. For example, a commercially available vector containing a polynucleotide of the invention (e.g., FIG. 10 sequences) is transcribed in the presence of an appropriate polymerase, such as T7 or SP6 polymerase, and at least one labeled nucleotide. Commercial kits are available for labeling and cleanup of such polynucleotides. Radioactive (Amersham Pharmacia Biotech (APB), Piscataway N.J.), fluorescent (Operon Technologies, Alameda Calif.), and chemiluminescent labeling (Promega, Madison Wis.) are well known in the art.

A polynucleotide may represent the complete coding region of the corresponding protein or be designed or derived from unique regions of the sequence. The polynucleotides of the invention, e.g., the mutated sequences of FIG. 10, may be used under hybridization conditions that allow binding only to an identical sequence, a naturally occurring molecule encoding the same protein, or an allelic variant. Generally, a polynucleotide for use in hybridizations may be from about 100 to about 6000 nucleotides long. Such molecules have high binding specificity in solution-based or substrate-based hybridizations. An oligonucleotide, a fragment of the polynucleotide, may be used to detect a polynucleotide in a sample using PCR.

The stringency of hybridization is determined by G+C content of the polynucleotide of the invention, salt concentration, and temperature. In particular, stringency is increased by reducing the concentration of salt or raising the hybridization temperature. In solutions used for some membrane based hybridizations, addition of an organic solvent such as formamide allows the reaction to occur at a lower temperature. Hybridization may be performed with buffers, such as 5× saline sodium citrate (SSC) with 1% sodium dodecyl sulfate (SDS) at 60 C, that permit the formation of a hybridization complex between nucleic acid sequences that contain some mismatches. Subsequent washes are performed with buffers such as 0.2×SSC with 0.1% SDS at either 45 C (medium stringency) or 65-68 C (high stringency). At high stringency, hybridization complexes will remain stable only where the nucleic acid molecules are completely complementary, i.e., no mismatches. In some membrane-based hybridizations, preferably 35% or most preferably 50%, formamide may be added to the hybridization solution to reduce the temperature at which hybridization is performed. Background signals may be reduced by the use of detergents such as Sarkosyl or Triton X-100 (Sigma Aldrich, St. Louis Mo.) and a blocking agent such as denatured salmon sperm DNA. Selection of components and conditions for hybridization are well known to those skilled in the art and are reviewed in Ausubel et al. (1997, Short Protocols in Molecular Biology, John Wiley & Sons, New York N.Y., Units 2.8-2.11, 3.18-3.19 and 4-64.9).

Dot-blot, slot-blot, low density and high density arrays can be prepared and analyzed using methods known in the art. Polynucleotides from about 18 consecutive nucleotides to about 5000 consecutive nucleotides in length are contemplated by the invention and used in array technologies. The preferred number of polynucleotides on an array is at least about 100,000, a more preferred number is at least about 40,000, an even more preferred number is at least about 10,000, and a most preferred number is at least about 600 to about 800. The array may be used to monitor the expression level of large numbers of genes simultaneously and to identify genetic variants, mutations, and SNPs. Such information may be used to determine gene function; to understand the genetic basis of a disorder; to diagnose a disorder; and to develop and monitor the activities of therapeutic agents being used to control or cure a disorder. (See, e.g., U.S. Pat. No. 5,474,796; WO95/11995; WO95/35505; U.S. Pat. No. 5,605,662; and U.S. Pat. No. 5,958,342.)

### Protein Expression

The invention further contemplates methods for producing the proteins encoded by the polynucleotides of the invention, e.g., the mutation variants of FIG. 10. The full length polynucleotides or fragments thereof may be used to produce purified proteins using recombinant DNA technologies described herein and taught in Ausubel et al. (supra; Units 16.1-16.62). One of the advantages of producing proteins by these procedures is the ability to obtain highly-enriched sources of the proteins thereby simplifying purification procedures.

The proteins may contain amino acid substitutions, deletions or insertions made on the basis of similarity in polarity, charge, solubility, hydrophobicity, hydrophilicity, and/or the amphipathic nature of the residues involved. Such substitutions may be conservative in nature when the substituted residue has structural or chemical properties similar to the original residue (e.g., replacement of leucine with isoleucine or valine) or they may be nonconservative when the replacement residue is radically different (e.g., a glycine replaced by a tryptophan). Computer programs included in LASERGENE software (DNASTAR, Madison Wis.), MACVECTOR software (Genetics Computer Group, Madison Wis.) and RasMol software (www.umass.edu/microbio/rasmol) may be used to help determine which and how many amino acid residues in a particular portion of the protein may be substituted, inserted, or deleted without abolishing biological or immunological activity.

Expression of a particular polynucleotide of the invention, e.g., those of FIG. 10, may be accomplished by cloning the polynucleotide into a vector and transforming this vector into a host cell. Expression vectors usually contain a promoter and a polylinker useful for cloning, priming, and transcription. An exemplary vector may also contain the promoter for beta-galactosidase, an amino-terminal methionine and the subsequent seven amino acid residues of beta-galactosidase. The vector may be transformed into competent E. coli cells or other suitable host. Induction of the isolated bacterial strain with isopropylthiogalactoside (IPTG) using standard methods will produce a fusion protein that contains an N terminal methionine, the first seven residues of beta-galactosidase, about 15 residues of linker, and the protein encoded by the cDNA.

The polynucleotide of interest may be shuttled into other vectors known to be useful for expression of protein in specific hosts. Oligonucleotides containing cloning sites and fragments of DNA sufficient to hybridize to stretches at both ends of the polynucleotide may be chemically synthesized by standard methods. These primers may then be used to amplify the desired fragments by PCR. The fragments may be digested with appropriate restriction enzymes under standard conditions and isolated using gel electrophoresis. Alternatively, similar fragments are produced by digestion of the cDNA with appropriate restriction enzymes and filled in with chemically synthesized oligonucleotides. Fragments of the coding sequence from more than one gene may be ligated together and expressed.

Signal sequences that dictate secretion of soluble proteins are particularly desirable as component parts of a recombinant sequence. For example, a chimeric protein may be expressed that includes one or more additional purification-facilitating domains. Such domains include, but are not limited to, metal-chelating domains that allow purification on immobilized metals, protein A domains that allow purification on immobilized immunoglobulin, and the domain utilized in the FLAGS extension/affinity purification system (Immunex, Seattle Wash.). The inclusion of a cleavable-linker sequence such as ENTEROKINASEMAX (Invitrogen, San Diego Calif.) between the protein and the purification domain may also be used to recover the protein.

Suitable host cells may include, but are not limited to, mammalian cells such as Chinese Hamster Ovary (CHO) and human 293 cells, insect cells such as Sf9 cells, plant cells such as Nicotiana tabacum, yeast cells such as Saccharomyces cerevisiae, and bacteria such as E. coli. For each of these cell systems, a useful vector may also include an origin of replication and one or two selectable markers to allow selection in bacteria as well as in a transformed eukaryotic host. Vectors for use in eukaryotic host cells may require the addition of 3′ poly(A) tail if the cDNA lacks poly(A).

Additionally, the vector may contain promoters or enhancers that increase gene expression. Many promoters are known and used in the art. Most promoters are host specific and exemplary promoters includes SV40 promoters for CHO cells; T7 promoters for bacterial hosts; viral promoters and enhancers for plant cells; and PGH promoters for yeast. Adenoviral vectors with the rous sarcoma virus enhancer or retroviral vectors with long terminal repeat promoters may be used to drive protein expression in mammalian cell lines. Once homogeneous cultures of recombinant cells are obtained, large quantities of secreted soluble protein may be recovered from the conditioned medium and analyzed using chromatographic methods well known in the art. An alternative method for the production of large amounts of secreted protein involves the transformation of mammalian embryos and the recovery of the recombinant protein from milk produced by transgenic cows, goats, sheep, and the like.

In addition to recombinant production, proteins or portions thereof may be produced manually, using solid-phase techniques (Stewart et al. (1969) Solid-Phase Peptide Synthesis, W H Freeman, San Francisco Calif.; Merrifield (1963) J Am Chem Soc 5:2149-2154), or using machines such as the ABI 431A peptide synthesizer (Applied Biosystems, Foster City Calif.).

### Antibodies

In a particular embodiment, the genetic variations discovered or identified by the present inventive method, and in particular, the disease-associated genetic variations, such as disease-associated SNPs, can be detected indirectly using antibodies if the genetic variation manifests in a polypeptide or other product encoded by the affected nucleotide sequence. By “manifests” in this context, it is meant that the genetic variation at the nucleotide level can result in altered expression of an encoded product, e.g. a single amino acid replacement in a polypeptide or a truncated polypeptide where the mutation results in truncated translation. Antibodies that recognize the particular mutation in the polynucleotide are also contemplated by the present invention, such that direct detection of a disease-associated genetic variation can be detected in a test cell or tissue or DNA sample.

Antibodies are well-known in the art and discussed, for example, in U.S. Pat. No. 6,391,589. Antibodies of the invention include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule.

Antigen-binding antibody fragments, including single-chain antibodies, may comprise the variable region(s) alone or in combination with the entirety or a portion of the following: hinge region, CH1, CH2, and CH3 domains. The antibodies of the invention may be obtained from any animal, including, but not limited to, any mammal or bird. Preferably, the antibodies are human, murine (e.g., mouse and rat), donkey, sheep, rabbit, goat, guinea pig, camel, horse, or chicken. The antibodies of the invention may be monospecific, bispecific, trispecific or of greater multispecificity.

The antibodies of the invention may also include synthetic antibodies (e.g. by peptide synthetic chemistries) or antibodies made by recombinant DNA technology. Any suitable method known in the art may be employed to make or obtain the antibodies of the invention.

Polyclonal antibodies to an antigen-of-interest, e.g. a polypeptide encoded by a gene having a genetic variation of the invention, such as, a gene containing a disease-associated SNP, can be produced by various procedures well known in the art. For example, a polypeptide of the invention can be administered to various host animals including, but not limited to, rabbits, mice, rats, etc. to induce the production of sera containing polyclonal antibodies specific for the antigen-of-interest. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example; in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The team “monoclonal antibody” as used herein is not limited to antibodies produced through hybridoma technology. The temrm “monoclonal antibody” refers to an antibody that is derived from a single clone, including any eukaryotic, prokaryotic, or phage clone, and not the method by which it is produced.

Where the particular genetic variation, e.g. a SNP, results in an amino acid change in an encoded polypeptide, the nucleotide occurrence can be identified indirectly by detecting the particular amino acid in the polypeptide. The method for determining the amino acid will depend, for example, on the structure of the polypeptide or on the position of the amino acid in the polypeptide. Where the polypeptide contains only a single occurrence of an amino acid resulting from the presence of the genetic variation, e.g. resulting from the particular SNP, the polypeptide can be examined for the presence or absence of that amino acid to determine the presence of the genetic variation. For example, where the amino acid is at or near the amino terminus or the carboxy terminus of the polypeptide, simple sequencing of the terminal amino acids can be performed. Alternatively, the polypeptide can be treated with one or more enzymes and a peptide fragment containing the amino acid position of interest can be examined, for example, by sequencing the peptide, or by detecting a particular migration of the peptide following electrophoresis. Where the particular amino acid comprises an epitope of the polypeptide, the specific binding, or absence thereof, of an antibody specific for the epitope can be detected. Other methods for detecting a particular amino acid in a polypeptide or peptide fragment resulting from or associated with the underlying genetic variation thereof are well known and can be selected based, for example, on convenience or availability of equipment such as a mass spectrometer, capillary electrophoresis system, magnetic resonance imaging equipment, and the like.

Protocols for detecting and measuring proteins and/or protein expression using either polyclonal or monoclonal antibodies are well known in the art. Examples include ELISA, RIA, and fluorescent activated cell sorting (FACS). Such immunoassays typically involve the formation of complexes between the protein and its specific antibody and the measurement of such complexes. The method may employ a two-site, monoclonal-based immunoassay utilizing monoclonal antibodies reactive to two non-interfering epitopes, or a competitive binding assay. (See, e.g., Coligan et al. (1997) Current Protocols in Immunology, Wiley-Interscience, New York N.Y.; Pound, supra).

### Kits

The invention also contemplates kits for detecting the genetic variations of the invention, in particular, those identified in FIGS. 9 and 10. The kits can include any suitable means for detecting the mutations in a target nucleic acid.

For example, the kits can include the herein described oligonucleotides. The oligonucleotides can be used as PCR primers to amplify a DNA fragment encompass the mutation, which can then be detected by Sanger sequencing or other by other method. The oligonucleotides can also include the genetic variation and thus, be used as a hydridization probe to detect the presence of the variation in a target nucleic acid.

The target nucleic acid can be from any source, including from any tissue or bodily fluid sample that may be at risk or is suspected to have a disease or disorder which is associated with a particular genetic variation of the invention. For example, a tumor suspected to be at risk for MPM can be obtained from a patient. The nucleic acid can be obtained from the sample and then analyzed with the kit of the invention. PCR primers that span the mutation can be used to amplify the mutation-containing region, which fragment can then be sequenced to identify the mutation. The nucleic acids can also be probed with an oligonucleotide of the invention to detect the mutation based on whether or not the oligo hydridizes with the target sequence.

Thus, in one embodiment, the oligonucleotides of the invention may be included in a genetic mutation detection kit. The genetic mutation detection kit of the present invention comprises one or more components necessary for practicing the present invention. The kit of the present invention comprises any and all components enzymes or components necessary (suitable) for an intended assay. Examples of such components include, but are not limited to, oligonucleotides, polymerases (e.g. Taq polymerase), buffers (e.g. Tris buffer), dNTPs, control reagents (e.g. tissue samples, target oligonucleotides for positive and negative controls, etc.), labeling and/or detection reagents (fluorescent dyes such as VIC, FAM), solid supports, manual, illustrative diagrams and/or product information, inhibitors, and packing environment adjusting agents (e.g. ice, desiccating agents).

The kit of the present invention may be a partial kit which comprises only a part of the necessary components. In this case, users may provide the remaining components. The kit of the present invention may comprise two or more separate containers, each containing a part of the components to be used. For example, the kit may comprise a first container containing an enzyme and a second container containing an oligonucleotide. Specific examples of the enzyme include a structure-specific cleaving enzyme contained in an appropriate storage buffer or a container.

One or more reaction components may be provided in such a manner that they are pre-divided into portions of a specific amount. Since such a kit contains components which have already been quantitatively determined for use in one step of the method of the present invention, it is not necessary to re-measure or re-divide. Selected reaction components may also be mixed and divided into portions of a specific amount. It is preferred that reaction components should be pre-divided into portions and contained in a reactor. Specific examples of the reactor include, but are not limited to, reaction tubes or wells, or microtiter plates. It is especially preferable that the pre-divided reaction component should be kept dry in a reactor by means of, for example, dehydration or freeze drying. Where the detection scheme involves the use of antibodies described herein, the kit may also include said antibodies, and any necessary secondary components, such as, labels, buffers, enzymes, substrates, instructions etc.

### EXAMPLES

### Example 1

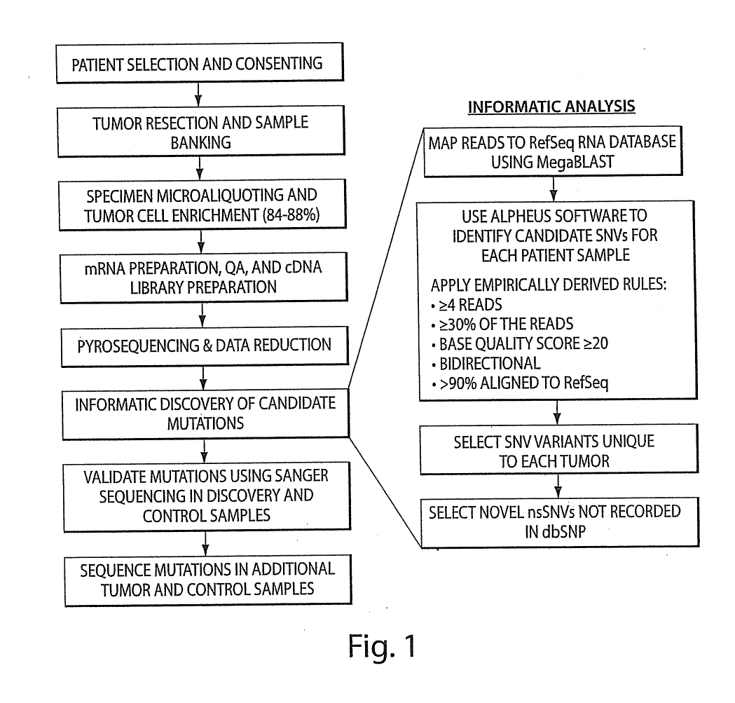

### Tumor Transcriptome Sequencing and Global Analysis (FIG. 1)

### Source of Tumor Samples

Tumors were harvested in the operating room from consenting patients and immediately dissected to generate high-quality fresh-frozen specimens. Samples were obtained from four patients, representing the clinical spectrum of MPM, who underwent pleurectomy/decortication or extrapleural pneumonectomy (Chang and Sugarbaker, Thoracic Surg Clin, 2004, 14:523-530). Patient 1 was a 75-year-old male with asbestos exposure history. Patient 2 had epithelial MPM and was a 39-year-old female with no history of asbestos exposure. Patients 3 had nonepithelial sarcomatoid MPM. Patient 4 had mixed epithelial and sarcomatoid (“mixed”) MPM. For comparison, a tumor was obtained from a patient (Patient 5) with adenocarcinoma (ADCA) of the lung and a normal nontumor lung tissue was obtained from a patient (Patient 6) with mixed MPM. To examine the prevalence of specific mutations discovered in these six tumors, 49 additional specimens were collected from MPM tumors representing the full spectrum of the disease in terms of stage, gender, asbestos exposure, and histology.

The selected tumor specimens were processed by using a microaliquoting technique to identify and subselect samples with high tumor cell content (>85%) and little necrosis using cryosections (described below). For each specimen, high-quality mRNA (Agilent Bioanalyzer RNA integrity number>7.8 for total RNA) was isolated and used for 12 runs of shotgun 454 sequencing with 454 Life Sciences GS20 technology (Margulies et al., Nature, 2005, 437: 376-380). An internet-based information resource was developed to perform MegaBLAST alignments against RefSeq RNA (www.ncbi.nlm.nih.gov/RefSeq) and to permit the selection and display of all sequence variants and relevant metadata (www.impmeso.org). A stringent MegaBLAST search was also conducted of reads that did not map to the 19,306 known RefSeq genes against the 52,935 “Main Genes” in AceView (www.ncbi.nlm.nih.gov/IEB/Research/Acembly), the human genome, and the Pan troglodytes (chimpanzee) genome. Rules were developed and applied to identify candidate mutations among the known RefSeq genes that were unique to each of the four MPM patients. All candidate mutations derived from the four MPM samples (Patients 1-4) were selected for confirmation and characterization by using conventional Sanger sequencing, both in the discovery samples and additional tumors (the validation set) and matched normal tissue from patients with MPM.

### Patient Selection and Consenting

Selected patients undergoing surgical resection at the Brigham and Women's Hospital were enrolled in a prospective protocol for banking and genomic research. Pre- and post-operative blood samples, as well as discarded surgical specimens, were collected. Informed consent was obtained, and all work was performed according to institutional guidelines and with IRB (Institutional Review Board) approval.

### Tumor Resection and Sample Banking

Resected specimens were taken rapidly to the frozen section room, where portions identified by pathology to be in excess of clinical needs were obtained for banking. These discarded tissues were further dissected to identify areas of grossly visible tumor to be aliquoted, frozen, and banked. Adjacent portions of the specimen that were not involved with tumor were also banked, when possible. Blood samples were collected in Vacutainer tubes containing anticoagulant. Plasma was separated by centrifugation, aliquoted, frozen, and banked.

The selected tumor specimens were processed using a recently developed microaliquoting technique (Richards et al., Biotech Histochem, in press, 2007) to identify and subselect, using cryosections, samples with high tumor cell content (>85%) and little necrosis.

### Specimen Microaliquoting and Tumor Cell Enrichment

RNA and DNA were prepared from aliquots of frozen tumor or adjacent tissue samples. Tumor aliquots were optimized for high tumor cellularity and low necrosis using a method of microaliquoting described by Richards, et al. (2007 Biotech Histochem In Press). Selected (˜4×4×4 mm) aliquots were grossly trimmed as required, embedded in OCT, and mounted on a cryostat. Alternating thin and thick sections were cut and stained with H&E or stored at −80° C., respectively.

The cross-sectional area of each stained section was estimated by computer analysis of a digitized image. Each slide was scored in random order by a pathologist for necrosis as a percentage of cross-sectional area and for tumor cells, fibroblasts, lymphocytes, normal, and other cells, each as a percentage of the total number of viable nucleated cells (totaling 100%). Histologic parameters for microaliquots were estimated by averaging estimates from the flanking slides. Similarly, microaliquot volume was estimated by multiplying the average of the flanking cross-sectional area estimates by cut depth (80 μM).

Custom sub-samples for RNA extraction that each consisted of 10 microaliquots were selected to maximize tumor cellularity and minimize necrosis. Histologic parameters for sub-samples were estimated by volume-weighted integration of estimates for the selected microaliquots. The microaliquots were rinsed briefly with RNAse-free water to remove OCT and then combined and homogenized in 1 mL Trizol reagent.

Two patients had epithelial MPM: one was a 75-year-old male with asbestos exposure history (Patient 1) and the other was a 39-year-old female with no history of asbestos exposure (Patient 2). The other two were males with non-epithelial tumor histology: sarcomatoid MPM (Patient 3) and mixed epithelial and sarcomatoid (“mixed”) MPM (Patient 4), both with a history of asbestos exposure. For comparative purposes, tumor from a male patient with adenocarcinoma (ADCA) of the lung (Patient 5) and normal non-tumor lung tissue from a female patient with mixed MPM (Patient 6) were studied.

For each specimen, high quality mRNA (Agilent Bioanalyzer RNA integrity number>7.8 for total RNA) was isolated and used for 12 runs of shotgun 454-sequencing with 454 Life Sciences (Branford, Conn. USA) GS20 technology.

### RNA Preparation and Quality Assessment

Total RNA was isolated in a blinded manner from multiple sub-samples per patient using Trizol reagent (Invitrogen, Carlsbad, Calif.) and 1-bromo-3-chloropropane (BCP) for phase separation (MRC, Cincinnati, Ohio). RNA in the aqueous phase was precipitated with 80% ethanol, purified using Qiagen RNeasy columns (Valencia, Calif.), and treated with 20 U DNAse (Promega, Madison, Wis.) with RNAse inhibitor (RNasin, Promega). DNA-free RNA was isolated using acid phenol chloroform (Ambion, Austin, Tex.) with precipitation of the aqueous phase in 1/10 volume 3M sodium acetate and 100% ethanol. The RNA pellet was washed in 70% ethanol and resuspended in RNAse-free water. RNA yield and purity were initially assessed using spectrophotometry.

The quality of total RNA from each sub-sample was assessed on an Agilent 2100 Bioanalyzer (Agilent, Palo Alto, Calif.) using an RNA 6000 Pico LabChip. RNA fractions with an RNA Integrity Number (MN) were selected for cDNA synthesis. Total RNA quantity was assessed by fluorometry using the Quant-iT RiboGreen RNA Assay kit (Invitrogen).

Sub-samples of RNA were selected that contained high quality RNA and high tumor content and combined to yield ≧350 μg total RNA. These parameters were calculated by integration across the selected sub-samples weighting each according to its relative contribution to the total quantity of RNA. The pathologic, demographic and RNA quality parameters of the resulting samples are presented in Table 1, below.

TABLE 1
Demographic and tumor information for the six patients in study

| | Patient 1 | Patient 2 | Patient 3 | Patient 4 | Patient 5 | Patient 6 |
|---|---|---|---|---|---|---|
| Pathology | MPM, | MPM, | MPM, | MPM, mixed | Lung | Normal lung |
| | Epithelial | Epithelial | Sarcomatoid | “Mixed” | Adeno- | from MPM |
| | | | | | carcinoma | Patient |
| Gender | Male | Female | Male | Male | Male | Female |
| Age | 75 | 39 | 61 | 65 | 68 | 63 |
| Operation | Pleu-rectomy | EPP | EPP | Pleurectomy | Lobectomy | EPP |
| Tumor cells | 86.8% | 86.1% | 84.8% | 83.6% | 87.7% | 0% |
| Lymphocytes | 6.4% | 4.8% | 5.7% | 4.3% | 1.6% | 0% |
| Fibroblasts | 6.7% | 9.0% | 9.5% | 12.2% | 10.7% | 0% |
| Necrosis | 3.5% | 0% | 1.5% | 1.5% | 0.4% | 0% |
| Total RNA | 353 μg | 426 μg | 375 μg | 495 μg | 438 μg | 368 μg |
| Yield | | | | | | |
| RIN | 8.8 | 8.6 | 8.5 | 9.1 | 8.7 | 8.5 |

cDNA Library Preparation

cDNA sample preparation and sequencing work was done at the 454 Life Sciences™ Sequencing Centre (Branford, Conn.). Poly-A+ RNA was prepared from 350 μg total RNA using oligo(dT) magnetic beads (PureBiotech, Middlesex, N.J.), and quantified with fluorometry. First-strand cDNA was prepared from 5 to 8 μg of poly(A)+ RNA with 200 μmol oligo(dT)25V (V=A, C or G) using 300 U of Superscript II reverse transcriptase (Invitrogen). Second-strand synthesis was performed at 16° C. for 2 h after addition of 10 U of E. coli DNA ligase, 40 U of E. coli DNA polymerase, and 2 U of RNase H (all from Invitrogen). T4 DNA polymerase (5 U) was added and incubated for 5 min at 16° C. cDNA was purified on Q1Aquick Spin Columns (Qiagen) and the yield was determined by fluorometry using the Quant-iT PicoGreen dsDNA Reagent (Invitrogen). Single-stranded template DNA (sstDNA) libraries were prepared using the GS20 DNA Library Preparation Kit (Roche Applied Science, Indianapolis, Ind.) following the manufacturer's recommendations. Library quality was assessed by RNA 6000 Pico LabChip.

For one sample (Patient 4), a second sstDNA library was prepared using a random priming protocol. The library was used for 4 of the 12 sequencing runs and did not materially affect our ability to discover variants, and thus was not extended to other samples. The random primed sstDNA library preparation was performed as follows: 200 ng of mRNA was fragmented for 2 min at 82° C. in 40 mM Tris-acetate pH 8.1, 100 mM potassium acetate, and 31.5 mM magnesium acetate. Following the fragmentation, the mRNA was cleaned up on Sephadex G50 mini Quick Spin RNA Columns (Roche Applied Science). First-strand cDNA was prepared from 400 μmol TNNT(N)6 oligo (Integrated DNA Technologies, Coralville, Iowa) using 200 U of Superscript II reverse transcriptase. cDNA/mRNA hybrids were melted by incubation in a 0.5 M NaOH pH 8.0 solution followed by purification using the AMPure purification kit (Agencourt Bioscience, Beverly Mass.) in a 1.4:1 bead volume to cDNA solution volume ratio.

Double-stranded adapters were ligated to the single-stranded cDNA using the following adapters SAD1 F (adapter A: 5′-GCCTCCCTCGCGCCATCAG-3′; adapter a: 5′-N*A*N*NACTGATGGCGCGAGGGA*G*G*/3ddC) and SAD1R (adapter B: 5′-Bio-GCCTTGCCAGCCCGCTCAGNNNN*N*N*-3′; adapter b′: 5′-CTGAGCGGGCTG GCAAGG/3ddC (Integrated DNA Technologies); where * are phosphorothioated bases, 3ddC is a dideoxynucleotide in 3′ position of the last C nucleotide, and 5′-Bio is a 5′ biotin) overnight at 22° C. using 400 U of T4 DNA Ligase (New England BioLabs, Ipswich, Mass.). Adapters-ligated cDNA library was isolated on Dynabeads® MyOne™ Streptavidin Cl (Invitrogen) and the single-stranded library was released by incubating in a 25 mM sodium hydroxide solution and purified using the using the AMPure purification kit.

### Pyrosequencing and Data Reduction

sstDNA libraries were clonally amplified in a bead-immobilized form using the GS20 emPCR kit (Roche Applied Science) following the manufacturer's recommendations. sstDNA libraries were sequenced on the 454 Genome Sequencer 20 instrument. For each sample, 12 independent sequencing runs were conducted. Data reduction was performed using standard 454 software procedures to generate nucleotide sequences and quality scores for all reads. Sequence data was FASTA formatted and ported to NCGR for further analysis.

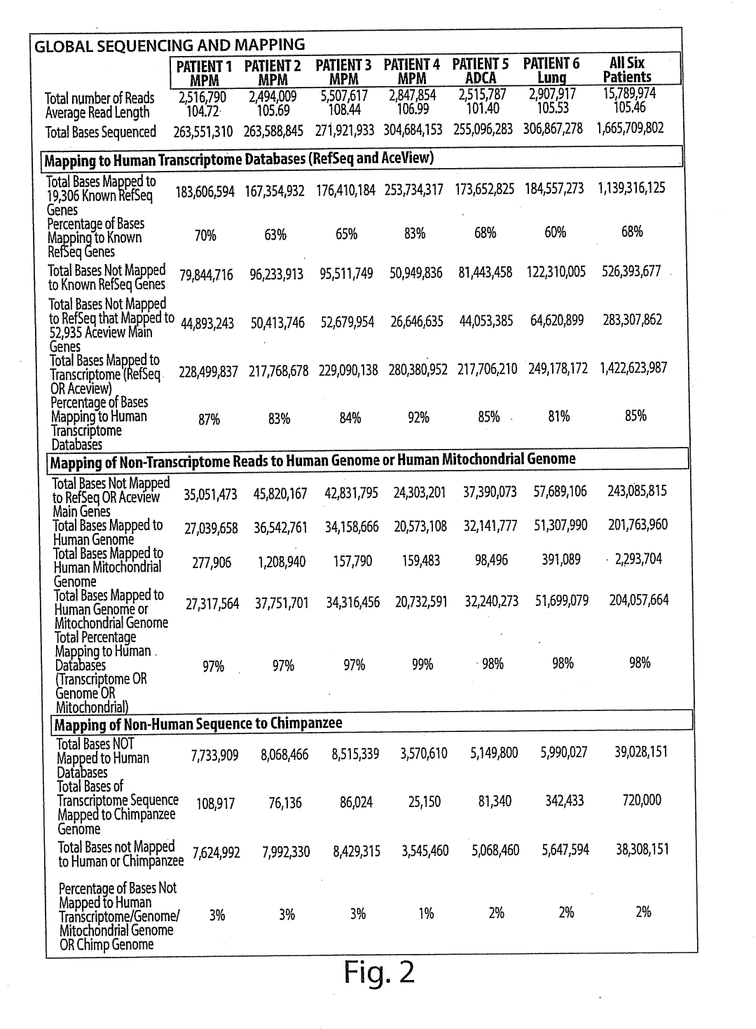

1.65 Gb of cDNA sequence were generated from polyadenylated RNA, representing ˜15.8 million reads with an average length of 105 nucleotides²⁰ (Table 2, see FIG. 2).

An internet-based information resource was developed to perform MegaBLAST alignments against RefSeq RNA (46) and to permit the selection and display of all sequence variants and relevant metadata (www.impmeso.org).

### Informatics and Global Sequence Analysis

Reference sequences and annotations used for variant detection and characterization were downloaded from NCBI's RefSeq transcript dataset for Homo sapiens on Apr. 21, 2006 from: ftp://ftp.ncbi.nih.gov/refseq/H sapiens/mRNAProt/human.rna.*. This set contains 40,545 transcripts, representing splice variants of 28,593 genes. Additional gene metadata for these entries was obtained from the Entrez Gene database representation at: ftp://ftp.ncbi.nih.qov/gene/DATA/ASN BINARY/Mammalia/Homo sapiens.aqs.qz Reference sequence data was imported into a relational database along with location based feature information (e.g. CDS annotations, variation records). Reference data was stored in an instance of the GMOD Chado schema (http://www.gmod.org) and subsequently translated into an application-specific schema.

Alignments of the 454 reads against the RefSeq transcript dataset were created with MegaBLAST version 2.2.13, using parameters established by NCBI for SNP genome-mapping (ftp://ftp.ncloi.nih.gov/snp/00readme.txt), with the following changes: wordsize was decreased to 14; expect value filter set to e-05; and low-complexity sequence were not allowed to seed alignments, but alignments were allowed to extend through such regions. BLAST compute jobs were performed on a 62-Dual-core Processor Dell 1855 Blade Cluster with 124 GB RAM and 2.4 TB disk. The best-match alignments for a given read were imported into the database using Java programs based on the BioJava BLAST parsing framework. The best matches were accepted only if their alignments had at least 35 identities and had an expect value <e-10. All alignments equivalent in quality to the best match were accepted (as in the case of hits to shared exons in splice variants).

For each of the six patient samples, over 250 megabases (Mb) of sequence were obtained, of which ˜68% mapped to the 19,306 non-LOC RefSeq genes (“Known RefSeq Genes”). An additional ˜10% of the sequence mapped to the 9,456 RefSeq “LOC” genes, which are provisional genes that have been identified informatically from the human genome sequence (21). The RefSeq LOC genes were not further used in the analysis, since putative variants within LOC genes were found to be unreliable as a consequence of: (1) uneven coverage (likely mis-annotation of splice variants); (2) an overabundance of putative SNPs; and/or (3) premature truncation of alignments. Therefore, the ˜32% of the sequence that did not align to the 19,306 Known RefSeq Genes was mapped, sequentially against four NCBI databases to deduce their identity: AceView, the human genome, the human mitochondrial genome, and the chimpanzee genome.

Stringent MegaBLAST searches of reads that did not map to the 19,306 Known RefSeq Genes against the 52,935 “Main Genes” in AceView,⁴⁷ the human genome, and the Pan troglodytes (chimpanzee) genome were conducted.

Custom software was created to sequentially map i) reads that did not show significant alignment to the NCBI RefSeq transcript database and ii) reads with alignments to “LOC” genes within the RefSeq transcript database to Aceview, the human genome, the human mitochondria genome, and the chimpanzee (P. troglodytes) genome. Scripts to automate these analyses and for data interpretation were composed using ActivePerl v.5.8.8. Build 820 for Windows 2000 and BioPerl v.1.4. and were run within the environment of the Pattners Healthcare HP XC v.3.0. computer cluster. BLAST results were automatically parsed for relevant information to determine the degree of alignment with relevant sequences. The specific BLAST analysis and parser parameters were as follows:

### Aceview “Main” Genes

Database: AceView Main genes (52,935 genes). Version: August 2005. http://www.ncbi.nlm.nih.gov/IEB/Research/Acembly/index.html
Alignment tools: standalone MEGABLAST 2.2.14 [May 7, 2006], arguments: -W 14-F “m D”, other arguments default. ftp://ftp.ncbi.nih.goviblast/
Blast report parser: length of hit participating in alignment minus gaps>=36, E-value<=1e-4

### Human Genome

Sequence database: NCBI human reference assembly. Build 36.
Alignment tools: standalone MEGABLAST 2.2.14 [May 7, 2006], arguments: -v5-b 5-W 14, other arguments default. ftp://ftp.ncbi.nih.gov/blast/
Blast report parser: length of hit participating in alignment minus gaps>=36, E-value<=1e-4

### Human Mitochondrial Genome

Sequence database: Homo sapiens mitochondrion, complete genome (NC_{—}001807.4). Build 36.
Alignment tools: standalone MEGABLAST 2.2.14 [May 7, 2006], arguments: -v5-b 5-W 14, other arguments default. ftp://ftp.ncbi.nih.gov/blast/
Blast report parser: length of hit participating in alignment minus gaps>=36, E-value<=1e-4
   Chimpanzee (P. troglodytes) Genome
Sequence database: Pan troglodytes genome, reference assembly based on Pan_troglodytes-2.1.
Alignment tools: standalone MEGABLAST 2.2.14 [May 7, 2006], arguments: -v5-b 5-W 14, other arguments default. ftp://ftp.ncbi.nih.gov/blast/Attorney
Blast report parser: length of hit participating in alignment minus gaps>=36, E-value<=1 e-4

Rules were developed and applied to identify candidate mutations among the Known RefSeq Genes that were unique to each of the 4 MPM patients.

### Discovery of Candidate Mutations

Only variants in reads with identity to mRNA transcripts in the NCBI RefSeq RNA database described above were used to identify candidate mutations. From these alignments, all mismatches between the reads and RefSeq transcripts were tabulated (counting each contiguous indel as a single event) and unified into candidate polymorphisms on the basis of the RefSeq position and variant allele observed. Statistics on frequency, alignment quality, base quality, and other attributes used to assess likelihood were calculated for each variant reported at each RefSeq transcript position. Variants were further characterized with respect to their context in the transcript (e.g. coding region) and, where relevant, the change caused in the protein sequence.

A web-based query and visualization interface was created to allow the dataset to be mined for variants meeting user-specified criteria on transcript abundance, positional coverage, variant frequencies, and variant putative functional properties (www.impmeso.org). The web interface was created using Java J2EE technologies (servlets, JSP and JDBC), XML and Flash.

All variants that fit the minimal filtering criteria (e.g., ≧4 reads calling variant, 0.30% of reads covering region show variant) in at least one of the samples were identified using Alpheus software and downloaded in spreadsheet format as a tab delimited text file which was imported into a Microsoft Access database. Data collected for each variant included the following: gene symbol, number and directionality of strands showing variant, whether the variant was in the coding region, the variant type (e.g., (non-)synonymous SNP, indel), BLOSUM score (for SNPs), variant description and nucleotide location (e.g., a423g), the associated NCBI RefSeq transcript Accession number(s), the ID for at least one read with >90% homology to the Reference Sequence, whether the gene was a member of a list of Known Cancer Genes (Futreal, P. A., et al. 2004 Nat Rev Cancer 4:177-183), and the number of reads calling the variant and the number of reads covering the region for each patient. Tabular data was dichotomized whenever possible and assigned a binary identifier to facilitate subsequent queries. This database was used to generate more refined variant lists based on subsequent queries. Select variants were chosen for validation purposes using specific criteria as described in the text.

SNPs were deemed to be “novel” if they were not previously recorded in either the RefSeq RNA or dbSNP databases at NCBI. The RefSeq database was downloaded from ftp://ftp.ncbi.nih.gov/blast/db/ on Sep. 18, 2006 and included 705,951 sequences. The dbSNP database (Homo sapiens Build 126) was downloaded from ftp://fic.ncbi.nih.gov/snp/ on Oct. 18, 2006 and included 12,702,095 RefSNPs. The alignment program blastall v.2.2.14. [May 7, 2006] was obtained from ftp://ftp.ncbi.nih.gov/blast/ and used to conduct automated BLAST analyses of nucleotide fragments, each of which contained a unique variant. These fragments were constructed using the RefSeq nucleotide Accession # and were 101 bp in length. The central nucleotide corresponded to the SNP of interest that was manually changed to the variant base. These sequences were then queried against each database using BLAST and default search parameters. Results were obtained automatically using custom software to parse the resulting data pages for matches where aligned segments were equal to at least 50 bp in length and covering the SNP site.

AceView is an NCBI database that collates cDNAs collected from multiple NCBI databases (e.g., db_EST and db_nr), as well as other global databases. AceView is more comprehensive than RefSeq in terms of splice variants of both known and novel genes. Using stringent parameters, 283 Mb of the ˜500 Mb that did not map to the 19,306 Known RefSeq Genes exhibited alignments to the 52,935 better annotated AceView “Main Genes” (22) indicating that ˜85%, or 1.4 Gb, of the total transcriptome sequences could be unambiguously aligned to RefSeq or Aceview, the two primary NCBI human transcriptome databases (FIG. 2).

Of the remaining reads, an additional 201 Mb of sequence mapped to the human genome, indicating the utility of shotgun clonal sequencing for identification of novel, transcribed sequences. A further 2.2 Mb mapped to the human mitochondrial DNA database. In total, ˜98% of reads mapped to human sequence databases (see FIG. 2). In view of the existence of gaps and plasticity within the reference human genome sequence that can be revealed through comparative alignment with other closely related species, the remaining reads containing ˜39 Mb were mapped to the chimpanzee genome, and it was observed that ˜720 kilobases (Kb) of sequence mapped, indicating the existence of additional, novel expressed sequences. The remaining ˜2% of sequence was unmapped. Of note, no reads aligned to SV40 sequences, further diminishing the purported role of this virus in the etiology of MPM. Nor did any reads align to any other viral and/or bacterial genomes.

### Discussion

To discover mutations in expressed genes of MPM specimens, cDNA from tumors of four MPM patients (Patients 1-4) were sequenced. For comparison, cDNA from an adenocarcinoma (ADCA) tumor of the lung (Patient 5) and from normal lung of a MPM patient (Patient 6) were also sequenced. The process used for mutation discovery and validation is schematically shown in FIG. 1. Briefly, polyadenylated RNA was prepared from microaliquoted tumor specimens to ensure >85% tumor cell content. For each of the six samples, >260 Mb of transcriptome sequence were obtained by shotgun, clonal pyrosequencing using 454 technology (FIG. 2). Approximately 15 million cDNA sequence reads with lengths of ≈105 bp each were informatically mapped by using BLAST to human mRNA and DNA databases, and overall 98% of the reads matched known human RNA, DNA, and mitochondrial DNA sequences (FIG. 2). Of the ≈39 Mb that did not map to human databases, ≈720 Kb mapped to chimpanzee, suggesting the existence of additional, previously uncharacterized expressed sequences, and preliminary analysis (data not shown) suggested that they were largely noncoding sequences. No reads aligned to SV40 sequences or to any other viral or bacterial genomes.

For variant and mutation discovery, only transcript sequences that mapped to the 19,306 well curated human reference mRNAs present in the “RefSeq mRNAs” database (www.ncbi.nlm.nih.gov/RefSeq/) were analyzed. The 9,456 LOC genes that have been identified informatically from the human genome sequence were excluded. The LOC genes have uneven coverage (likely misannotation of splice variants in RefSeq), an overabundance of putative single-nucleotide polymorphisms (SNPs), and premature truncation of alignments. Alignment of sequence reads to all 29,761 RefSeq mRNAs can be visually inspected (www.impmeso.org).

### Example 2

### Gene Coverage and Expression

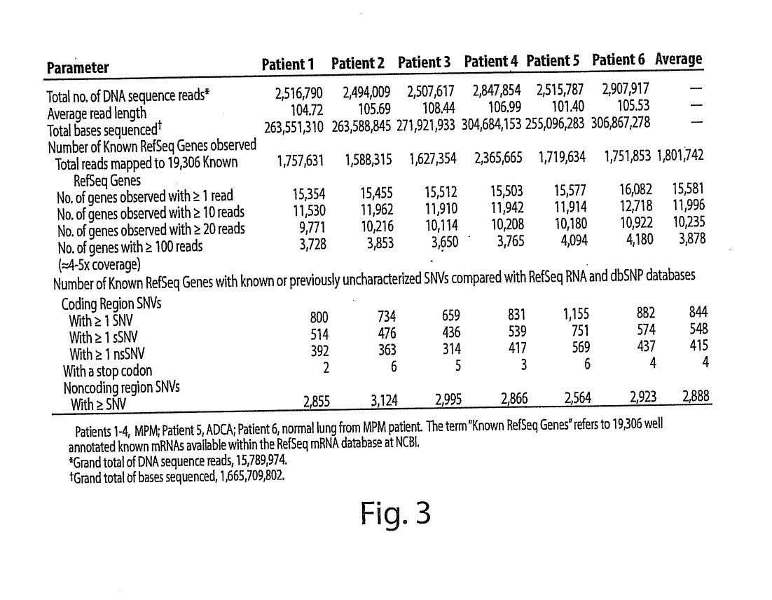

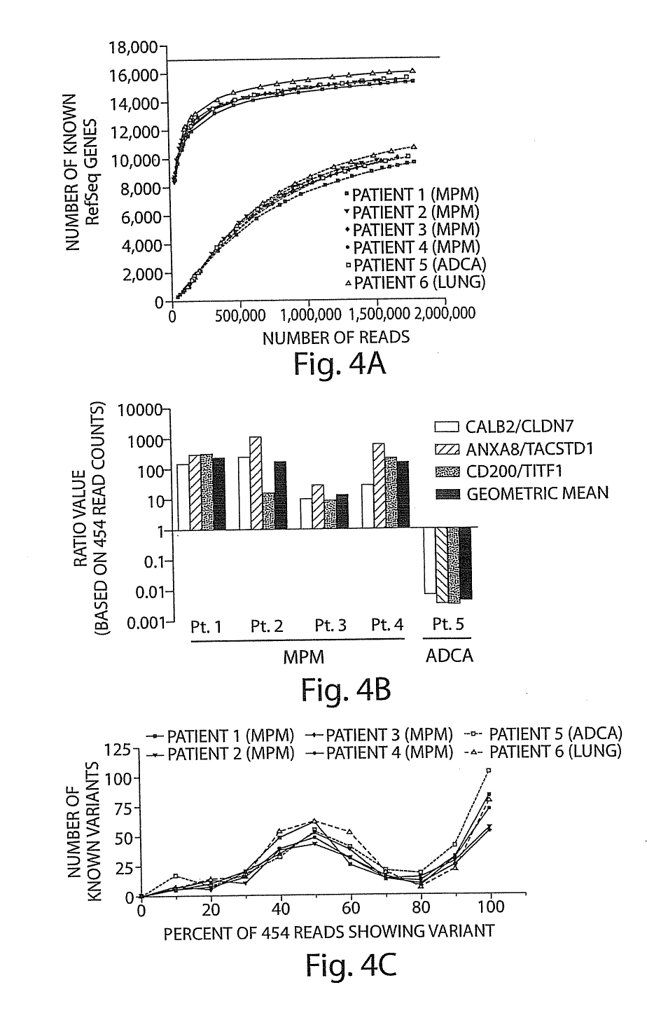

In each sample, ˜15,000 Known RefSeq Genes were detected by alignment of one or more reads, indicating expression of 75% of the Known RefSeq mRNA entries (FIG. 2, FIG. 3 and FIG. 4A). Furthermore, when combined, a total of 17,500 non-redundant Known RefSeq Genes were detected in the 6 samples; approximately 17,000 non-redundant Known RefSeq Genes were similarly observed when the data from only the 4 MPM cases were combined (data not shown). At the depth of coverage selected, few additional transcripts were identified by further increasing the numbers of reads (FIG. 4A), indicating that >90% of the expressed genes in each of the samples were detected. Of the 15,000 observed transcripts, many are represented by only a few reads which would arise either from low abundance cell types in the sample or transcriptional “leakage”.

A prior study using massive parallel signature sequencing (MPSS) of mRNAs in human tissues concluded that only 50% of the genome is expressed in a human tissue (23). Based on this observation, approximately 10,000 of the 19,306 RefSeq genes should be considered functionally expressed in a given sample. In this context, ˜10,000 genes were observed at a threshold of 20 reads per gene (FIG. 3 and FIG. 4A), corresponding to a minimum of ˜1× coverage, assuming an average transcript length of 2 Kb. Independent analysis with GE CodeLink microarrays (not shown) supported the interpretation that using this threshold identified the expressed genes in the tumor samples.

Pilot studies indicated that a minimum of 4-5× coverage or ˜100 reads were necessary for reliable sequence variant discovery. Approximately 3,800 Known RefSeq transcripts per patient were detected with 100 read coverage (see FIG. 3). Upon inclusion of the AceView Main Genes with 100 read coverage, a total of more than 5,000 genes per patient were sequenced in sufficient depth for variant discovery by 250 Mb of shotgun, transcriptome sequencing. However, the incidence of putative variants was much higher in reads mapped to AceView genes than those mapped to the Known RefSeq genes. Thus, the search described herein for sequence level mutations was confined to those Known RefSeq Genes with at least four reads covering the variant base position, which represents ˜3,800 genes or ˜38% of the functionally expressed Known RefSeq Genes (FIG. 3).

The molecular differentiation of MPM from ADCA was previously demonstrated using ratios of expression levels of six genes or their geometric mean obtained using quantitative reverse transcription-polymerase chain reaction or microarrays (12, 13). This technique was applied to the tumor samples using 454 sequencing aligned read counts as a proxy of expression levels (FIG. 4B). The data indicated that ratios of aligned read counts correctly distinguished MPM (ratios>1) from ADCA (ratios<1) specimens, providing a validation for the use of high-throughput 454-sequencing to quantitatively characterize the tumor transcriptome. These ratio results were independently confirmed in the same specimens by expression analysis with GE CodeLink microarrays (data not shown). Thus, transcriptome pyrosequencing can also provide quantitative gene expression information of tumors.

### Example 3

### Bioinformatic Discovery of SNP Variants (Rules for SNP Discovery)

Software systems for DNA sequence variant discovery that are based upon Sanger chemistry and base-calling algorithms are inadequate for novel DNA sequencing technologies that feature short read lengths, novel base-calling and quality score determination methods, and relatively poorly characterized error profiles (20). Alpheus, an interne-accessible software system that maps individual reads to the NCBI RefSeq RNA database and identifies sequence level variants (accessible at www.impmeso.org), was developed to facilitate visualization and automated analysis of high-throughput 454 sequencing data. Filter parameters include patient sample, gene name, read coverage, variant frequency, variant type, variant location and hyperlinks to NCBI sequence and gene function databases.

Assessment of putative sequence variants identified by analysis of unfiltered 454 sequencing data revealed an unacceptably high number of false positive SNPs. To minimize this problem, an empiric rule was developed, set for use with high-throughput 454 sequencing technology as a tool for mutation discovery in human tumors. These rules required that the variant must be: (1) present in at least 4 reads (this effectively requires 4-5× gene coverage); (2) present in at least 30% of the total number of reads covering the variant; (3) of GS20 quality score ≧20 for the relevant nucleotide (20); (4) observed in reads obtained from both orientations; and (5) within a read that is >90% identical along its entire length to the target RefSeq mRNA sequence.

These rules exhibited 96% sensitivity in identification of 2,465 well-annotated SNPs among 1,415 genes with ≧4× coverage of 454 sequencing reads in the normal lung control sample (Supplement 2). Less stringent rules identified additional putative variants (higher sensitivity) but with diminished likelihood of being true-positives (lower specificity). These rules exhibited 100% specificity by confirmation of 94 SNPs discovered by the pipeline described herein using the conventional Sanger method. Thus, these rules appeared to provide sufficient specificity and sensitivity for discovery of true mutations among thousands of potential candidate variants and should assist others in discovery and validation of genetic variants and tumor mutations.

In addition to SNPs, indel variants can represent a common form of somatic mutation in the human genome, but are currently less well characterized (24). While high throughput pyrosequencing technology is suitable for identification of indels, additional rule refinement and improved alignment algorithms are required to remove homopolymer-related indel base calling errors.

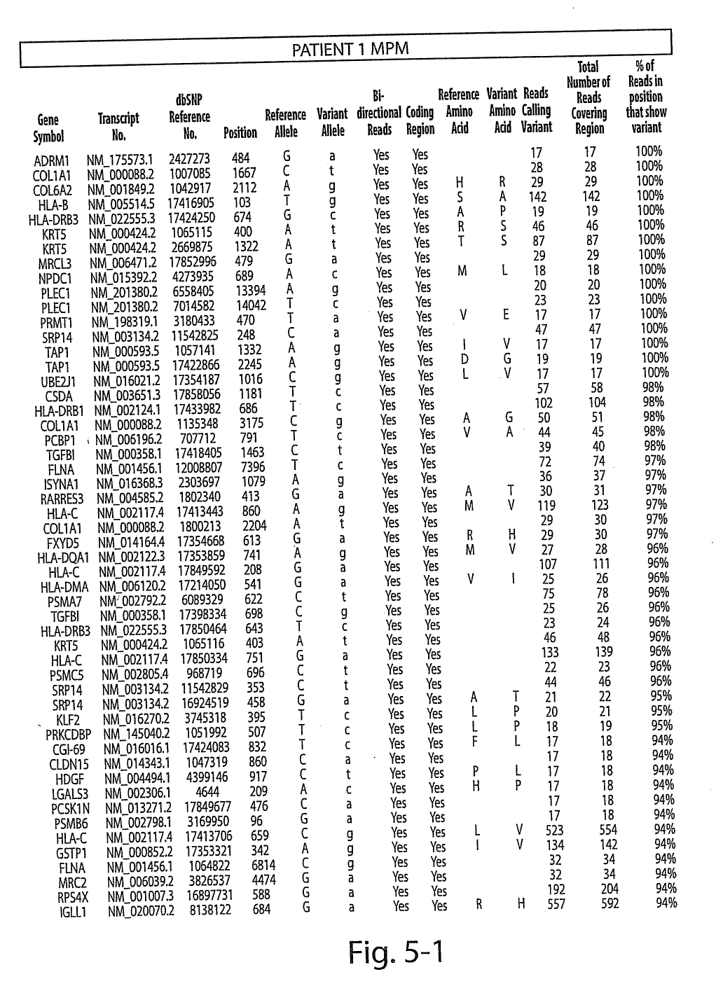

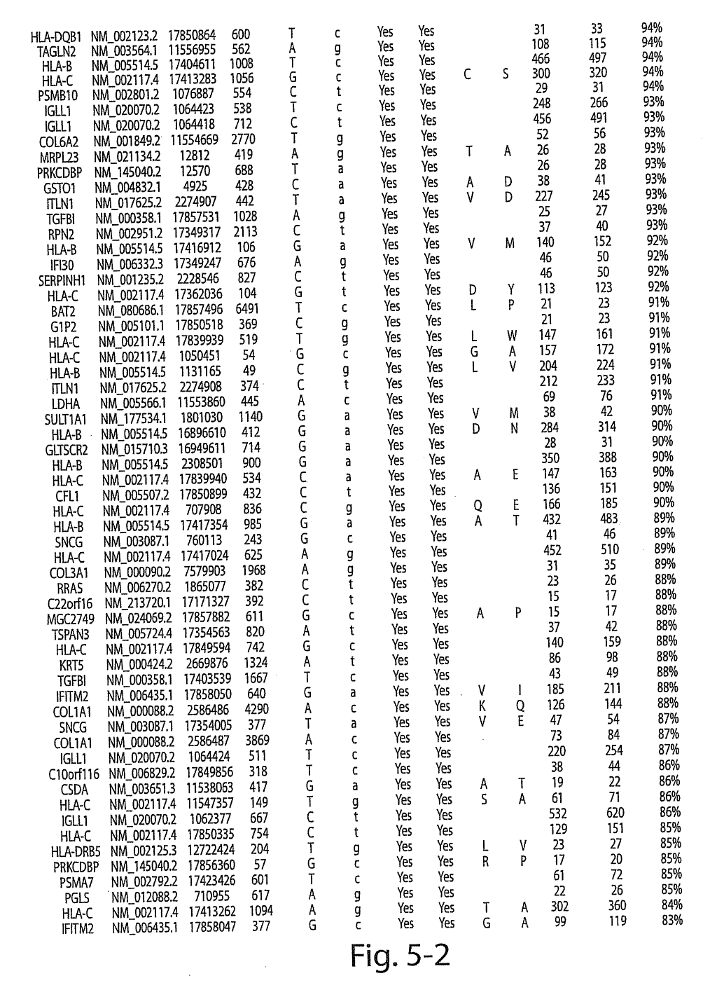

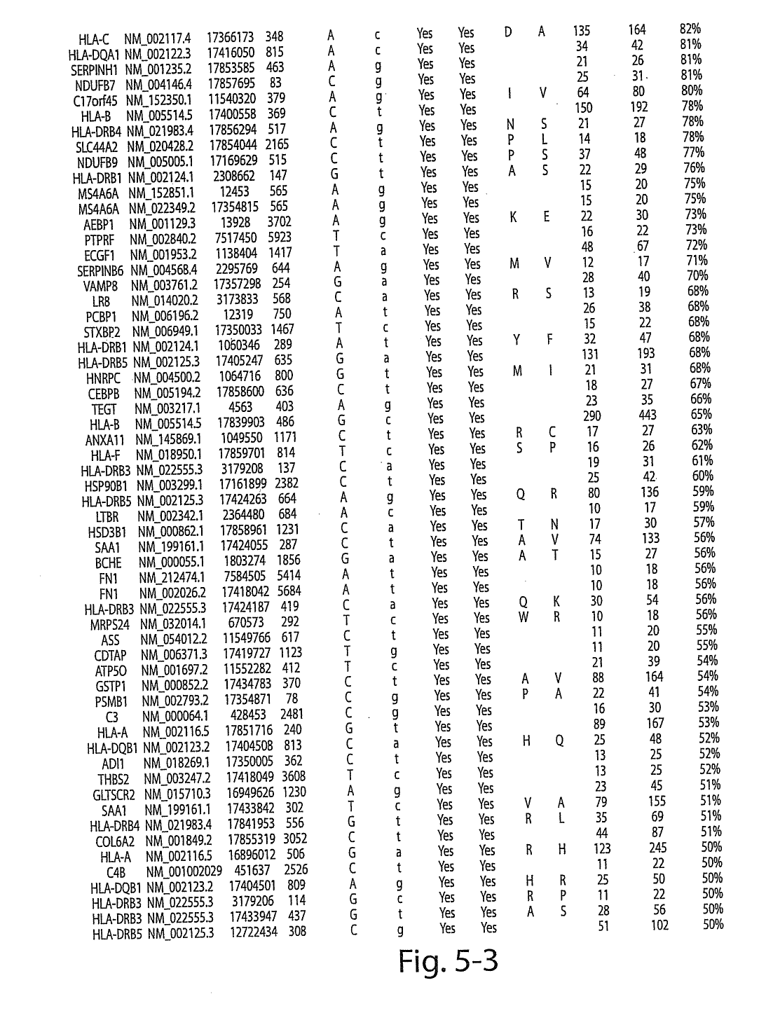

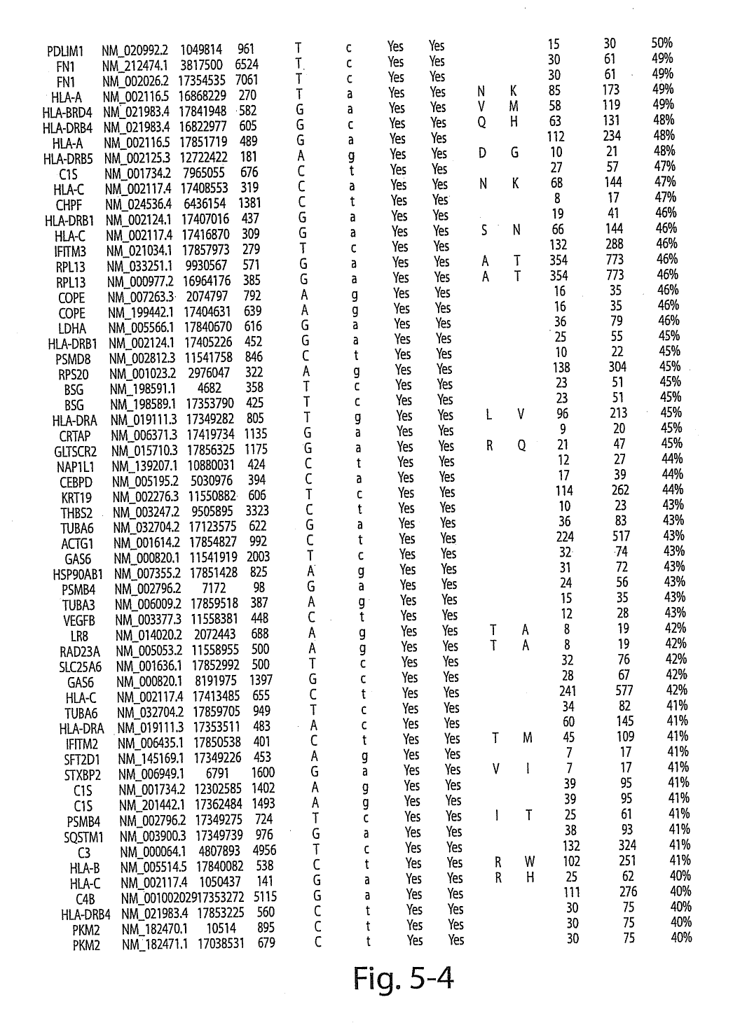

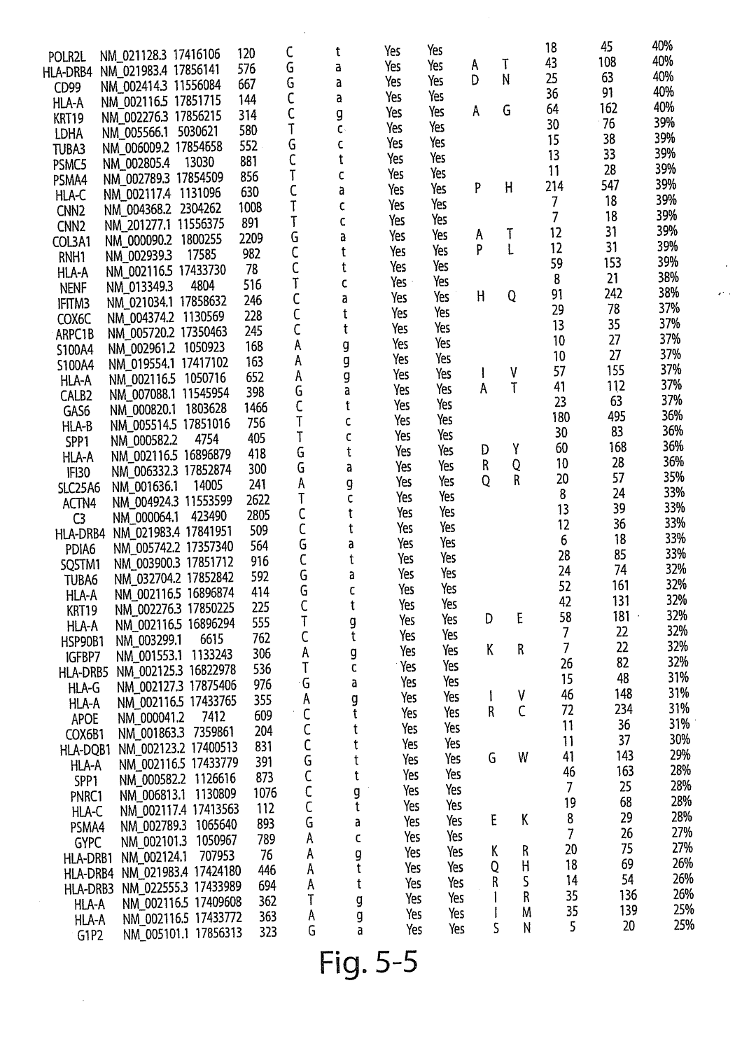

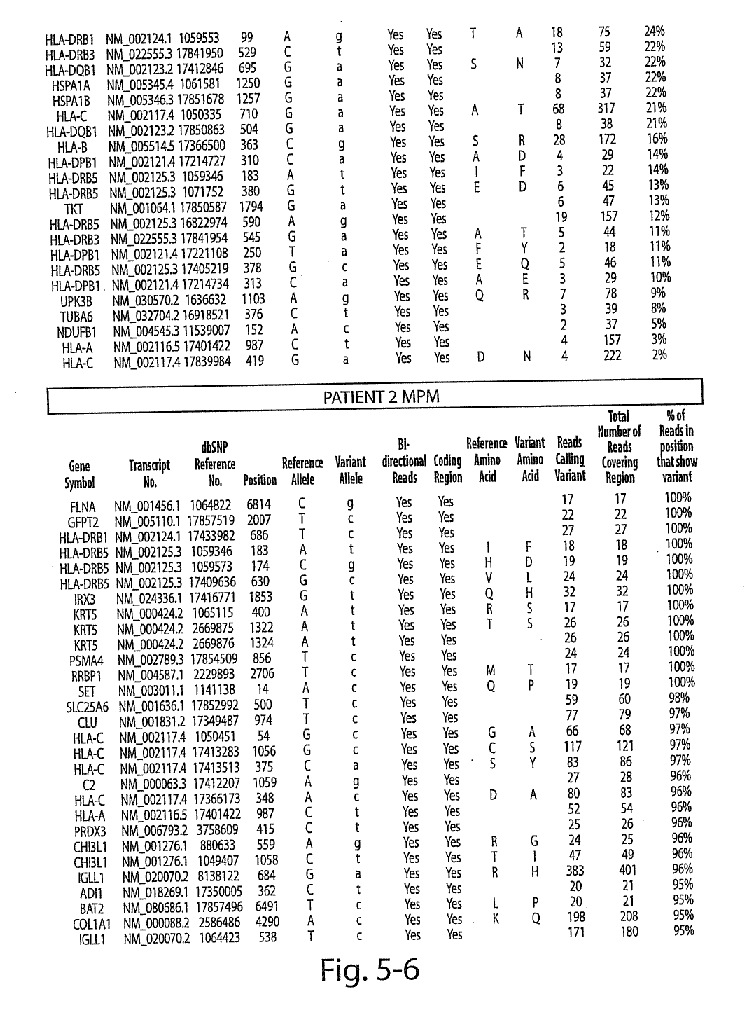

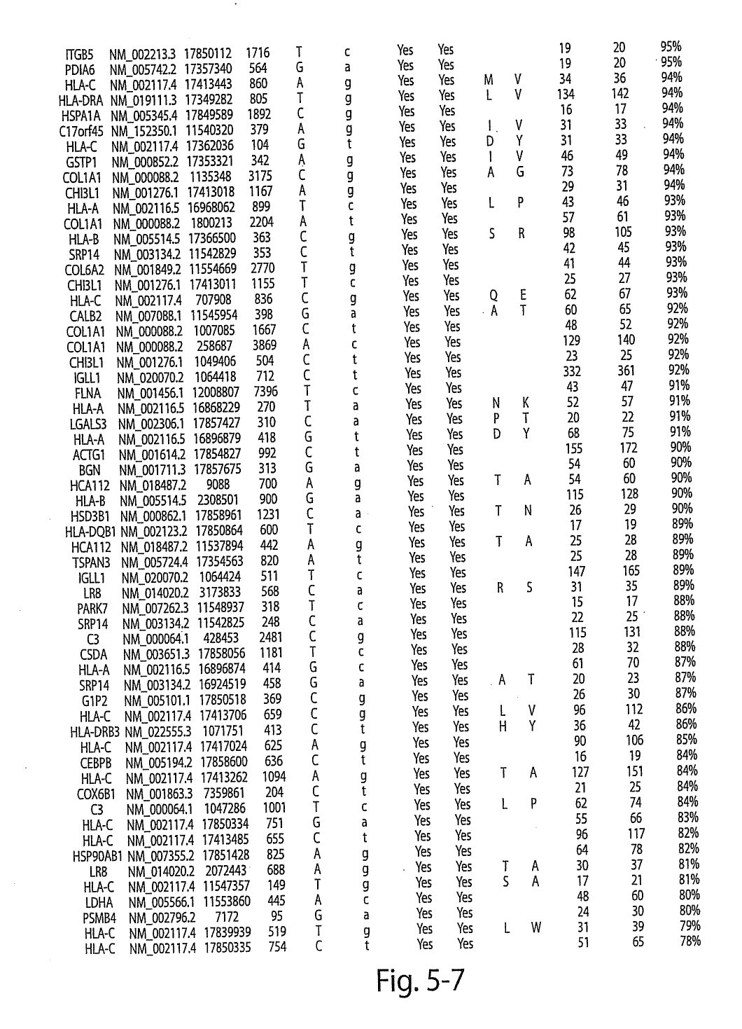

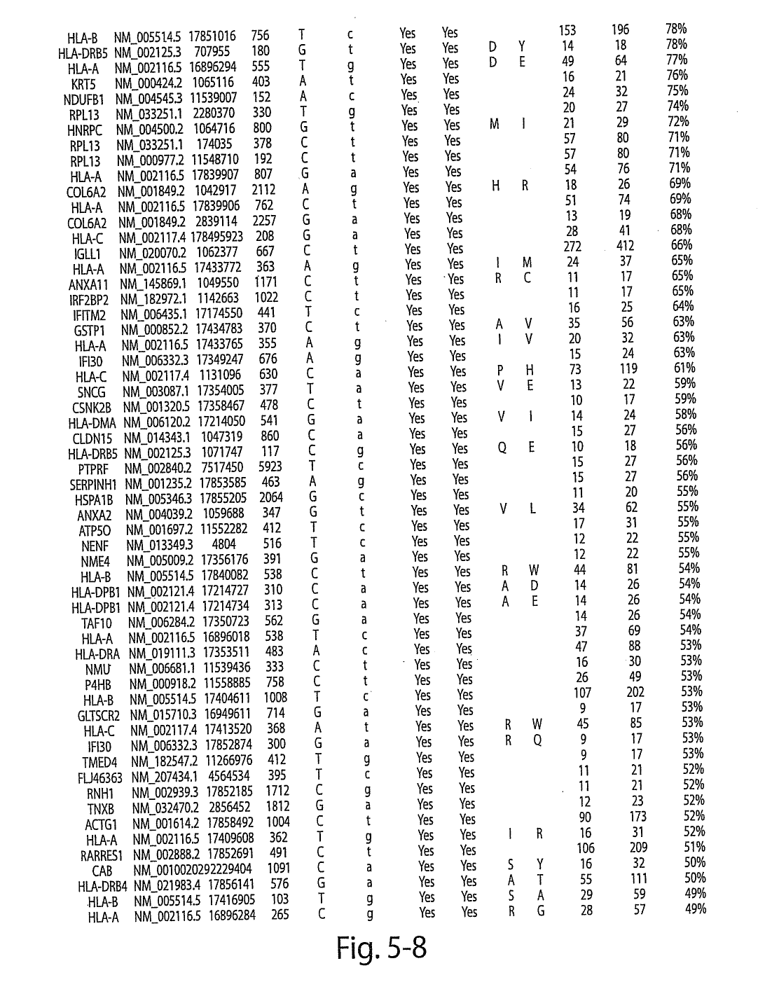

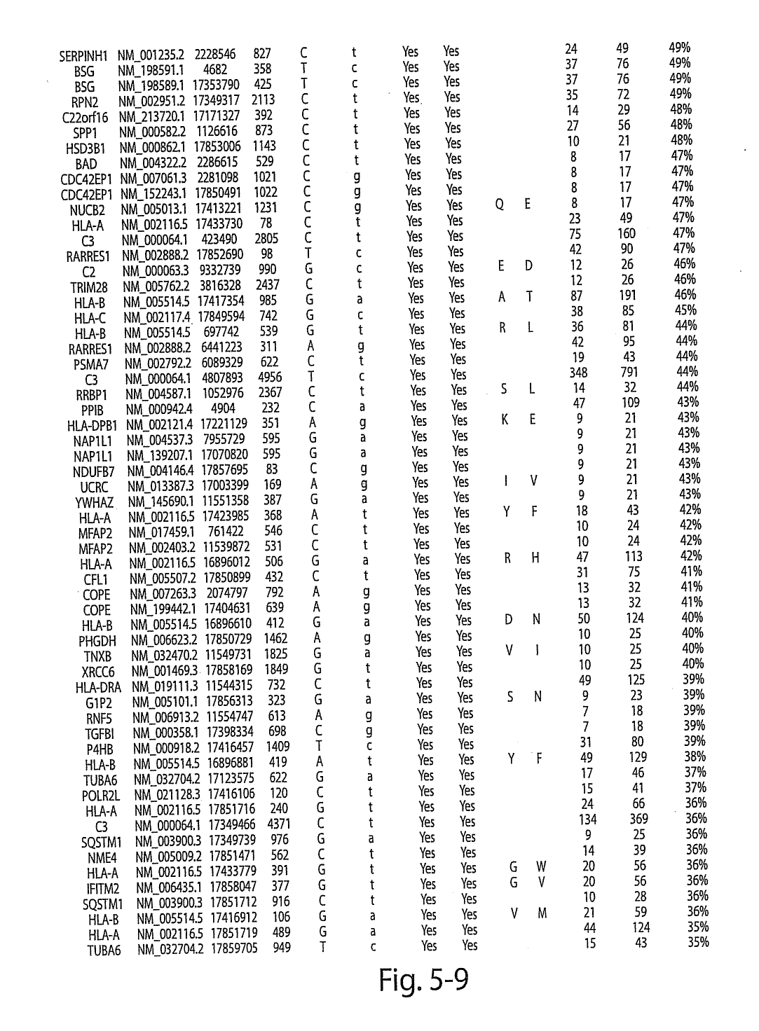

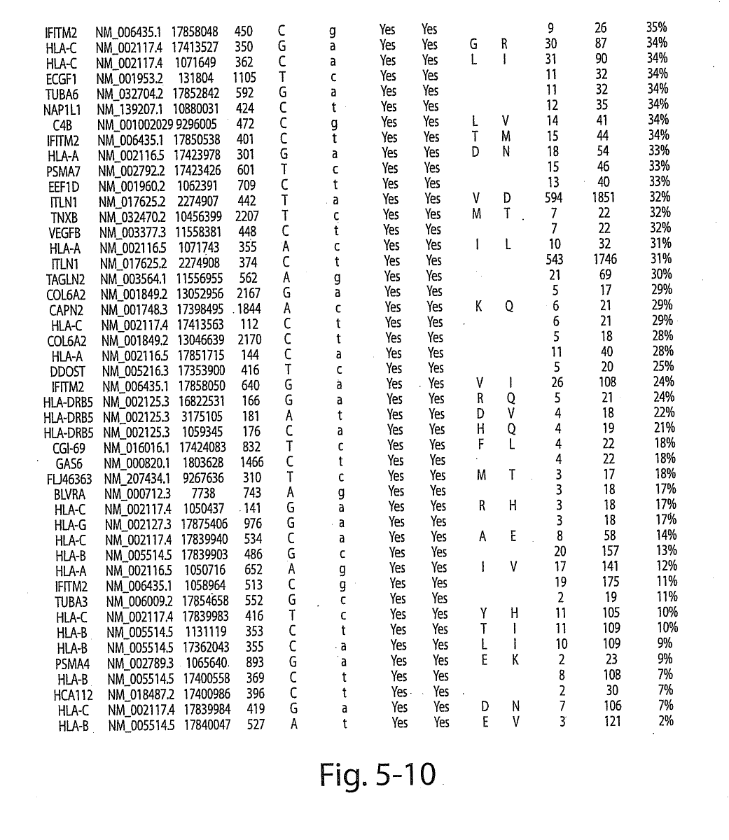

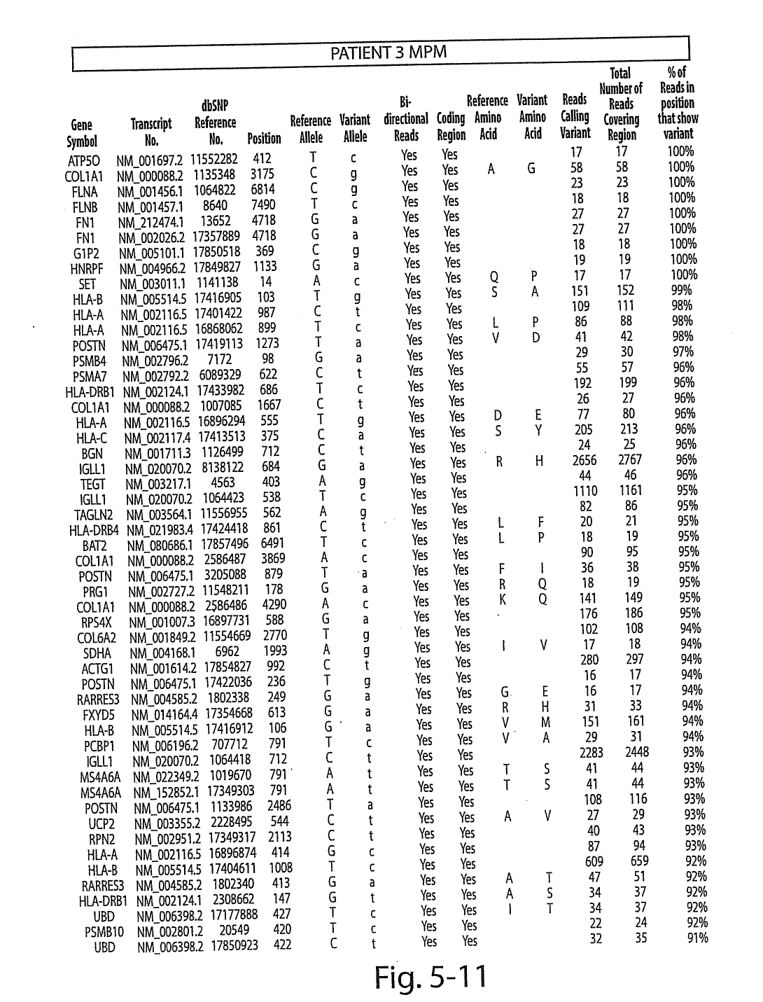

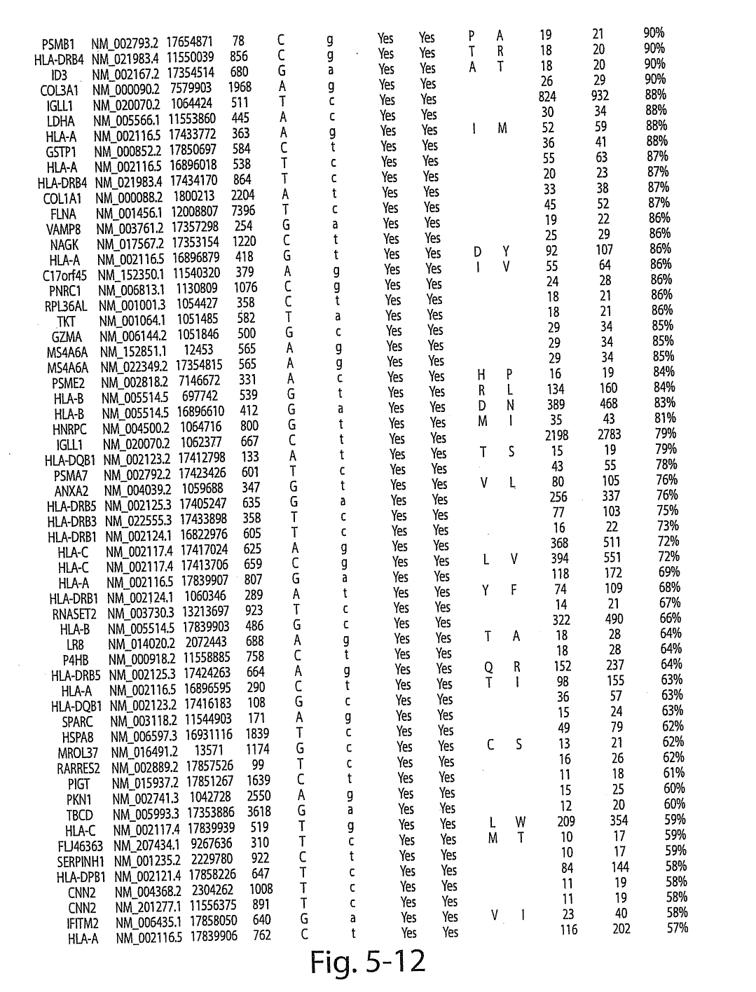

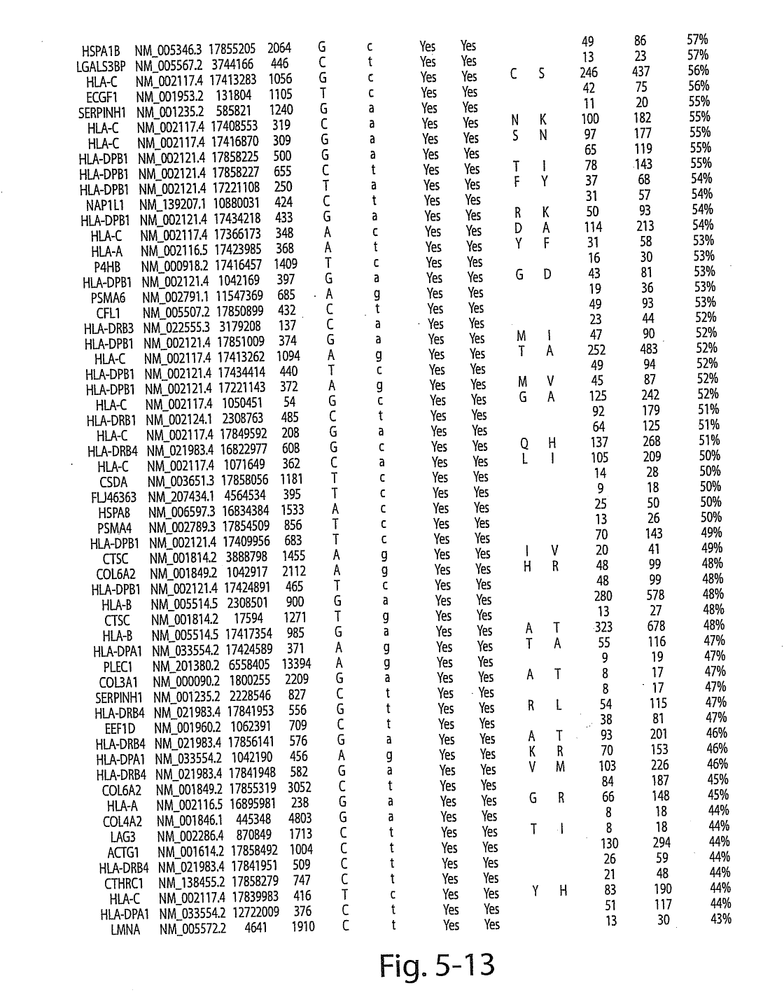

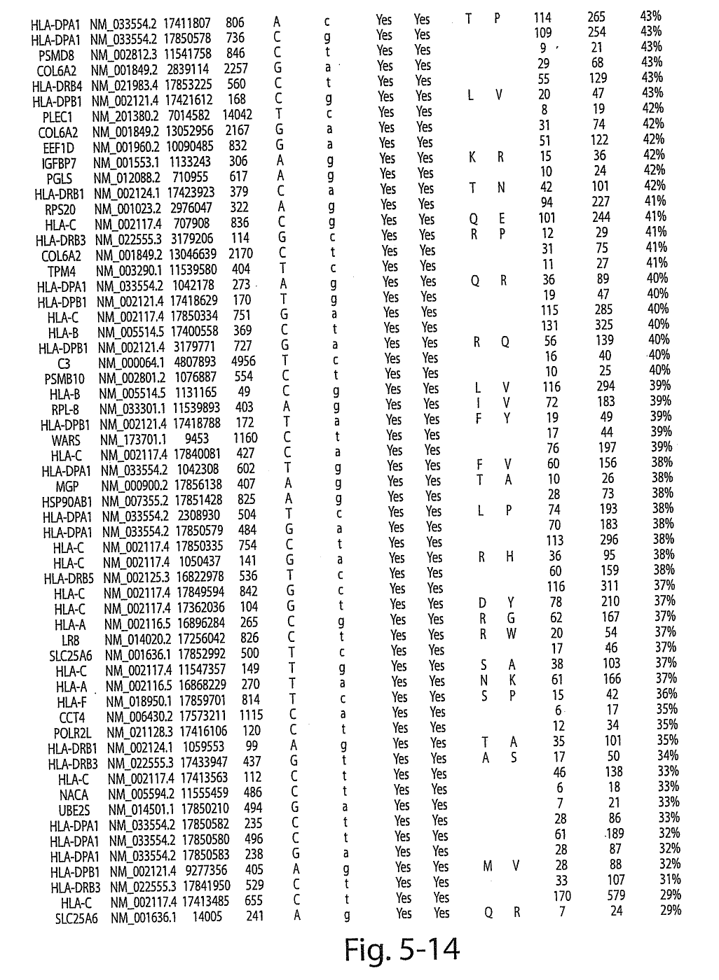

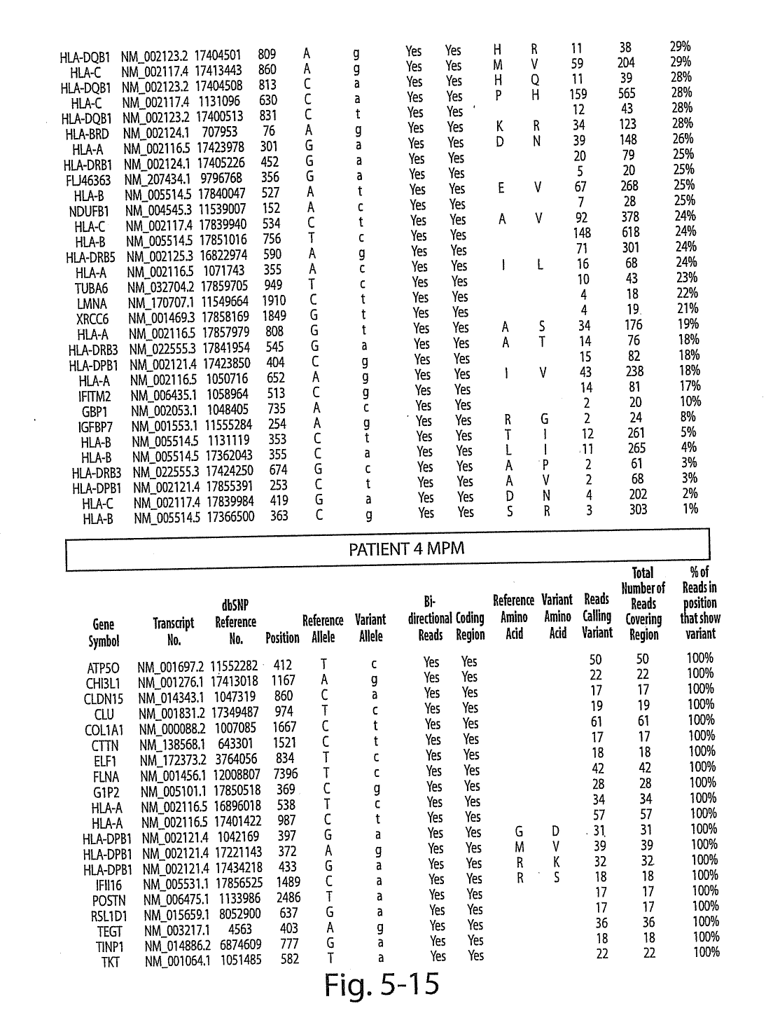

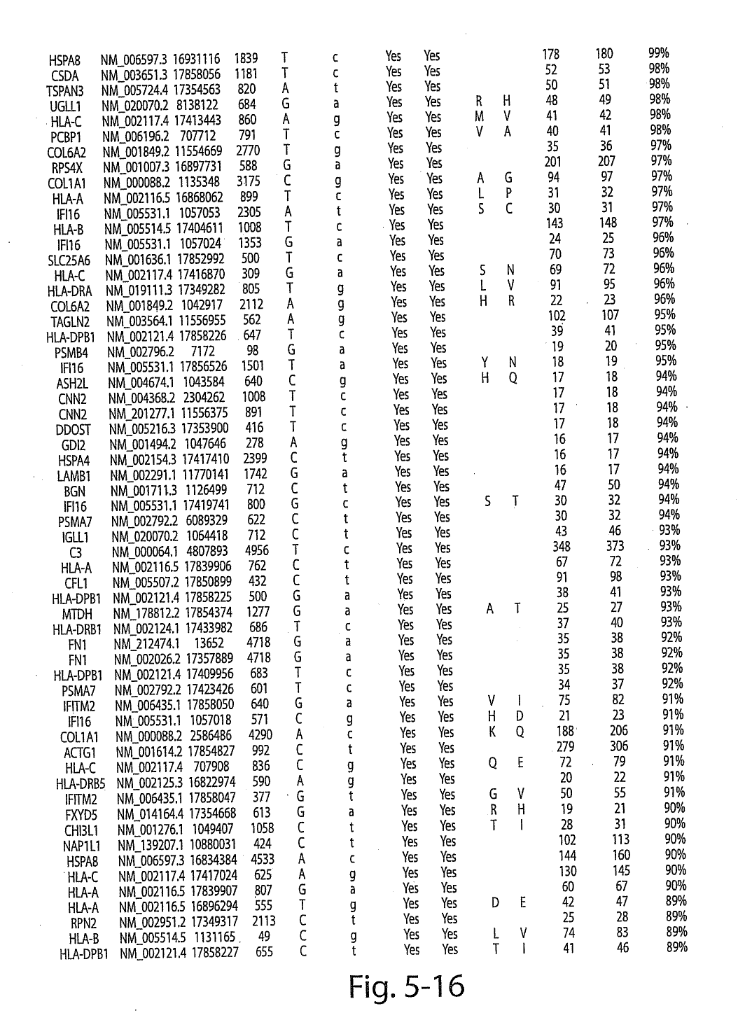

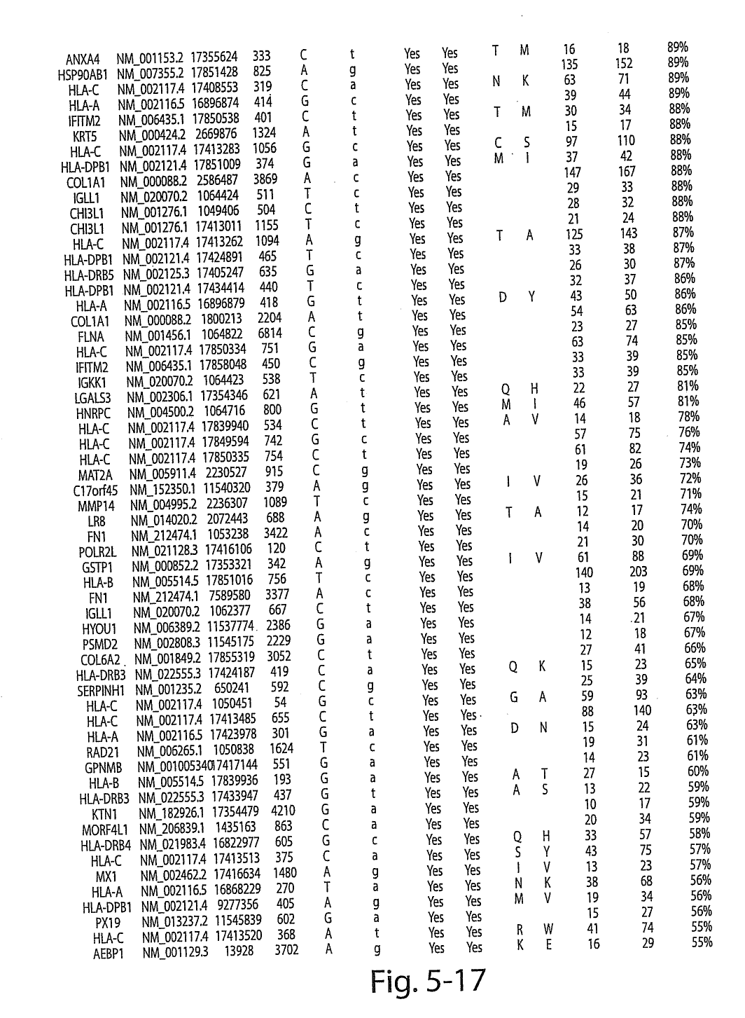

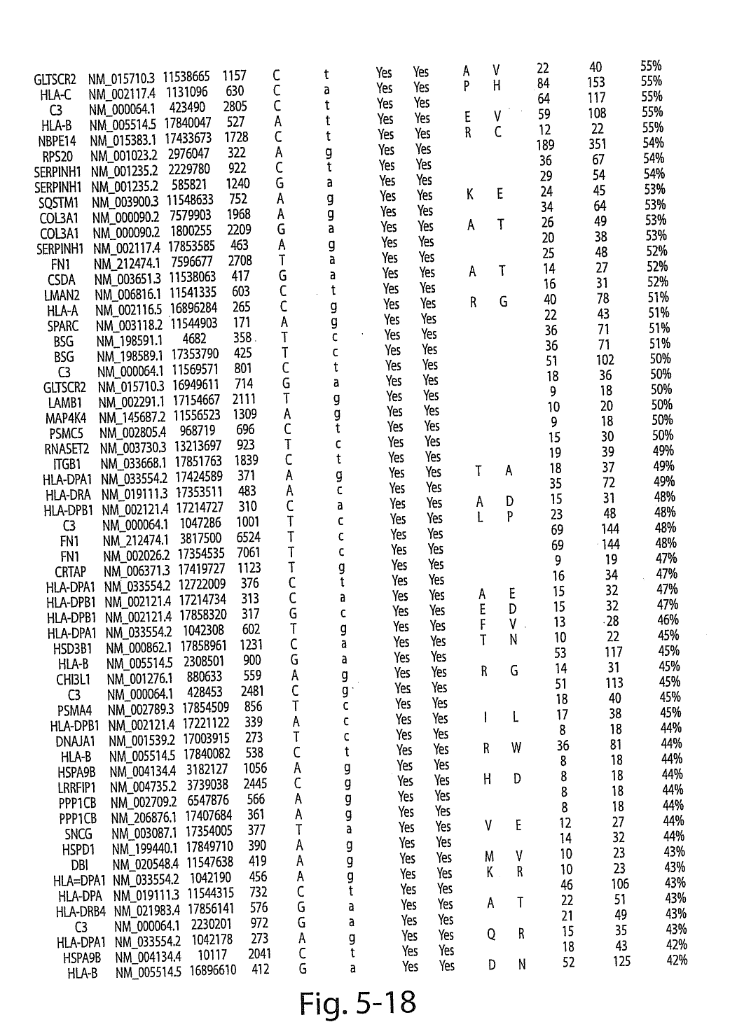

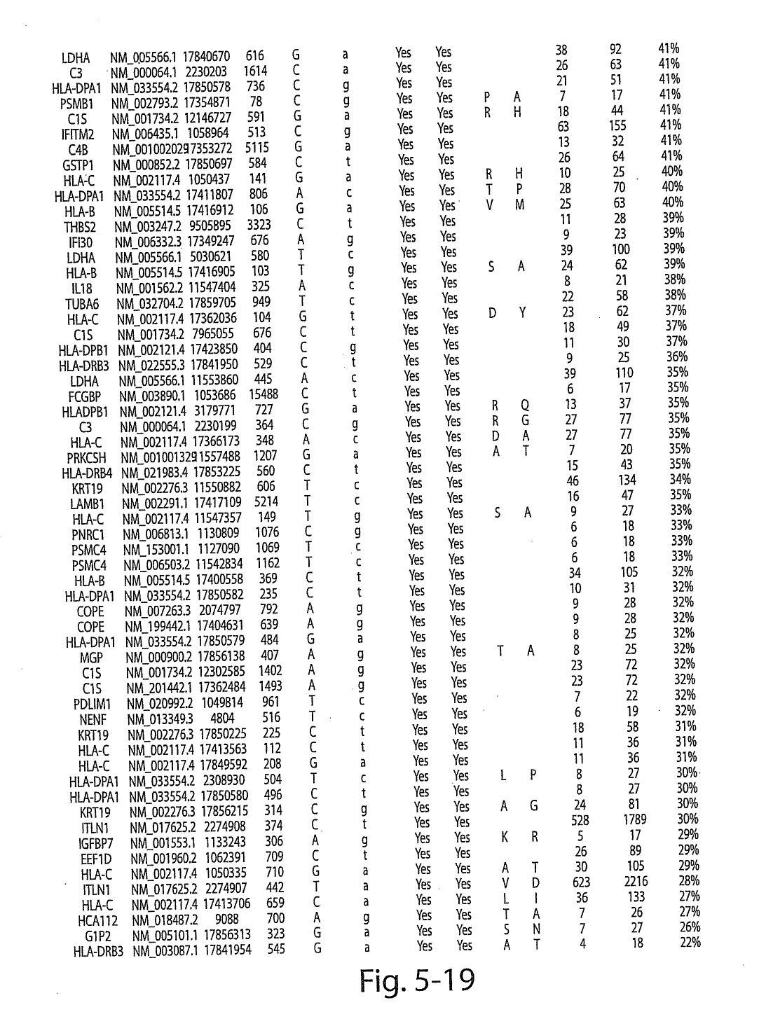

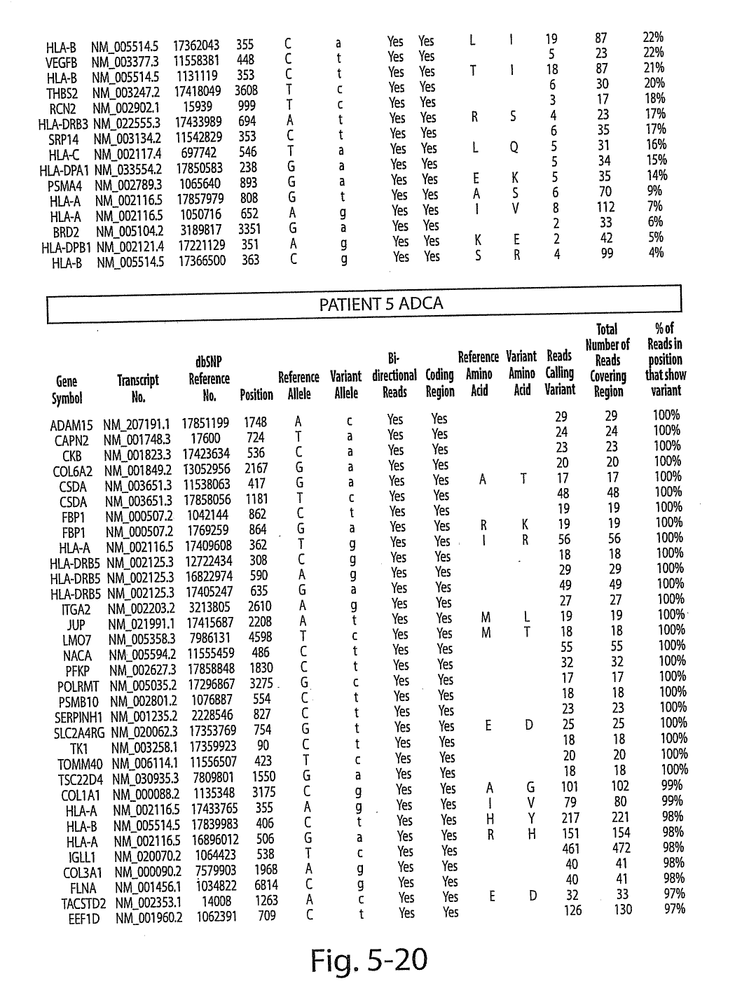

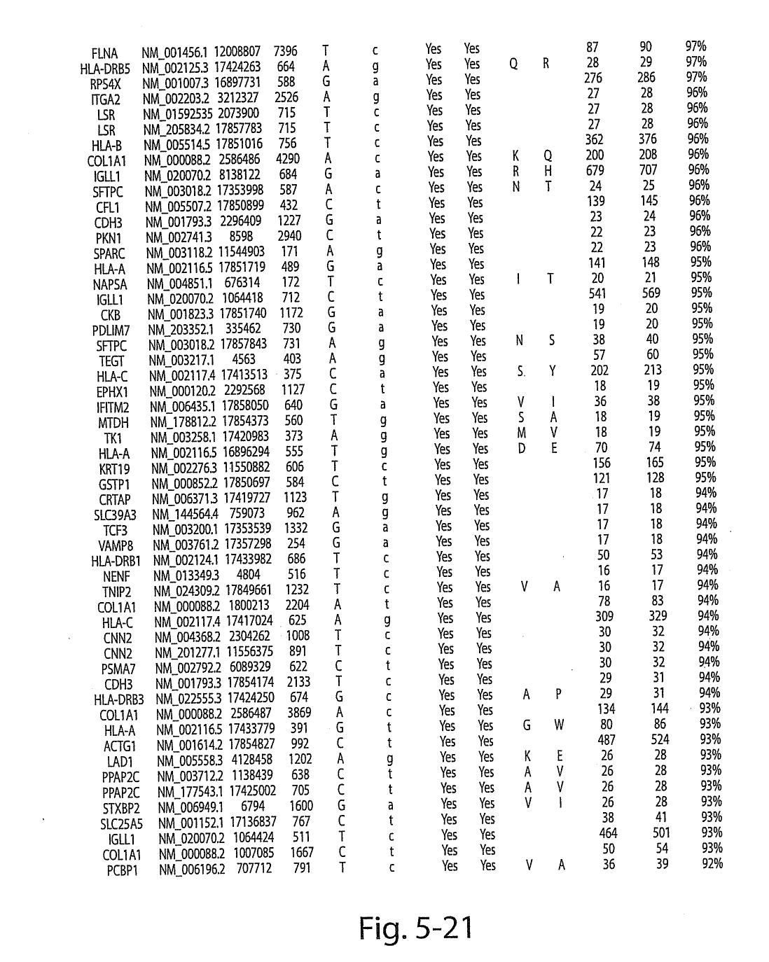

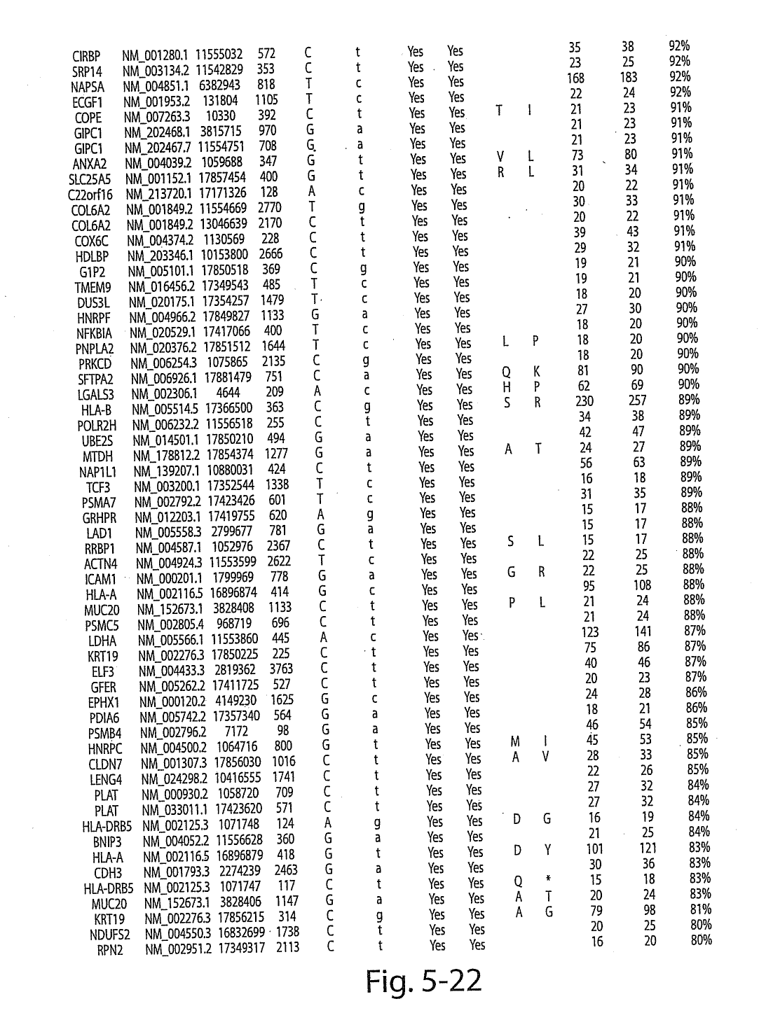

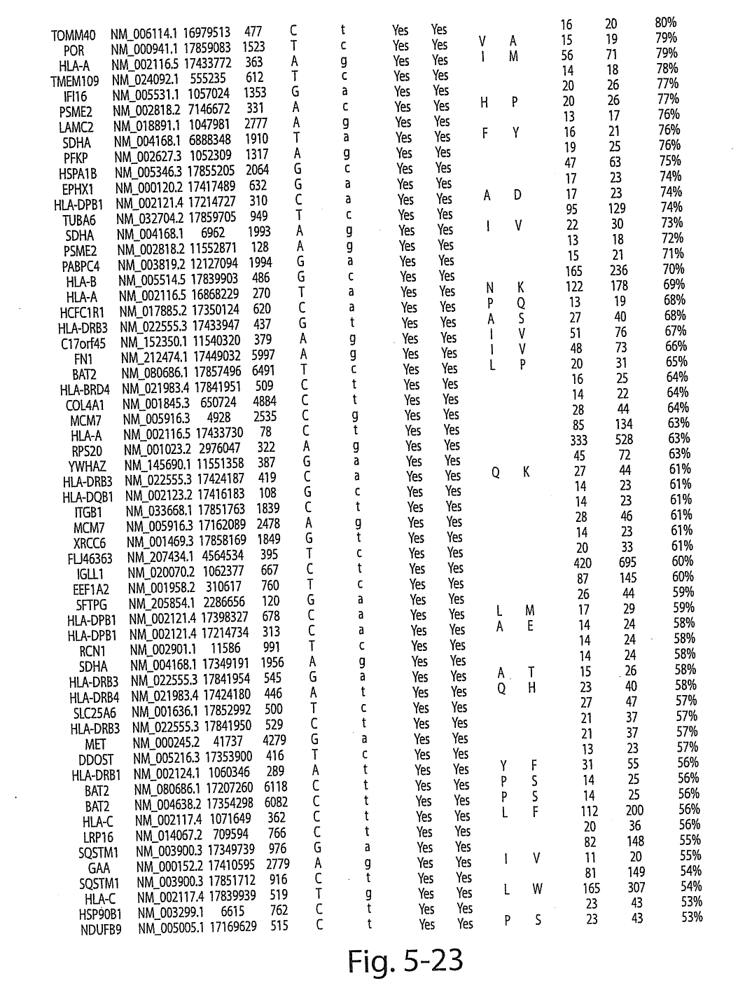

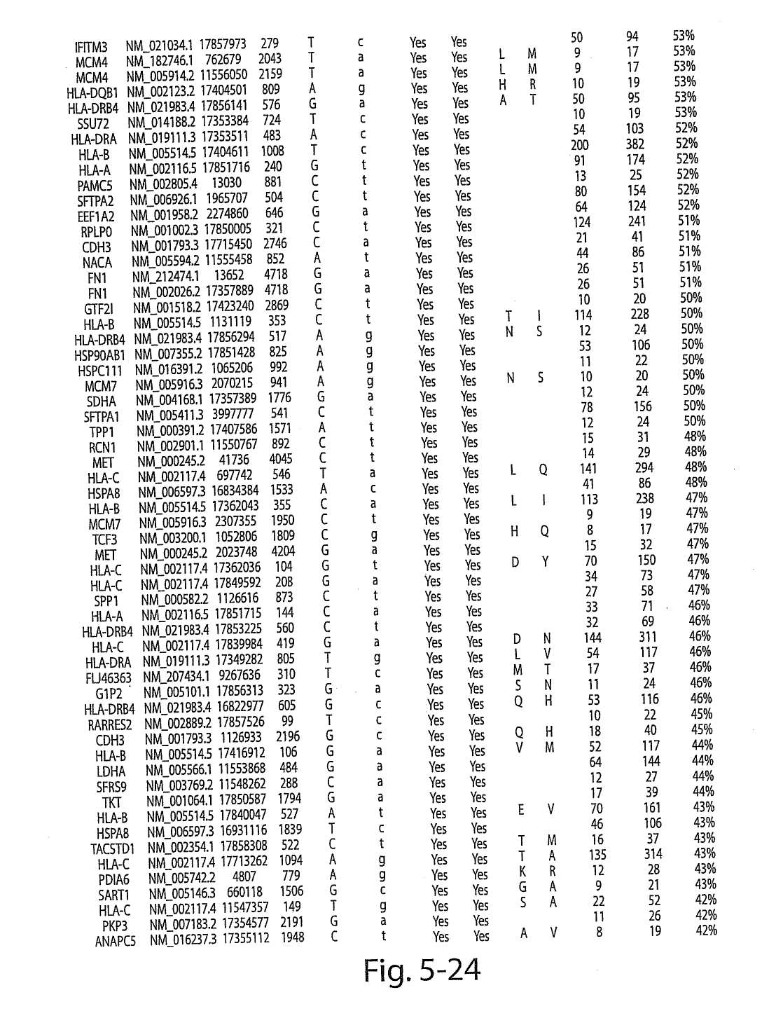

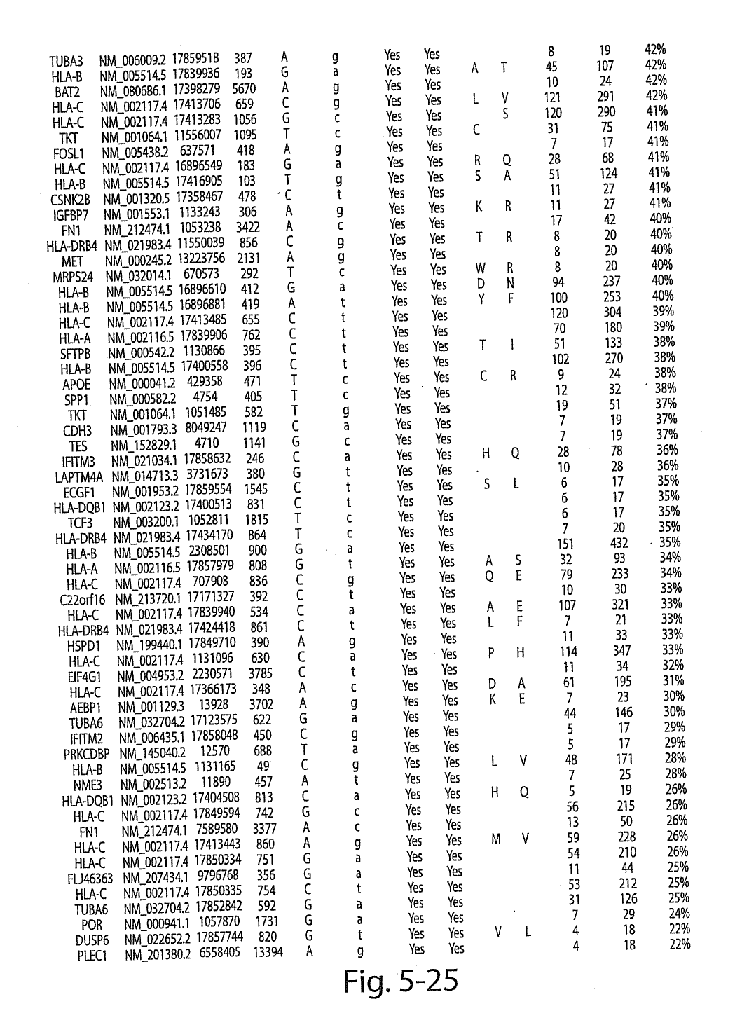

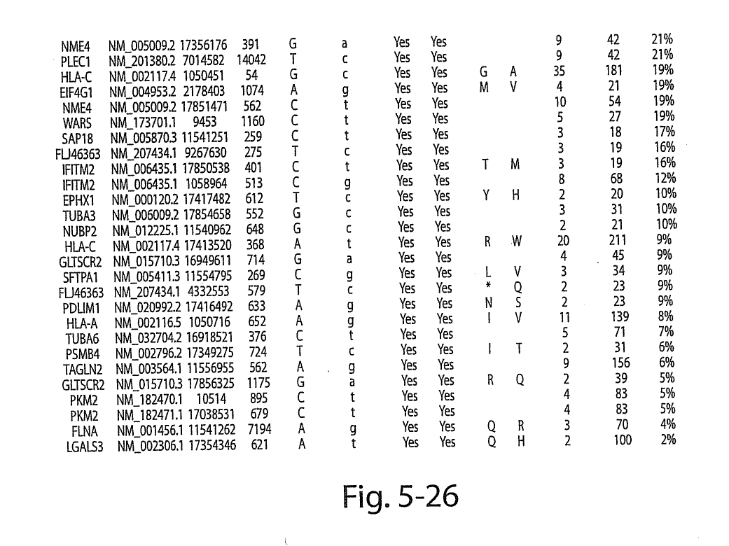

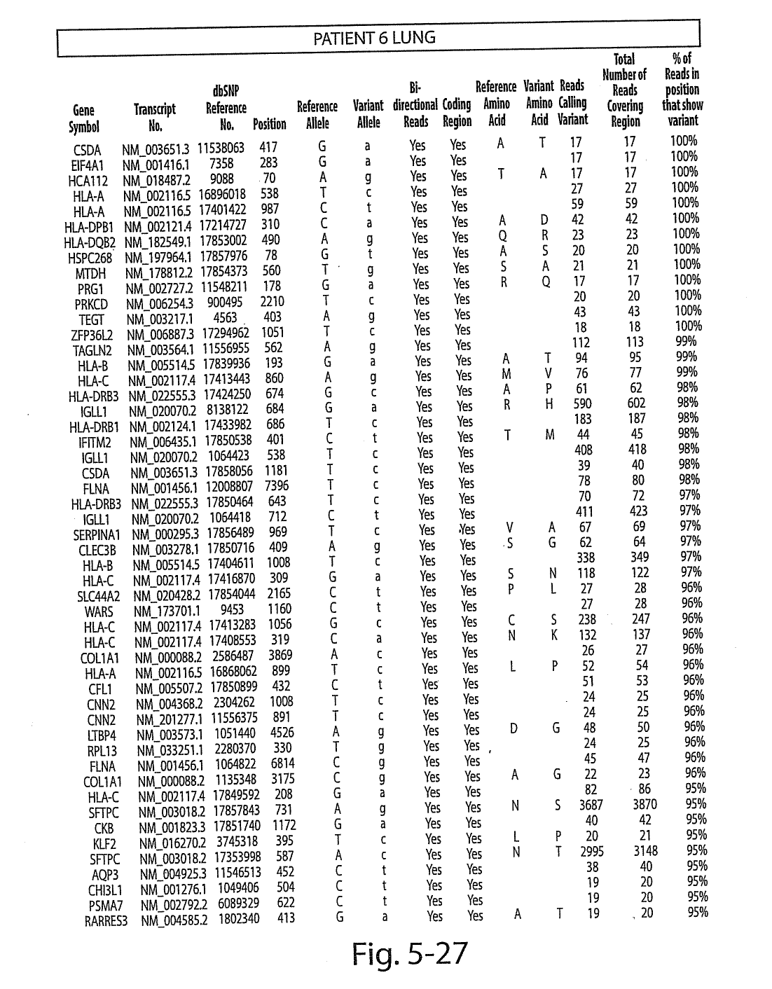

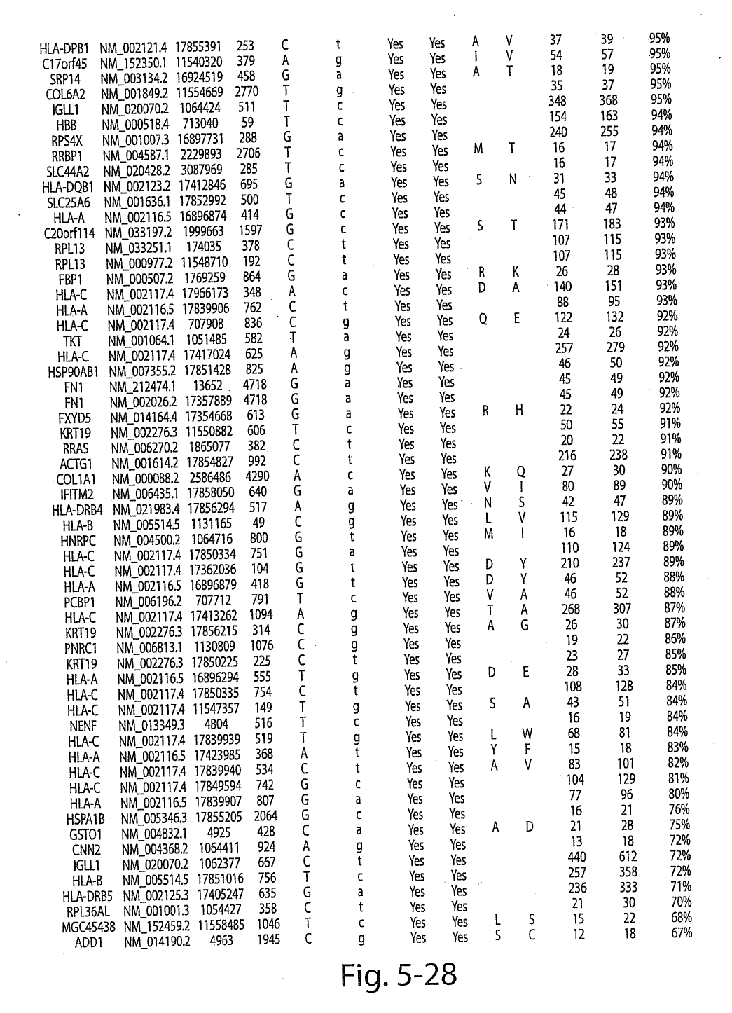

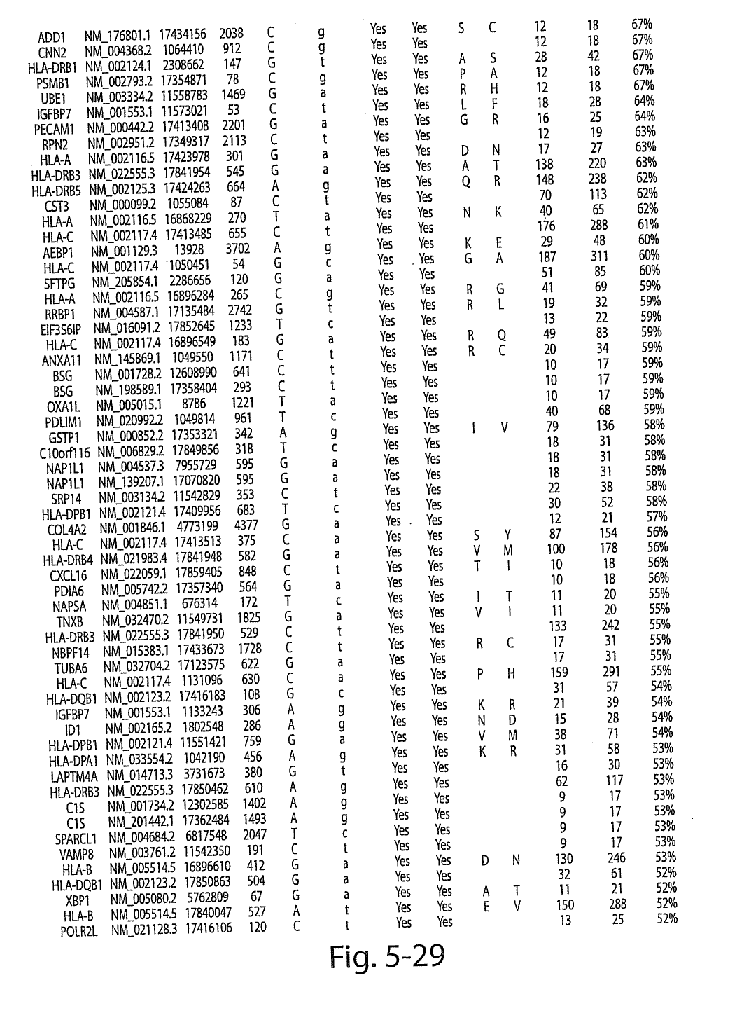

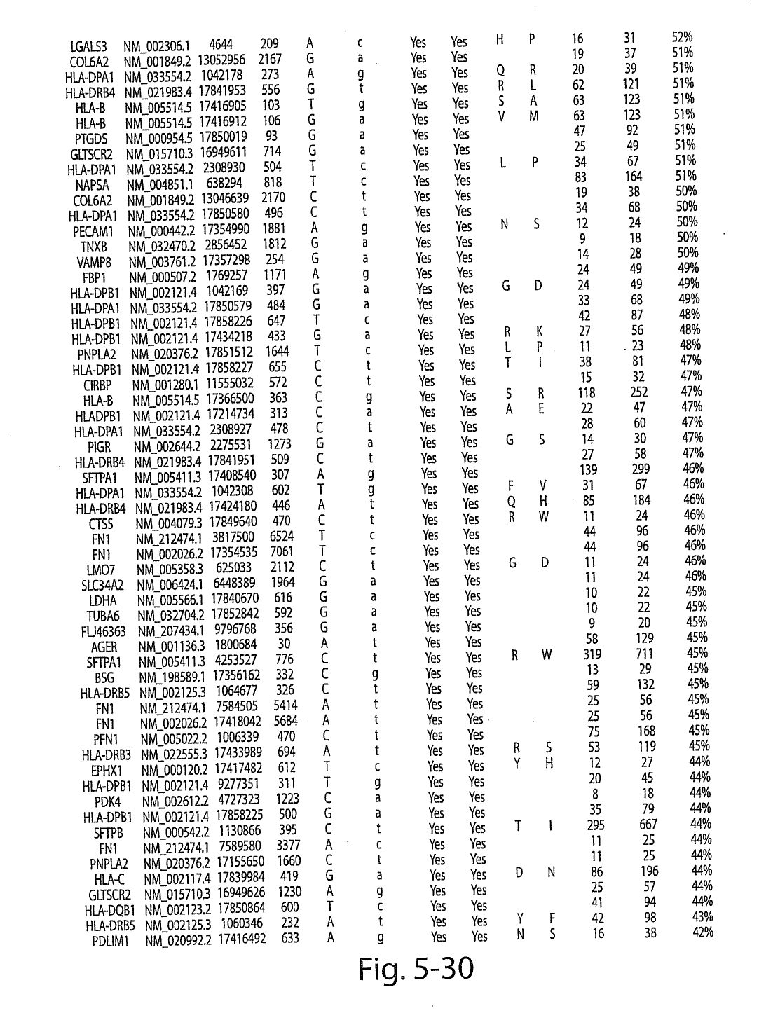

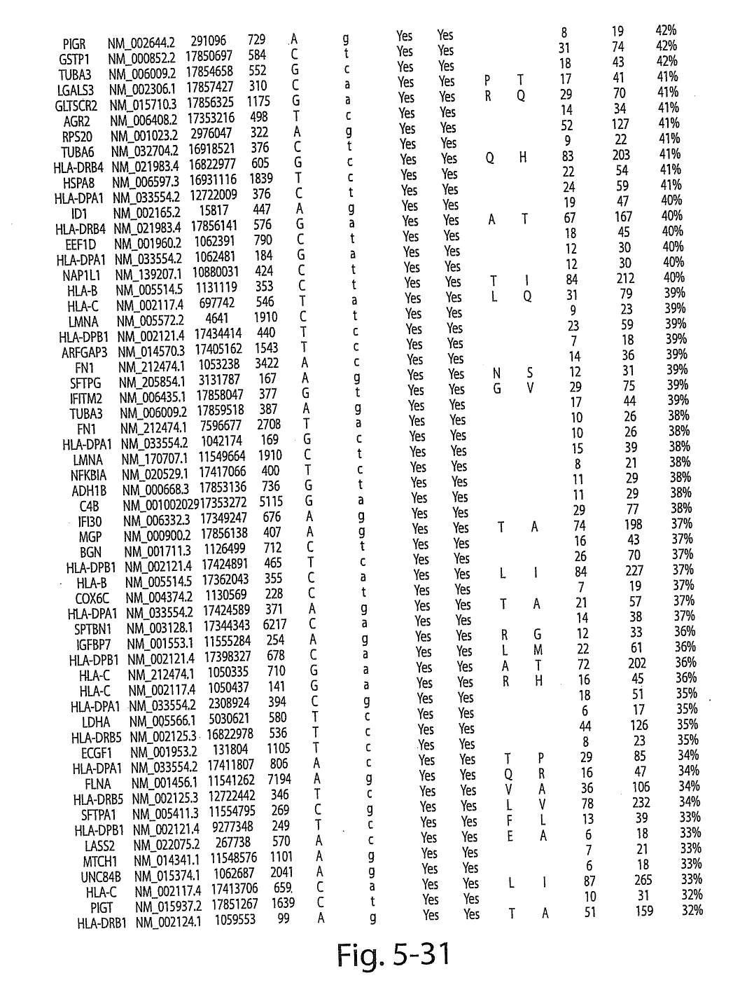

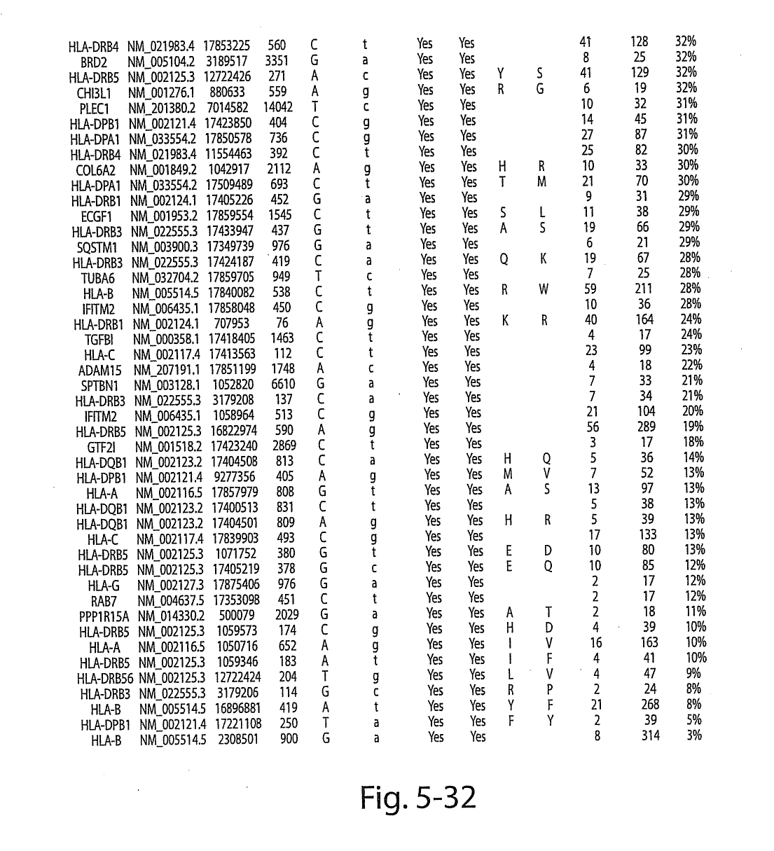

Since high-throughput 454 sequencing technology generates clonal sequences from individual mRNA molecules, enumeration of aligned reads allows estimation of the abundance of transcripts and relative expressed copy numbers of heterozygous alleles. An analysis of the transcript-based allele frequencies of 350 well-annotated coding region SNPs in abundant transcripts (>16 reads covering the SNP) in each specimen indicated the ability to detect homozygosity/LOH and heterozygosity (FIG. 4C, FIG. 5). In most cases, when both wild type and variant alleles were observed, they were expressed at similar levels (peak at 50%, FIG. 4C). However, a substantial subset of genes was also observed in which one allele was preferentially expressed (i.e., >80% of the reads). These observations were confirmed for a subset genes using Sanger sequencing. Asymmetric allele expression may variously reflect the homozygous presence of a SNP variant, preferential transcription/stability of one allele (25), or copy number variation (26). While the small number of tumors evaluated precludes generalization, transcriptome analysis with 454 sequencing technology should be suitable for gross analysis of allele copy number, particularly LOH or duplication for heterozygous alleles.

### Example 4

### Sequence Variants and Candidate MPM Mutations

All candidate mutations derived from the four MPM samples (Patients 1-4) were selected for confirmation and characterization using conventional Sanger sequencing, both in the discovery samples and additional tumors (the validation set) and matched normal tissue from patients with MPM.

### Validation of Candidate Mutations

The NCBI RefSeq RNA sequence was used as input for the web based primer3 program (http://frodo.wi.mit.edu/) to design primers that amplify approximately 500-700 bp fragments of cDNA centered on the variant of interest and span an intron (if possible) to minimize problems associated with potential genomic DNA contamination. Other settings used for primer input included: Mispriming library (Human); Primer Size (18 to 20 nucleotides); Primer Tm (59 to 61° C.); and Primer GC % (45-55%). Candidate primers were further analyzed for specificity by comparison to all publicly available sequence information (http://www.ncbi.nlm.nih.gov/BLAST/).

For each variant, at least 3 primer pairs were chosen for additional optimization with (RT-)PCR to assess priming specificity using cDNA, RNA, genomic DNA and water as templates. Final primer selection was based on specificity, absence of primer dimers, absence of product in negative controls, and visualization of single amplicons of expected size using agarose gel electrophoresis and standard protocols. Whenever possible, primers were chosen that amplify both cDNA and genomic DNA. If this was not feasible (e.g., due to excessively large introns), additional primers were designed to amplify specifically genomic DNA using a similar approach. Primers were synthesized by Invitrogen Life Technologies (Carlsbad, Calif.) and are listed individually in FIG. 6.

### Reverse Transcribed PCR (RT-PCR)

Total RNA (2μ) was reverse-transcribed into cDNA using Applied Biosystems Reverse Transcription reagents and random hexamers as the primer (Applied Biosystems, Foster City, Calif.). RT was performed in a final volume of 100 μL, a portion of which was used as a template for PCR amplification using the Roche FastStart High Fidelity PCR System (Indianapolis, Ind.). Each PCR reaction (50 μL total) contained 2 μL template, 2 μL forward primer (10 μM stock), 2 μL reverse primer (10 μM stock), 5 μL 10× buffer (containing 18 mM MgCl₂), 1 μl dNTP mix (containing 10 μM dNTP), 0.5 μl FastStart High Fidelity Enzyme Blend (2.5 U total), and 37.5 μL water. PCR cycling parameters were: 1 cycle at 95° C. for 2 min., 35 cycles of 95° C. for 30 s, 60° C. for 30 s, and 72° C. for 1 min., followed by a final extension at 72° C. for 5 min. After PCR, 5 μL of each reaction was subjected to agarose gel electrophoresis to confirm a single product of the expected size. The remainder of the reaction was purified using a QiaQUICK PCR Purification Kit (Qiagen).

### PCR of Genomic DNA

Genomic DNA was obtained from peripheral blood lymphocytes and microaliquoted tumors using a QiaAMP DNA Mini Kit (Qiagen) and the manufacturer's protocols. PCR was performed exactly as for cDNA using 50-100 ng genomic DNA as a template. Primers used in the reactions are described in FIG. 6.

### Sanger Sequencing

Purified PCR products were quantified using an ND-1000 Spectrophotometer (Nanodrop Technologies, Wilmington, Del.), and 120 ng were used for sequencing using the forward and reverse PCR primers (1.6 μL) in separate wells. Sequencing was performed using an Applied Biosystems 96-well capillary 3730XL DNA analyzer. Base calls were made by the instrument software, and forward and reverse chromatograms were inspected manually. Bioedit Software v.7.0.5.1. (Hall, T. A. 1999 Nucl Acids Symp Ser 41:95-98) was used to align and compare the experimental sequences of PCR products and the reference sequence. A variant was called “present” if it could be documented in duplicate experiments using at least 2 different sets of PCR primers. Since tumor content was ˜85%, contamination with non-tumor sequences of up to 15% was expected.

### Validation Set

A validation set of multiple frozen tumors and controls was used as follows: first, genomic amplification and sequencing primers were prepared for mutations validated in the discovery set. Then the relevant amplified regions in the genomic tumor DNA of 49 MPM tumors (26 epithelial, 17 mixed and 6 sarcomatoid) were bidirectionally sequenced. Thus, a total of 53 MPM tumors were used, 49 for validation and 4 for discovery. For specimens where the mutant allele was present, the tumor cDNA (with different appropriate set of primers), as well as the PBL genomic DNA and adjacent normal tissue genomic and cDNA when available, were also sequenced.

Cataloging of all high likelihood, true positive SNPs in the 6 samples using the rule set revealed between 659 and 1,155 Known RefSeq Genes per tumor sample with at least one coding domain SNP (FIG. 3). Approximately 20% of the well covered genes contained at least one known or novel SNP. In this context, a “known” SNP was defined as having been previously recorded in either RefSeq RNA or dbSNP at NCBI. (In fact, many of the unknown SNPs identified using this criteria were also not previously recorded in either the nr database or EST human database at NCBI.) The prevalence of SNPs in untranslated regions (UTRs) was substantially greater than in coding regions, but these were not examined further. Novel, rather than previously described, coding SNPs in the four MPM samples were explored further, as they have the highest likelihood of functional relevance in oncogenesis.

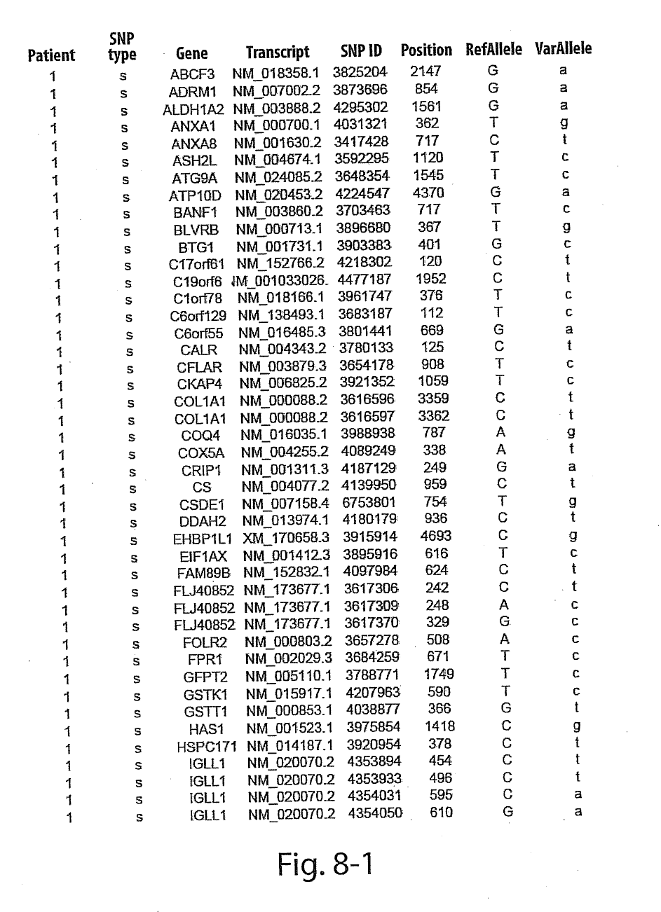

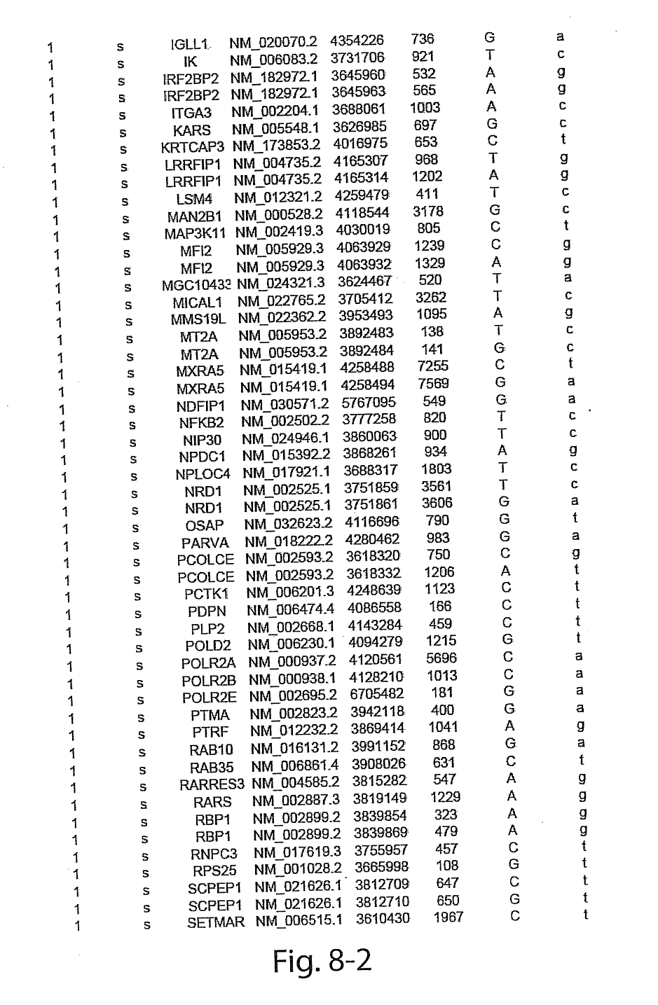

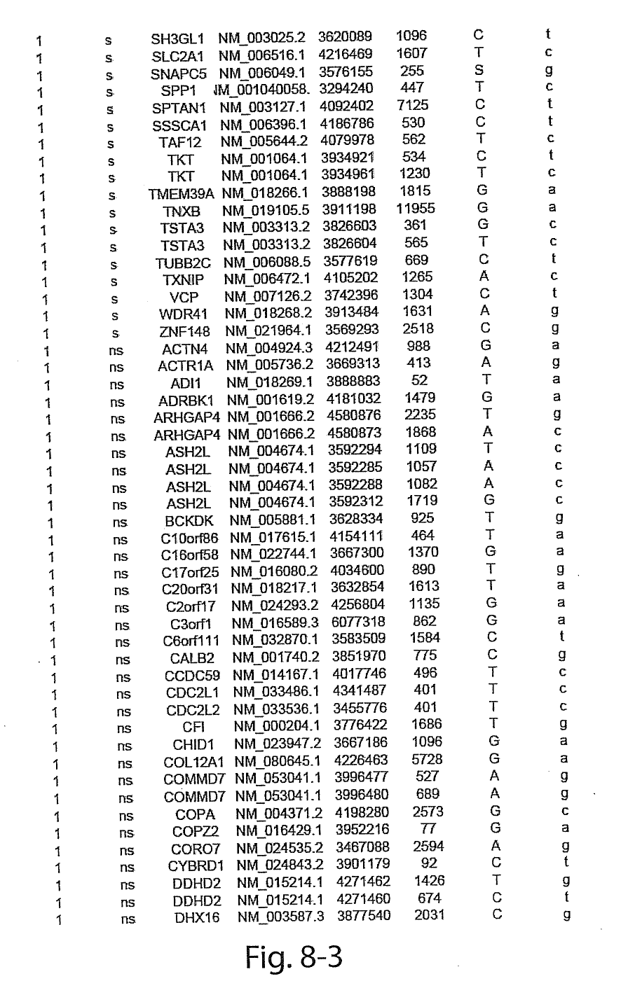

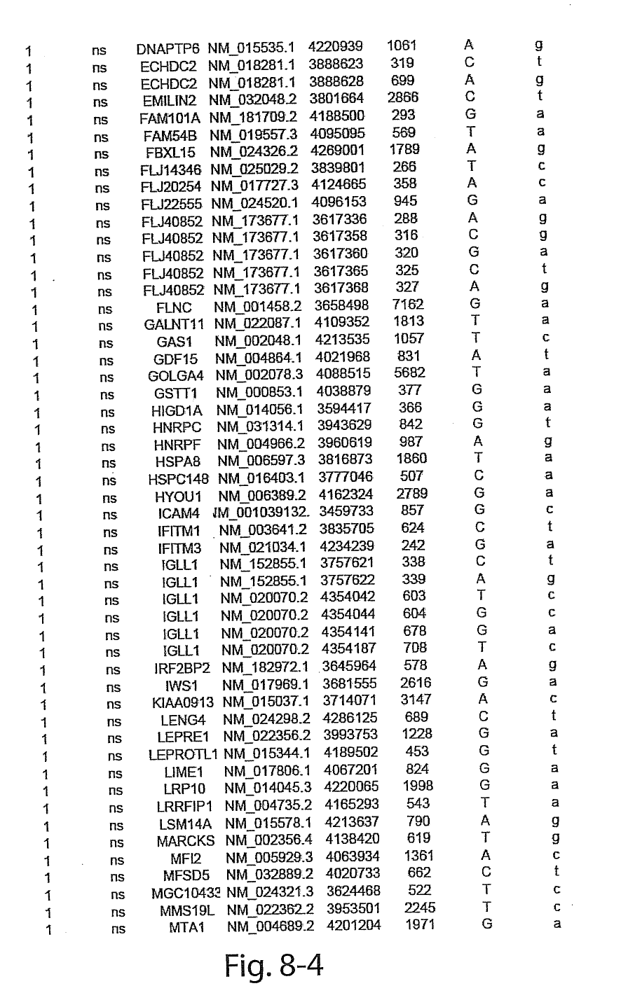

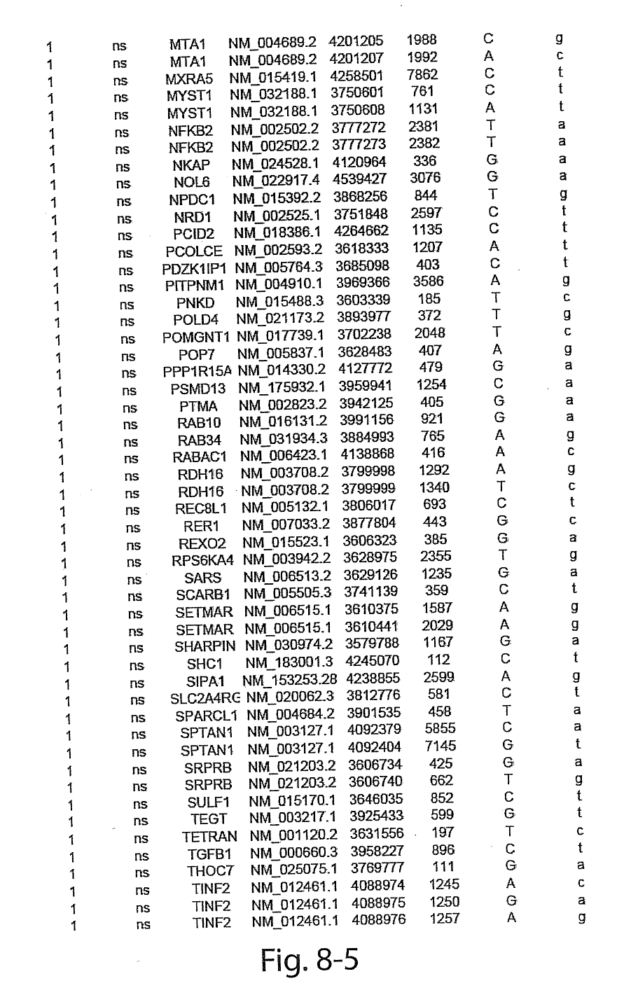

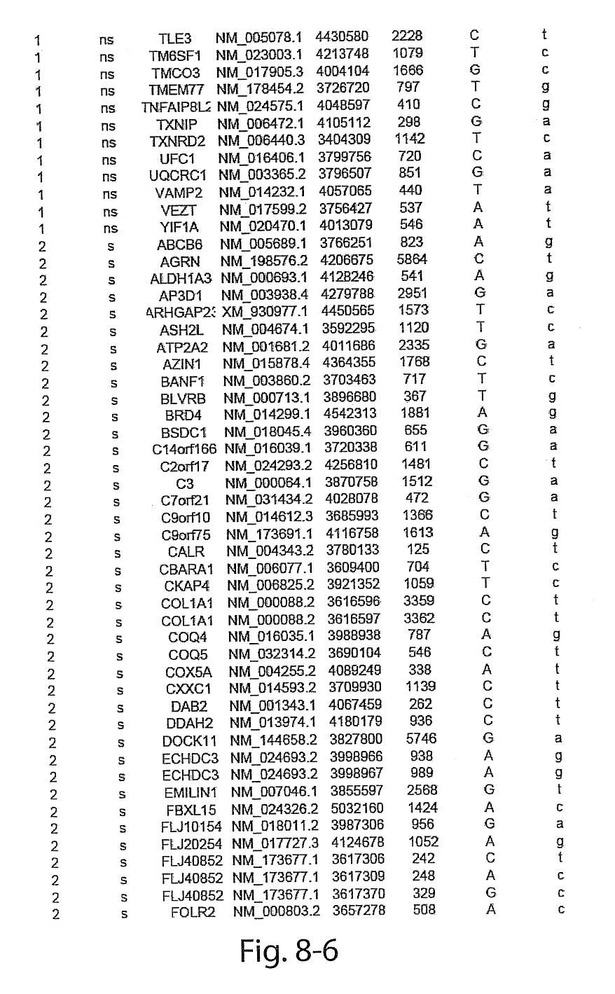

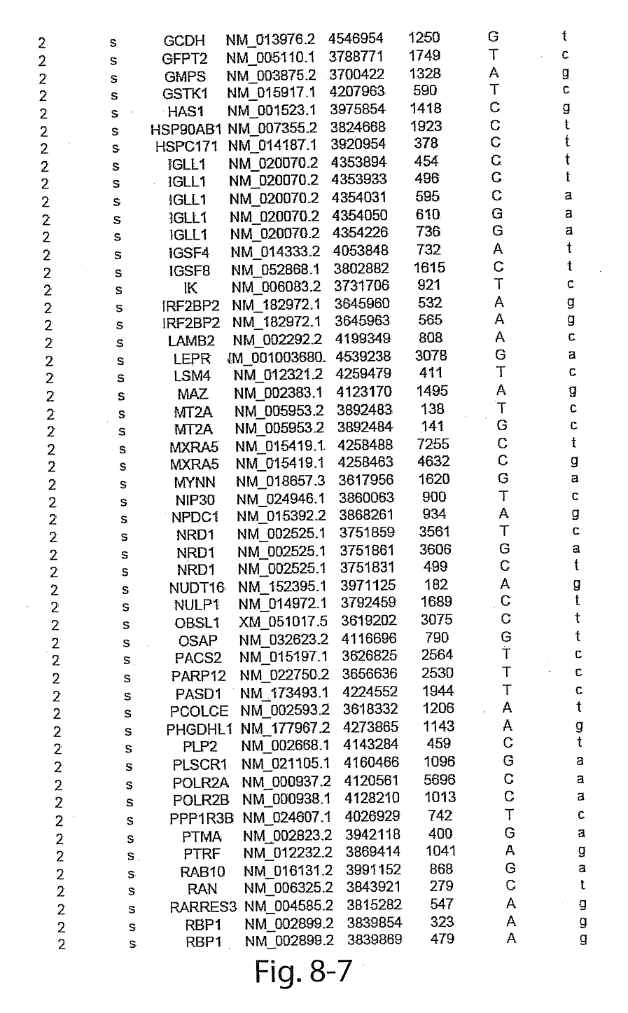

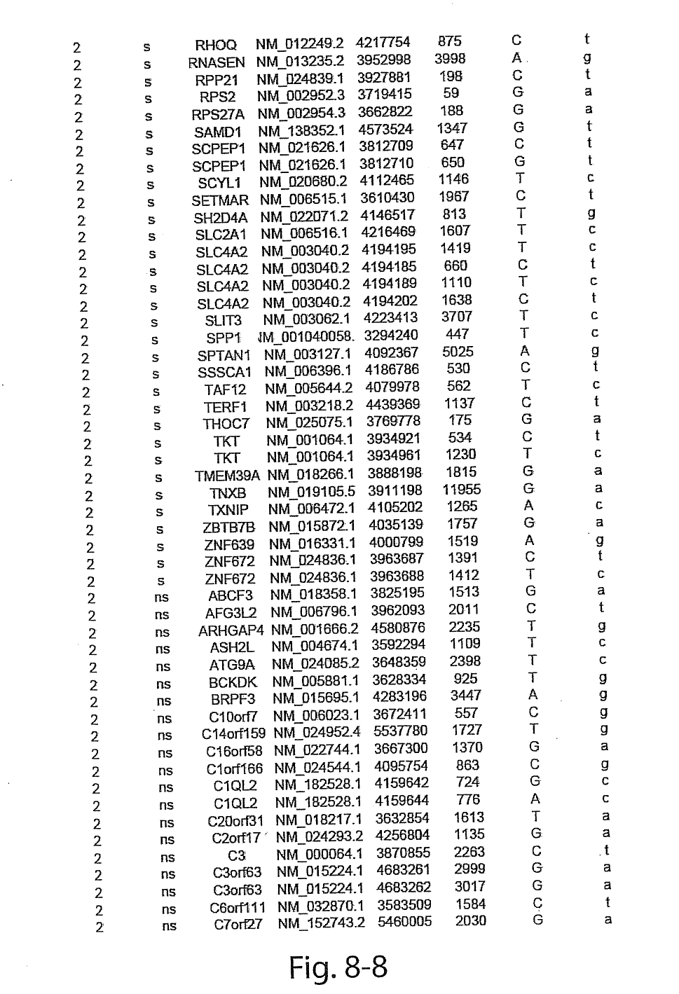

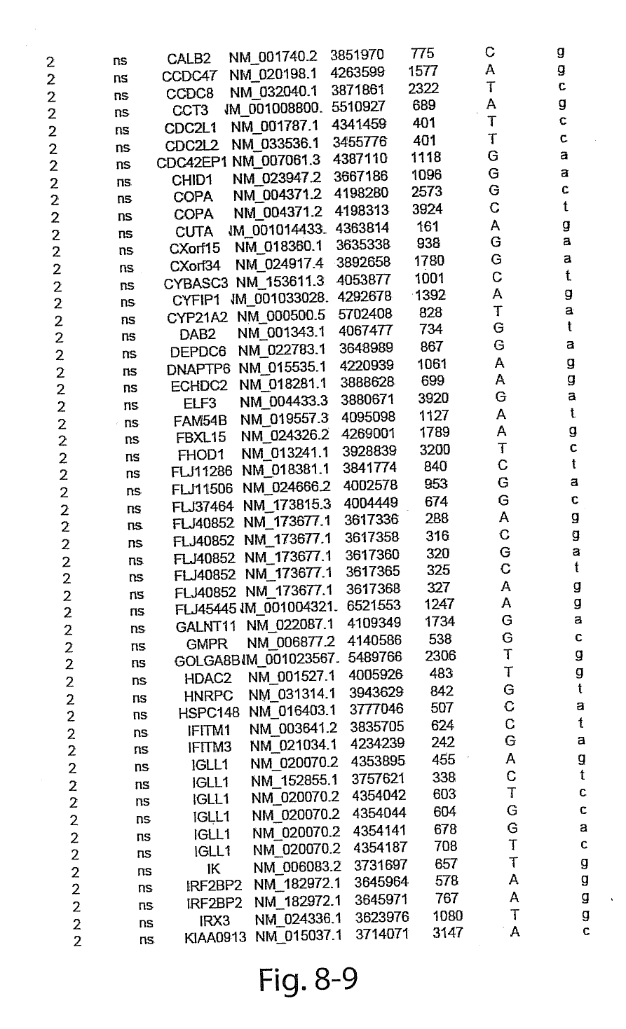

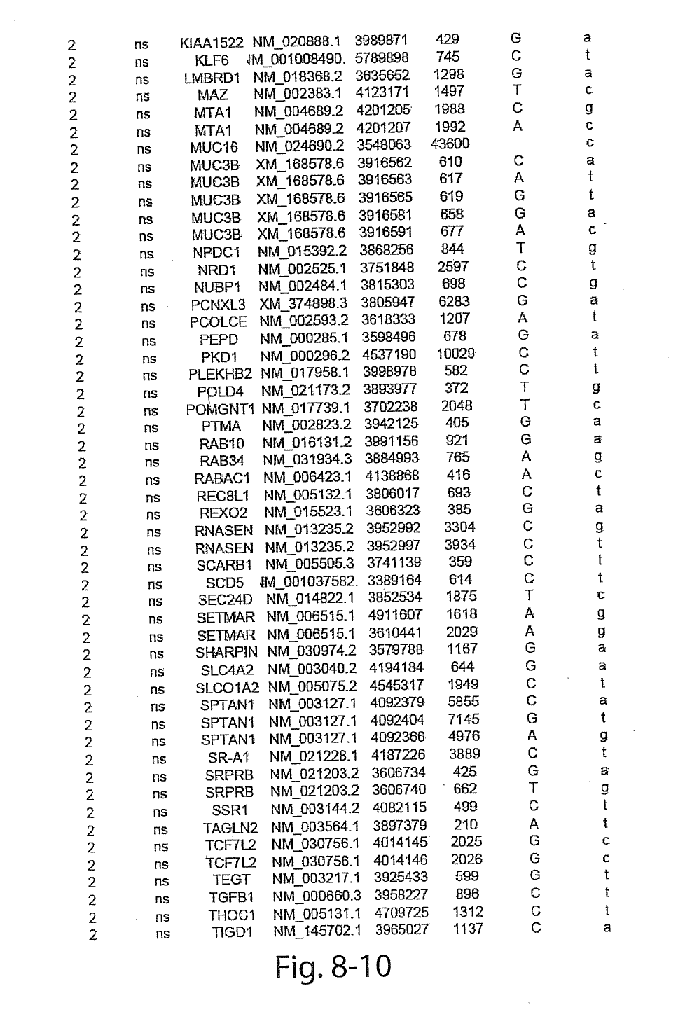

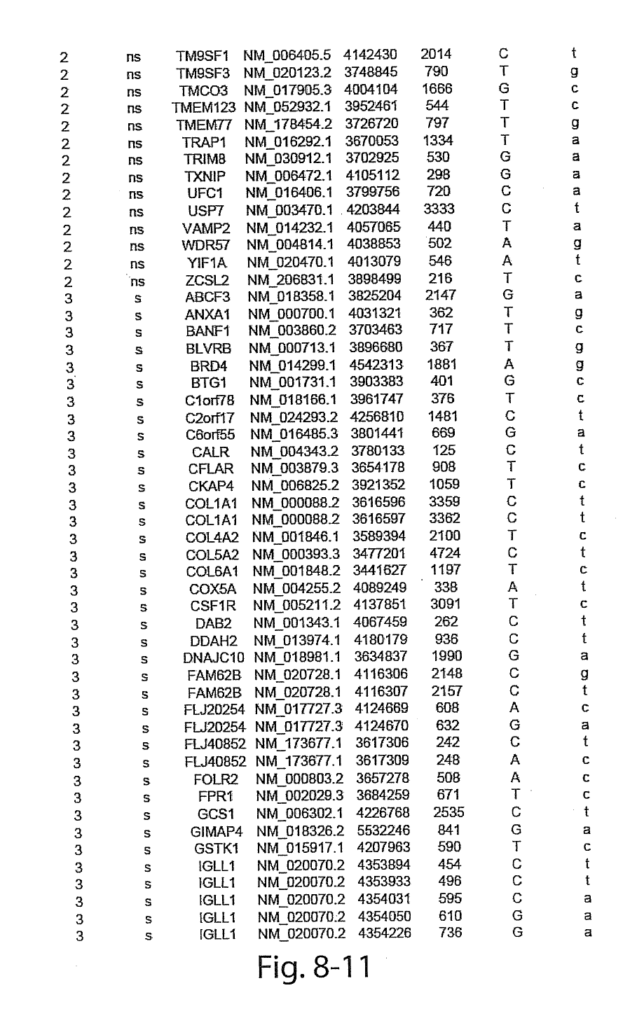

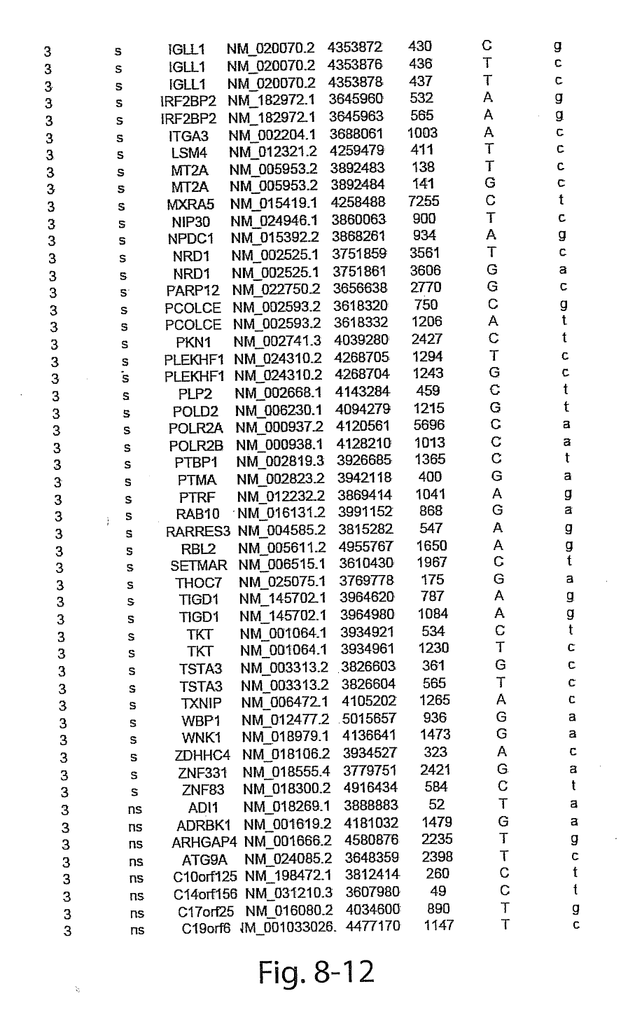

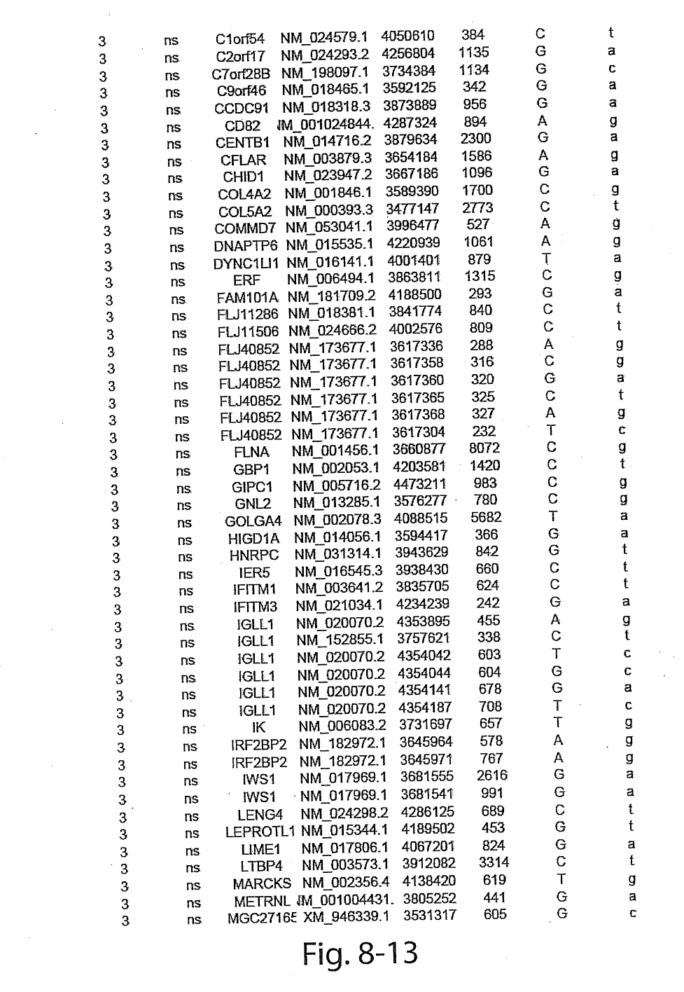

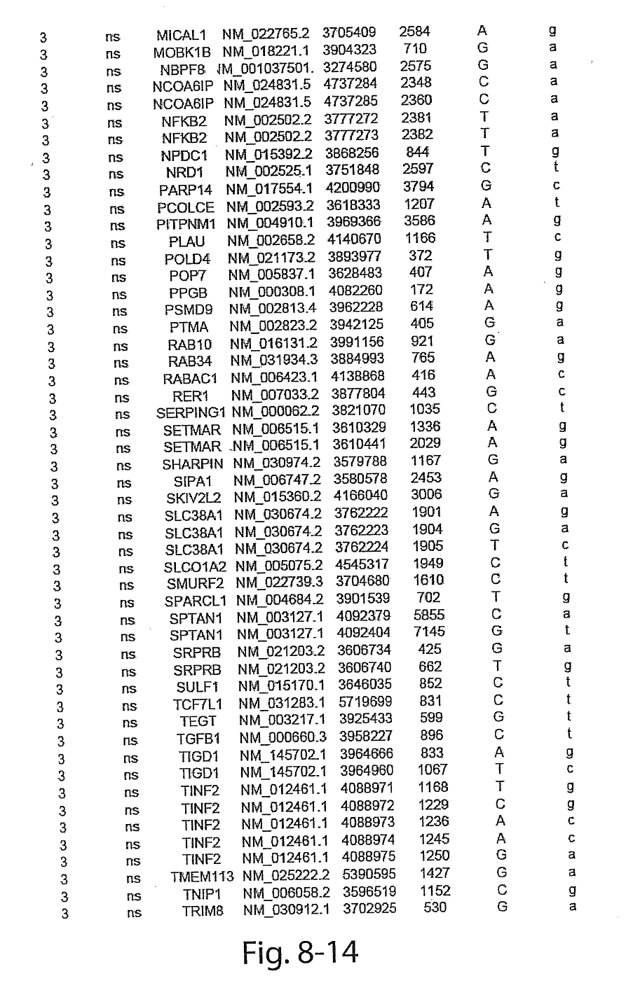

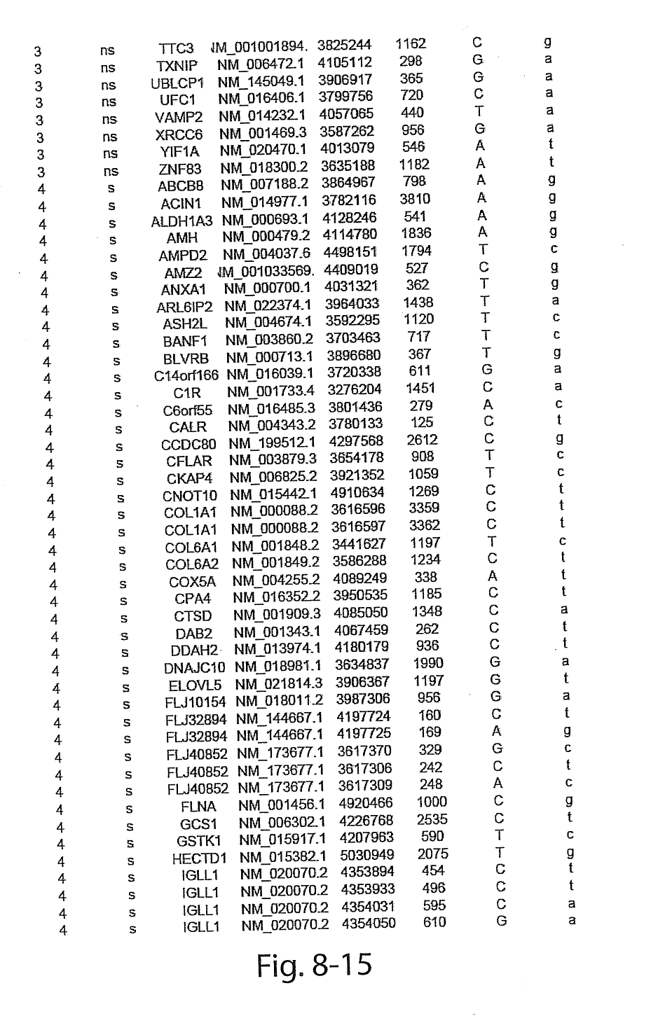

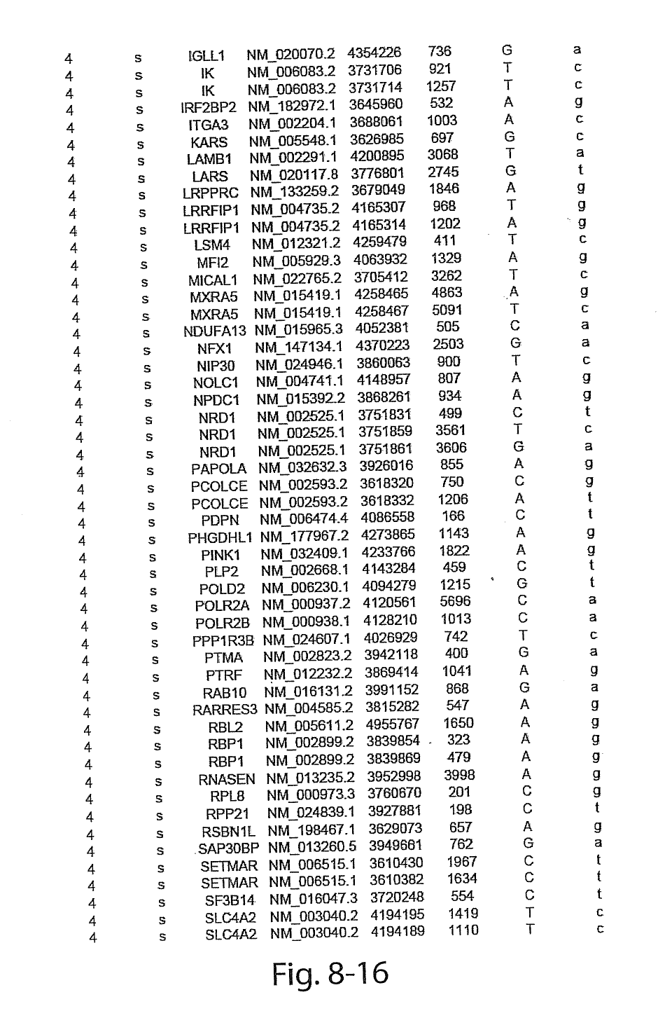

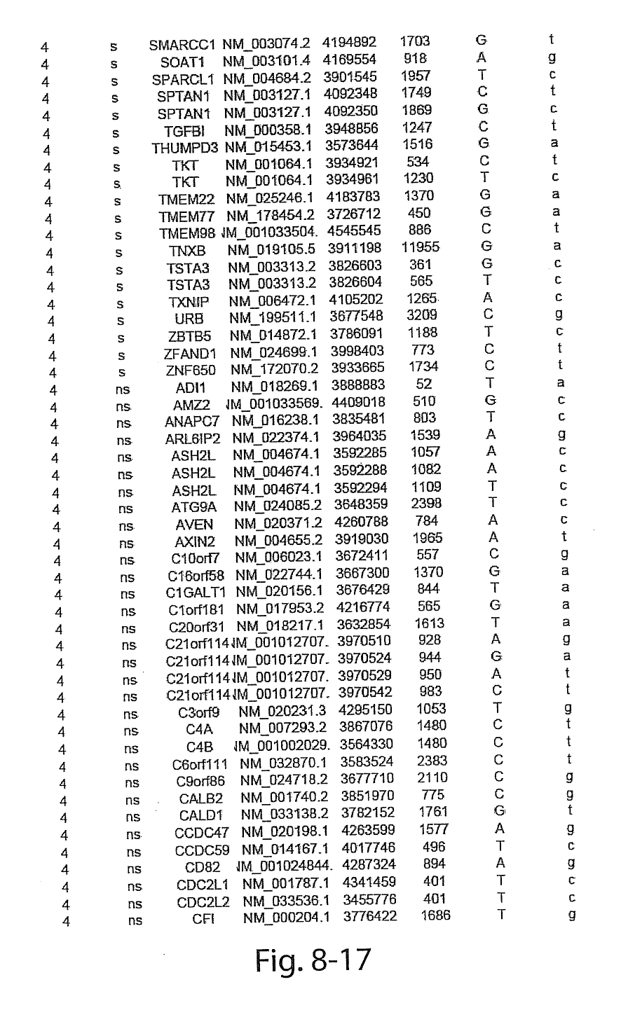

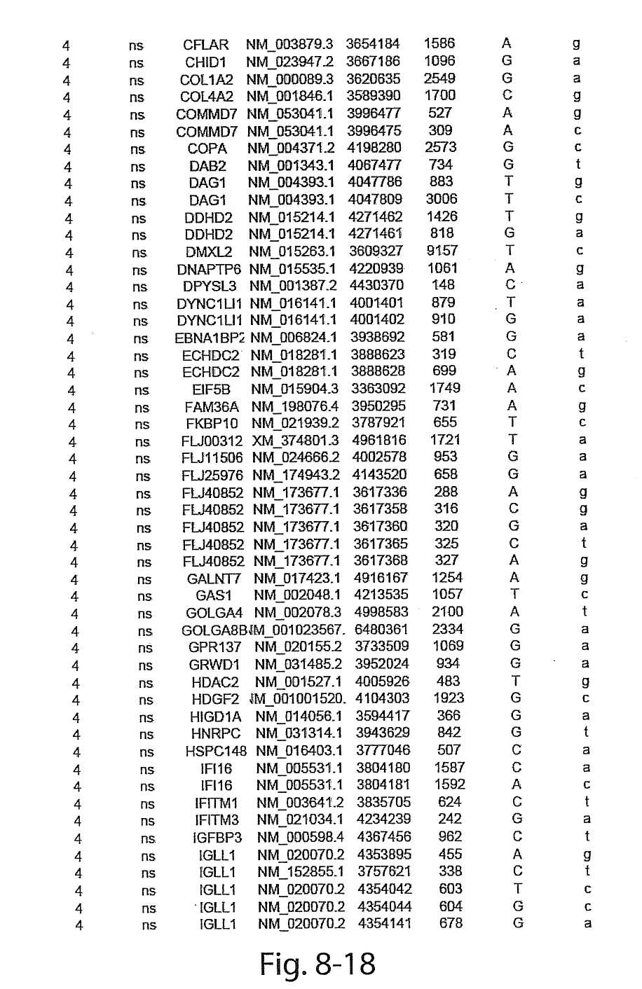

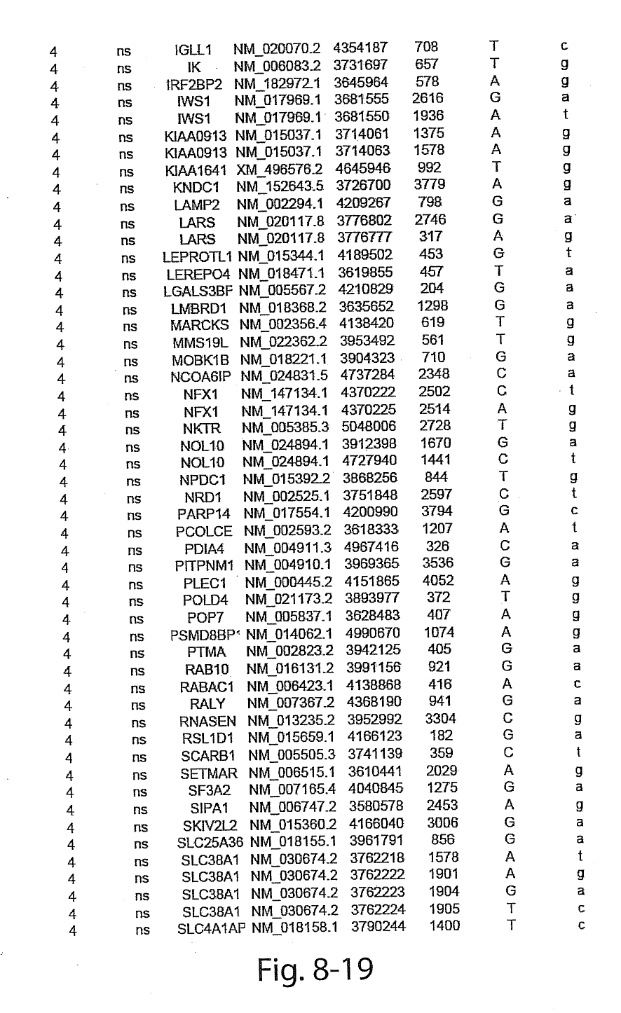

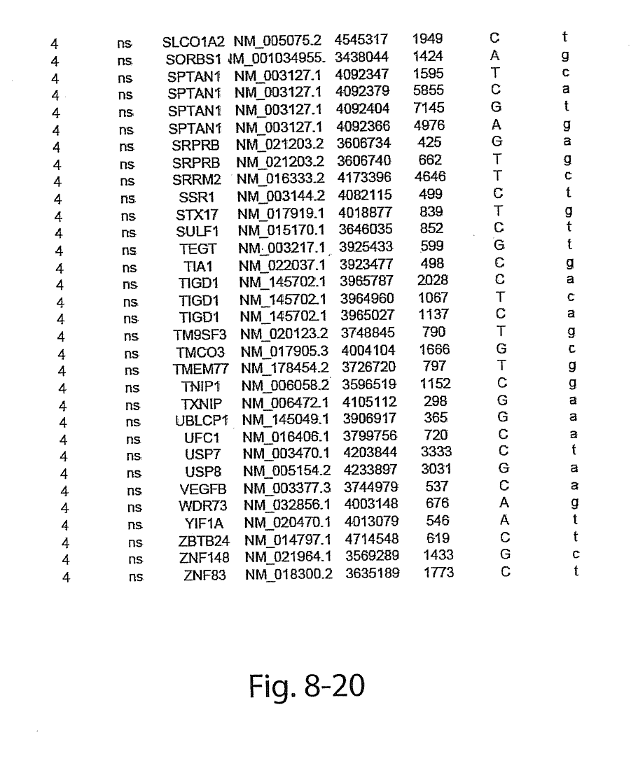

In each MPM tumor, 153-220 genes contained at least one novel SNP (FIG. 7). The total number of non-redundant novel coding region SNPs was 619 for all 4 MPM tumors combined (FIG. 8) compared to 2,369 previously known SNPs found in these samples. This relatively large number of novel coding domain SNPs likely represents both candidate mutations and novel, germ line SNPs. Of note, the ratio of nsSNPs to synonymous SNPs (sSNPs) in the four MPM tumor samples was, on average, 1.5 for novel SNPs and 0.75 for known SNPs (FIG. 7). Thus, nsSNPs, which potentially represent candidate mutations, were more prevalent among novel SNPs.

The novel nsSNPs are most likely to be functionally relevant, tumor-related mutations (17). On the basis of recent findings in breast and colorectal tumor cell lines that specific somatic point mutations are typically unique to each individual tumor,¹⁵ novel nsSNPs that were not present in normal lung or lung ADCA transcriptomes and were also unique in each MPM specimen were further investigated. Of ˜100-150 genes per specimen with at least one novel nsSNP, 67 genes (12-20 per MPM sample) were found to contain a total of 69 patient-specific novel nsSNPs (FIG. 7) following exclusion of highly polymorphic HLA and ABO loci (FIG. 9). Of note, only a single gene (GOLGA8B) contained novel nsSNPs in multiple (n=2) patients in different regions of the transcript. Sanger sequencing following PCR amplification of appropriate samples was used to determine whether these 69 novel nsSNPs represent somatic tumor mutations in MPM. All 69 variants were verified in the tumor cDNA and in tumor gDNA.

Subsequently, variants were tested for in cDNA and/or gDNA obtained from normal adjacent tissues and/or host peripheral blood lymphocytes (PBL) to differentiate between somatic mutations and germline variants. All MPM tumors contained mutations of some type, but each had a unique mutational profile (See FIG. 10). Of the 69 nsSNPs, 54 (78%) were present in the gDNA, indicating that they were polymorphisms and not mutations. These 54 nsSNPs may still have a role in predisposing MPM. The remaining 15 (22%) were found to be tumor-specific variants representing multiple types of mutations including: somatic mutations (n=7), LOH due to chromosomal deletions (n=3), epigenetic silencing (n=3) including X chromosome inactivation (n=1, likely due to clonality), and RNA editing (n=1). For the 7 nsSNPs defined above to represent LOH mutations, the variant was, in all cases, determined to be homozygous in the tumor mRNA and heterozygous in the PBL gDNA. Each variant's status in the tumor gDNA determined whether it was designated silencing (heterozygous) or deletion (homozygous). These observations highlight the diversity of mutations that exist within MPM tumors, and emphasize the power of this approach for uncovering them.

Functionally, none of these 15 genes has previously been directly implicated in tumorigenesis either in native or mutated form, but several appear to be likely candidates. Among the 7 genes affected by somatic mutations (FIG. 10), ACTR1A encodes the most abundant subunit of dynactin, a macromolecular complex that can bind to cytoplasmic dynein via physical interaction of dynamitin, another dynactin subunit. This complex, in association with other proteins, is responsible for dynein-dependent transport of p53 to the nucleus in vivo (27). Potential disruption of this complex via mutations in ACTR1A may have functional consequences on the p53 pathway in MPM providing a possible mechanism for p53 inactivation, given the absence of known inactivating mutations in p53 itself in MPM tumors.

MXRA5 and UQCRC 1 are reportedly over-expressed in colon cancer (28) and breast/ovarian cancer (29), respectively. PDZK1IP1 is over-expressed in human carcinomas of diverse origin and is associated with a tumor suppressor phenotype in cultured colon cancer cells by negatively affecting cellular proliferation and tumor growth (30-32). The PSMD13 gene encodes subunit 11 of the 26S proteasome (33), a large catalytic complex that is responsible for most non-lysosomal intracellular protein degradation and is a target of a new class of anti-cancer drugs (34). Functional mutations in this gene would be consistent with the diverse molecular and physiologic response of MPM cells to proteasome inhibition (35). COL5A2 encodes the alpha chain for one of the low abundance fibrillar collagens. Though mutations in the COL5A2 gene have previously been associated only with non-neoplastic diseases, COL5A2 is up-regulated in colon cancer (36) and normally has anti-tumor effects in breast cancer including the induction of apoptosis (37). XRCC6 (a.k.a. Ku70) forms a heterodimer with XRCC5 (a.k.a. Ku80) and mediates the repair of DNA double-strand breaks via non-homologous end-joining. The g956a mutation that was observed in the XRCC6 gene results in a V296M amino acid substitution that directly contacts Ku80 (38). While the functional significance of this mutation is unknown, it is likely to be a cancer driver mutation as predicted through computational analysis of protein domain structure (39).

One predominant genetic lesion in MPM was found to be LOH (7 of 15 lesions) due to deletions and epigenetic silencing, which is in accord with previous observations (5-11). In some cases, specific regions of LOH previously identified in MPM⁶ harbored silenced genes such as C14orf159 on chromosome 14 from the current study (FIG. 10). It is unclear whether, in addition to the allele loss demonstrated for the 7 LOH genes, the novel variant alleles have an altered cancer related function, but many of these genes have functions (FIG. 10) that could be implicated in cancer. While less widely studied compared to somatic mutations and LOH, both X chromosome inactivation (40, 41) and RNA editing (42) (i.e., a post-transcriptional process that alters the information encoded in gene transcripts, in this case a nsSNP present in the mRNA but not the gDNA) have been previously linked to cancer but not MPM. Interestingly, RNA editing has also been postulated to be responsible for at least some proportion of the SNPs deposited into dbSNP at NCBI (43).

The frequency of the seven nsSNP somatic mutations was evaluated in 49 additional MPM tumors (i.e., an independent validation set) by genotyping of cDNA and gDNA. The COL5A2 mutation (c2773t, NM_{—}000393.3) discovered in Patient 3 was found in 2 of 49 additional patients, both of who had MPM tumors with non-epithelial histology (i.e., total frequency 3/53 or ˜6%). The UQCRC1 mutation (g851a, NM_{—}003365.2) was also found in 2 of 49 additional MPM patients (−6% of patients overall), and the MXRA5 mutation (c7862a, NM_{—}015419.1) was found in 1 of 49 additional MPM patients (−4% of patients overall). No patient in the validation set was found to have more than one of these mutations. Thus, despite being relatively uncommon, at least 3 of these mutations are actually not unique when a larger cohort of MPM tumors is surveyed, further implicating them as likely driver mutations in MPM tumorigenesis.

A total of seven somatic mutations were identified in four cases, representing ˜1.75 nsSNP somatic mutation per 4,000 expressed genes with ˜4-5× coverage. By extrapolation, it is estimated that MPM tumors harbor, on average, ˜6 somatic mutations in transcribed sequences, or approximately 10-14 non-synonymous point mutations in coding sequences of the entire genome which is in accord with a recent survey of cancers (17). The approach described herein of selecting nsSNPs uniquely present in each MPM specimen resulted in a relatively enriched pool of validated mutations, compared to similar discovery efforts involving gDNA exon resequencing. Of 69 candidate nsSNP mutations, 15 were validated as mutations, including 7 of 69 somatic mutations. In addition, at least 3 of the 7 somatic mutations (42%) are likely to be driver mutations based upon cancer-relevant functions and their presence in at least one other MPM tumor. This frequency of putative driver mutations among all nsSNPs exceeds that posited by Greenman, et al. (17) in a survey of exons from tumor gDNA. Thus, approaches based upon transcriptome sequencing have a greater likelihood of identifying driver mutations, and evidence for them being driver mutations can be further strengthened by their occurrence, albeit relatively rarely, in additional tumors, as seen in MPM.

In addition to these 69 case-specific nsSNPs, 12 additional candidate nsSNP mutations were identified that were common to all five tumor samples, but absent from the normal lung, and 4 were common to the four MPM tumors only. These were all validated as germline SNPs; none was found to be a mutation further supporting recent observations that most somatic mutations are specific to individual tumors, at least in small tumor sets (15, 17). Virtually all tumor-related SNPs (i.e., known or unknown, nsSNP or sSNP) identified in this study remain candidate genes for determining predisposition to developing MPM. Of note, 275 non-redundant novel sSNPs in all MPMs combined were identified which might be considered less likely to be associated with functional consequences (15, 17). However, a recent study showed that sSNP mutations can be primary driver mutations or environmentally sensitive predisposing variations for disease development by virtue of changes in the secondary structures of the mRNA (44).

### Discussion

The above Examples together demonstrate that transcriptome sequencing of patient tumors can result in discovery of previously uncharacterized human cancer mutations. By using an integrated approach that includes specimen enrichment for tumor cells, pyrosequencing, and rule-driven informatics, rare mutations were discovered among thousands of expressed genes. In addition to the advantages of speed and cost, this approach enriches for mutations in expressed genes and identifies multiple classes of mutations. The use of tumor tissue avoids artifactual mutations generated in cell culture. In addition, transcriptome sequencing provides information about mRNA expression levels not available with exon resequencing

The identification of multiple types of genetic variants contributes to an expanded understanding of MPM and demonstrates that such an approach is essential to discovering the full complement of genomic changes associated with tumorigenesis. The four MPM patients had unique mutational profiles. Patient 1 had five somatic mutations and one LOH mutation, whereas Patient 2 had LOH mutations due to deletions in chromosome 14 and an X inactivation mutation that may have been a clonal event. Patient 3 had two somatic mutations only, one of which was present in two other patients' tumors. Patient 4 had three LOH mutations due to silencing and one due to RNA editing. This diversity of mutations emphasizes that defining correlations between tumor genotypes, histology, and various risk factors, such as, asbestos exposure, will require sequencing a much larger cohort of MPM patients. Of 15 mutations, seven were somatic point mutations, representing ≈1.75 ns somatic mutations per ≈3,800 Known RefSeq Genes sequenced with 4-5× coverage. By extrapolation to the ≈10,000 expressed Known RefSeq Genes detected in MPM transcriptomes (FIG. 2) it is estimated that individual MPM tumors harbor, on average, 6 transcribed genes with somatic mutations, or ≈10-14 genes with a nsSNV in the entire genome, in accord with a recent exon-resequencing survey of cancers. At this depth of sequencing, ≈138% of the expressed genes could be exhaustively analyzed for mutations. Additional sequencing and better characterization of the LOC transcripts will be necessary to characterize the full mutational spectrum of each tumor.

For the 15 mutated genes observed in MPM, this study provides the evidence that they can be mutated in cancer, in keeping with recent mutational surveys that also uncovered many previously uncharacterized mutated genes in other tumor types, and thus, useful as cancer markers. Mutated genes often exhibit abnormal levels of expression, and a retrospective analysis of published MPM profiling data revealed that most of these 15 mutated genes are over-expressed in a majority of MPM tumors. A literature survey of gene function and expression in tumor cells reveals that the seven genes affected by somatic mutations (FIG. 10) are plausibly related to oncogenesis. Of particular note, the protein product of XRCC6 (Ku70) forms a heterodimer with that of XRCC5 (Ku80) and mediates the repair of DNA double-strand breaks via nonhomologous end-joining. In non-small-cell lung cancer XRCC5 is often hypermethylated and underexpressed at the mRNA and protein levels and is generally correlated with p53 changes. The g956a mutation that was observed in the XRCC6 gene results in a V296M amino acid substitution in a protein region that directly contacts Ku80. This specific amino acid substitution could be a cancer-driver mutation based on computational analysis of protein domain structure.

ACTR1A encodes the most abundant subunit of dynactin, which is associated with transport of p53 to the nucleus. Disruption of this complex via mutations in ACTR1A could potentially result in p53 inactivation, which is intriguing, given the absence of known inactivating mutations in p53 in MPM tumors. PDZK1IP1 is overexpressed in human carcinomas of diverse origin and exhibits a tumor-suppressor phenotype in cultured colon cancer cells by negatively affecting proliferation and tumor growth.

COL5A2 encodes the α-chain for a low-abundance fibrillar collagen that is up-regulated in colon cancer and normally has antitumor effects in breast cancer, including the induction of apoptosis UQCRC1 is a component of the mitochondrial ubiquinol-cytochrome-c reductase complex that was mutated in three MPM patients. It is known to be overexpressed in breast and ovarian cancer and has been suggested to play a role in tumorigenesis. The PSMD13 gene encodes subunit 11 of the 26S proteasome, which is the target of a new class of anticancer drugs. Although functionally uncharacterized, MXRA5 is overexpressed in colon cancer.

In addition to somatic mutations, gene deletions, gene silencing, and RNA editing were identified as common lesions in the MPM tumors. Among these, LOH mutations were observed in three genes situated on chromosome 14 in Patient 2. This genomic region was previously implicated in MPM tumors, and a notable gene within this region is AVEN. The genetic lesion identified in AVEN is of potential interest because this gene impairs Apaf-1-mediated activation of caspases, and thus apoptosis. The present inventors and others have previously identified other (non-Bcl-2) antiapoptotic survival pathways as being particularly important in MPM tumorigenesis and drug resistance, and AVEN was recently implicated in acute leukemias. Elucidating the functional relevance of the previously uncharacterized variant alleles rendered homozygous by LOH in MPM (FIG. 10) is a promising avenue for further exploration.

Although less well studied than somatic mutations and LOH, both X chromosome inactivation and RNA editing (i.e., a posttranscriptional process that alters the information encoded in gene transcripts, in this case a nsSNV present in the mRNA but not the gDNA) have been previously linked to cancer but not MPM. The actual editing is site-specific and occurs through specific mechanisms that are thought to be altered in cancers. Interestingly, RNA editing has also been postulated to be responsible for at least some proportion of the SNPs deposited into dbSNP at NCBI.

Transcriptome pyrosequencing permits comprehensive, unbiased, mutational analysis of expressed genes. This technique can also provide additional genetic information, such as insertion and deletion (indel) variant identification, read-count-based gene expression profiling, SNP allele frequencies, haplotype frequencies, novel isoform identification, and relative isoform abundance. In addition, transcriptome sequencing yields a more comprehensive set of gene-tagging SNPs that will be of considerable utility in disease-association studies.

These Examples confirm the accuracy of pyrosequencing for 94 of 94 previously uncharacterized variants when the empirically derived filtering rules for variation discovery were used and suggested that this overall approach could become a standard for discovery and validation of genetic variants and tumor mutations. Solid tumors represent a major cause of morbidity and mortality in developed nations. Therapies for advanced cancer are limited because of the genetic complexity and variability among tumors. Large-scale pyrosequencing of the tumor transcriptome may be useful for determination of patient-specific mutational profiles enabling discoveries that have the potential to impact individual patient care. This approach may ultimately form the basis of molecular subtyping of patients with cancer, allowing combinational multitherapy designed individually for each patient based on mutational profile. Further discussion of these Examples can be found in Sugarbaker et al., “Transcriptome sequencing of malignant pleural mesothelioma tumors,” PNAS, Mar. 4, 2008, Vol. 105, No. 9, pp 3521-3526, the entire contents of which are hereby incorporated by reference.

Although the foregoing invention has been described in some detail by way of illustration and example for purposes of clarity and understanding, it will be apparent to those skilled in the art that certain changes and modifications can be practiced. Therefore, the description and examples should not be construed as limiting the scope of the invention, which is delineated by the appended numbered claims.

### REFERENCES

1. Britton, M. The Epidemiology of Mesothelioma. Semin Surg Oncol 29, 18-25 (2002).
2. Chang, M. Y. & Sugarbaker, D. J. Extrapleural pneumonectomy for diffuse malignant pleural mesothelioma: Techniques and complications. Thoracic Surg Clin 14, 523-530 (2004).
3. Ordonez, N. G. The immunohistochemical diagnosis of mesothelioma: a comparative study of epithelioid mesothelioma and lung adenocarcinoma. Am J Surg Pathol 27, 1031-1051 (2003).
4. http://www.cancer.org
5. Balsara, B. R. et al. Comparative genomic hybridization and loss of heterozygosity analyses identify a common region of deletion at 15q11.1-15 in human malignant mesothelioma. Cancer Res 59, 450-454 (1999).
6. De Rienzo, A., Jhanwar, S. C. & Testa, J. R. Loss of heterozygosity analysis of 13q and 14q in human malignant mesothelioma. Genes Chromosomes and Cancer 28, 337-341 (2000).
7. Huncharek, M. Genetic factors in the aetiology of malignant mesothelioma. Eur J Cancer 31A, 1741-1747 (1995).
8. Lee, W. C. & Testa, J. R. Somatic genetic alterations in human malignant mesothelioma. Int J Oncol 14, 181-188 (1999).
9. Musti, M. et al. Cytogenetic and molecular genetic changes in malignant mesothelioma. Cancer Genet Cytogenet 170, 9-15 (2006).
10. Pylkkanen, L. et al. Concurrent LOH at multiple loci in human malignant mesothelioma with preferential loss of NF2 gene region. Oncol Rep 9, 955-959 (2002).
11. Whitson, B. A. & Kratzke, R. A. Molecular pathways in malignant pleural mesothelioma. Cancer Lett 239, 183-189 (2006).
12. Gordon, G. J. et al. Translation of microarray data into clinically relevant cancer diagnostic tests using gene expression ratios in lung cancer and mesothelioma. Cancer Res 62, 4963-4967 (2002).
13. Gordon, G. J. et al. Identification of novel candidate oncogenes and tumor suppressors in malignant pleural mesothelioma using large-scale transcriptional profiling. Am J Pathol 166, 1827-1840 (2005).
14. Futreal, P. A. et al. A census of human cancer genes. Nat Rev Cancer 4, 177-183 (2004).
15. Sjoblom, T. et al. The consensus coding sequences of human breast and colorectal cancers. Science 314, 268-274 (2006).
16. Futreal, P. A., Wooster, R. & Stratton, M. R. Somatic mutations in human cancer: insights from resequencing the protein kinase gene family. Cold Spring Harb Symp Quant Biol 70, 43-49 (2006).
17. Greenman, C. et al. Patterns of somatic mutation in human cancer genomes. Nature 446, 153-158 (2007).
18. Haber, D. A. & Settleman, J. Cancer: drivers and passengers. Nature 446, 145-456 (2007).
19. Green, R. E. et al. Analysis of one million base pairs of Neanderthal DNA. Nature 444, 330-336 (2006).
20. Margulies, M. et al. Genome sequencing in microfabricated high-density picolitre reactors. Nature 437, 376-380 (2005).
21. http://www.ncbi.nlm.nih.gov/entrez/query/static/help/genefaq.html#faq_g4
22. http://www.ncbi.nlm.nih.gov/IEB/Research/AceMbly
23. Jongeneel, C. V. et al. An atlas of human gene expression from massively parallel signature sequencing (MPSS). 15, 1007-1014 (2005).
24. Mills, R. E. et al. An initial map of insertion and deletion (INDEL) variation in the human genome. Genome Res 16, 1182-1190 (2006).
25. Benz, C. C. et al. Altered promoter usage characterizes monoallelic transcription arising with ERBB2 amplification in human breast cancers. Genes Chromosomes Cancer 45, 983-994 (2006).
26. Redon, R. et al. Global variation in copy number in the human genome. 444, 444-454 (2006).
27. Galigniana, M. D., Harrell, J. M., O'Hagen, H. M., Ljungman, M. & Pratt, W. B. Hsp90-binding immunophilins link p53 to dynein during p53 transport to the nucleus. J Biol Chem 279, 22483-22489 (2004).
28. Zou, T. T. et al. Application of cDNA microarrays to generate a molecular taxonomy capable of distinguishing between colon cancer and normal colon. Oncogene 21, 4855-4862 (2002).
29. Kulawiec, M. et al. Proteomic analysis of mitochondria-to-nucleus retrograde response in human cancer. Cancer Biol Ther 5, 967-975 (2006).
30. Kocher, O., Cheresh, P., Brown, L. F. & Lee, S. W. Identification of a novel gene, selectively up-regulated in human carcinomas, using the differential display technique. Clin Cancer Res 1, 1209-1215 (1995).
31. Kocher, 0., Cheresh, P. & Lee, S. W. Identification and partial characterization of a novel membrane-associated protein (MAP17) up-regulated in human carcinomas and modulating cell replication and tumor growth. Am J Pathol 149, 493-500 (1996).
32. Kocher, O. et al. PDZK1, a novel PDZ domain-containing protein up-regulated in carcinomas and mapped to chromosome 1q21, interacts with cMOAT (MRP2), the multidrug resistance-associated protein. Lab Invest 79, 1161-1170 (1999).
33. Hoffman, L., Gorbea, C. & Rechsteiner, M. Identification, molecular cloning, and characterization of subunit 11 of the human 26S proteasome. FEBS Lett 449, 88-92 (1999).
34. Adams, J. The proteosome: A suitable antineoplastic target. Nat Rev Cancer 4, 349-360 (2004).
35. Gordon, G. J. et al. Preclinical studies of the proteasome inhibitor bortezomib in malignant pleural mesothelioma. Cancer Chemother Pharmacol In Press (2007).
36. Fischer, H., Stenling, R., Rubio, C. & Lindblom, A. Colorectal carcinogenesis is associated with stromal expression of COL11A1 and COL5A2. Carcinogenesis 22, 875-878 (2001).
37. Luparello, C. & Sirchia, R. Type V collagen regulates the expression of apoptotic and stress response genes by breast cancer cells. J Cell Physiol 202, 411-421 (2005).
38. Walker, J. R., Corpina, R. A. & Goldberg, J. Structure of the Ku heterodimer bound to DNA and its implications for double-strand break repair. Nature 412, 607-614 (2001).
39. Kaminker, J. S. et al. Distinguishing cancer-associated missense mutations from common polymorphisms. Cancer Res 67, 465-473 (2007).
40. Kristiansen, M. et al. High incidence of skewed X chromosome inactivation in young patients with familial non-BRCA1/BRCA2 breast cancer. J Med Genet. 42, 877-880 (2005).
41. Li, G. et al. Skewed X chromosome inactivation of blood cells is associated with early development of lung cancer in females. Oncol Rep 16, 859-864 (2006).
42. Scholzova, E., Malik, R., Sevcik, J. & Kleibl, Z. RNA regulation and cancer development. Cancer Lett 246, 12-23 (2007).
43. Eisenberg, E. et al. Identification of RNA editing sites in the SNP database. Nucleic Acids Res 33, 4612-4617 (2005).
44. Kimchi-Sarfaty, C. et al. A “silent” polymorphism in the MDR1 gene changes substrate specificity. Science 315, 525-528 (2007).
45. Richards, W. G. et al. Microaliquoting: A technique for precision histologic annotation and cell content optimization of frozen tissue specimens. Biotech Histochem In Press (2007).
46. http://www.ncbi.nlm.nih.gov/RefSeq
47. http://www.ncbi.nlm.nih.gov/AceView
48. Gramza et. al. “Efficient Method for Preparing Normal and Tumor Tissue for RNA Extraction” BioTechniques, volume 18, page 218 (1995) 49. Margulies et al., “Genome sequencing in microfabricated high-density picolitre reactors,” Nature, 437:326-7 (2005)
50. Ng et al., “Multiplex sequencing of paired-end ditags (MS-PET): a strategy for the ultra-high-throughput analysis of transcriptomes and genomes,” Nucleic Acids Research, 34: published 2006.
51. Pinard et al., “Assessment of whole genome amplification-induced bias through high-throughput, massively parallel whole genome sequencing,” BMC Genomics, 7:216 (2006)
52. Leamon et al., “High-throughput, massively parallel DNA sequencing technology for the era of personalized medicine,” 3:15-31 (2007)
53. Trombetti et al., “Data handling strategies for high throughput pyrosequencers,” BMC Bioinformatics, 8:S22 (2007)
54. Sugarbaker et al., “Transcriptome sequencing of malignant pleural mesothelioma tumors,” PNAS, Mar. 4, 2008, Vol. 105, No. 9, pp 3521-3526.

We claim:

1. An isolated nucleic acid molecule comprising a nucleotide sequence selected from the group consisting of SEQ ID NOS: 1, 3, 5, 7, 9, 11, 13, 15, 17, 19, 21, 23, 25, 27, 29 and a homolog thereof, wherein the nucleotide sequences each further comprise at least one genetic variation which predisposes a person to malignant pleural mesothelioma.

2. The isolated nucleic acid molecule of claim 1, wherein the at least one genetic variation is a single nucleotide polymorphism.

3. The isolated nucleic acid molecule of claim 1, wherein the at least one genetic variation is a single nucleotide polymorphism, a somatic mutation, an inversion, a deletion, an insertion, or an LOH mutation.

4. The isolated nucleic acid molecule of claim 3, wherein the LOH mutation is due to a deletion, epigenetic silencing, X inactivation, or RNA editing.

5. The isolated nucleic acid molecule of claim 1, wherein the homolog has at least about 85% sequence identity.

6. The isolated nucleic acid molecule of claim 1, wherein the homolog has at least about 90% sequence identity.

7. The isolated nucleic acid molecule of claim 1, wherein the homolog has at least about 95% sequence identity.

8. An isolated nucleic acid molecule comprising a nucleotide sequence selected from the group consisting of SEQ ID NOS: 2, 4, 6, 8, 10, 12, 14, 16, 18, 20, 22, 24, 26, 28 and 30, wherein each nucleotide sequence comprises a genetic variation which predisposes a person to malignant pleural mesothelioma.

9. An expression vector comprising a nucleic acid molecule of claim 1, wherein the nucleotide sequence is operably linked to a promoter.

10. A host cell comprising the expression vector of claim 9.

11. A method for detecting disease-associated genetic variations in a sample, comprising the steps of: (a) obtaining the nucleic acids from the sample; (b) pyrosequencing the nucleic acids to generate a sequence data set; (c) analyzing the sequence data set using parameters capable of identifying candidate genetic variations; (d) validating the candidate genetic variations to identify the disease-associated variations, wherein the parameters comprise one or more of the following criteria: a genetic variation must (1) be present in at least 4 reads, (2) be present in at least 30% of the total reads covering genetic variation, and (3) be present in a read that is at least 90% identical to a reference sequence.

12. The method of claim 11, wherein the genetic variations are selected from the group consisting of: single nucleotide polymorphisms, loss of heterozygosity mutations, inversions, deletions and insertions.

13. The method of claim 12, wherein the loss of heterozygosity mutations are due to a deletion, epigenetic silencing, or X inactivation.

14. The method of claim 11, wherein the diseased specimen is cancer.

15. The method of claim 14, wherein the cancer is selected from the group consisting of: malignant pleural mesothelioma, leukemia, brain cancer, prostate cancer, liver cancer, ovarian cancer, stomach cancer, colorectal cancer, throat cancer, breast cancer, skin cancer, melanoma, lung cancer, lung adenocarcinoma, sarcoma, cervical cancer, testicular cancer, bladder cancer, endocrine cancer, endometrial cancer, esophageal cancer, glioma, lymphoma, neuroblastoma, osteosarcoma, pancreatic cancer, pituitary cancer, and renal cancer.

16. The method of claim 11, wherein the sequence data set comprises 4-5× gene coverage.

17. The method of claim 11, wherein the pyrosequencing is carried out by a GS20 pyrosequencer.

18. The method of claim 11, wherein the parameters further comprise the criteria that the genetic variation must have a GS20 quality score of at least 20.

19. The method of claim 11, wherein the parameters further comprise the criteria that the genetic variation must be observed in both orientations.

20. The method of claim 11, wherein the validating step is achieved by re-sequencing the genetic variation in the specimen by Sanger sequencing.
